(19) United States
(12) Patent Application Publication
(10) Pub. No.: US 2024/0173367 A1
(43) Pub. Date: May 30, 2024

(54) COMPOSITIONS AND METHODS FOR TREATING CHOLESTATIC DISEASE

(71) Applicant: Seres Therapeutics, Inc., Cambridge, MA (US)

(72) Inventors: Madhumitha NANDAKUMAR, Arlington, MA (US); Alice Peiyu LIOU, Somerville, MA (US); Elizabeth Moritz HALVORSEN, Malden, MA (US); Gregory MCKENZIE, Arlington, MA (US); Edward J. O'BRIEN, Arlington, MA (US); David COOK, Brookline, MA (US)

(73) Assignee: Seres Therapeutics, Inc., Cambridge, MA (US)

(21) Appl. No.: 18/319,086

(22) Filed: May 17, 2023

Related U.S. Application Data
(63) Continuation of application No. 16/639,492, filed on Feb. 14, 2020, now Pat. No. 11,701,394.
(60) Provisional application No. 62/545,298, filed on Aug. 14, 2017.

(51) Int. Cl. A61K 35/745; A61K 31/58; A61P 1/16

(52) U.S. Cl. CPC A61K 35/745; A61K 31/58; A61P 1/16

(57) ABSTRACT

The invention provides compositions and methods for use in the treatment and prevention of cholestatic diseases.

### CROSS REFERENCE TO RELATED APPLICATIONS

This application is a continuation of U.S. patent application Ser. No. 16/639,492, 371(c) Date of Feb. 14, 2020 (issued as U.S. Pat. No. 11,701,394), which is the National Stage of International Application No. PCT/US2018/046769, filed Aug. 14, 2018, which claims the benefit of U.S. Provisional Application No. 62/545,298, filed Aug. 14, 2017, each of which is incorporated herein by reference in its entirety.

### REFERENCE TO A SEQUENCE LISTING SUBMITTED ELECTRONICALLY

The content of the electronically submitted sequence listing in ASCII text file (Name: 4268_0430002_SequenceListing_ST26; Size: 909,720 bytes; and Date of Creation: Dec. 5, 2023) filed with the application is herein incorporated by reference in its entirety.

### FIELD OF THE INVENTION

This invention relates to compositions and methods for use in the treatment and prevention of cholestatic diseases.

### BACKGROUND

Cholestatic diseases are conditions that result in a toxic accumulation of bile in the liver, and impaired liver function as evidenced by elevated levels of liver enzymes in serum. This can be caused through direct blockage of extrahepatic bile ducts (e.g., by gallstone, inflammatory stricture, cancer, or pancreatitis) or damage to intrahepatic bile ducts caused by conditions such as primary biliary cirrhosis (PBC), primary sclerosing cirrhosis (PSC), progressive familial intrahepatic cholestasis, cholestasis of pregnancy, cholangitis, liver diseases, such as hepatitis, alcoholic liver disease, non-alcoholic steatohepatitis (NASH), and hepatocellular carcinoma, and other causes of liver cirrhosis, such as cystic fibrosis and graft vs. host disease.

Ursodeoxycholic acid (UDCA) has been reported as a treatment for cholestatic liver diseases such as PBC and PSC. UDCA is believed to delay disease progression through its anti-cholestatic, anti-inflammatory, anti-apoptotic, and protective properties (Paumgartner et al., Hepatology 36:525-531, 2002). However, for PBC, about 40% of patients fail to respond to UDCA treatment (Pares et al., Gastroenterology 130:715-720, 2006). Furthermore, the treatment can have severe side effects, particularly when administered at high dosages. In PSC, high doses were associated with risk of significant adverse events. Obeticholic acid, a non-natural bile acid derivative, has been approved for treating primary biliary cholangitis (PBC) in combination with UDCA in adults with an inadequate response to UDCA, or as monotherapy in adults unable to tolerate UDCA, and is being studied in clinical trials for treating PSC. However, the treatment can be associated with undesirable side effects including severe pruritus. Patients whose disease is not controlled by pharmacologic intervention often require liver transplantation.

The gastrointestinal (GI) microbiome plays a role in bile acid metabolism, altering liver synthesized conjugated primary bile acids to a range of primary and secondary bile acids that influence metabolism, inflammation, immunity, and bile acid synthesis in the liver and the gastrointestinal (GI) tract. Liver synthesized bile salts include glycine or taurine conjugated cholic acid (CA) and chenodeoxycholic acid (CDCA), which are amphipathic and have detergent properties that help solubilize lipids and other hydrophobic molecules for absorption (Ridlon et al., J. Lipid Res. 47:247-259, 2006). The conjugated primary bile salts (optionally referred to herein as “conjugated primary bile acids”) are deconjugated by certain gut bacteria to form deconjugated primary bile acids (referred to herein as “primary bile acids”) that can be further metabolized into secondary bile acids through a series of microbially catalyzed reactions, including oxidation, isomerization, and 7α-dehydroxylation (Ridlon et al., J. Lipid Res. 47:247-259, 2006). In addition, the liver produces conjugated forms of these bile acid metabolites (referred to herein as “conjugated secondary bile acids”). In total, more than 45 species of bile acids have been reported to be found in humans (Bathena et al., J. Chromatography B 942-943:53-62, 2013). Changes in bile acid content and signaling have been associated with a number of disease outcomes including cholestatic disease, NASH, and inflammatory diseases such as inflammatory bowel disease (Hofmann, Arch Intern Med 159:2647-2658, 1999; Duboc et al., Gut 63:531-539, 2013; Kohli et al., Dig. Dis. 33:440-446, 2015).

While conjugated primary bile salts are important for proper nutrient absorption, when present at high concentrations or when unable to form micelles, they can cause damage to hepatocytes and biliary epithelial cells (Monte et al., World J. Gastroenterol. 15(7):804-816, 2009). Elevated levels of bile acids have also been shown to cause oxidative stress and apoptosis in the liver (Sokol et al. Hepatology 17:869-881, 1993; Faubion et al., Fas. J. Clin. Invest. 103:137-145, 1999) and the more hydrophobic bile acids have been associated with carcinogenesis in the colon (Debruyne et al., Mutat. Res. 480-481:359-369, 2001). Disrupted FXR signaling and bile acid content has also been associated with liver cancer (Kim et al., Carcinogenesis 28:940-946, 2007). In humans, a defect in the MDR3 gene results in a type of cholestasis known as Progressive Familial Intrahepatic Cholestasis (Deleuze et al., Hepatology 23:904-908, 1996).

Given the limited availability of effective treatment options and chronic progression of disease, there is a need for treatments to ameliorate or prevent cholestatic diseases and their signs and symptoms.

### SUMMARY

The invention provides formulations including a plurality of viable bacteria, wherein the formulation includes at least one bacterial OTU or species that can exhibit a first bile metabolizing activity (e.g., a bile acid or bile salt hydrolase activity; also see below), and a pharmaceutically acceptable excipient.

In some embodiments, the 16S rDNA sequence, or a fragment thereof, of the OTU or species is at least 95% or at least 97% identical (e.g., at least 98%, at least 99%, or 100% identical) to a sequence in FIG. 16, or a portion thereof (see, e.g., below).

In some embodiments, the formulations further include at least one viable bacterial OTU or species that can exhibit an activity selected from the group consisting of a second bile acid or bile salt hydrolase activity having a specificity different from the first bile acid or bile salt hydrolase activity, deconjugation, oxidation, and dehydroxylation.

In some embodiments, the formulations includes at least two different bacterial OTUs or species.

In some embodiments, the formulations include two different bacterial OTUs or species and the formulation can exhibit oxidation and dihydroxylation activities.

In some embodiments, the formulations include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 OTUs or bacterial species selected from at least two clades of Table 1. In various examples, the 16S rDNA of each of the OTUs or bacterial species of the composition of Table 1 has at least 95% or 97% sequence identity (e.g., at least 98%, at least 99%, or 100% identity) to at least one sequence of FIG. 16, or a portion thereof (see, e.g., below).

In some embodiments, the formulations include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, or 50 OTUs or bacterial species selected from at least two clades of Table 2.

In some embodiments, the formulations include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, or 50 OTUs or bacterial species of part B or C of Table 1, Table 2, or Table 3.

In some embodiments, the formulations include one or more OTUs or bacterial species from 5, 10, 15, or 20 clades selected from the group consisting of: 1, 6, 86, 87, 90, 100, 101, 164, 195, 196, 197, 203, 204, and 297.

In some embodiments, the number of different OTUs or species in the formulations is fewer than 60, 50, 30, 20, or 15.

In some embodiments, the bile acid or bile salt hydrolase, deconjugation, oxidation, or dehydroxylation activity of one or more bacterial OTU or species of the formulation is detected using an animal-based assay, a cell-based assay, an in vitro assay, by sequencing, or using a combination of these types of assays.

In some embodiments, each bacterial OTU or species of the formulations has a bile acid or bile salt metabolism activity selected from the group consisting of hydrolysis, deconjugation, oxidation, or dehydroxylation.

The invention also includes therapeutic formulations or compositions including a formulation described above or elsewhere herein. In various embodiments, the viable bacteria of the therapeutic formulations are delivered to the small intestine, the colon, or both.

Also provided by the invention are methods of treating a subject diagnosed with or at risk for a cholestatic disease or condition, the methods including administering to the subject a microbial composition or formulation (see, e.g., the formulations described above), wherein at least one bacterial OTU or species in the microbial composition can deconjugate a primary bile acid or bile salt. In various embodiments, at least one OTU or bacterial species in the microbial composition can metabolize a primary bile acid or salt to a secondary bile acid or salt. In various embodiments, the 16S rDNA sequence of the OTU is at least 95% identical (e.g., at least 98%, at least 99%, or 100% identical) to a sequence in FIG. 16, or a portion thereof (see, e.g., below).

The invention further provides methods of treating a subject diagnosed with or at risk for a cholestatic disease or condition, the methods including administering to the subject a formulation as described above or elsewhere herein.

In various embodiments of the methods described herein, the subject is diagnosed with or at risk for general cholestasis (GC), primary sclerosing cirrhosis (PSC), primary biliary cirrhosis (PBS), progressive familial intrahepatic cholestasis (PFIC), non-alcoholic fatty liver disease (NAFLD), non-alcoholic steatohepatitis (NASH), cholestasis of pregnancy, cholangitis, hepatitis, alcoholic liver disease, hepatocellular carcinoma, liver cirrhosis, cystic fibrosis, graft-versus-host disease (GVHD), or blockage of an extrahepatic bile duct. In various embodiments, the blockage of an extrahepatic bile duct is due to gall stone, inflammatory stricture, cancer, or pancreatitis.

The invention also provides methods of treating a subject diagnosed with or at risk for a cholestatic disease or condition (see, e.g., the list set forth above) and prescribed obeticholic acid (OCA), ursodeoxycholic acid (UDCA), or a derivative of obeticholic acid or UDCA, the method including administering to the subject: (i) a composition including one or more of CDCA, a bacterium that has BSH activity, or a compound that can inhibit one or both of and/or activity; and (ii) a pharmaceutically acceptable excipient.

The invention additional includes compositions containing hyocholic acid or a bacterium that can increase the concentration of hyocholic acid.

The invention further includes methods of treating a subject diagnosed with or at risk for a cholestatic disease or condition (see, e.g., the list set forth above), the methods including administering to the subject UDCA and a composition including a bacterium that has BSH activity, but does not increase lithocholic acid (LCA) levels. In various embodiments, the composition further includes a bacterium that can metabolize a primary bile acid or salt to a secondary bile acid or salt.

Also provided in the invention are compositions including a bacterium that has BSH activity, for use in treating a subject being treated with UDCA, wherein the bacterium does not increase LCA levels.

In various embodiments, the composition further includes a bacterium that can metabolize a primary bile acid or salt to a secondary bile acid or salt.

In some embodiments, the methods further include administering OCA to the subject.

In some embodiments of any of the formulations and methods described herein, the microbial composition is directly derived from human feces, is a designed composition, comprises bacterial spores, or comprises spore forming bacteria.

The invention further provides compositions including a formulation as described above and elsewhere herein for use in treating a subject diagnosed with or at risk for a cholestatic disease or condition (see, e.g., the list set forth above).

Also, the invention provides methods for identifying a bacterial species for use in a composition for altering bile acid metabolism in a subject. These methods include comparing protein coding sequences of a bacterial strain to reference sequences in a database of proteins that catalyze a desired bile acid activity, wherein identification of a bacterial strain including a sequence with homology to a reference sequence indicates the identification of a bacterial strain for use in the composition.

In various embodiments, the methods further include testing the bile acid metabolizing activity of a bacterial species using an in vitro assay or an animal model-based assay.

In further embodiments, the level of sequence homology is at least 75%, 80%, 85%, 90%, 93%, 95%, 97%, 98%, 99%, or 100% identity.

As used herein, “augmentation” refers to the establishment or significant increase of one or more types of microbes (e.g., bacteria) that are (i) absent or undetectable (as determined by a method such as genomic sequencing or microbiological techniques) from a therapeutic microbial composition, (ii) absent, undetectable, or present in low frequencies in a host niche (for example, in the gastrointestinal (GI) tract, e.g., the lumen of the GI tract, the mucosa of the GI tract, the colon, the small intestine) prior to administration of the microbial composition, and (iii) are detectable after the administration of the microbial composition or, in cases where the microbes were present at low frequencies prior to administration, significantly increase after administration of the microbial composition; for example 2-fold, 5-fold, 1×10²-fold, 1×10³-fold, 1×10⁴-fold, 1×10⁵-fold, 1×10⁶-fold, 1×10⁷-fold, or greater than 1×10⁸-fold.

The microbes comprising an augmented ecology can be derived from exogenous sources such as food or other environmental sources, or can be derived from niches within the host where they reside at low frequency. The increase may be in number of a particular type of bacteria, an increase in the diversity of types (e.g., clades, OTUs or species of bacteria), or both. In some embodiments, a reference level is established for comparison to determine “undetectable” or “low” frequencies.

“Clade” refers to the operational taxonomic units (OTUs or members of a phylogenetic tree) that are downstream of a statistically valid node in a phylogenetic tree. Clades are defined based on the topology of a phylogenetic tree that is constructed from full-length 16S rDNA sequences using maximum likelihood methods. Clades are constructed to ensure that all OTUs in a given clade are within a specified number of bootstrap supported nodes from one another, and have genetic similarity based on full-length 16S rDNA sequence. OTUs that are within the same clade can be distinguished as genetically and phylogenetically distinct from OTUs in a different clade based on, e.g., 16S rDNA sequences. Accordingly, species within a single clade are likely to have conserved ecological function and may be interchangeable in a composition.

“Dysbiosis” refers to a state of the microbiota of the GI tract or other body area in a subject, including mucosal or skin surfaces, in which the normal or healthy diversity and/or function of the microbial ecological network is disrupted. The disruption results in an unhealthy state of the microbiome that can be due to, e.g., a decrease in diversity of the microbiome, the overgrowth of one or more pathogens or pathobionts, the presence of symbiotic organisms that are able to cause disease only when certain genetic and/or environmental conditions are present in a subject, or the shift to microbiome that no longer provides one or more essential functions to the host subject, and therefore no longer promotes health, or a change in the balance of one or more metabolic functions. A dysbiosis can be caused, e.g., by the use of antibiotics to treat or prevent an infection. In some cases, a dysbiosis is related to an alteration in host physiology, for example, a decrease in enterohepatic bile circulation, e.g., due to dysfunction of hepatobiliary system. In some cases, a dysbiosis is associated with an inflammatory state, e.g., in PSC or ulcerative colitis.

“Engraftment” refers to the establishment of a bacterial type (e.g., a bacterial clade, OTU, or species) present in a therapeutic composition in a target niche such as the GI tract (e.g., the small intestine or the large intestine) of a host treated with the composition, and wherein the bacterial type was absent or undetected in the treated host prior to treatment. Engrafted species or OTUs can establish as measured from administration of the final dosage or a treatment (for example, 1 day, 2 days, 3 days, 4 days, 5 days, 6 days, 1 week, 2 weeks, 3 weeks, 4 weeks, 8 weeks, 12 weeks, 3 months, or 6 months). Methods of detection are known in the art and include qPCR, 16S v4 Next-Generation Sequencing (NGS) and Whole Metagenomic Sequencing (WMS) and other methods of high throughput sequencing. Detection limits can be, for example, detection of one bacterium in 10e6, one in 10e7, or one in 10e8. In some embodiments, the method of detection can selectively detect a strain of bacterium provided in a composition. In some embodiments, the method of detection can selectively detect a species or OTU of a bacterium provided in a composition. Without committing to any particular theory, an engrafted population of microbes may induce an environmental shift in the target niche, thereby promoting favorable conditions for the growth of commensal microbes capable of catalyzing a shift from a dysbiotic ecology to one more representative of a healthy state.

As used herein, “treating” and “treatment” refer to the administration of an agent, composition, or formulation to an individual diagnosed with or predicted to be at risk for a disease to prevent or ameliorate at least one sign or symptom of the disease. The terms “disorder” and “disease” are used interchangeably herein. The terms “preventing” and “prevention” refer to the administration of an agent or composition to a clinically asymptomatic individual who is susceptible to a particular adverse condition, disorder, or disease, and thus relates to the prevention of the occurrence of at least one sign or symptom of a disease. As used herein, unless indicated otherwise, the term “symptom” includes signs and symptoms.

A “therapeutically effective amount” or an “effective amount” of an agent, composition, formulation, or combination thereof is a sufficient amount of the agent, composition, formulation, or combination thereof to prevent or ameliorate at least one symptom of a disorder. A therapeutically effective amount of a therapeutic composition described herein can vary according to factors such as the disease state, age, sex, and weight of the individual, and the ability of the therapeutic composition to elicit a desired response in the individual, e.g., amelioration of at least one disorder parameter, or amelioration of at least one sign or symptom of the disorder (and optionally, the effect of any additional agents being administered). A therapeutically effective amount is also one in which any toxic or detrimental effects of the composition are outweighed by the therapeutically beneficial effects. A composition as described herein is generally administered in a therapeutically effective amount. Symptoms of, or associated with, cholestatic diseases are known in the art. For example, symptoms of general cholestasis are known in the art and can include, e.g., pruritus, jaundice, enlarged liver and spleen, fatigue, nausea and vomiting, cirrhosis, liver failure, liver cancer, gallstones, and biochemical markers of such symptoms.

“Types” of bacteria refers to bacteria grouped by strain, species, clade, family, or other organizational categories. In some embodiments, a bacterial species is defined as bacteria having at least one 16S rDNA sequence that is at least 95%, at least 97%, at least 98%, at least 99%, or at least 100% identical to a 16S rDNA sequence of a reference bacterium. In some cases, a bacterial species is defined as bacteria having at least one variable region of a 16S rDNA sequence (V1-V8), e.g., a V4 or V6 region, that is at least 95%, at least 97%, at least 98%, at least 99%, or at least 100% identical to a corresponding 16S rDNA variable region of a reference bacterium.

The entire disclosure of each patent document and scientific article referred to herein, and those patent documents and scientific articles cited thereby, is expressly incorporated by reference herein for all purposes.

Additional features and advantages of the invention are more particularly described below.

### BRIEF DESCRIPTION OF THE DRAWINGS

FIGS. 1A and 1B depict results of in vitro screening of human bacterial isolates and designed compositions for bile acid metabolism. FIG. 1A depicts data illustrating diversity of enzymatic activities and substrate specificities in bacterial species and individual strains. Each row corresponds to an individual strain tested. Columns depict the five categories of enzymatic activities encompassed by the screening approach, and bile acid substrates used for screening. Darkened squares indicate the presence of enzymatic activity on the indicated substrate, while blanks indicate no activity. FIG. 1B depicts designed compositions and their constituent strains and bile acid activities. Rows indicate individual strains within indicated compositions. Columns indicate enzymatic activity and substrate specificity for each strain. Strains were also tested as mixtures of complete compositions to confirm activities. At least one 16S rDNA sequence useful for identifying species is provided in FIG. 16.

FIG. 2 depicts NGS data from an experiment in which germ-free mice were colonized with a No BA composition. Fecal samples from five mice, collected prior to dosing and 1 day, 3 days, and 7 days post-dosing with a “no bile acid activity” composition were analyzed by Next-Generation Sequencing (NGS). The y-axis indicates the number of reads observed up to 20,000 reads for each sample. Individual clades are represented by different shading in the bar graphs. The three clades belonging to the bacteria in the “no bile acid” composition are shown as dark gray, white, and light gray.

FIG. 3 depicts data from experiments testing whether colonization with designed compositions can alter fecal bile acid content in germ-free mice. Data illustrate the fecal bile acid profiles of germ-free mice colonized with a ‘No bile acid (No BA) activity’ composition, ‘BSH only activity’ composition, ‘Max BA activity’ composition, or ‘Conventionalized’ (mouse-derived FMT). Five mice were tested per treatment and bile acid profiles are depicted as percent of the total assayed bile acid pool (assayed bile acid pool means the sum of all detected bile acids in a sample). * indicates a significant difference compared to conventionalized mice, based on a two-way Anova with a Tukey's multiple comparisons test. P-values are as indicated: ** p≤0.01, **** p≤0.001.

FIG. 4 depicts the results of experiments examining whether catabolism of conjugated primary bile salts by bacteria can decrease liver bile acid pools. Total liver bile acid pools were measured in wild-type, conventionalized, and germ-free mice, as well as mice colonized with ‘No BA activity’, ‘BSH only activity,’ or ‘Max BA activity’ compositions. Five mice were tested in each arm, total liver bile acids were assayed using LC-MS, and results were normalized to sample tissue weight (nM/mg). Total bile acid pools were determined as the sum of all detected bile acids in liver tissue and quantified using calibration curves with pure standards. * indicates a significant difference in total bile acid levels compared to germ-free mice based on a paired t-test. P-values are as indicated: *** p<0.001, **** p≤0.001.

FIG. 5A illustrates the results of experiments examining whether colonization of germ-free mice with bacterial compositions with differing bile acid catabolic activities can significantly alter ileal FXR gene expression. Gene expression was assessed by Taqman qPCR in ex-germ-free mice colonized with either a no bile acid activity (No BA Activity), BSH only activity, or Max BA activity composition. Controls include ex-germ-free mice colonized with murine stool via oral gavage (“FMT) and conventional mice with a murine microbiota. Expression is shown as relative to the housekeeping gene, beta-actin. **** p≤0.0001 by Tukey's multiple comparisons (n=4-8).

FIG. 5B illustrates the results of experiments demonstrating that colonization of germ-free mice with bacterial compositions that have distinct bile acid activities differentially affected ileal FGF15 gene expression. Ileal FGF15 expression was assessed in germ-free mice, conventionally housed mice, germ-free mice colonized with a murine-derived FMT as well as mice colonized with a ‘No BA activity composition’, a ‘BSH only activity’ composition or a ‘Max BA activity’ composition. Expression is shown as relative to the housekeeping gene, beta actin. **** P<0.0001 by Tukey's multiple comparisons (n=4-8).

FIGS. 6A-6D depict the results of experiments testing the ability of obeticholic acid (OCA) to mediate FXR-activated gene expression in the presence of CDCA. The ability of obeticholic acid (OCA) to stimulate expression of downstream target genes was enhanced 20-fold in the presence of CDCA. FIG. 6A: hFXR reporter cells were incubated for 24 hours with serial dilutions of either CDCA (0-100 μM), OCA (0-10 μM), or OCA (0-10 μM) with a constant concentration of 50 μM CDCA. FXR activation was assessed by measurement of luciferase activity and is represented as fold change over vehicle control (0.2% DMSO). Each dose was performed in triplicate. FIG. 7B: Dietary supplementation with a cholic acid diet resulted in increased FXR signaling in vivo. FIGS. 7C and 7D. OCA activated FXR signaling and modulated expression of FXR-dependent genes in a dose dependent manner. FXR signaling was assessed by measurement of expression of down-stream genes included Cyp7A1 in the liver and FGF15 in the ileum.

FIG. 7A depicts the results of experiments demonstrating that CDCA, but not HCA, activates FXR signaling in-vitro. Dose response curves of selected bile acids alone in an hFXR luciferase cell reporter assay indicate full agonist activity of CDCA, low agonist activity of LCA and DCA (maximum concentration tested was 100 μM) and no agonist activity of hyocholic acid (HCA; up to 200 μM) on FXR signaling.

FIG. 7B depicts the results of experiments demonstrating synergistic activation of FXR by hyocholic acid (HCA) the presence of 50 μM CDCA (EC₅₀ of CDCA=50 μM as shown in FIG. 7A) in a luciferase based in-vitro FXR activation assay. DCA and LCA showed no significant synergistic effects with HCA. LCA at 100 μM with CDCA at 50 μM showed no difference in fold activation due to increased toxicity to cells. Data are expressed as fold change relative to 50 μM CDCA alone.

FIGS. 8A and 8B depict the results of experiments showing changes in total bile acid levels and bile acid composition in patients with Primary Sclerosing Cholangitis (PSC). FIG. 8A: Patients show decreased total bile acid levels in bile with corresponding increases in total portal blood and peripheral blood bile levels. FIG. 8B: PSC patients show decreases in the proportions of secondary and conjugated secondary bile acids typically generated by the gut microbiome, with significant increases in the corresponding upstream conjugated primary bile acids. Bile acid concentrations were measure using LC-MS with appropriate reference standards for quantitation. Data is depicted as mean±standard deviation. * indicates a significant difference between PSC and healthy patients, based on a two-way Anova with a Tukey's multiple comparisons test. P-values are as indicated: * p≤0.1, ** p≤0.01, *** p≤0.001 **** p≤0.0001.

FIGS. 9A-9G show the results of experiments in which conventionally housed mice treated with DDC-diet induced cholestatic disease showed altered bile acid content and gut microbial profile as compared to healthy controls. FIG. 9A: Significantly increased levels of serum biomarkers in DDC treated conventionally housed Swiss albino mice, including Alkaline Phosphatase (ALP), Alanine Aminotransferase (ALT), Total Bilirubin (TBIL) and Cholesterol (CHOL), confirming the presence of cholestatic liver disease. FIGS. 9B-9D: Mice with DDC-diet induced cholestatic disease show decreased levels of 2° and conjugated 2° bile acids, with corresponding increases in upstream conjugated 1° bile acids compared to untreated controls. FIG. 9E: Alpha diversity and beta diversity indices show distinct microbial populations in mice pre (d0) and post (d21) DDC treatment to induce cholestatic disease. Alpha diversity (left graph) is a measure of species richness based on a Shannon diversity index, while beta-diversity (right graph) is depicted as distance of separation on two weighted axis based on a principle coordinate analysis (PCoA). FIG. 9F: List of species with significantly different prevalence (p<0.2) between mice pre (d0) and post (d21) treatment with DDC to induce cholestatic disease. FIG. 9G: Significant loss of strains with known 7α-dehydroxylation activity with cholestatic disease development in conventional Swiss albino mice. * indicates a significant difference between DDC treated and control mice (A-D) or between mice pre (d0) and post (d21) DDC treatment. Significance analysis based on a two-way Anova with a Tukey's multiple comparisons test. P-values are as indicated: * p≤0.1, ** p≤0.01, *** p≤0.001 **** p≤0.0001

FIGS. 10A-10E show that the absence of a gut microbiome results in increased susceptibility to DDC-diet induced cholestatic disease in Swiss albino mice, a condition that can be reversed by colonization with a mouse-derived FMT. Germ-free (GF) mice show more rapid weight loss (FIG. 10A) and significantly higher levels of serum biomarkers such as alkaline phosphatase (FIG. 10B), alanine aminotransferase (FIG. 10C), and total bilirubin (FIG. 10D) compared to conventionally-housed (CH) mice when treated with the DDC diet. Colonization for 4 weeks with a Swiss albino mouse-derived self-FMT in GF mice reverses this susceptibility to DDC-induced disease, with body weight loss and levels of ALP, ALT and TBIL reduced to levels comparable to CH mice given DDC-diet. FIG. 10E shows the results of bile acid profiling, which determined that FMT treatment was sufficient to restore bile acid composition in the gut compared to germ-free mice. * indicates a significant difference relative to GF mice on DDC treatment. Significance analysis based on a two-way Anova with a Tukey's multiple comparisons test. P-values are as indicated: * p≤0.1, ** p≤0.01, *** p≤0.001 **** p≤0.0001

FIGS. 11A-11E show that microbial composition affects susceptibility to DDC-diet induced cholestatic liver disease. FIGS. 11A-11B: Germ-free C57Bl/6 and Swiss albino mice show comparable susceptibility to liver disease induced body weight loss despite significant resistance of conventional Swiss albino mice to disease. FIG. 11C: On a DDC diet, colonization of germ-free Swiss albino mice with conventional C57Bl/6 donor mice FMT results in more rapid weight loss than in Swiss albino mice receiving FMT from conventional Swiss albino donor mice. FIG. 11D: Serum ALP levels remain comparable between mice colonized with FMTs from different donors, despite differences in body weight loss prior to terminal collection. FIG. 11E: C57Bl/6 donor FMT colonized mice have significantly higher levels of 1° bile acids and lower levels of the 2° bile acids compared to Swiss albino donor FMT colonized mice. (*Note, for FIG. 11A, GF-C57 mice were part of a separate experiment from the other three arms.)

FIGS. 12A-12C show that restoring the microbiome rescues the susceptibility of germ-free mice to DDC diet induced liver disease. Treatment with either a complex conventional Swiss albino mouse derived FMT or a less complex designed composition that restores bile acid metabolism (Max BA) reduced body weight loss (FIG. 12A) and ALP levels (FIG. 12B) in response to DDC diet in germ-free Swiss albino mice. FIG. 12C: Colonization with either a conventional mouse derived FMT or a designed composition (Max BA) restored the majority of fecal bile acid subsets in germ-free mice. * indicates a significant difference relative to GF mice on DDC treatment. Significance analysis based on a two-way Anova with a Tukey's multiple comparisons test. P-values are as indicated: * p≤0.1, ** p≤0.01, *** p≤0.001 **** p≤0.0001

FIG. 13 demonstrates the beneficial effect of dietary supplementation with secondary bile acids (deoxycholic acid+lithocholic acid) on susceptibility to DDC-diet induced cholestatic disease. Germ-free Swiss albino mice on a DCA+LCA supplemented diet show a decreased rate of body weight loss compared to untreated germ-free mice on when exposed to the DDC diet.

FIG. 14 is a set of graphs and tables showing the results of comparing bioinformatic prediction of BSH activity to in vitro assayed activity.

FIG. 15 is a graph and table showing the results of comparing bioinformatics prediction of 7α-dehydroxylation activity to in vitro assayed activity.

FIG. 16 is a listing of full-length 16S rDNA sequences from exemplary bacteria useful in various compositions and methods of the invention.

### DETAILED DESCRIPTION

The invention provides methods and compositions for use in the prevention, amelioration, and treatment of cholestatic diseases. According to the methods of the invention, the microbiome of a treated subject is altered to affect bile acid metabolism by the administration of a bacterial composition, such as a composition described herein. In some embodiments, the bacterial composition is used in combination with a pharmaceutical. In some embodiments, the pharmaceutical dosage or regime, when administered with the composition, is reduced in amount as compared to treatment with the pharmaceutical alone. In some embodiments, the efficacy of the pharmaceutical is increased and/or side effects are reduced by treatment with a composition of the invention.

Microbiome mediated bile acid metabolism in the GI tract involves deconjugation of conjugated primary bile acids, a process by which the polar taurine or glycine groups are removed from conjugated primary bile salts, generating primary bile acids (Ridlon et al., J. Lipid Res. 47:247-259, 2006). Decreasing concentrations of conjugated primary bile salts can significantly influence the effects and/or progression of cholestatic diseases in which a patient has an undesirable level of conjugated bile acids. Furthermore, a composition can cause additional effects due to increased levels of primary and/or secondary bile acids, which have signaling properties, e.g., in epithelial cells, immune cells, and the liver. Manipulating bile acid pathways can have a therapeutic benefit, as evidenced by reports that OCA can be of therapeutic benefit to patients with cholestasis. As described below, in some embodiments, a composition of the invention comprises one or more bacterial species or OTUs that can express at least one bile salt hydrolase (BSH).

Primary bile acids are the products of deconjugation of conjugated primary bile salts. Examples of primary bile acids are cholic acid (CA) and chenodeoxycholic acid (CDCA). CA and CDCA are ligands for farnesoid X receptor (FXR), a nuclear hormone receptor that regulates bile acid production in the liver. FXR regulates bile acid homeostasis by downregulating bile acid synthesizing enzymes CYP7A1 and CYP8B1 (Sinal et al., Cell 102:731-744, 2000). Activation of FXR signaling represses bile acid synthesis and increases bile acid export from the liver, thereby reducing hepatic accumulation and liver damage by potentially toxic bile acids (Chiang, Compr. Physiol. 3:1191-1212, 2013). Reduction in bile acid synthesis can be mediated both directly in the liver and indirectly through the GI tract by an FXR-FGF15/19-FGF4R pathway, both of which are mediated by bile acid signaling. FGF19 is the human ortholog of murine FGF15. FXR signaling is also believed to have an anti-inflammatory component through its actions on NF-kB signaling (Chiang, Compr. Physiol. 3:1191-1212, 2013). Molecules targeting the nuclear hormone receptor FXR (NR1H4) are in development for treating cholestatic liver diseases, e.g., OCA. Accordingly, in some embodiments, compositions and methods of the invention relate to compositions that include one or more bacteria that can metabolize conjugated primary bile salts to primary bile acids and, optionally, can also metabolize primary bile acids to secondary bile acids.

In some embodiments, microbiome synthesized primary bile acids, such as cholic acid (CA) and chenodeoxycholic acid (CDCA), are the primary endogenous ligands for FXR and therefore play a significant role in reducing the concentrations of conjugated primary bile acids and their synthesis. The resulting primary and secondary bile acids can function to prevent hepatic accumulation of conjugated bile acids, as well as damage from potentially toxic bile acids. The invention thus provides bacterial compositions, including designed compositions, which alter bile acid metabolism along targeted pathways which influence endogenous signaling in the ileum and liver. The compositions and methods of the invention are described in more detail, as follows.

### Compositions

Compositions of the invention comprise microbes, e.g., bacteria, which have been identified in the gastrointestinal tract of a healthy mammal, e.g., a human. In some embodiments, the types of bacteria useful in a composition are types identified in the small intestine (e.g., the human small intestine). In some cases, the types of bacteria are those identified primarily in the colon. Some embodiments include mixed populations of bacteria (e.g., small intestine and colon derived bacteria). In some cases, the composition is derived from a fecal preparation, e.g., a preparation directly derived from human feces. “Directly derived” from human feces means that the bacteria of the composition are isolated from human feces with little or no culturing of such bacteria.

In some embodiments, the composition comprises bacteria derived from single species axenic cultures. Selected species from such cultures are combined to produce a composition. Such a composition is termed herein, a “designed composition.” In some cases, the bacteria from cultures are induced to form spores and such spores are used in the composition. Bacteria in designed compositions are generally species that have been identified in healthy human feces. Examples of designed compositions are described infra.

In some embodiments, the composition comprises bacteria that can metabolize one or more conjugated primary bile salts to primary bile acids by removing taurine and/or glycine conjugations and/or bacteria that can metabolize one or more primary bile acids to secondary bile acids through hydrolysis, oxidation, reduction, hydroxylation, epimerization, 7-alpha-dehydroxylation (through a series of CoA-ligation, oxidation, and/or dehydration reactions), desulfation, as well as dimerization of bile acids. For example, certain compositions of the invention include bacteria that can express bile salt hydrolase (BSH) activity, and therefore can be used to increase deconjugation of conjugated primary bile salts, e.g., in the gastrointestinal tract. Other compositions include bacteria that have activities including not only bile salt hydrolase activity, but also bile acid oxidation and 7-alpha-dehydroxylation. Bacteria able to express specific functions can be selected to achieve the desired alteration in bile acids. For example, bacteria can be selected for their ability to express functional BSH, 7α-dehydroxylase, α-hydroxysteroid dehydrogenase (α-HSDH), β-hydroxysteroid dehydrogenase, or other enzymes able to metabolize a bile acid. It is to be noted that in some cases the activity is effected by an operon having more than one specific activity, e.g., 7α-dehydroxylation, resulting in metabolism of a hydroxyl bile acid to a dehydroxy bile acid. Bacteria or combinations of bacteria are selected for their ability to, e.g., decrease the amount of one or more conjugated primary bile salts (e.g., glycocholic acid, taurocholic acid, glycochenodeoxycholic acid, taurochenodeoxycholic acid, tauro-α-muricholic acid, or tauro-β-muricholic acid). In some embodiments, bacteria or combinations of bacteria are selected for their ability to decrease or increase the amount of one or more primary or secondary bile acids, (e.g., cholic acid, deoxycholic acid, oxocholic acid (3-, 7-, or 12-), isocholic acid, chenodeoxycholic acid, Iithocholic acid, oxochenodeoxycholic acid (3- or 7-), isochonedeoxycholic acid, α-muricholic acid, β-muricholic acid, γ-muricholic acid (also known as hyocholic acid). Bile acids can be assayed using LC-MS as described herein and is known in the art, thin layer chromatography, GC-mass spectrometry, or other methods known in the art.

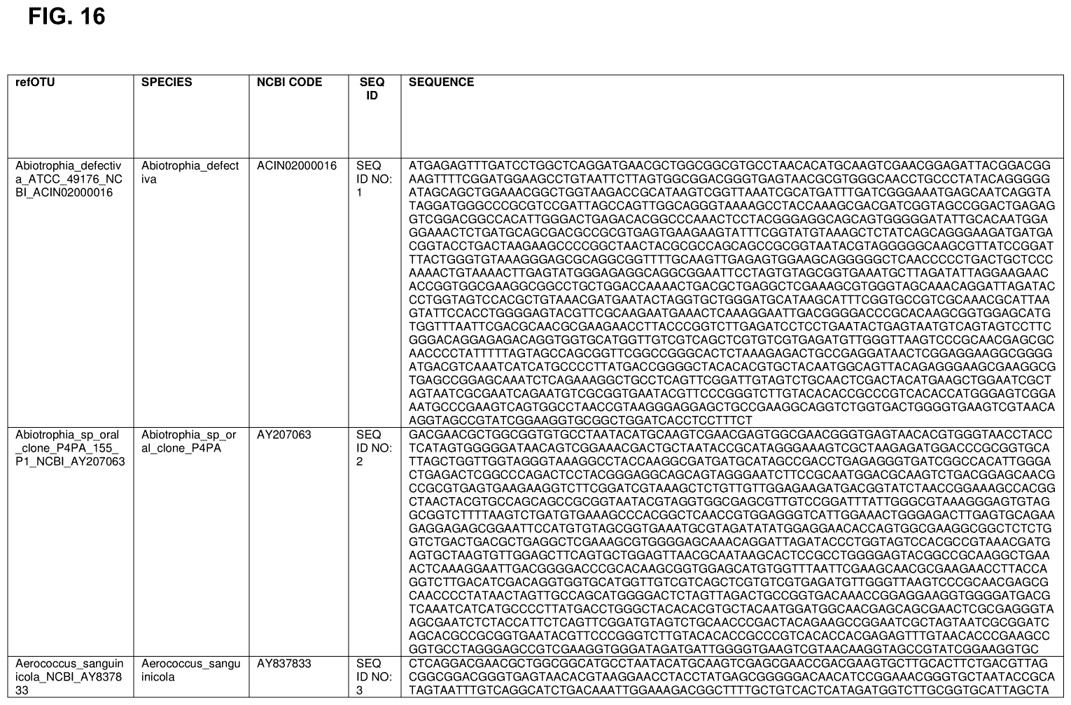

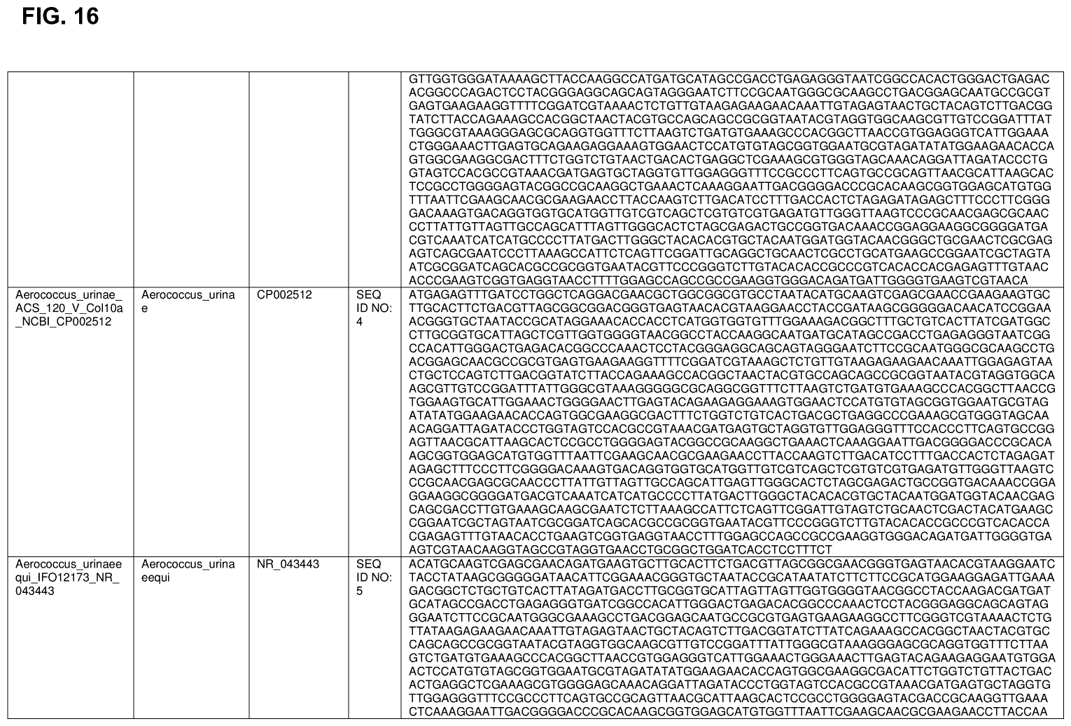

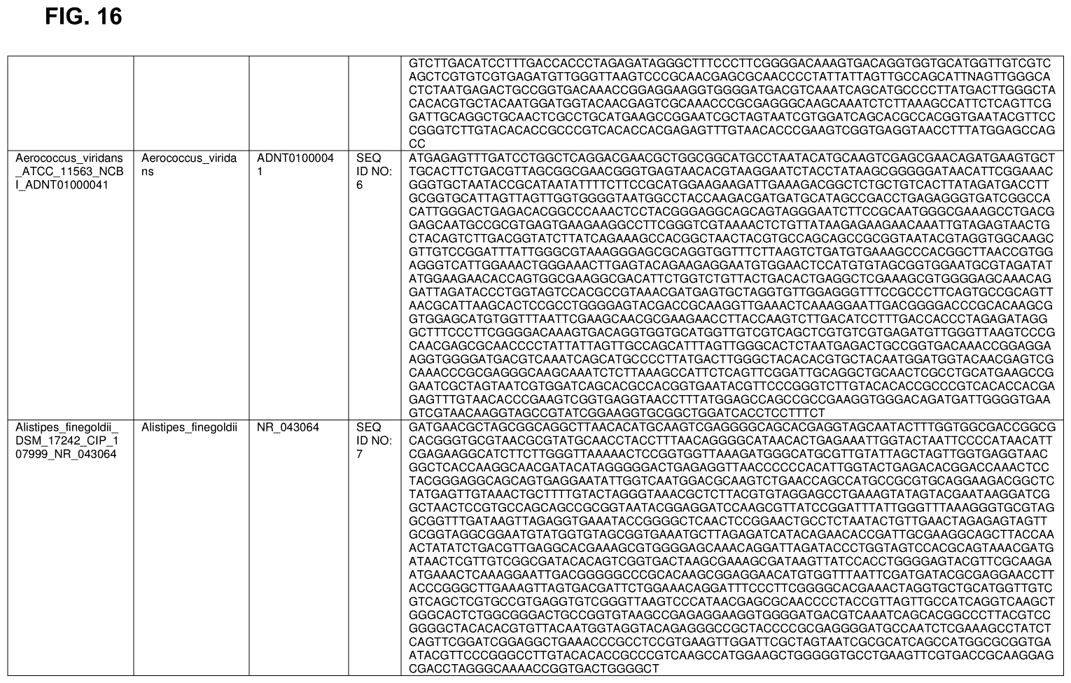

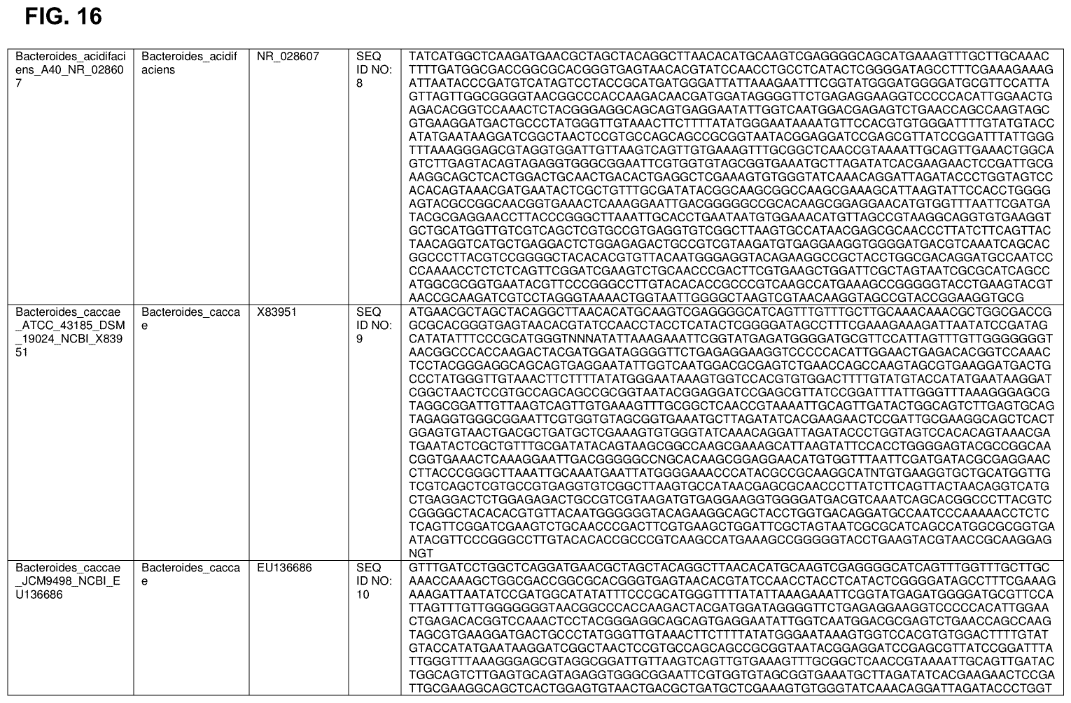

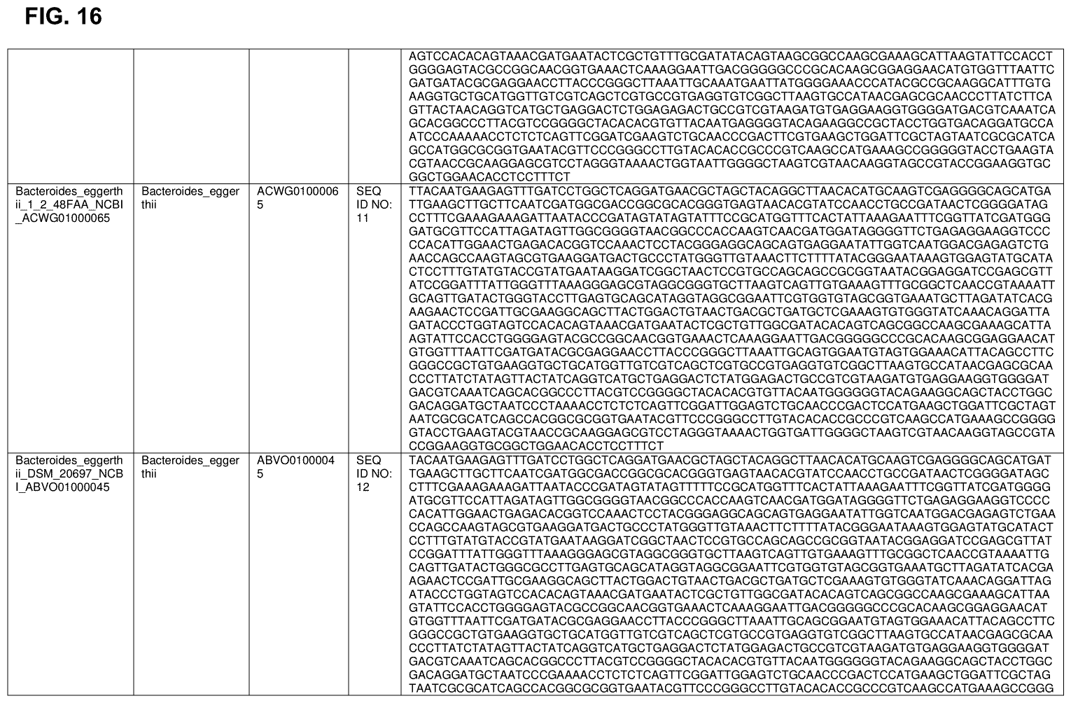

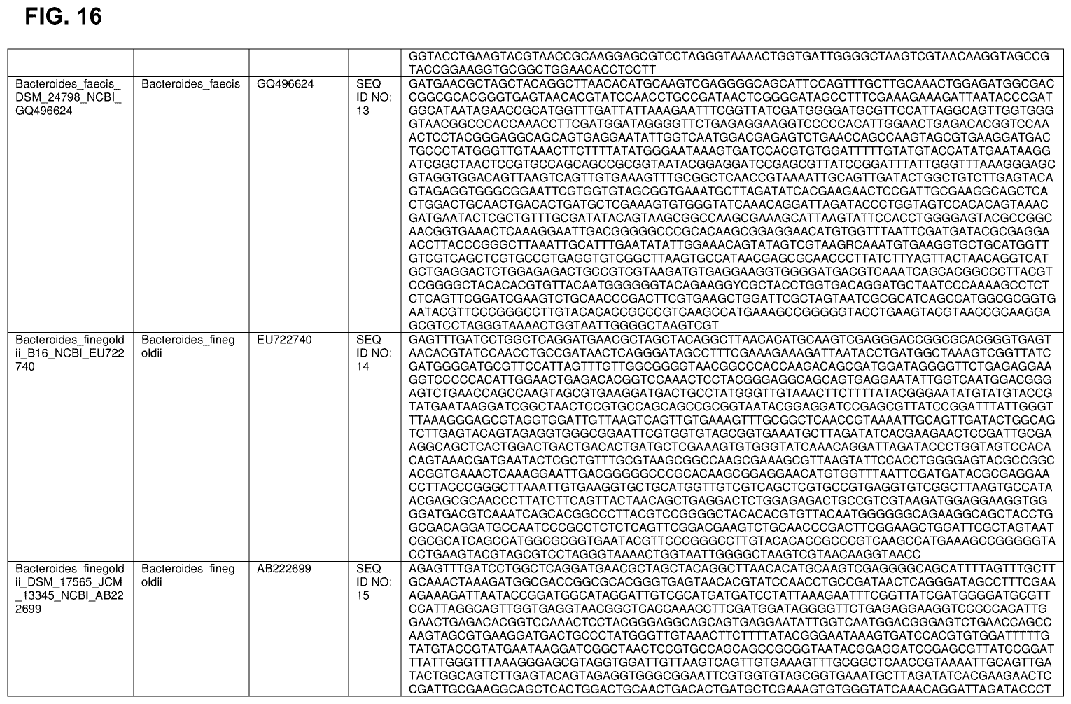

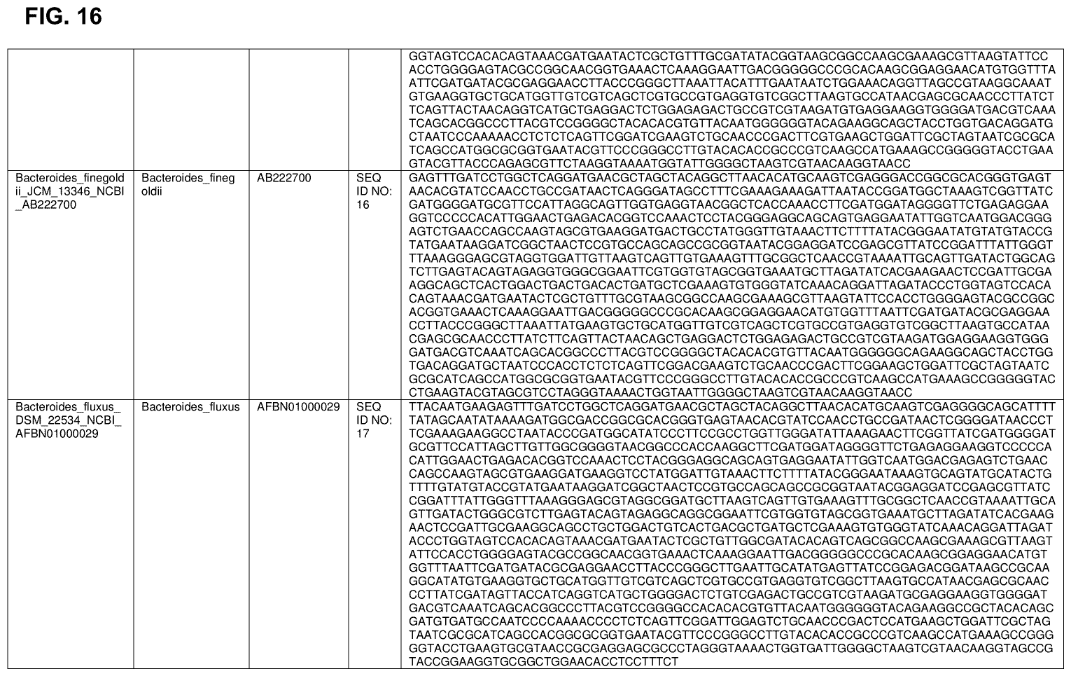

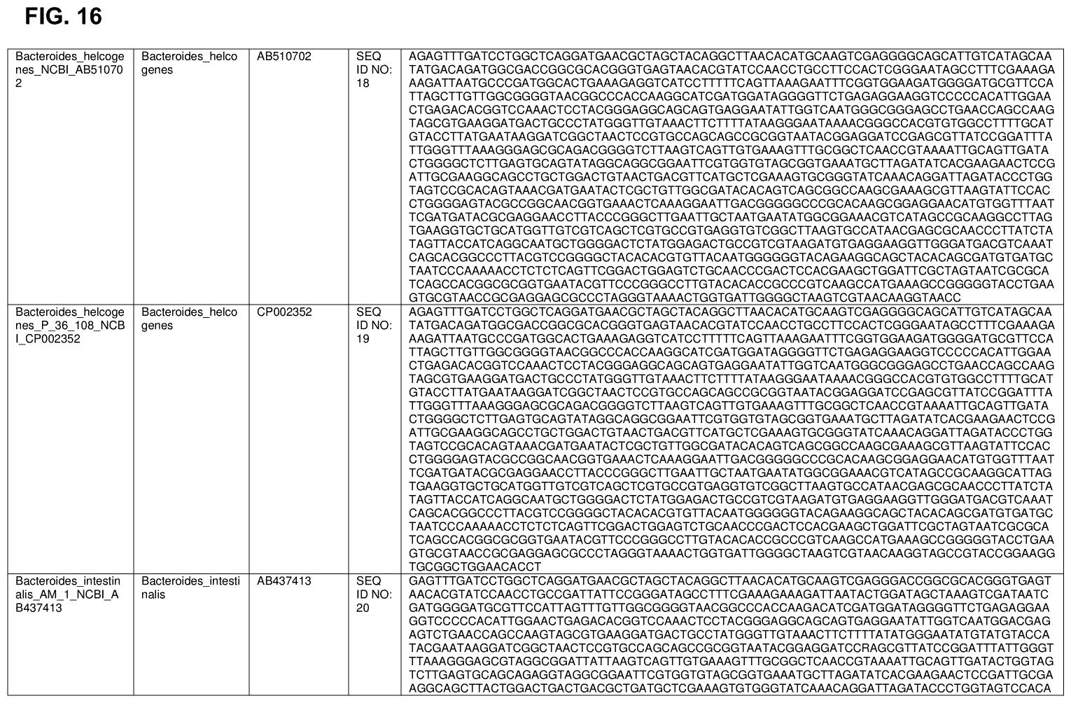

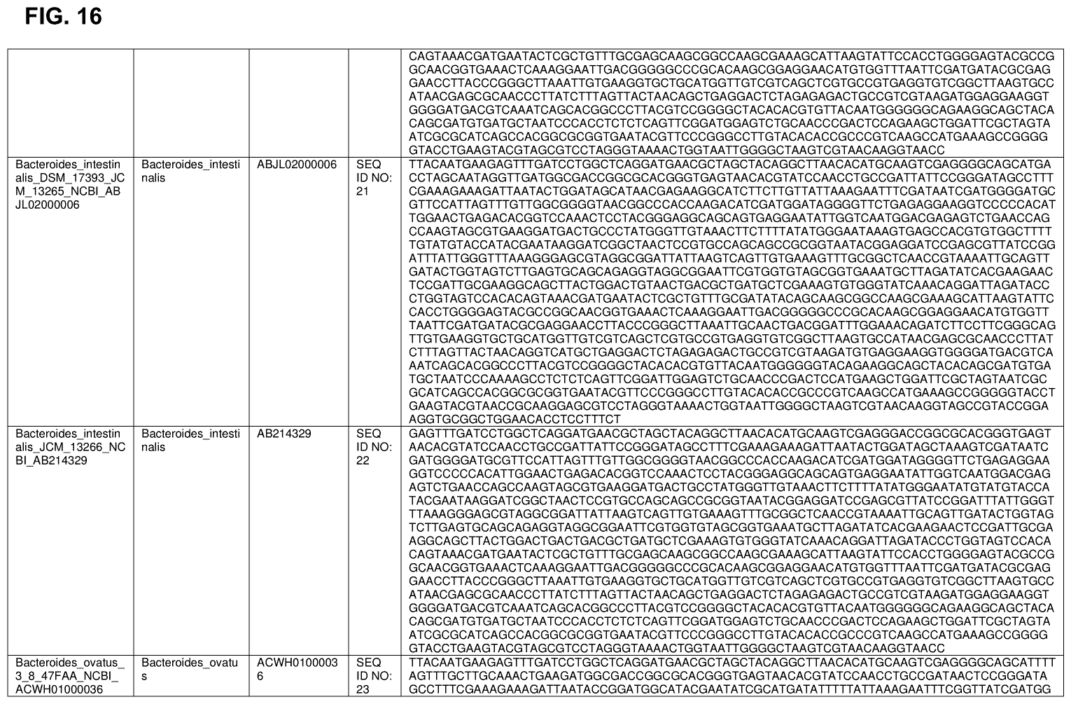

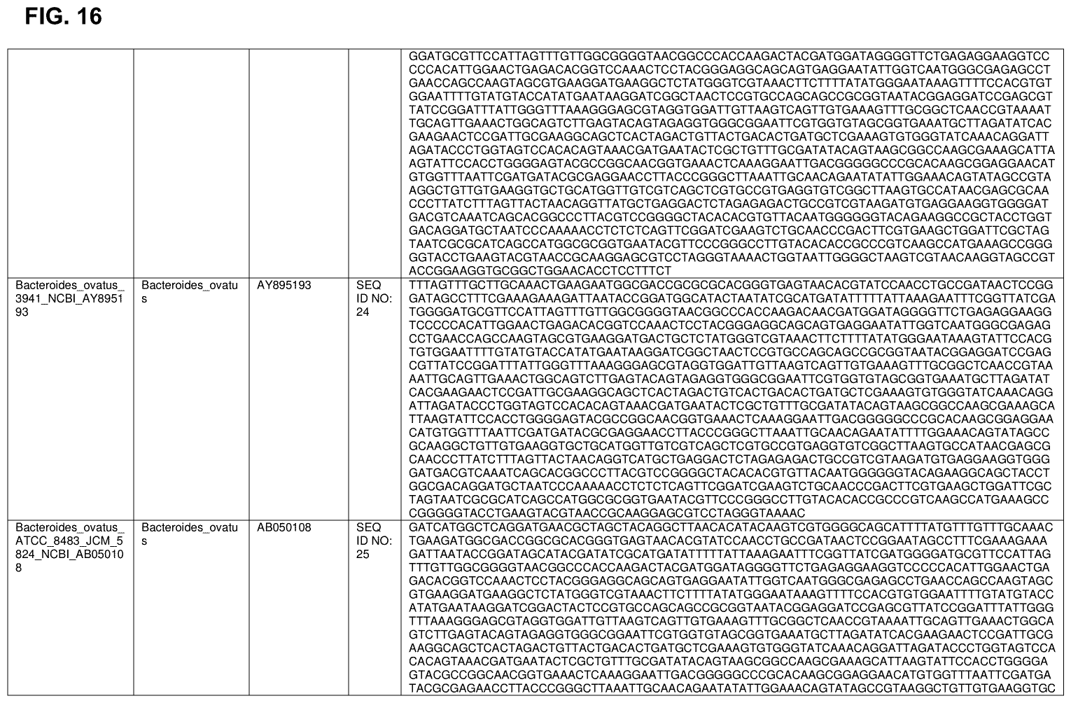

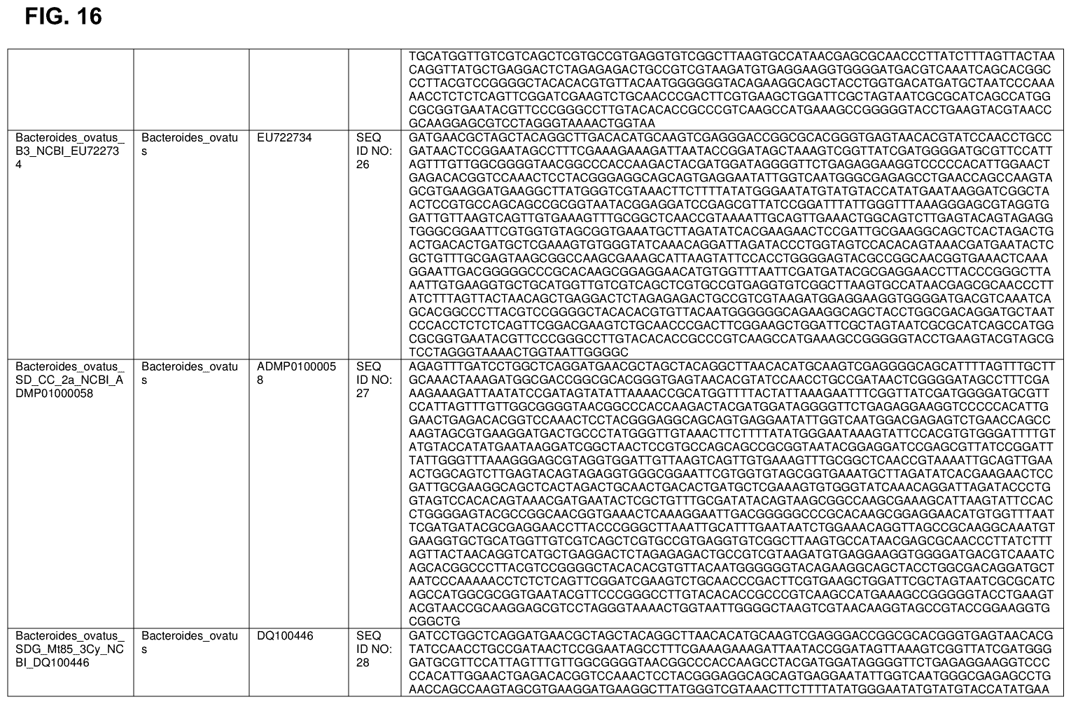

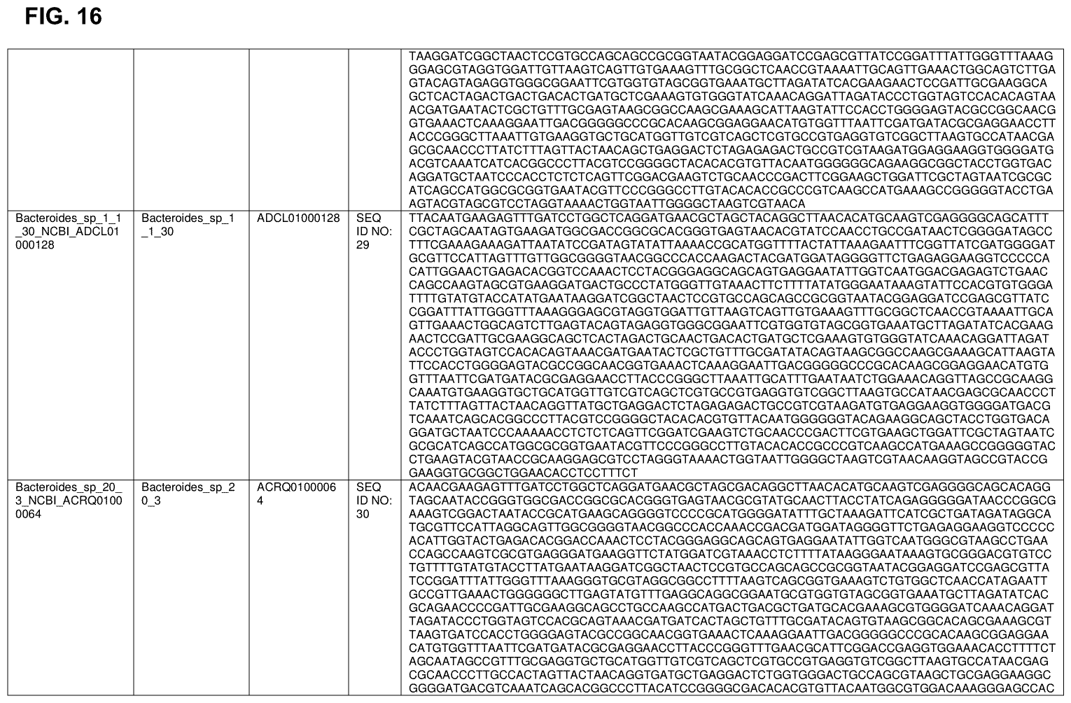

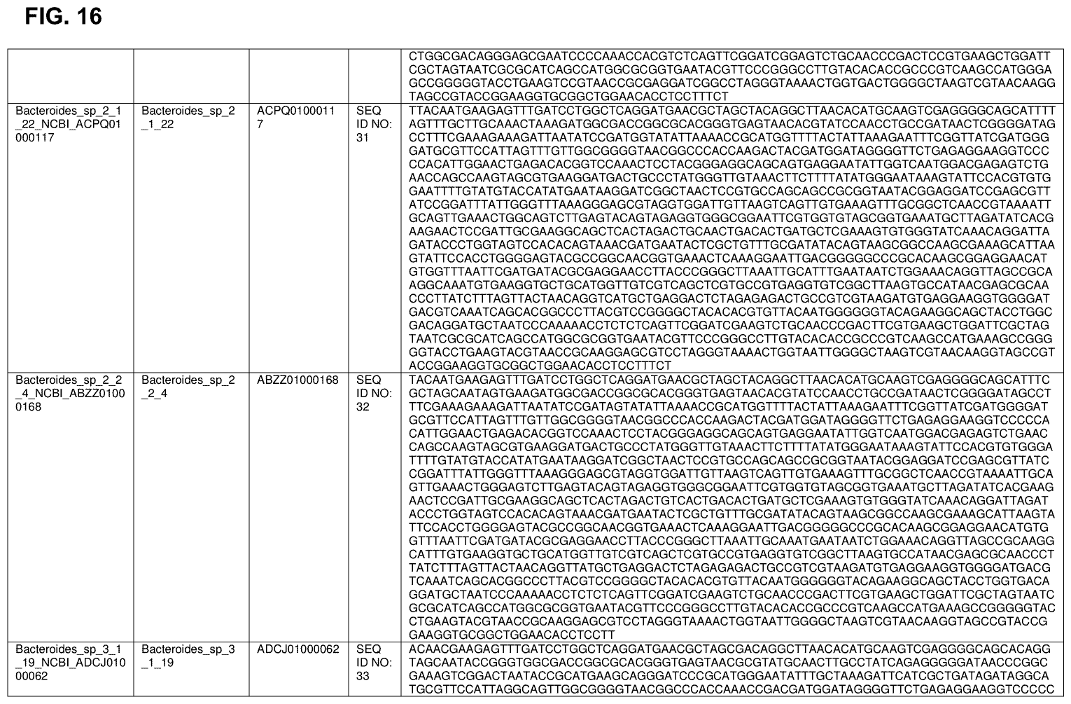

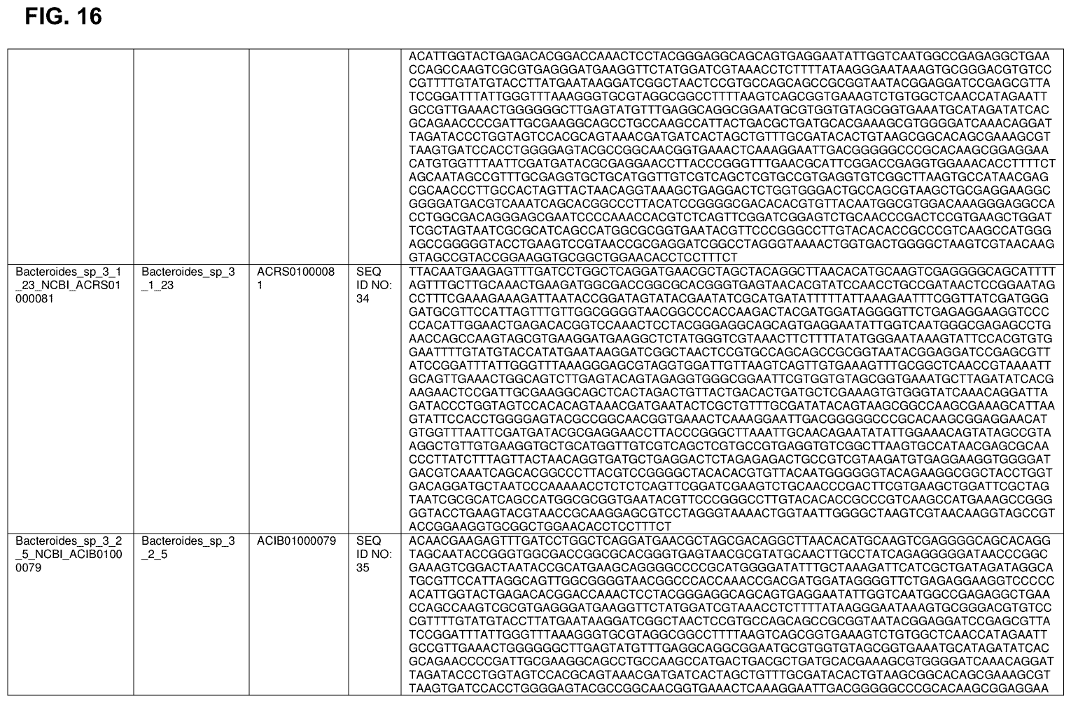

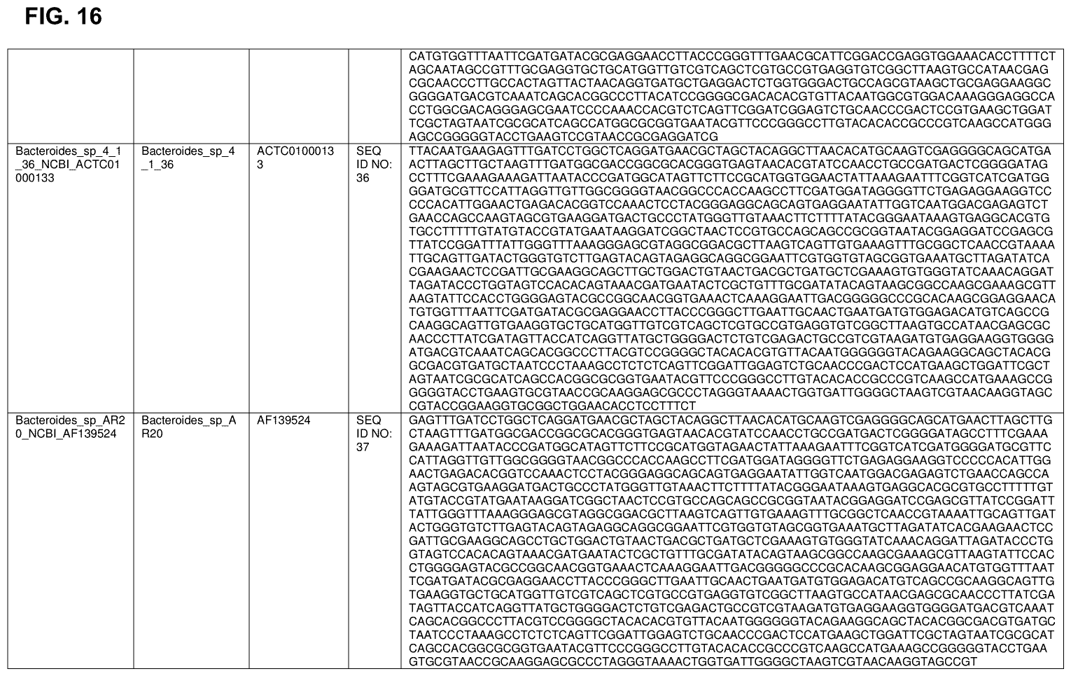

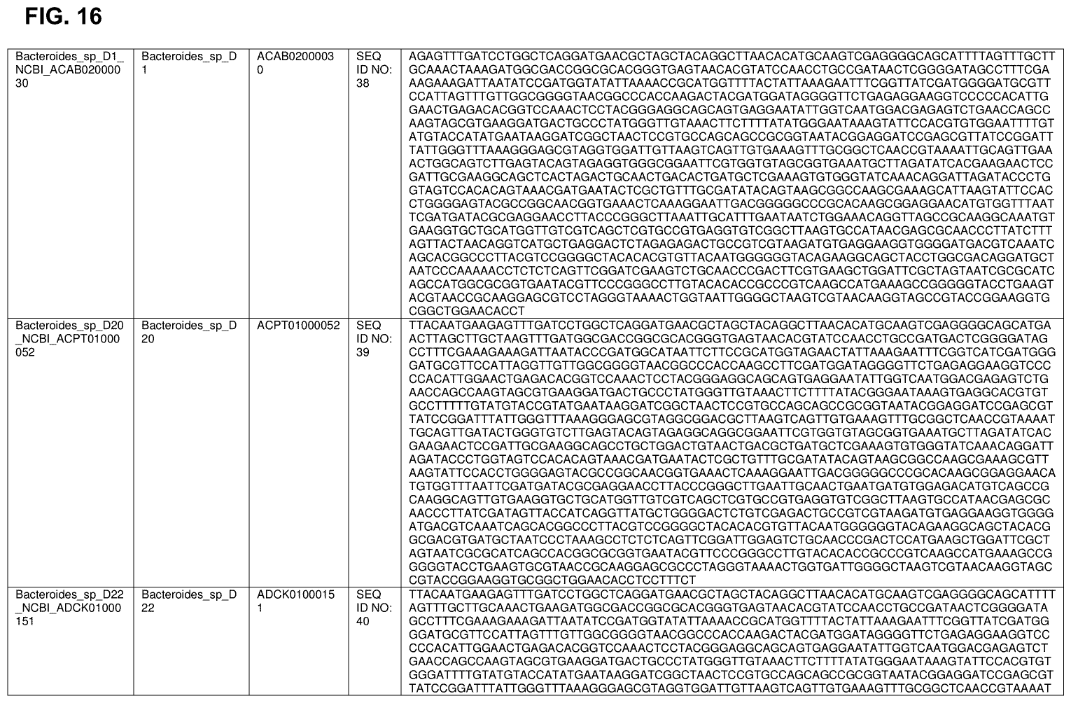

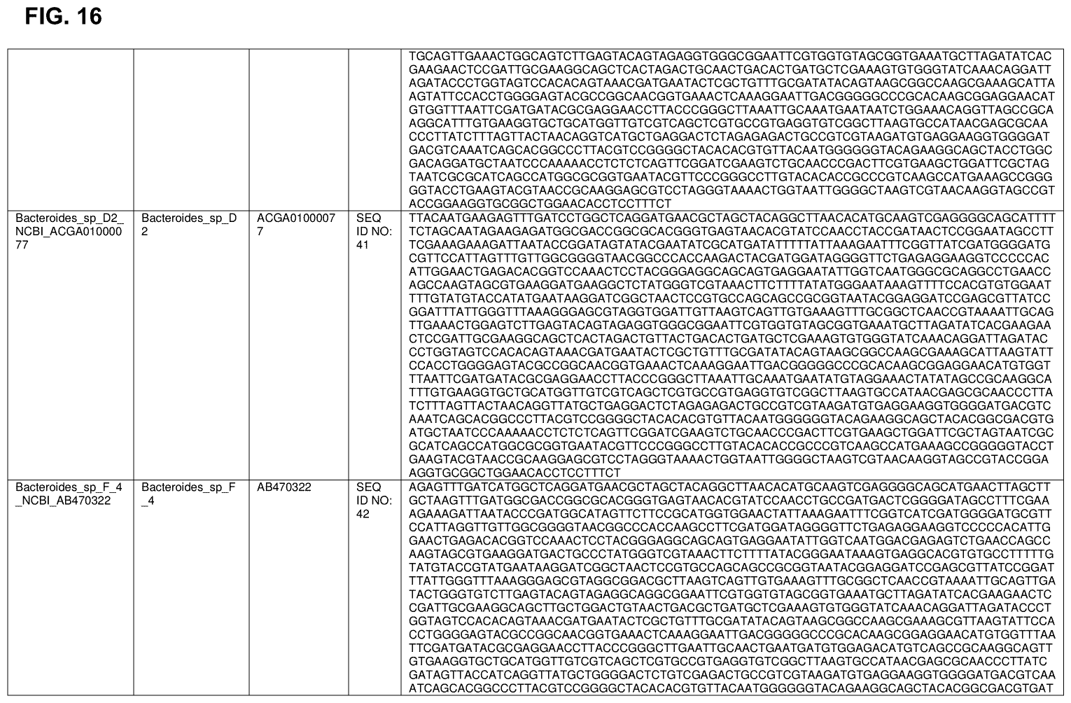

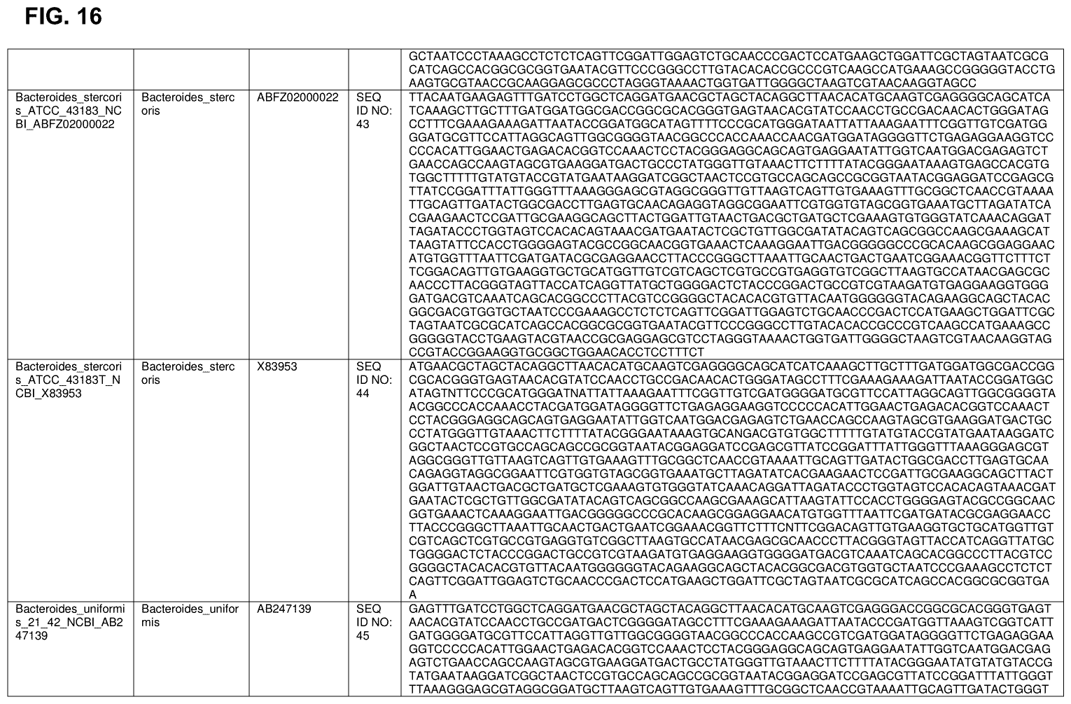

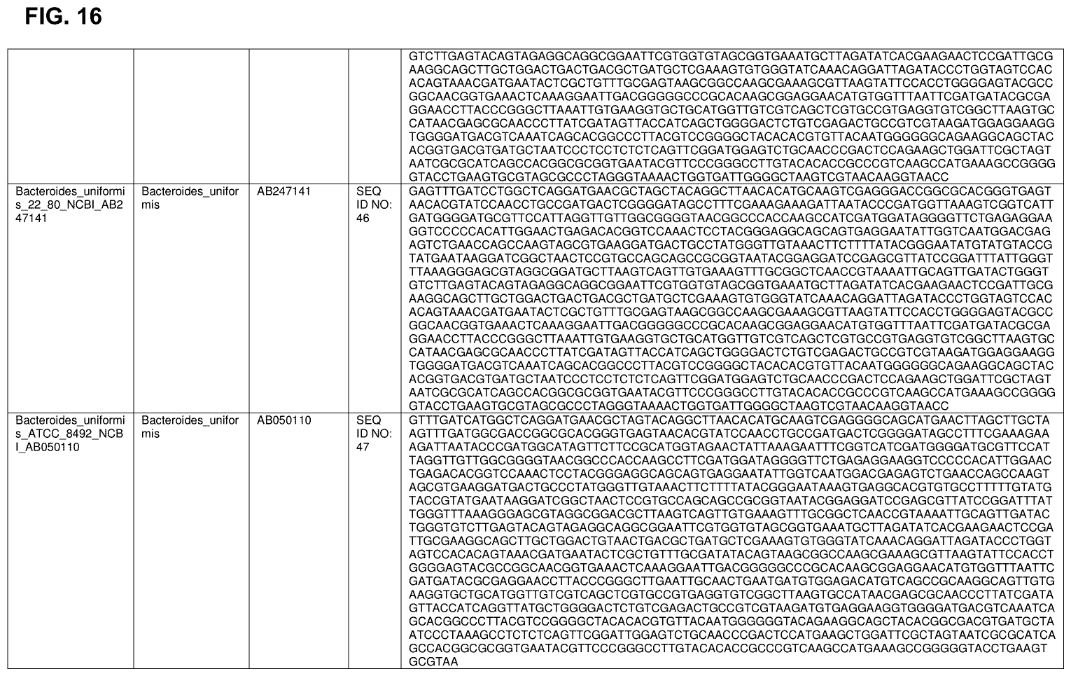

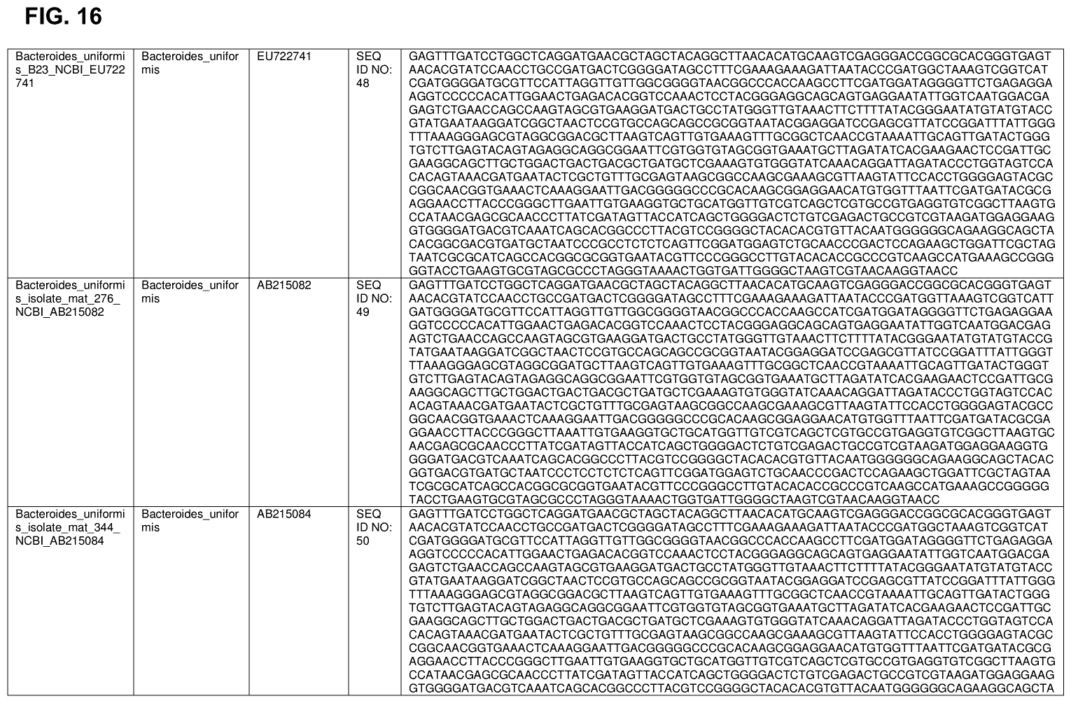

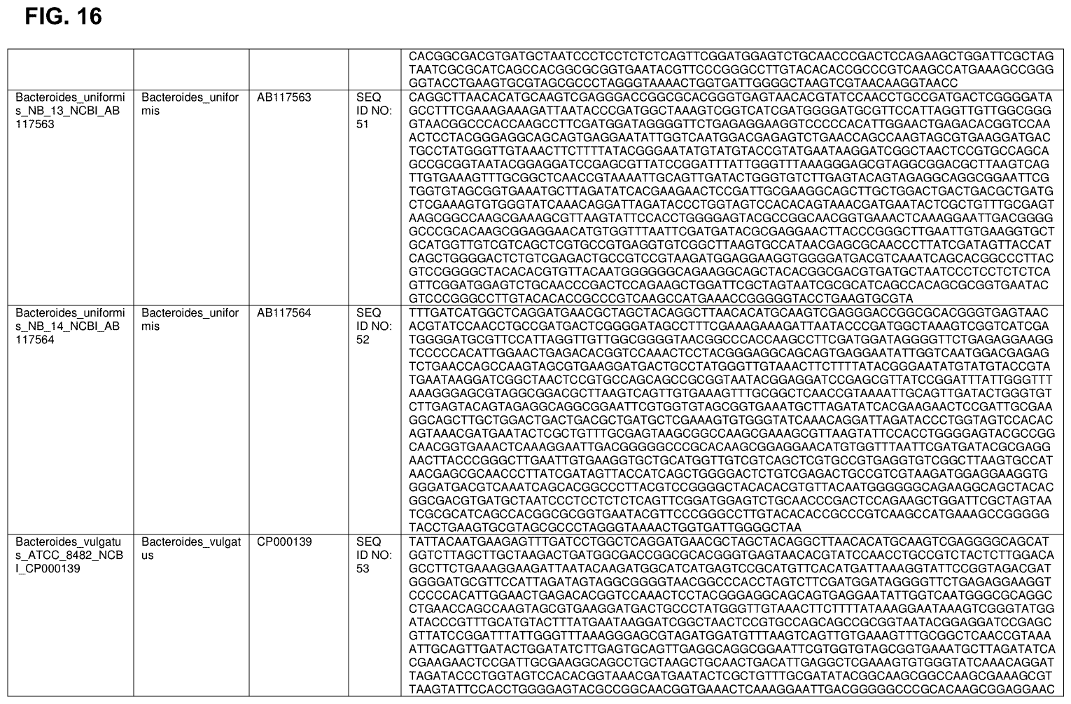

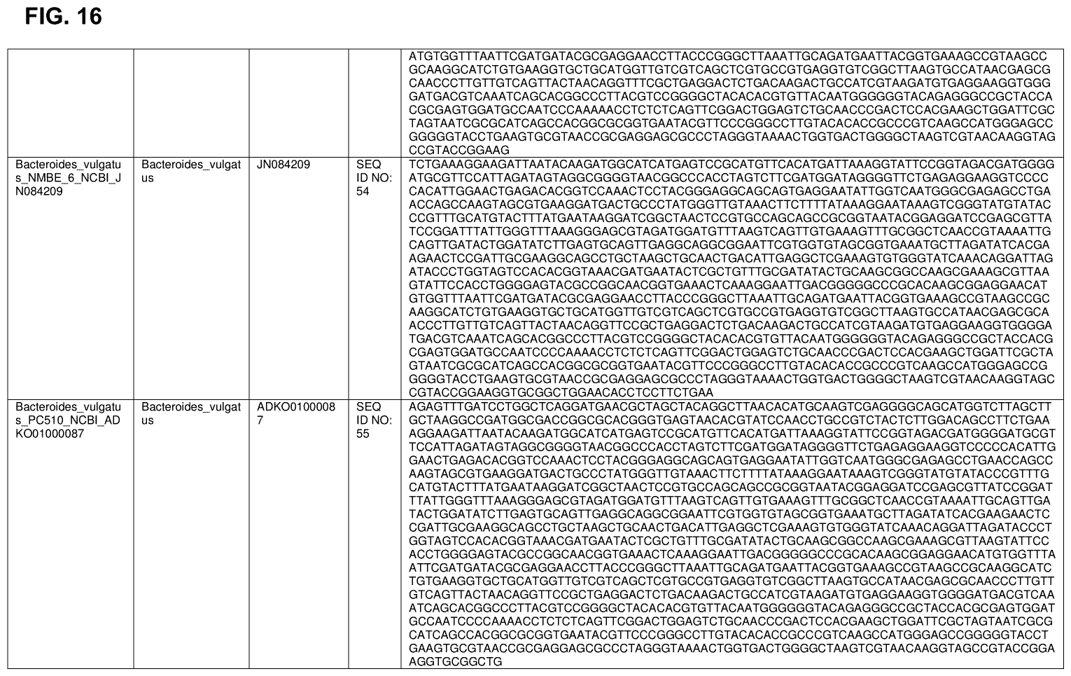

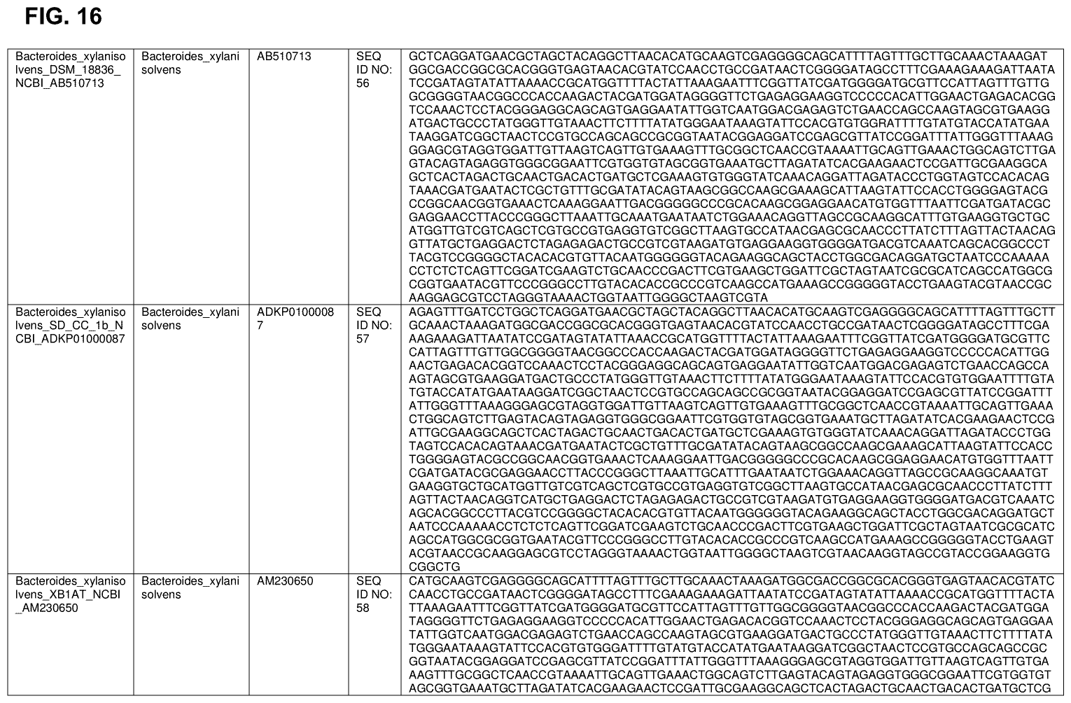

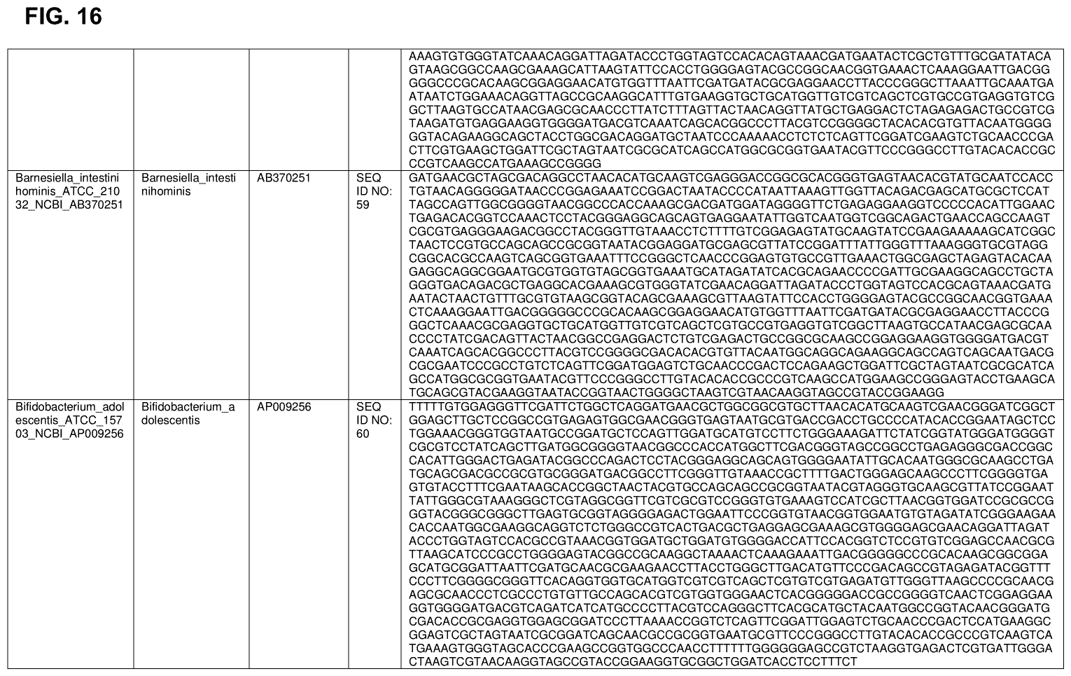

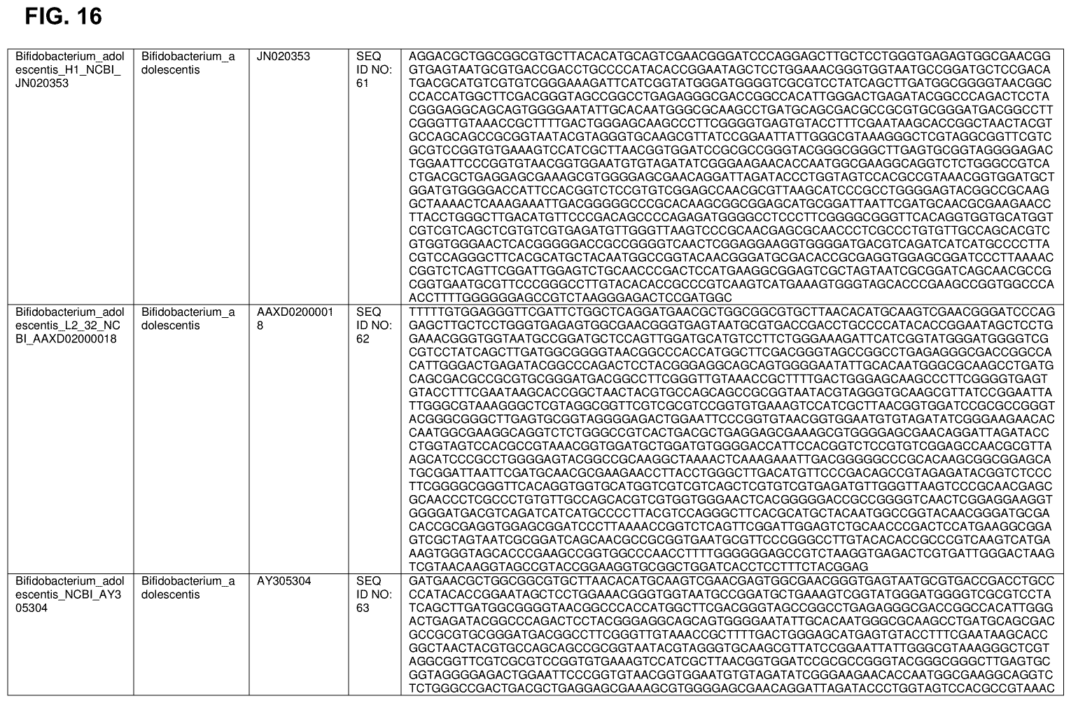

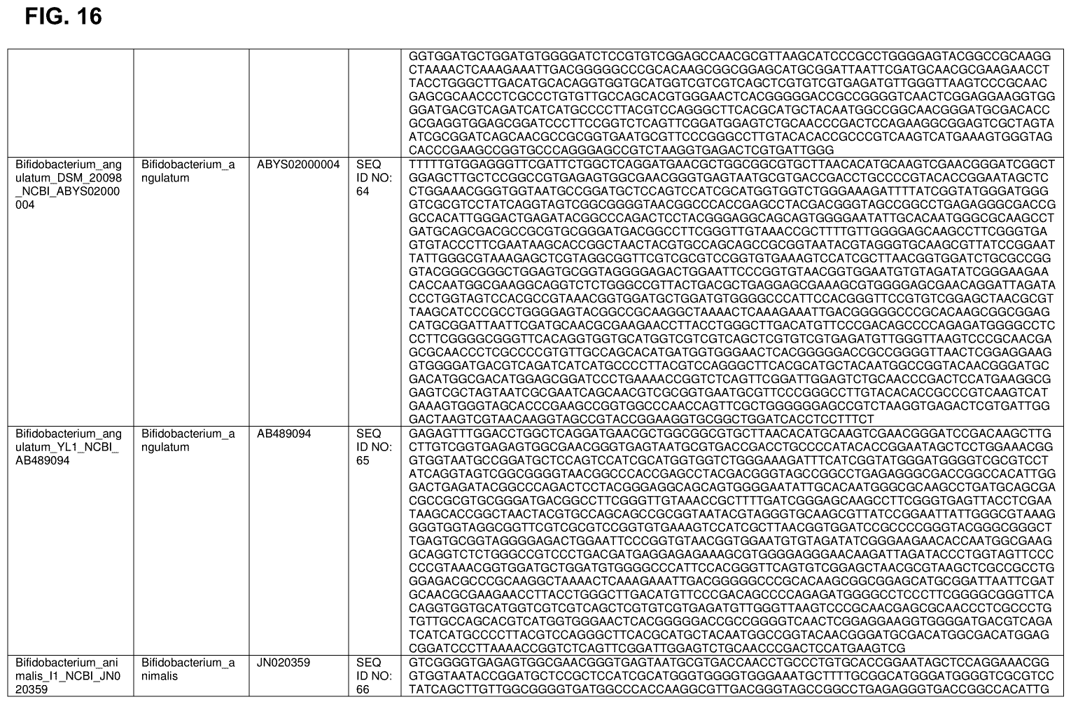

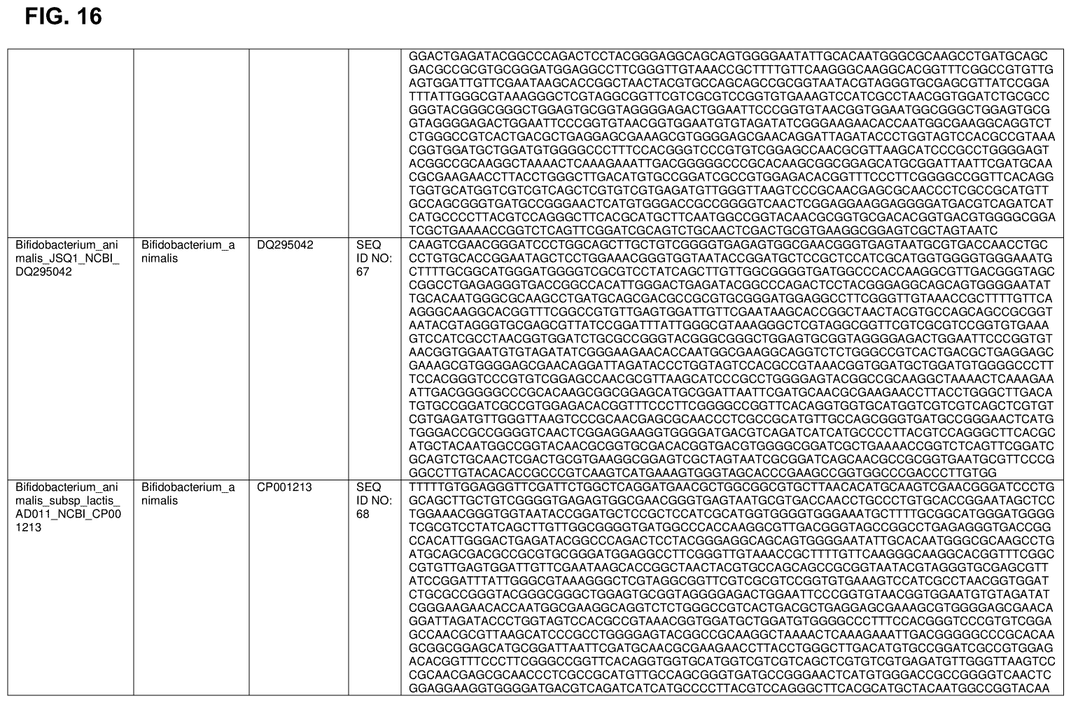

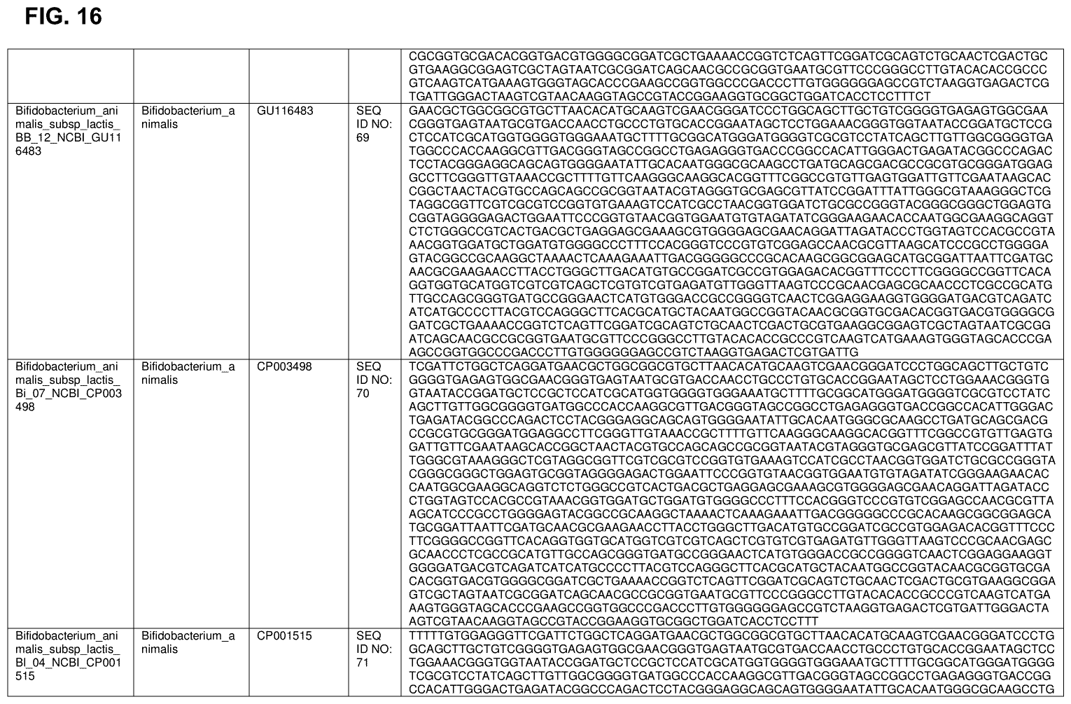

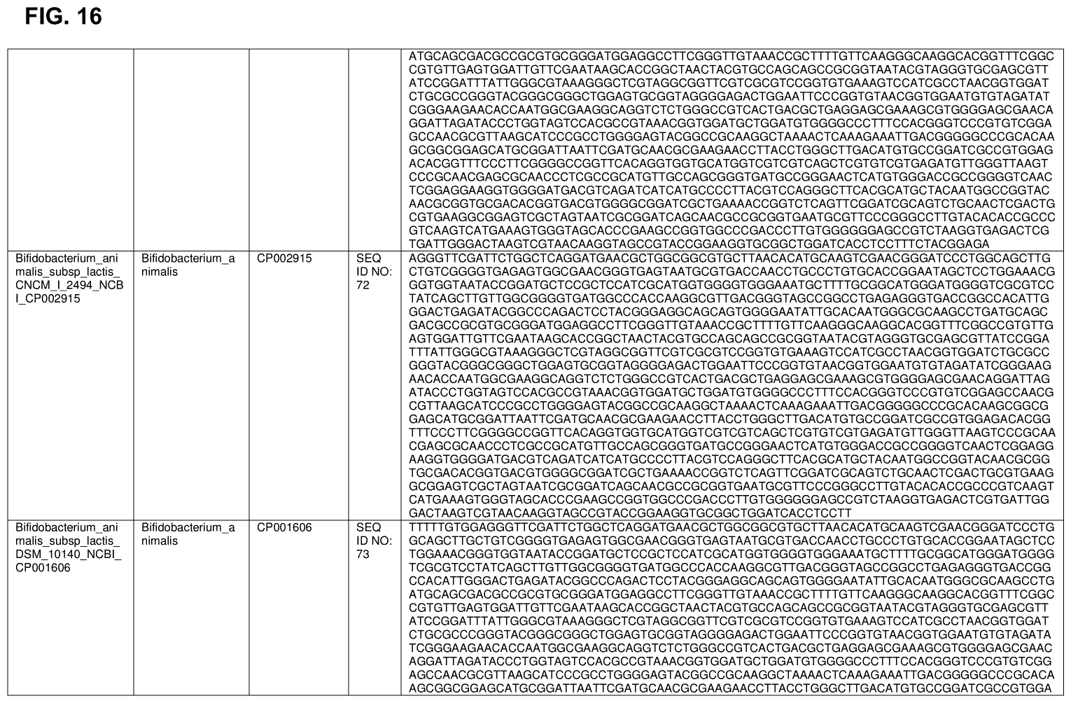

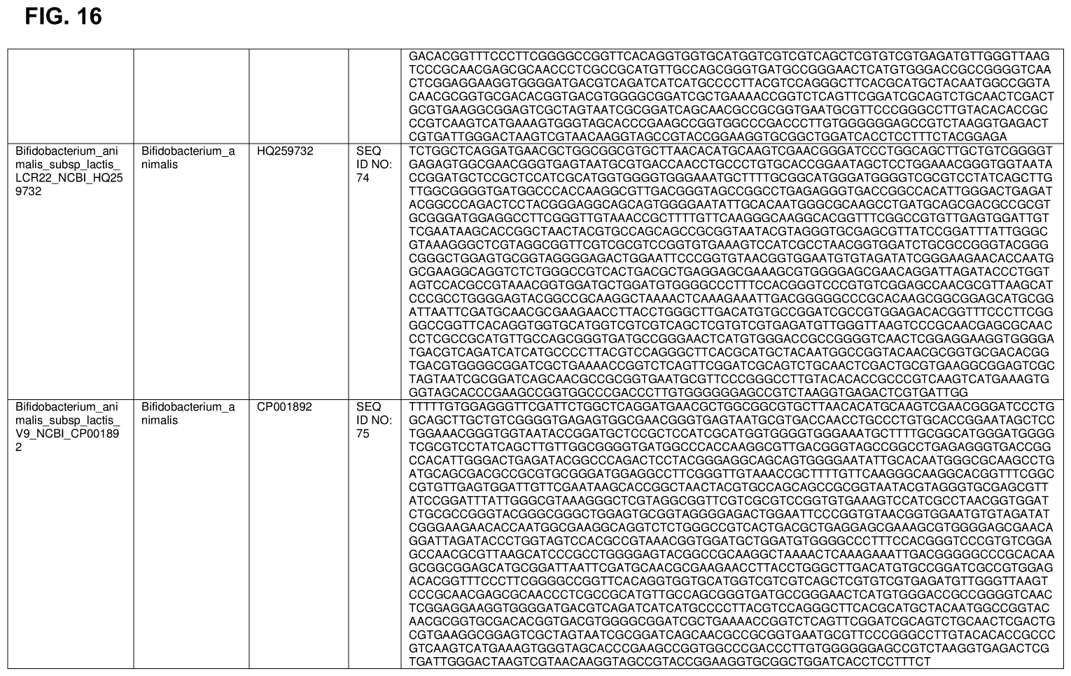

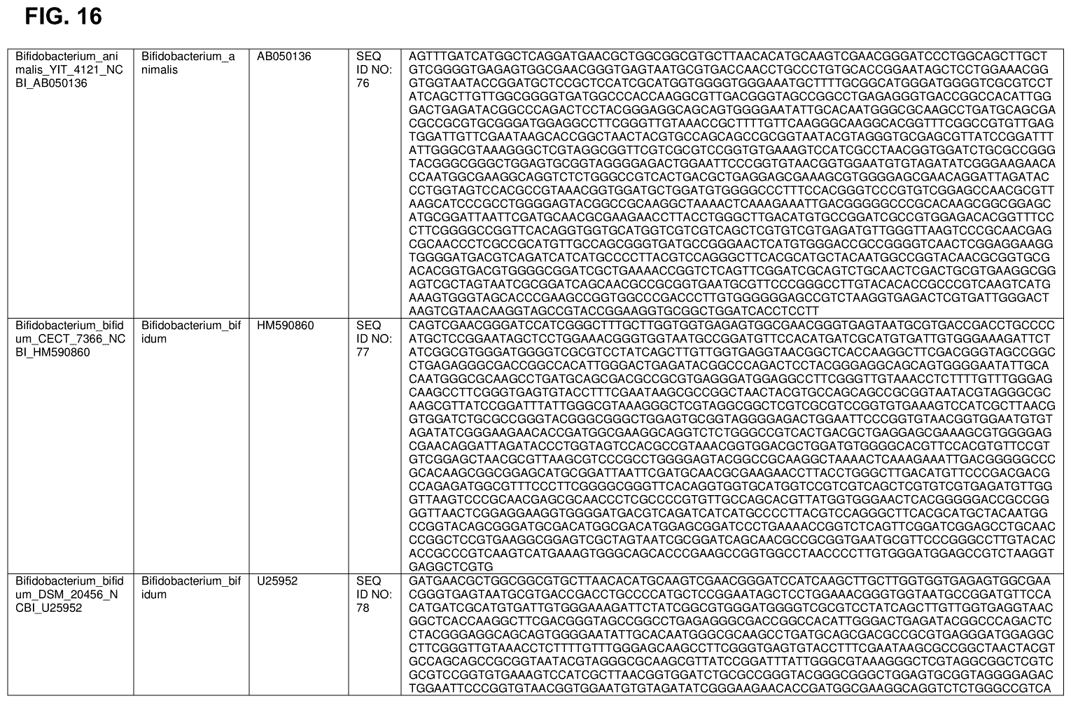

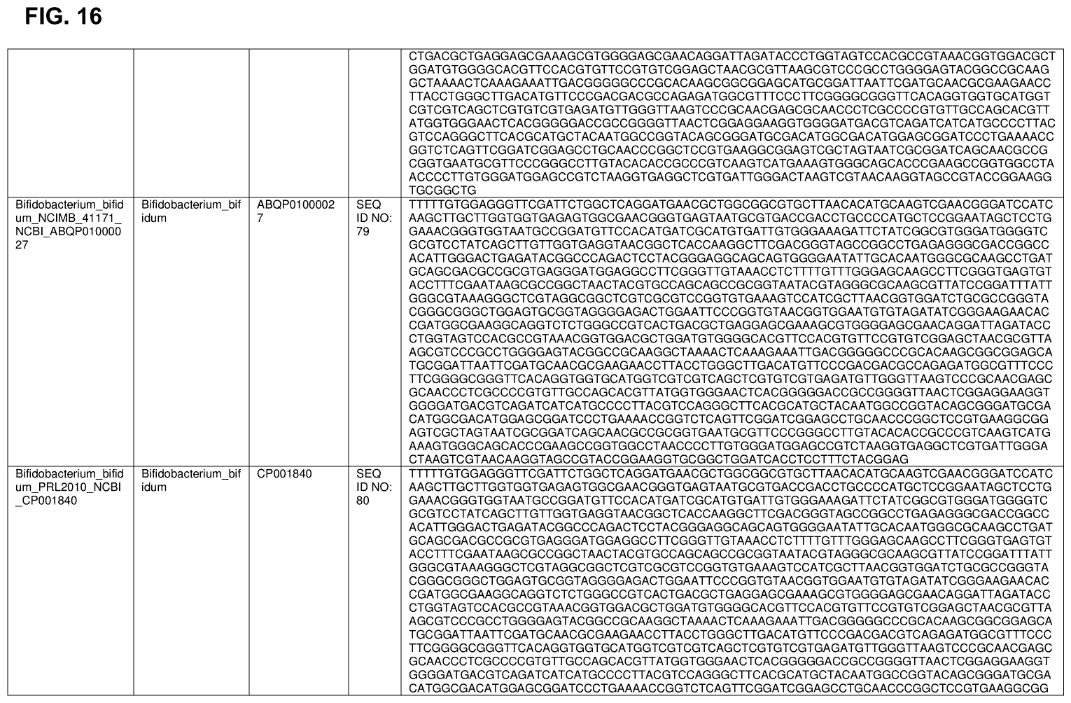

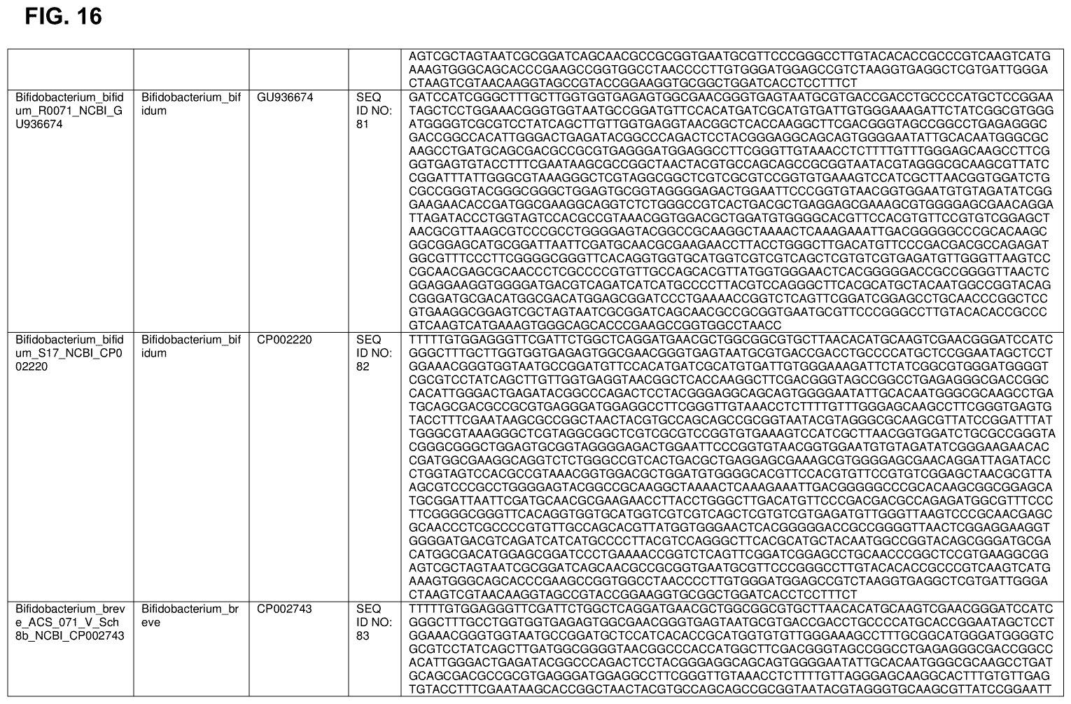

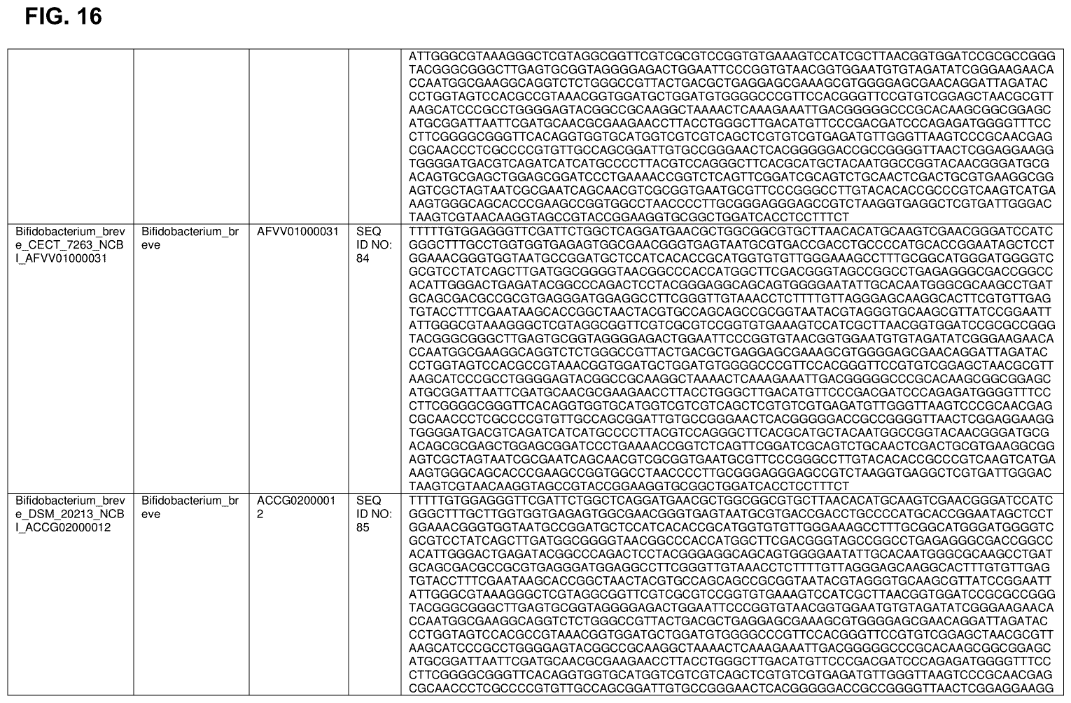

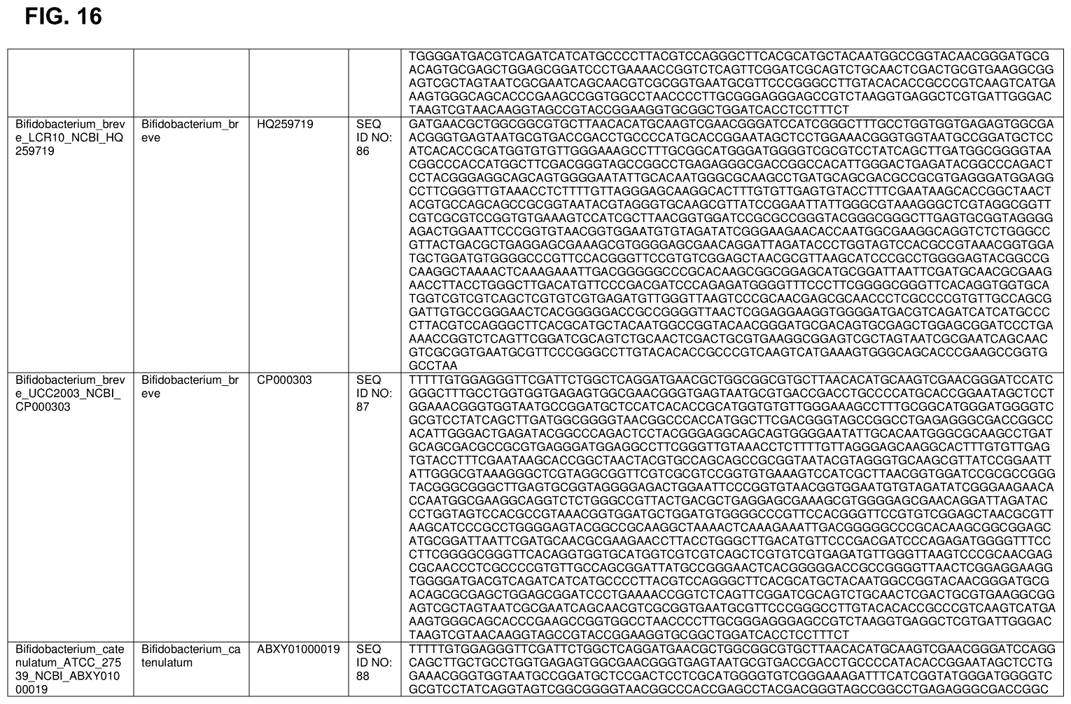

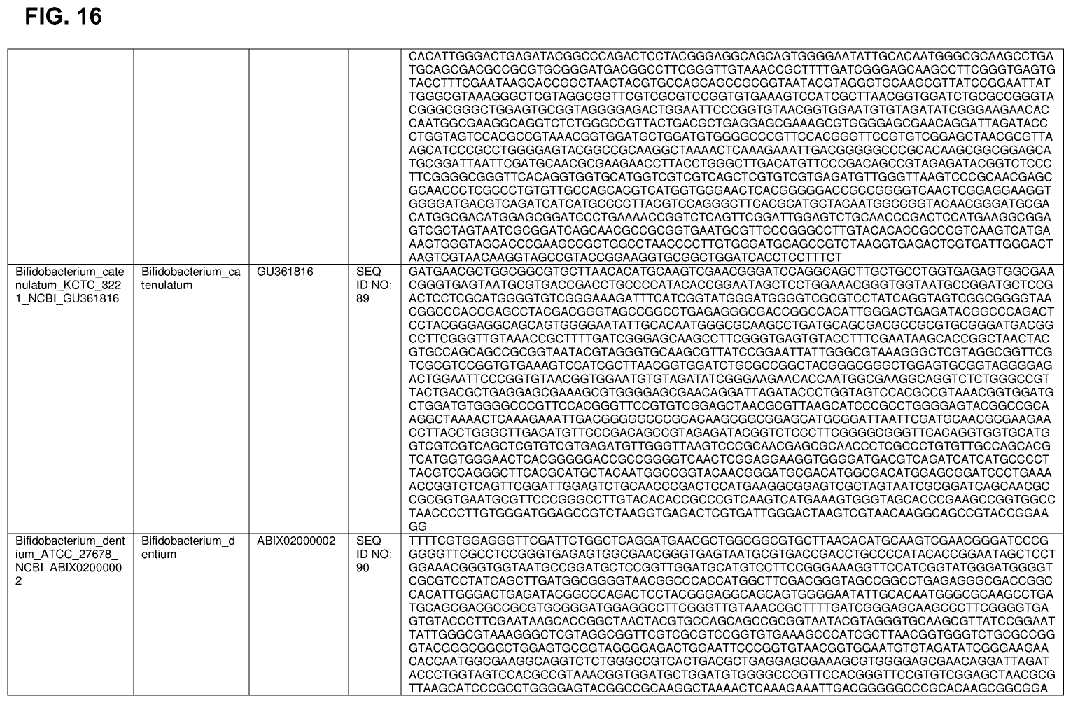

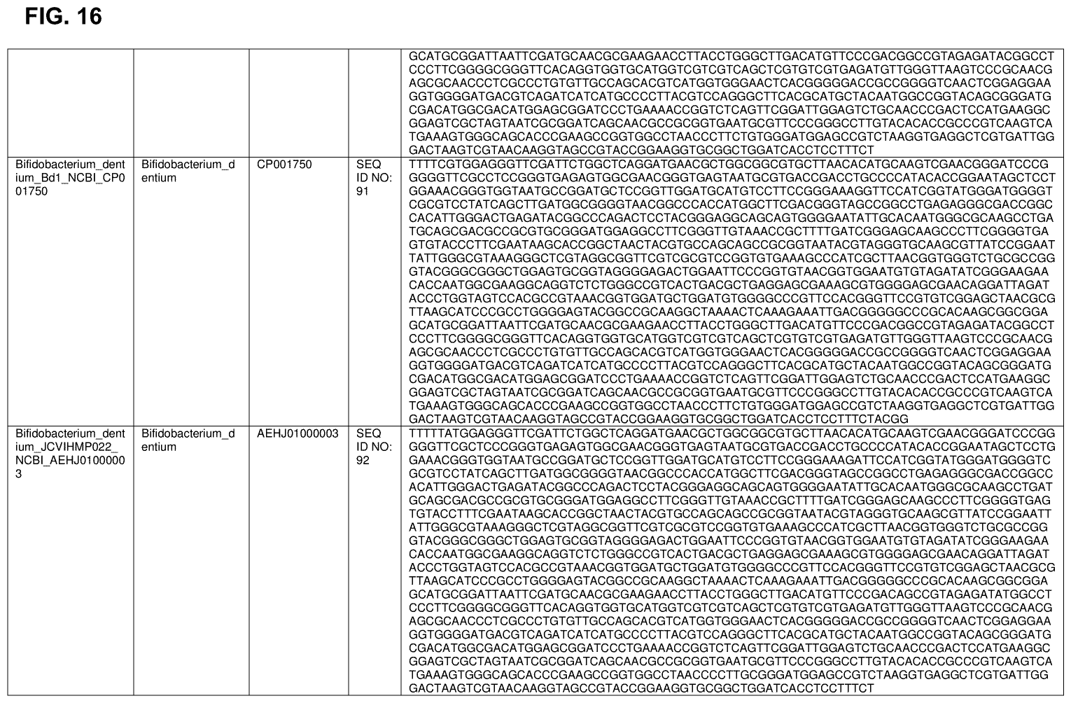

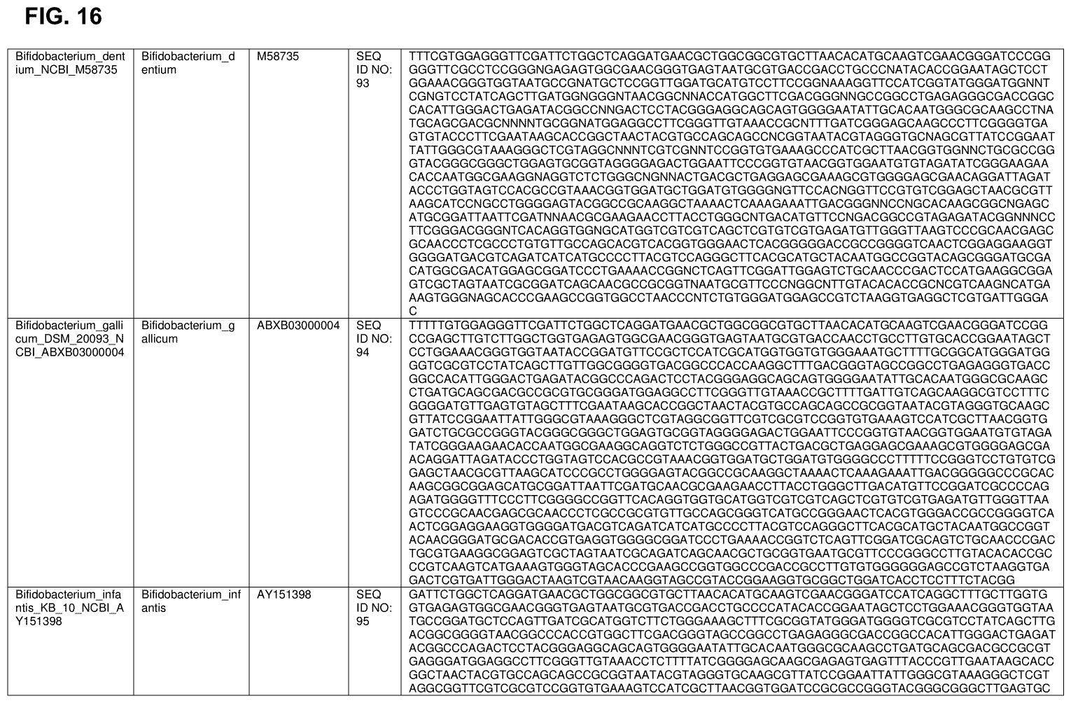

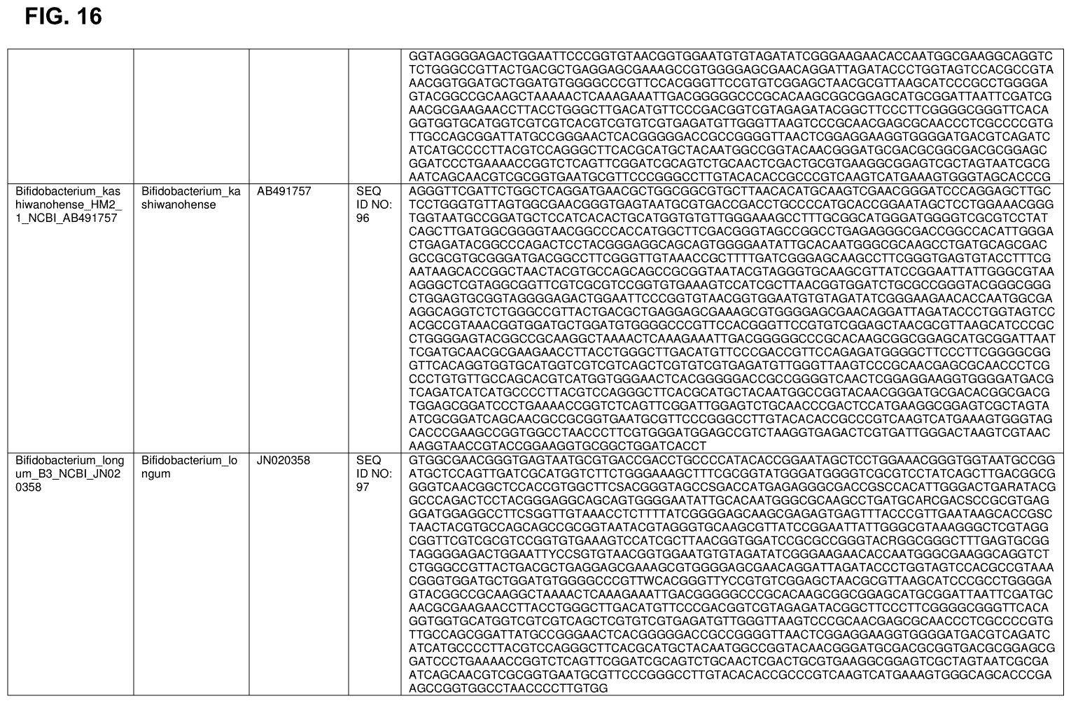

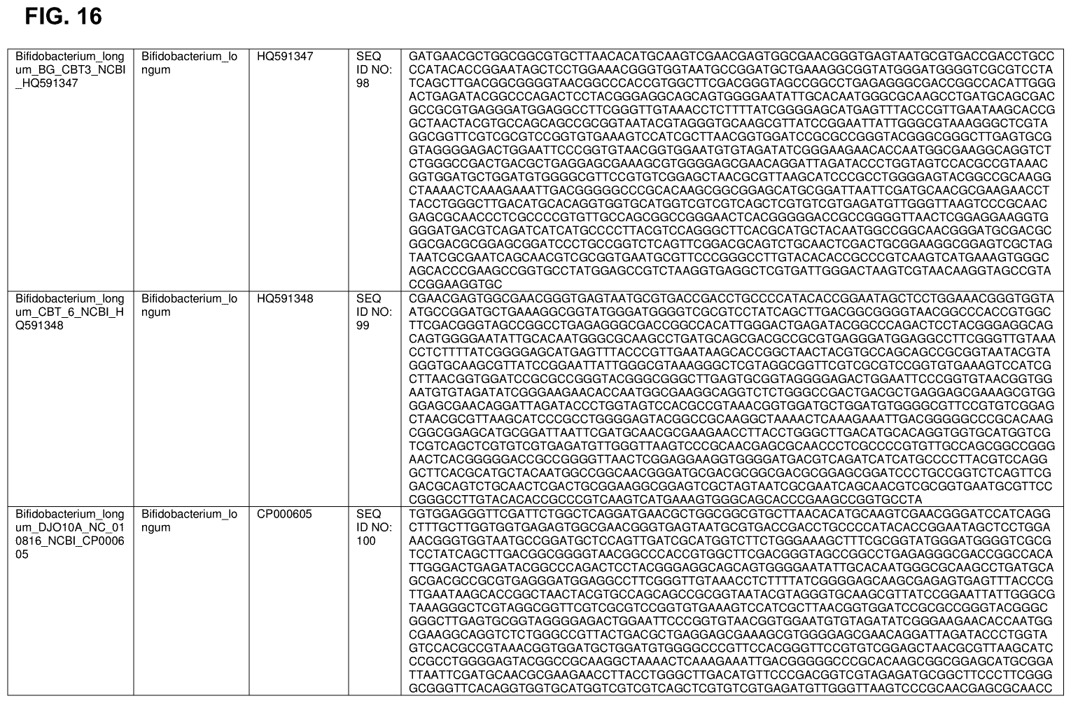

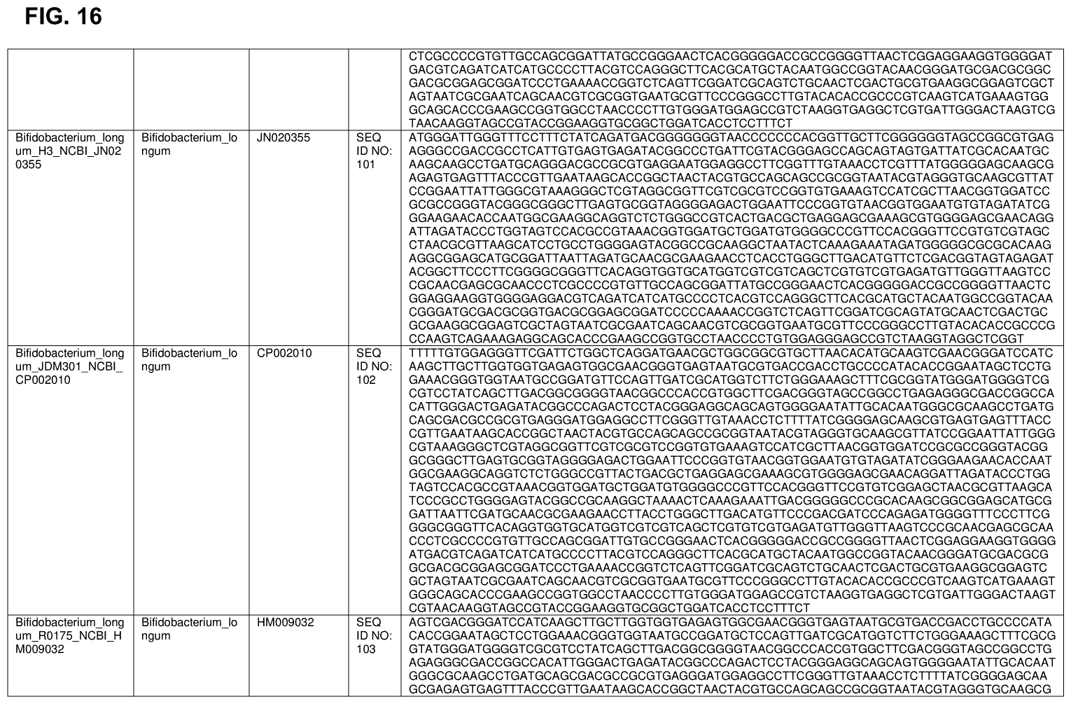

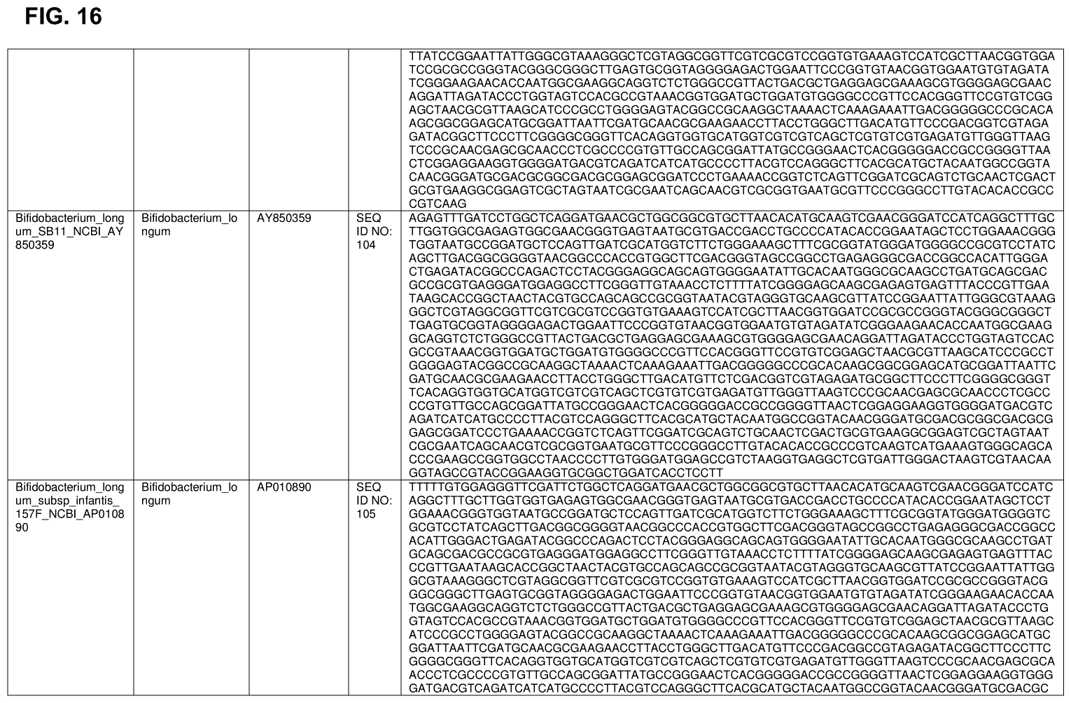

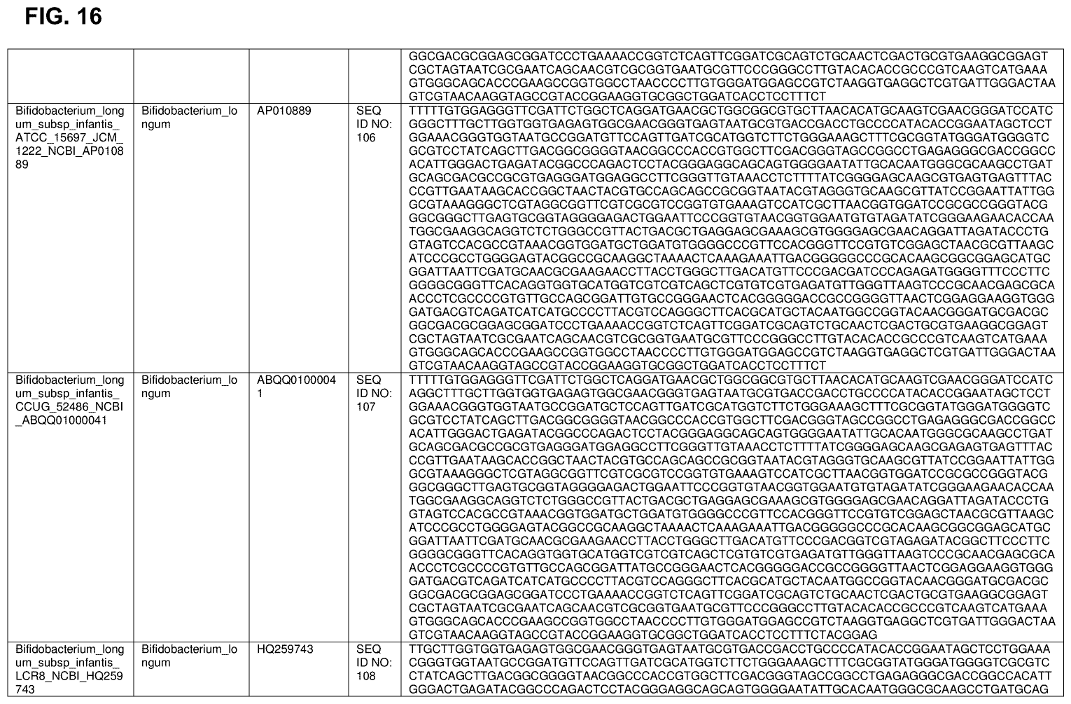

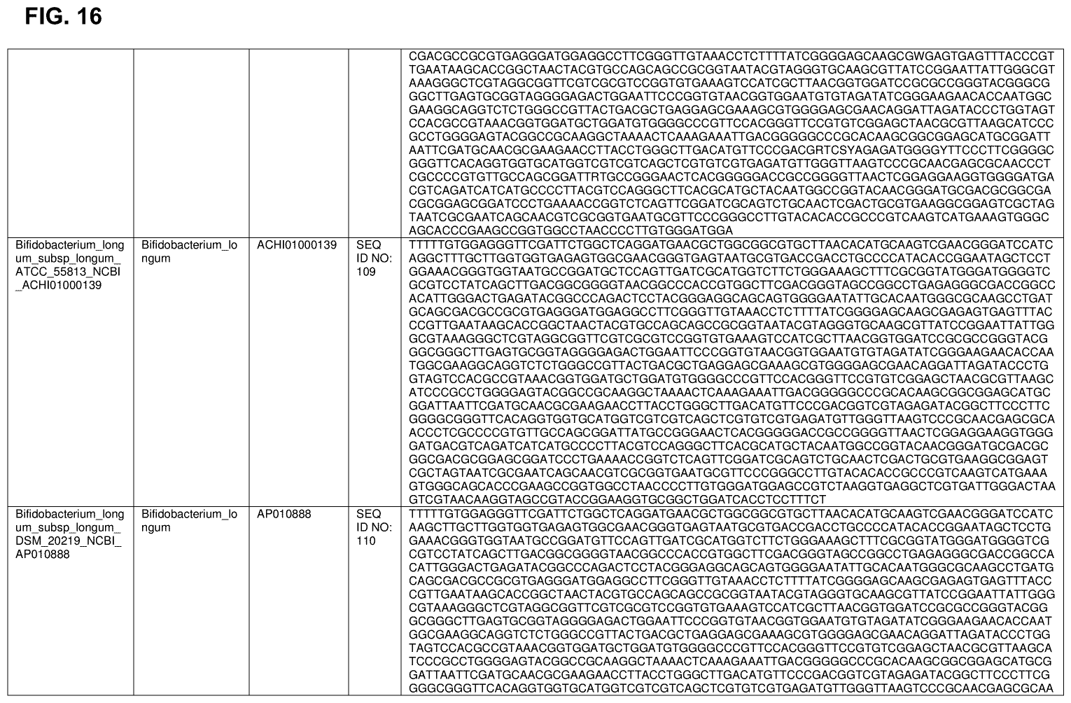

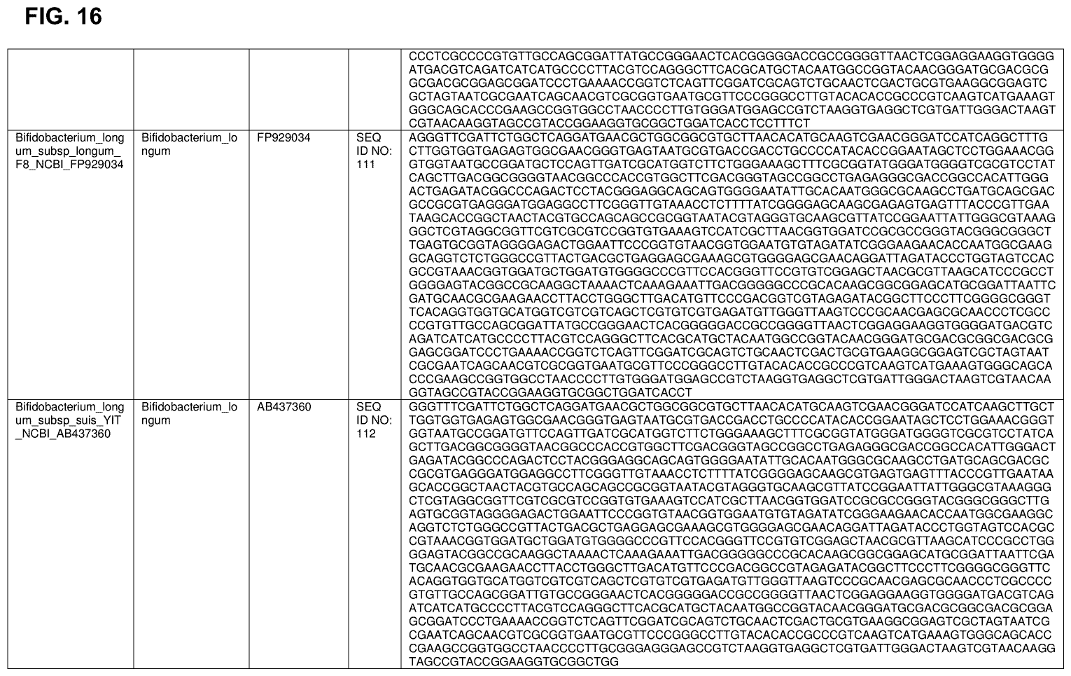

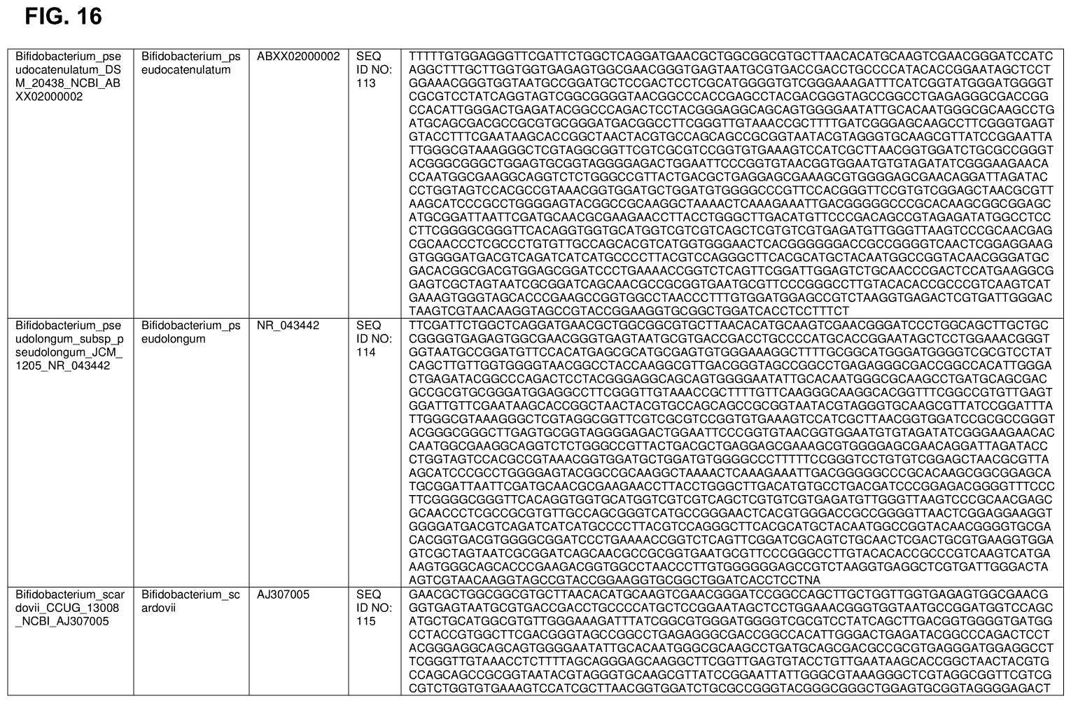

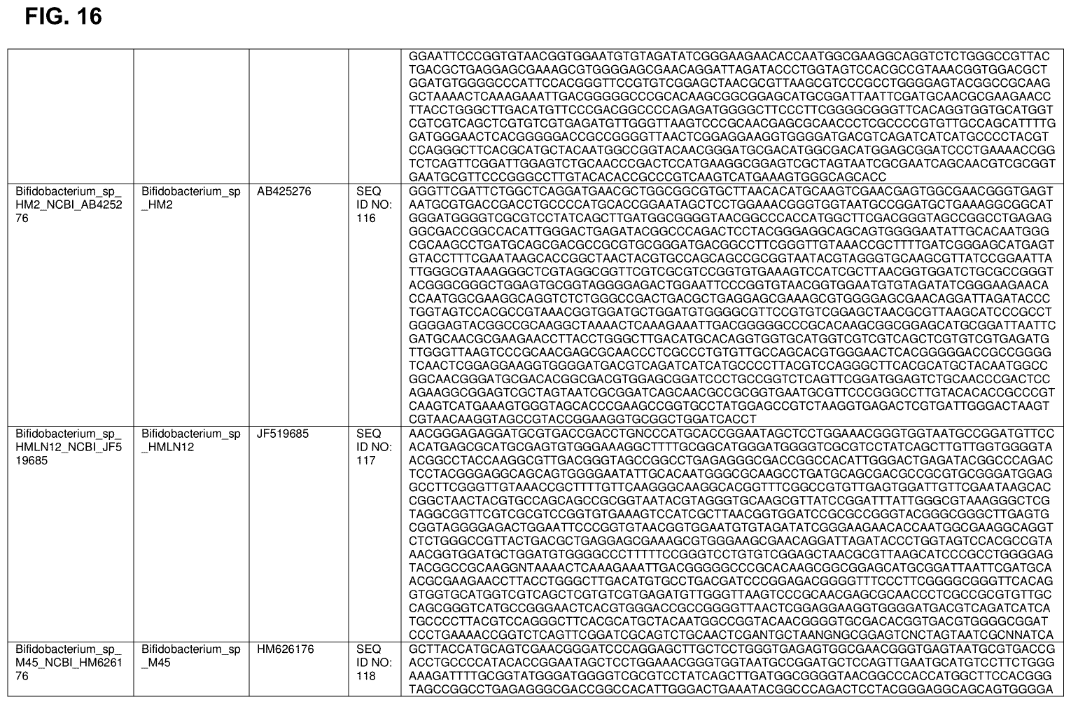

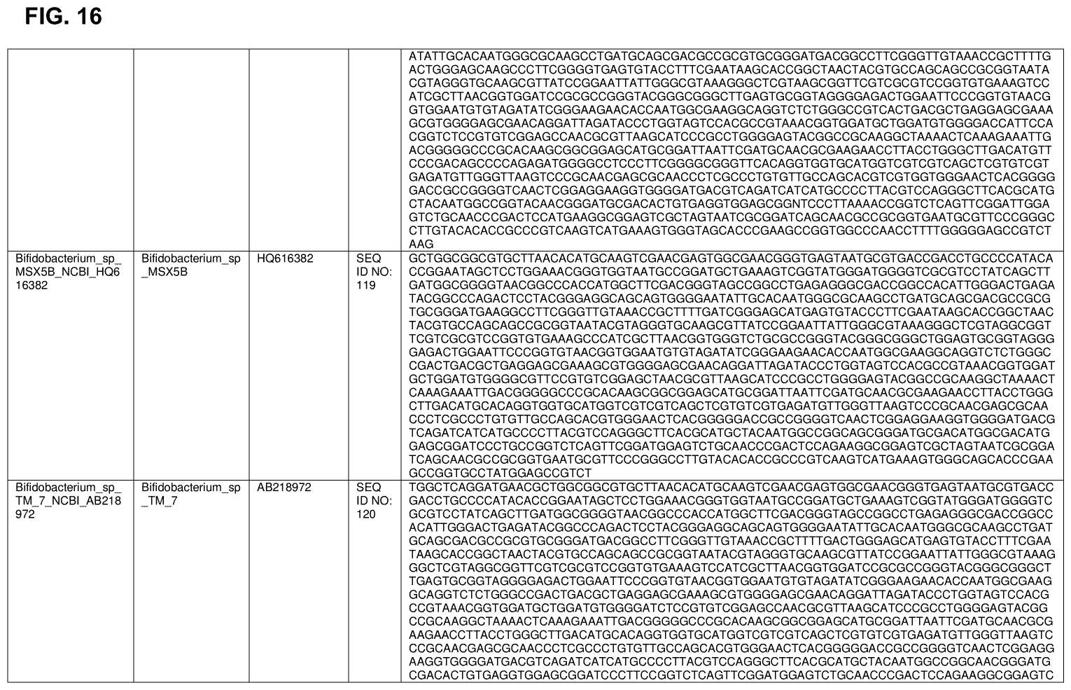

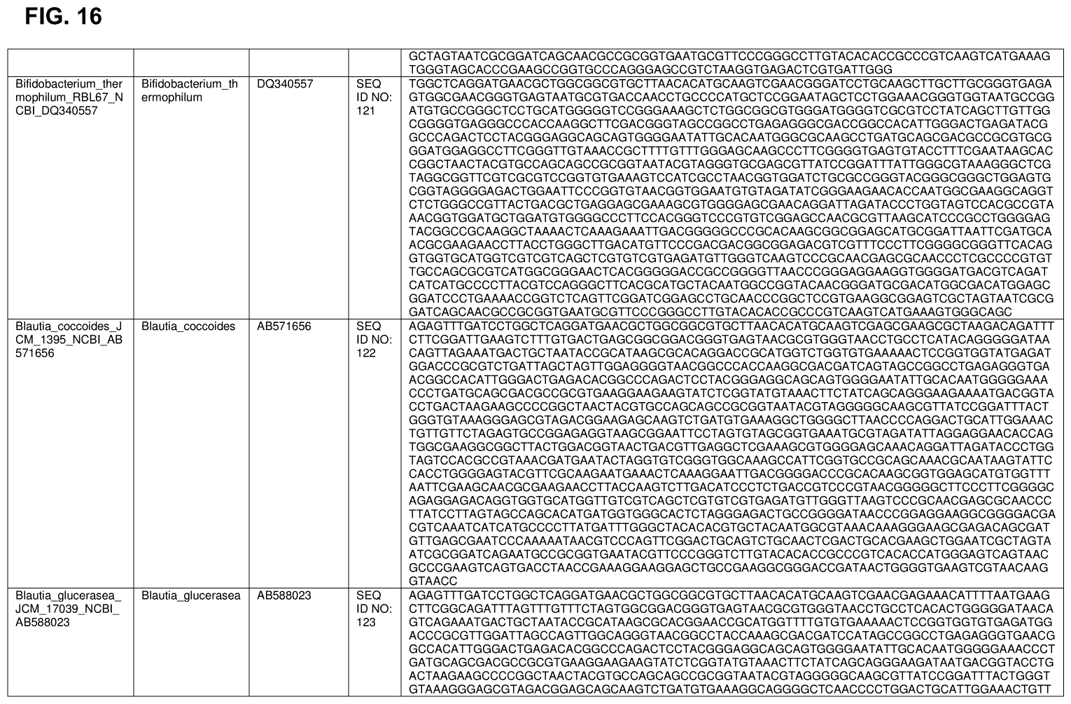

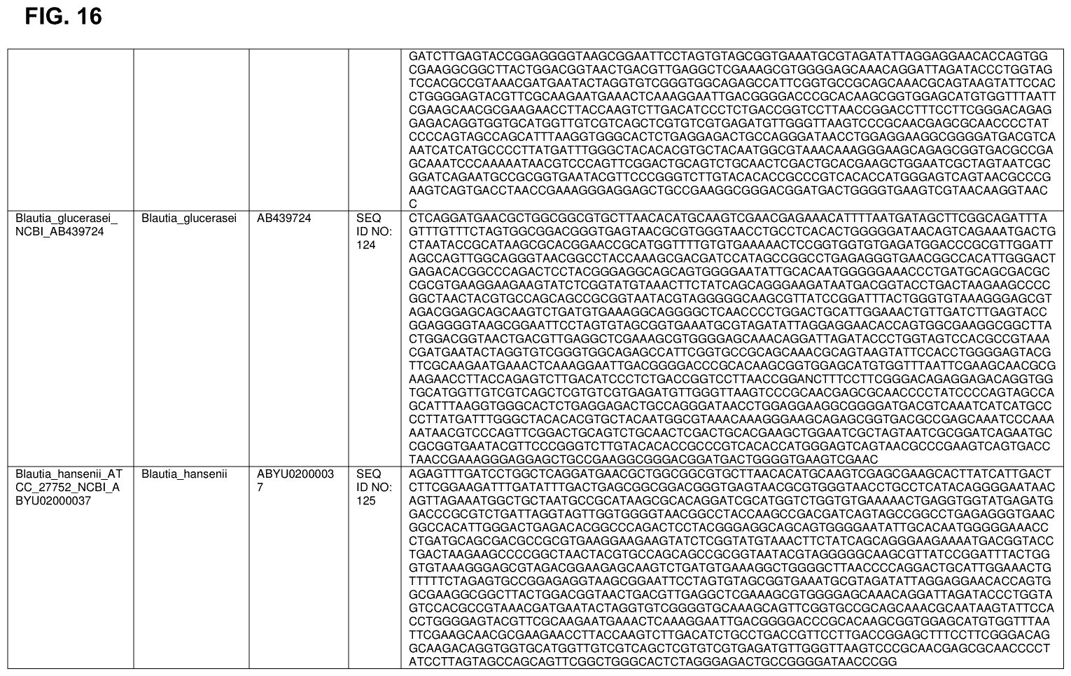

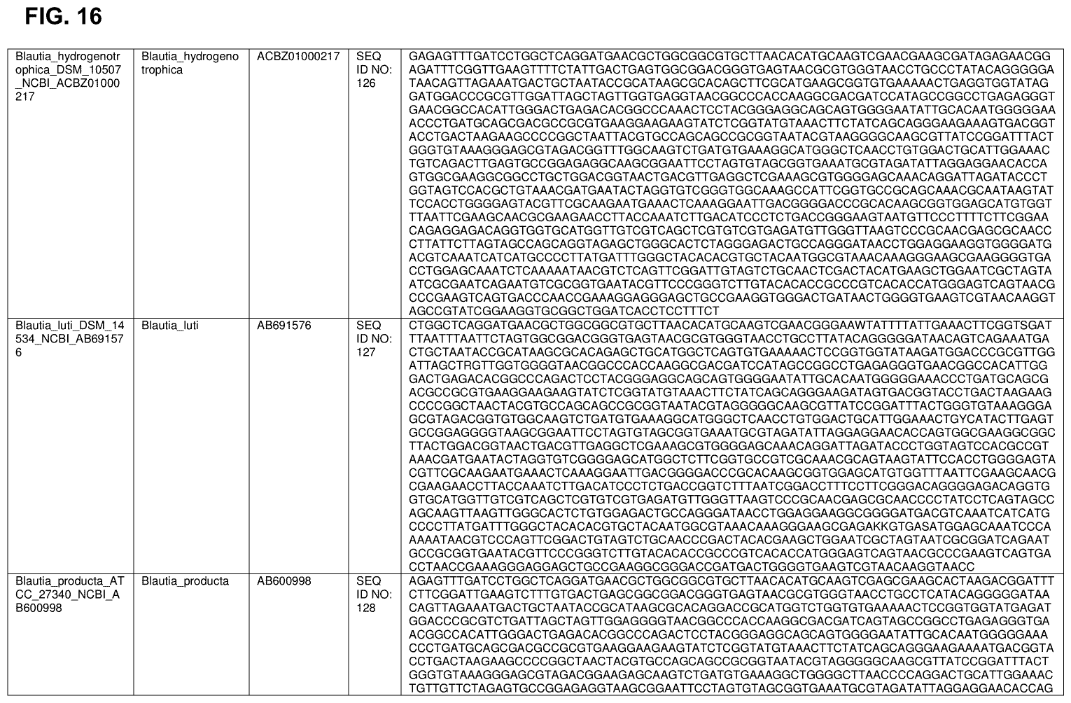

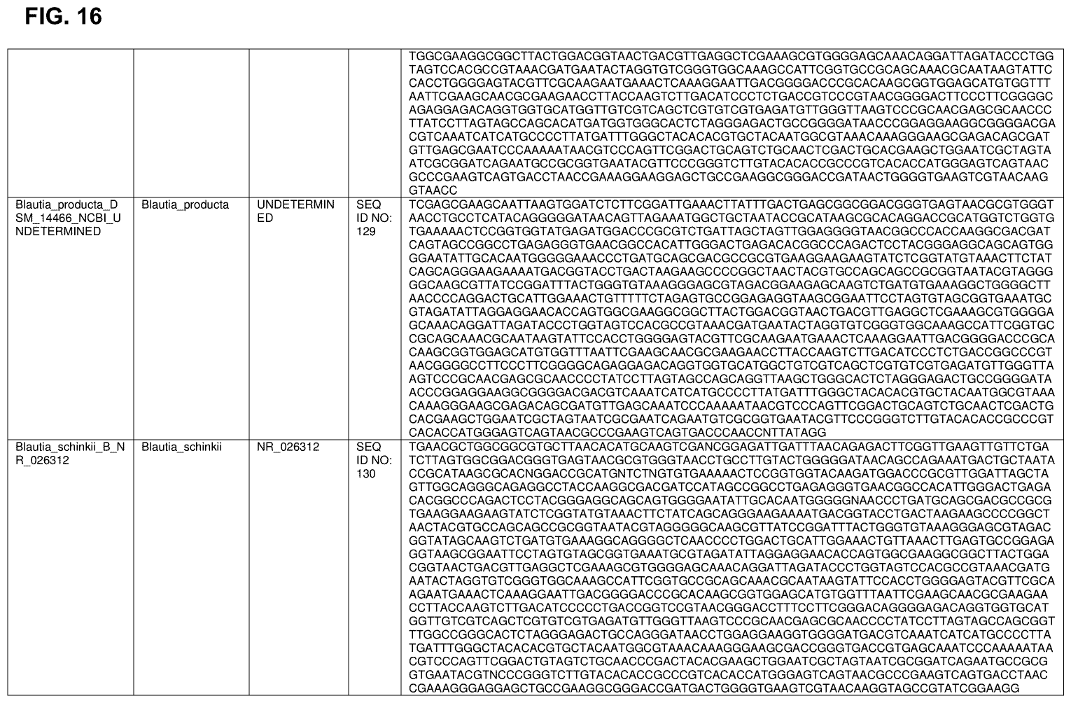

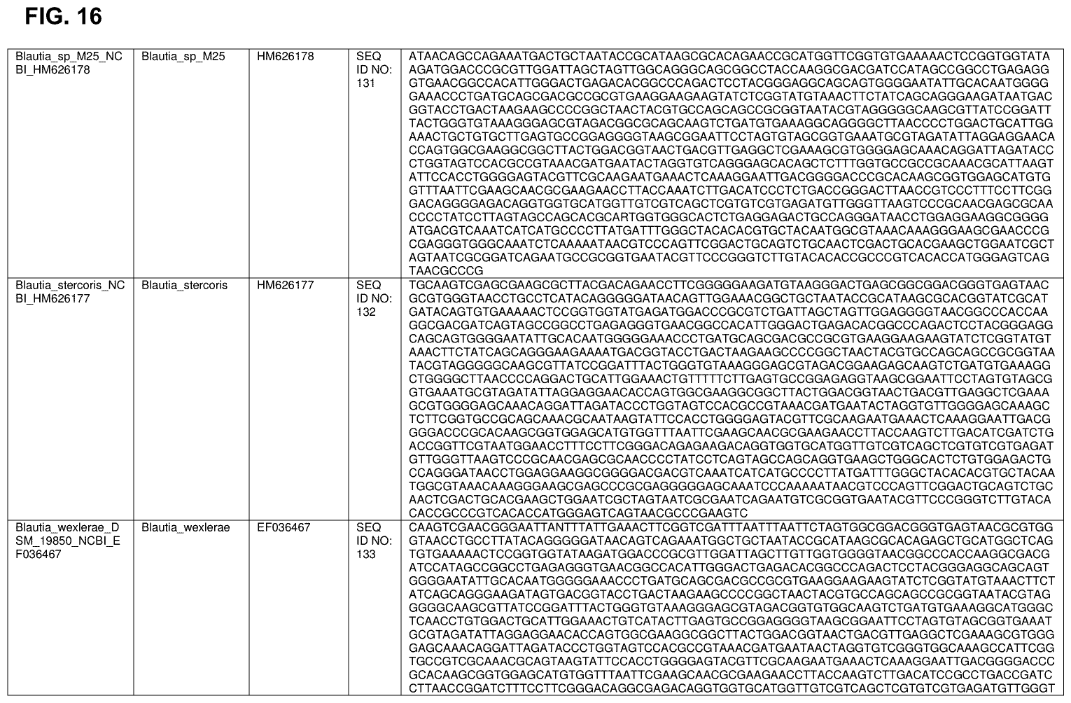

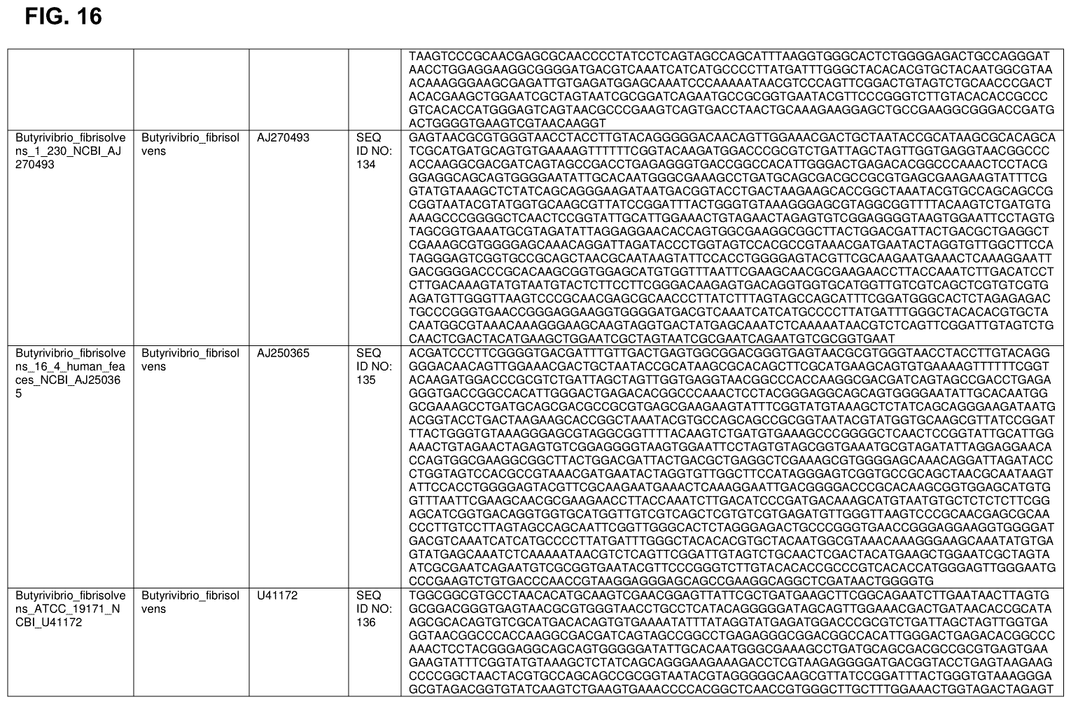

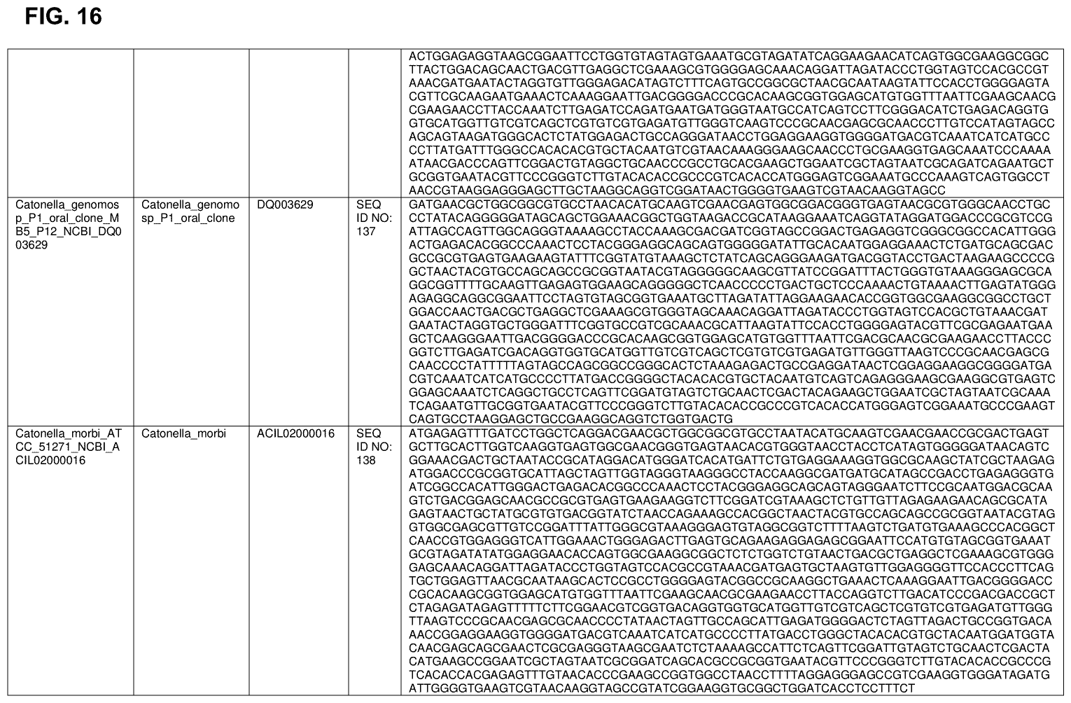

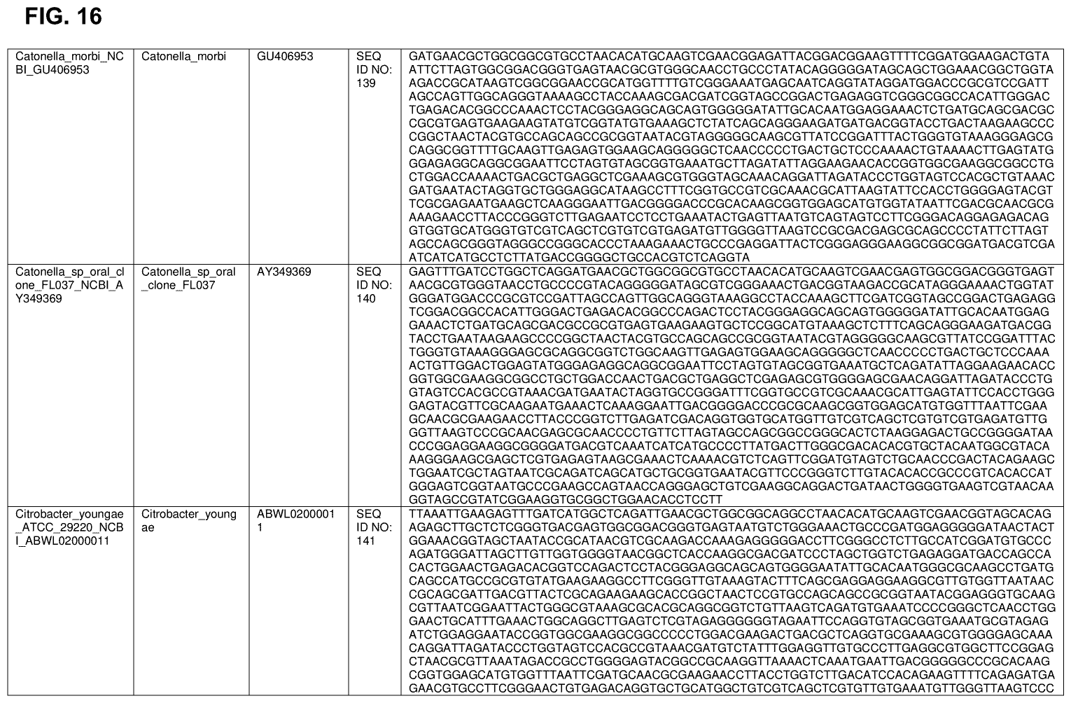

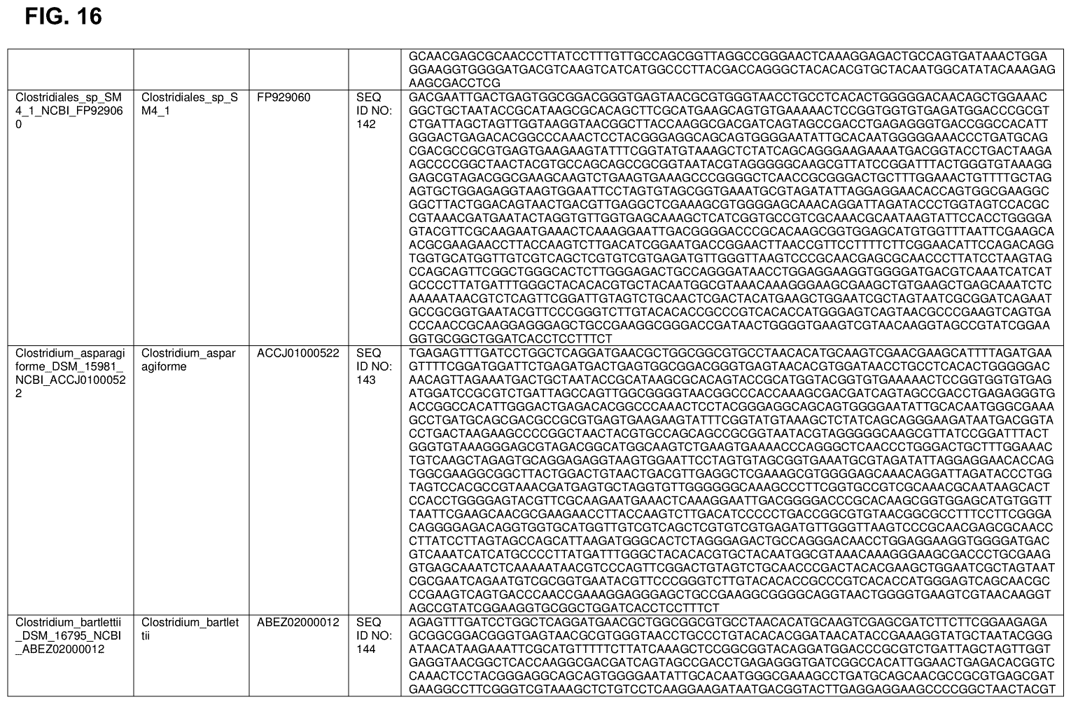

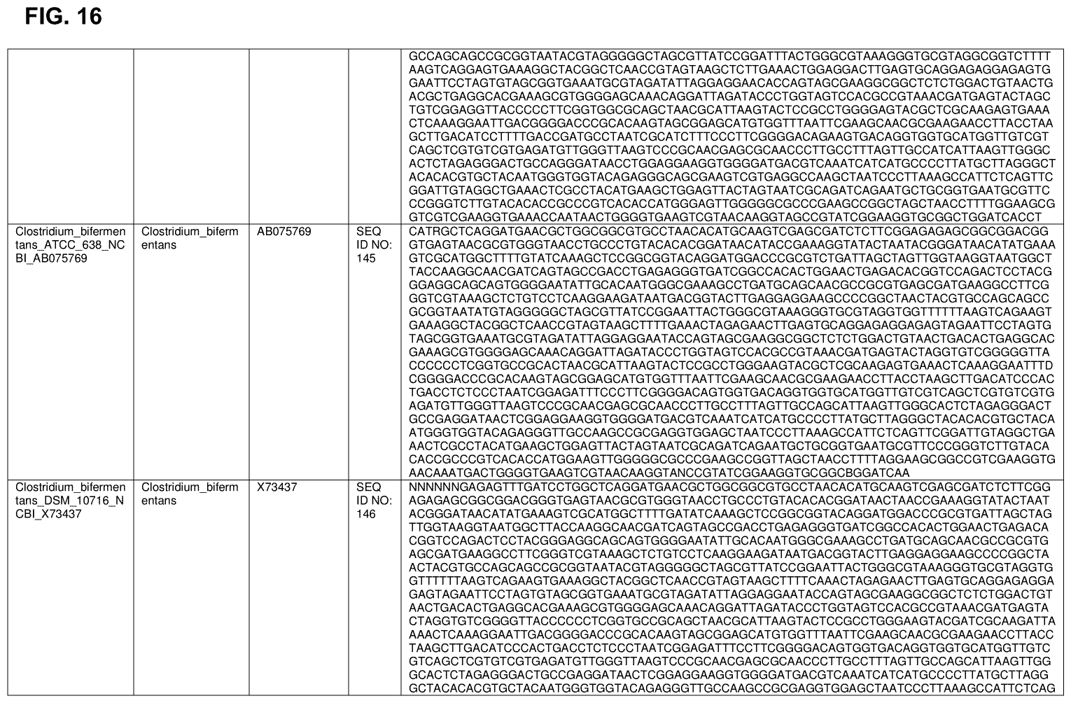

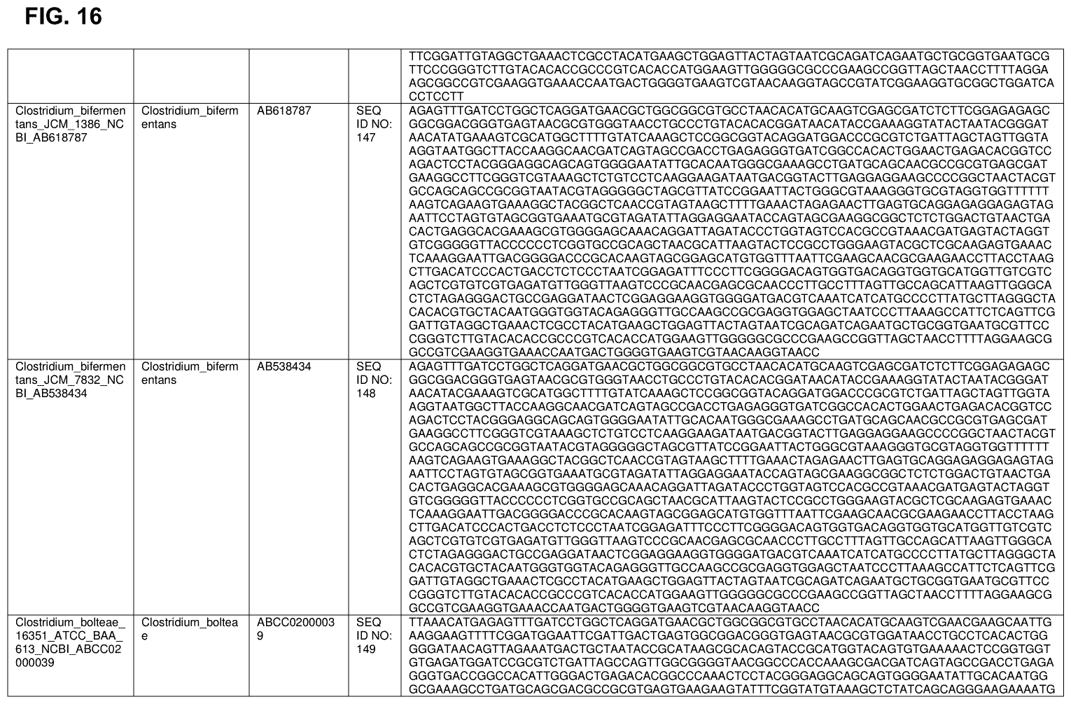

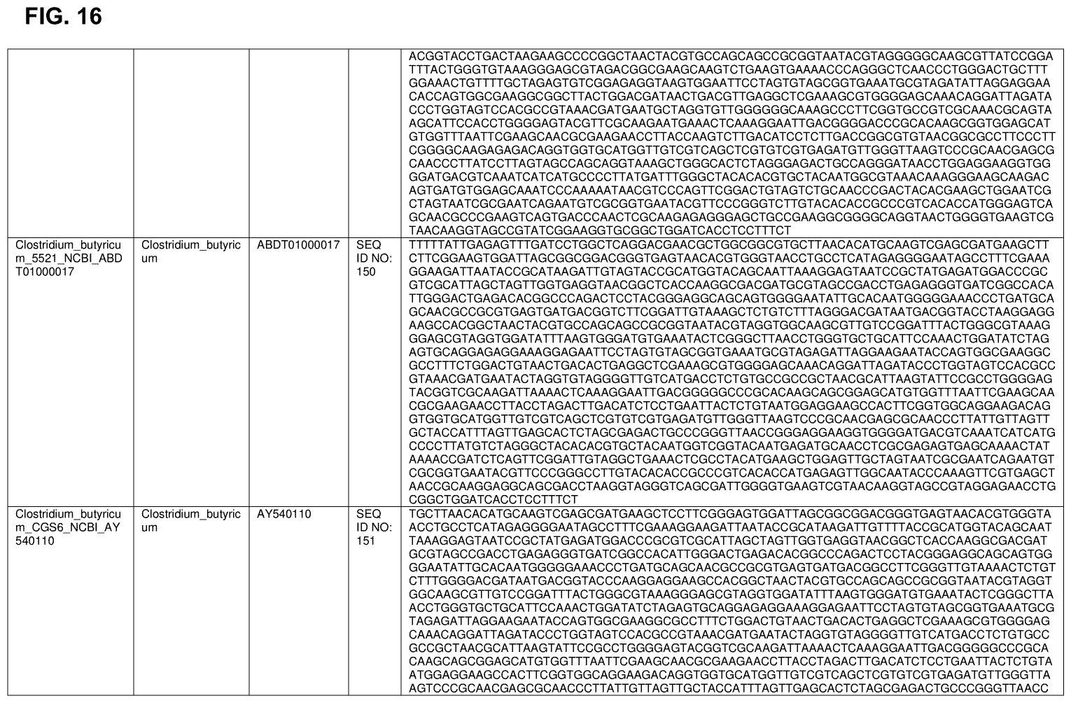

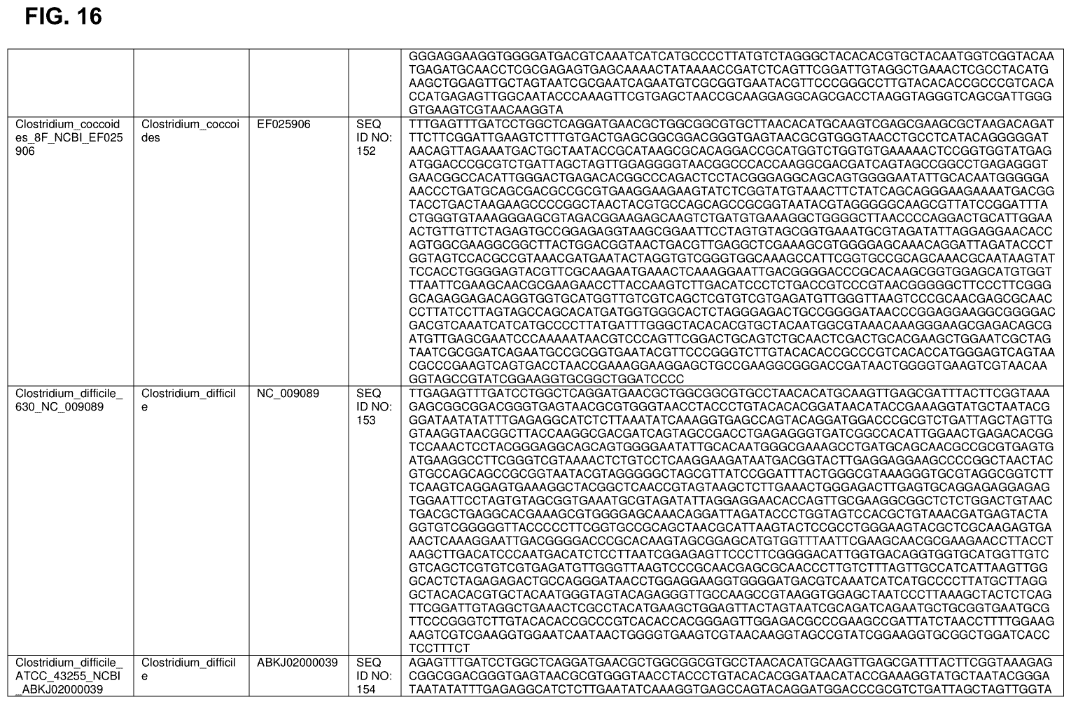

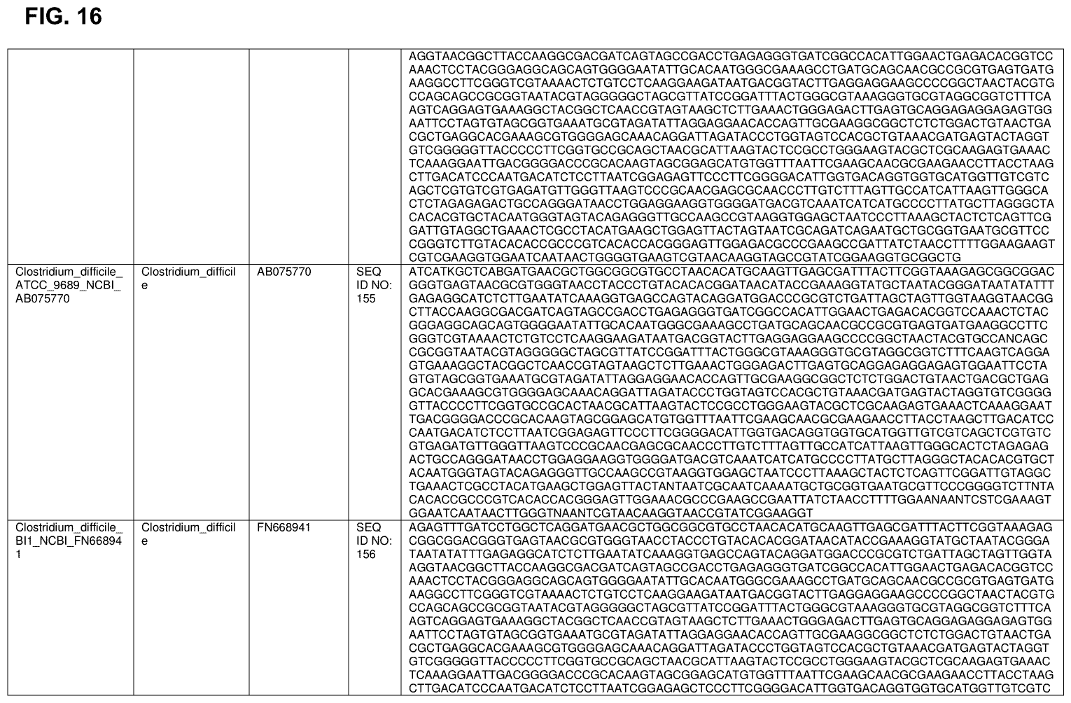

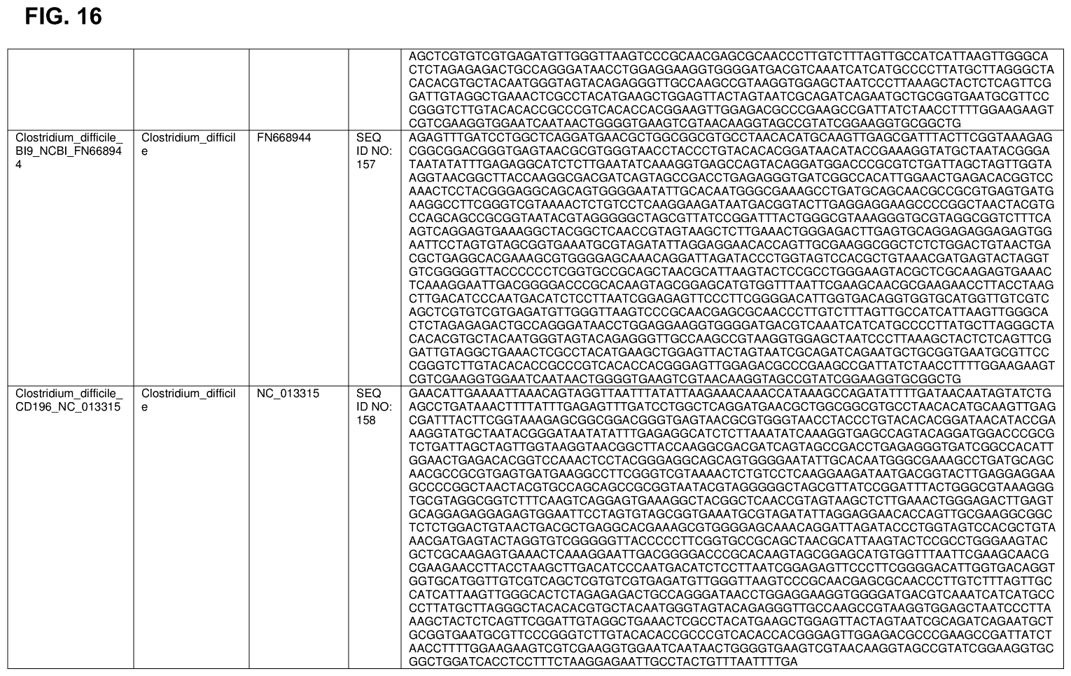

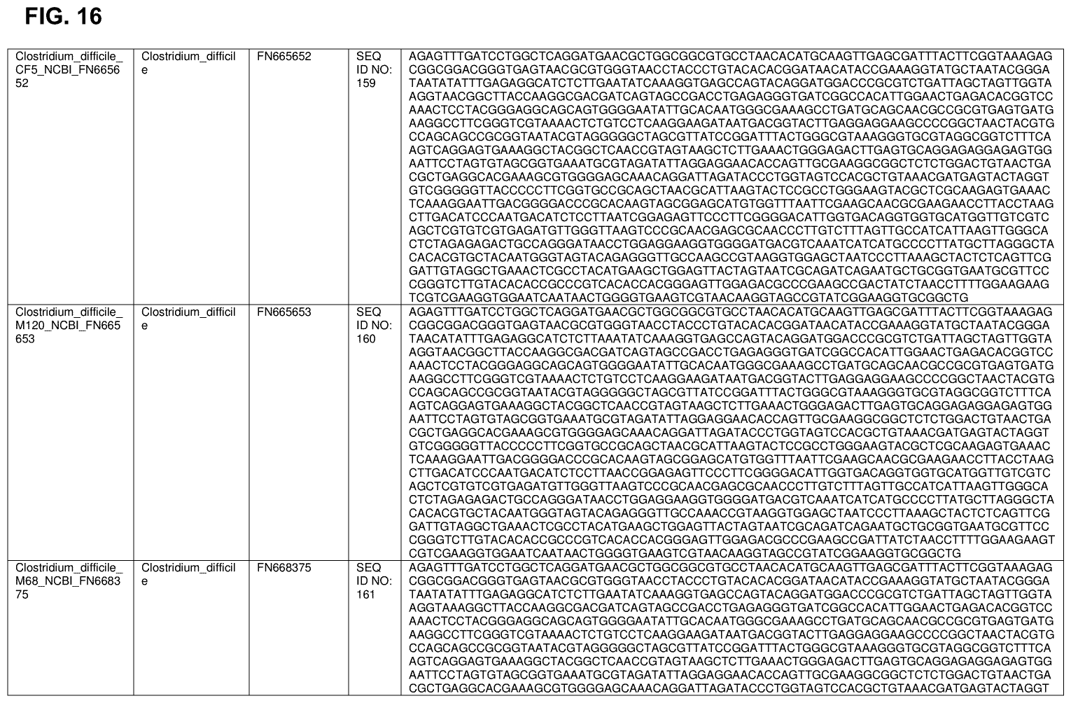

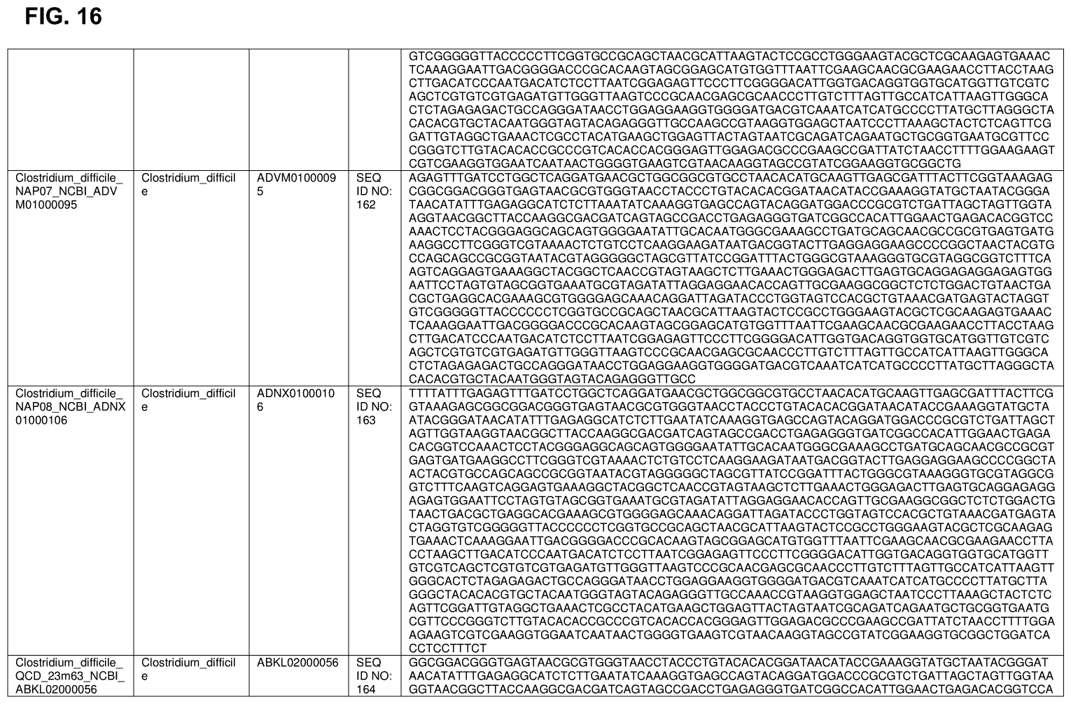

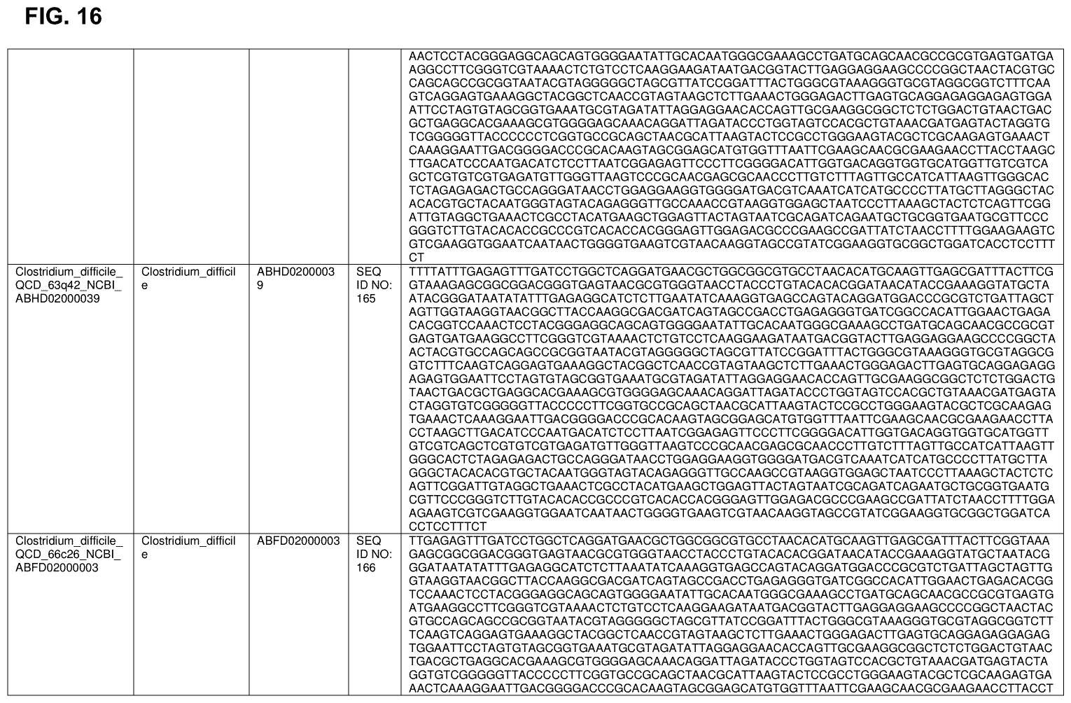

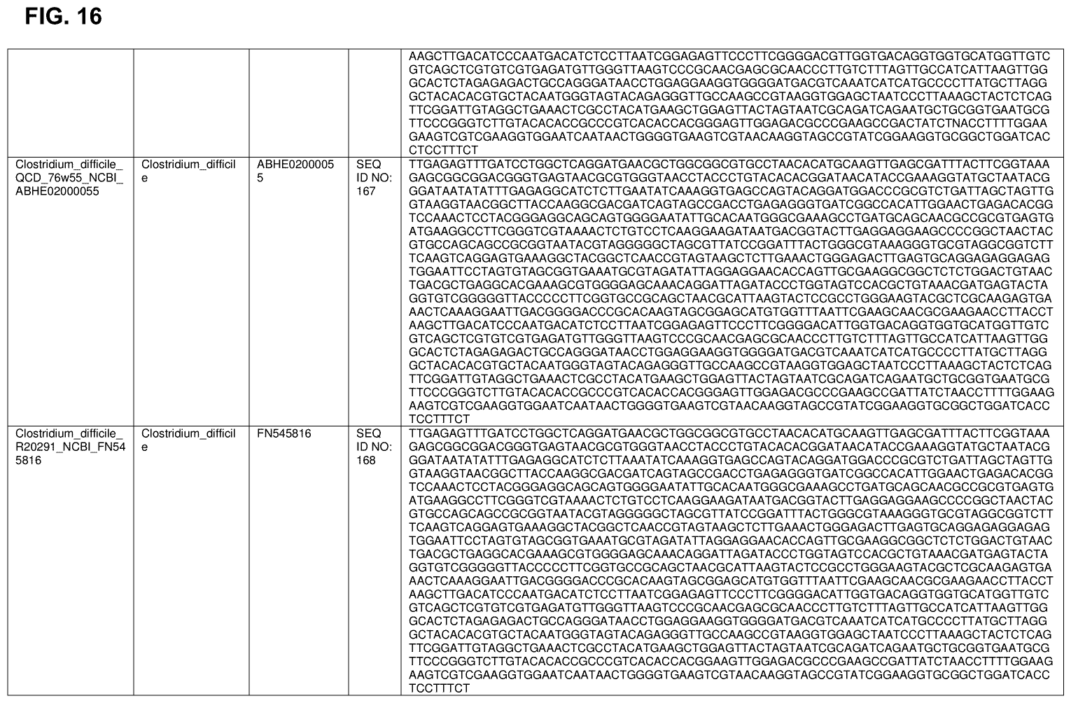

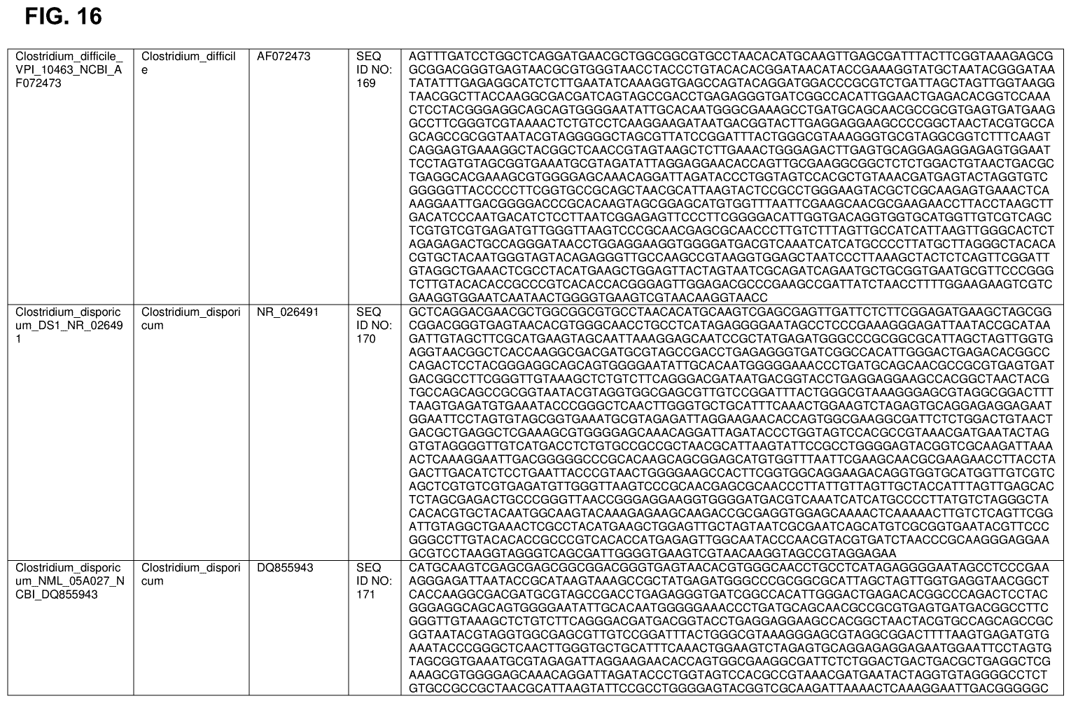

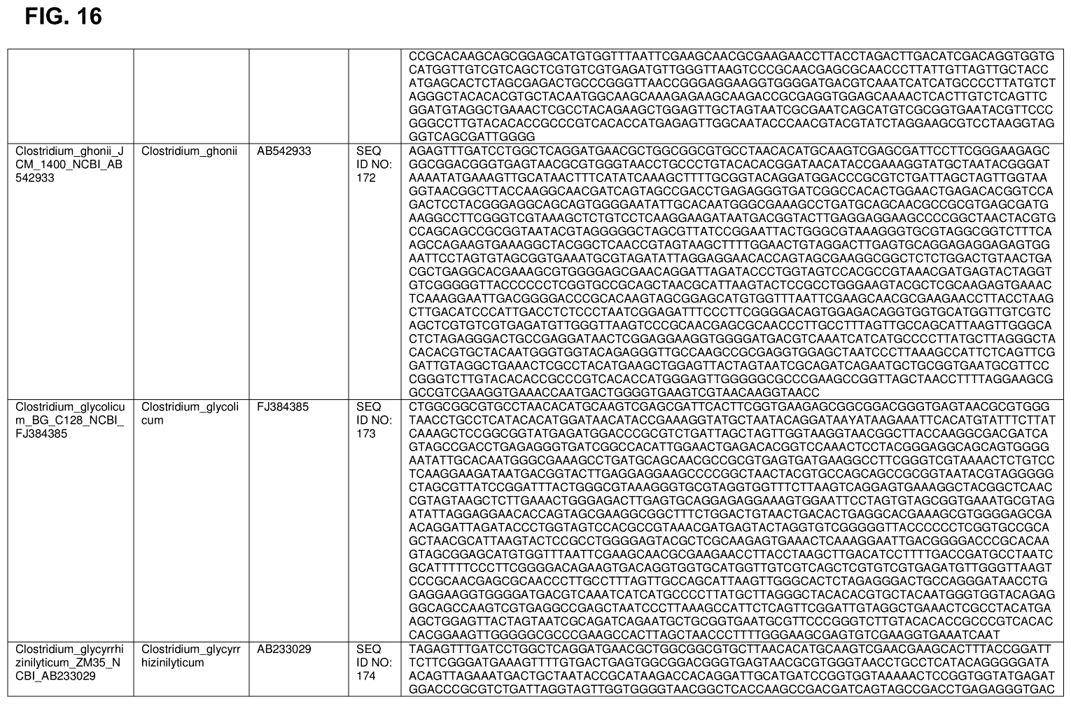

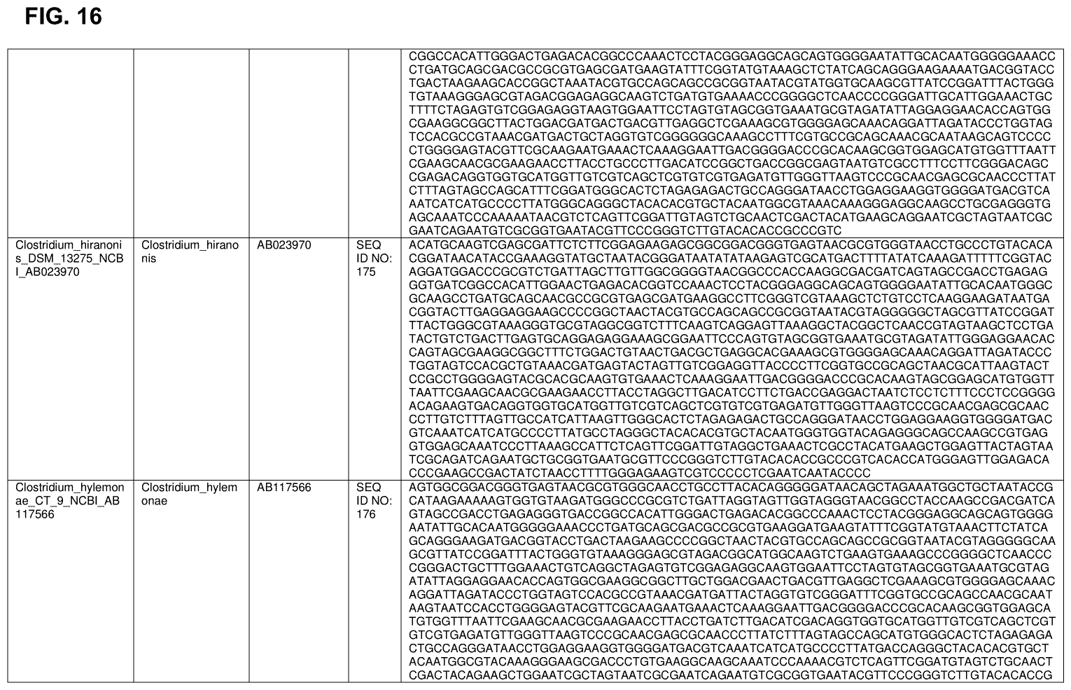

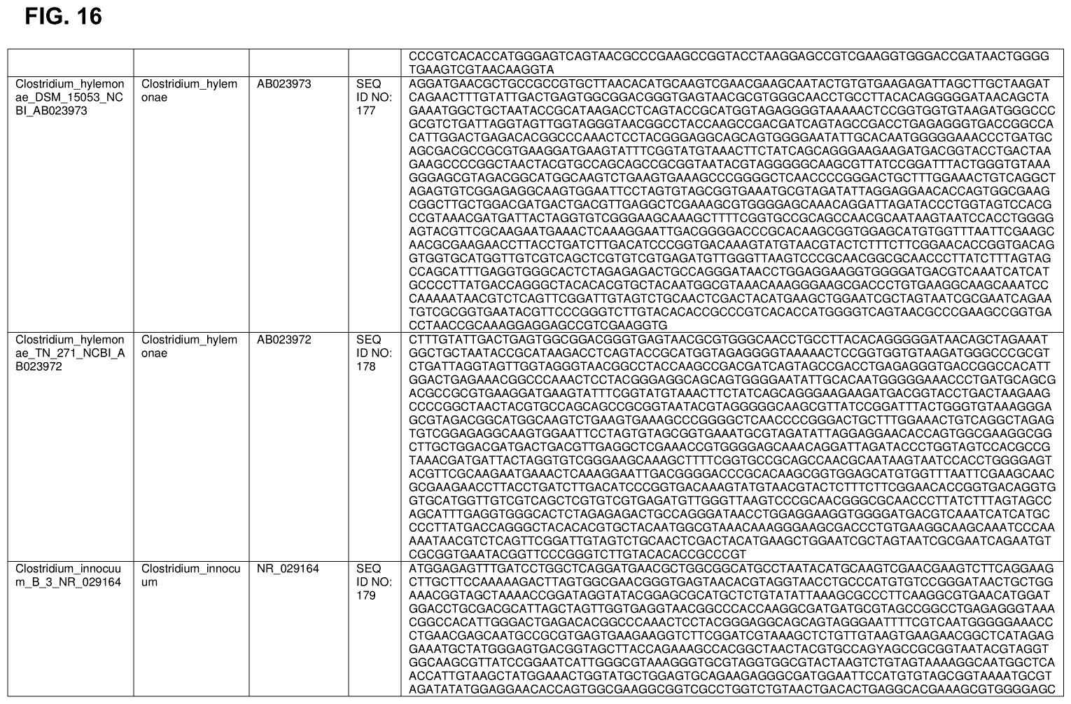

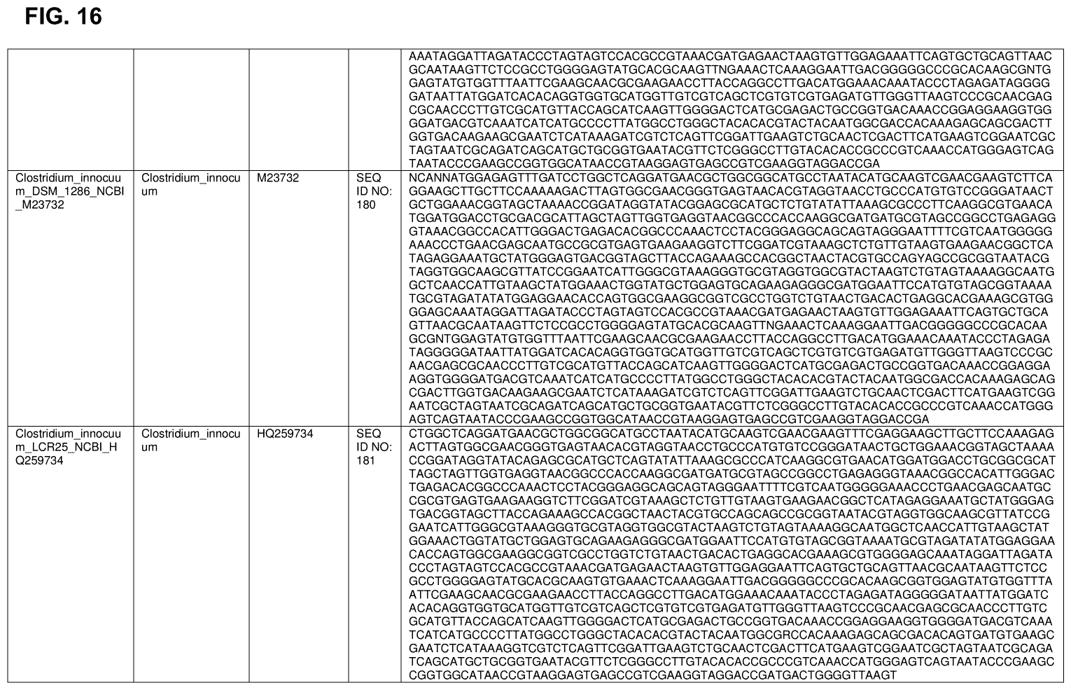

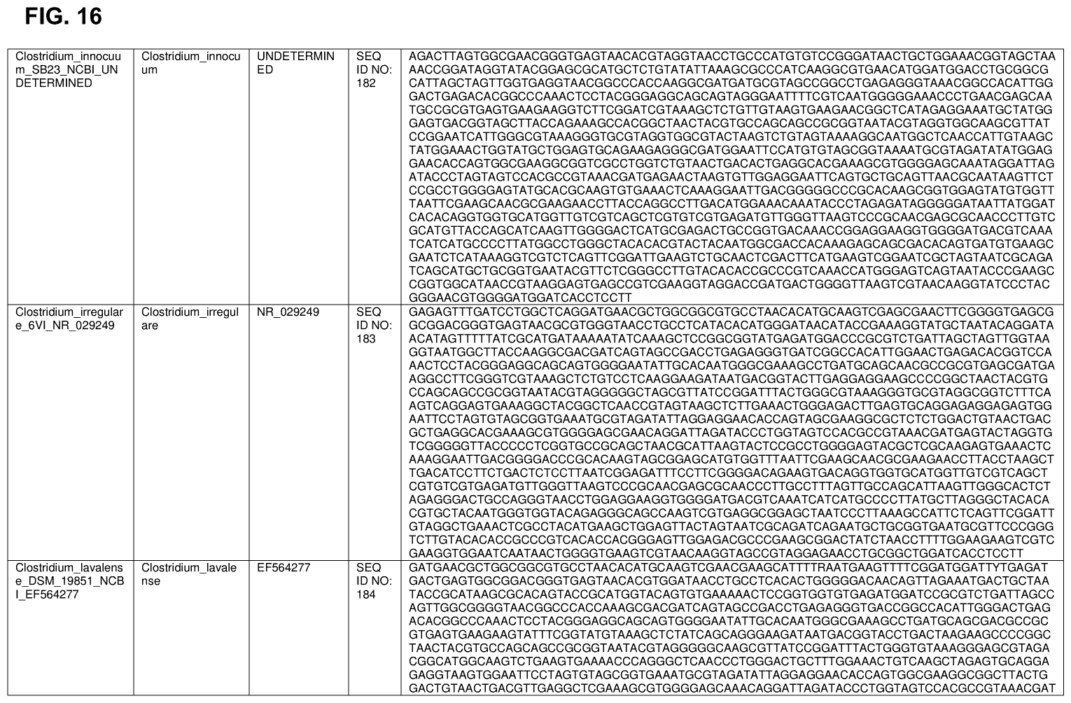

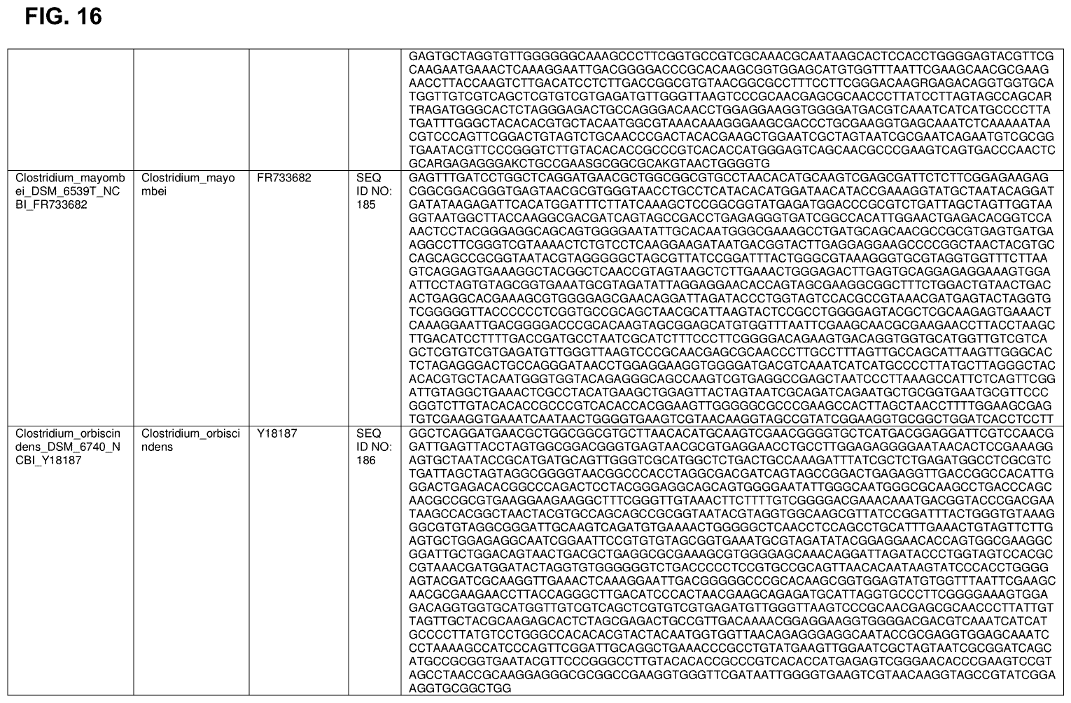

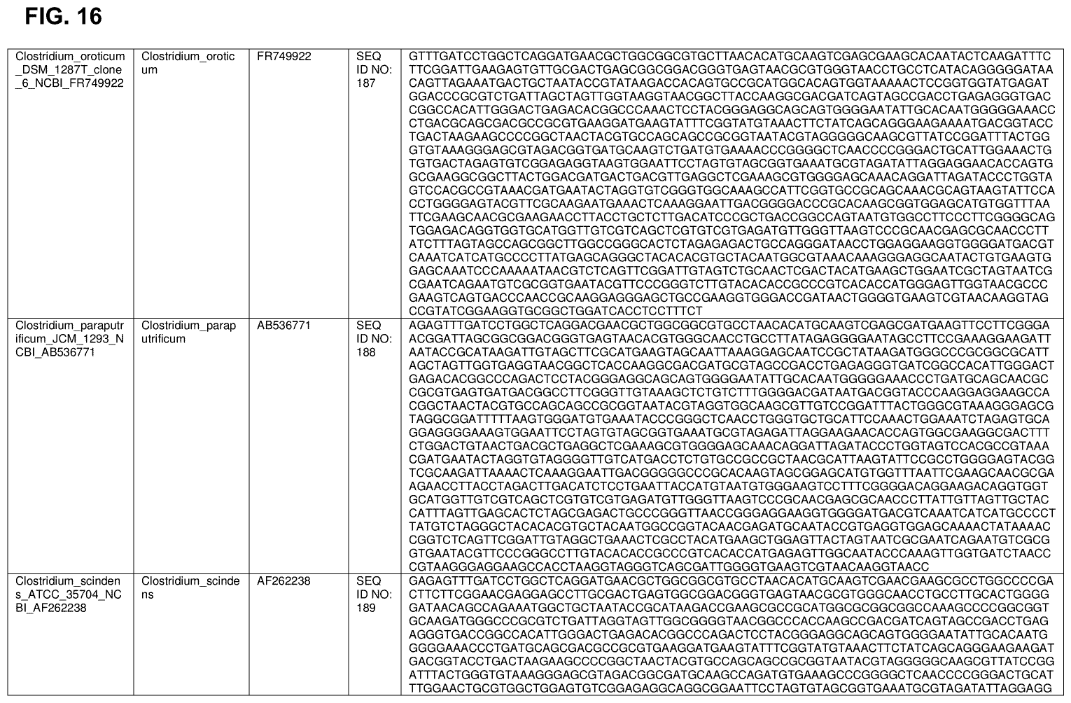

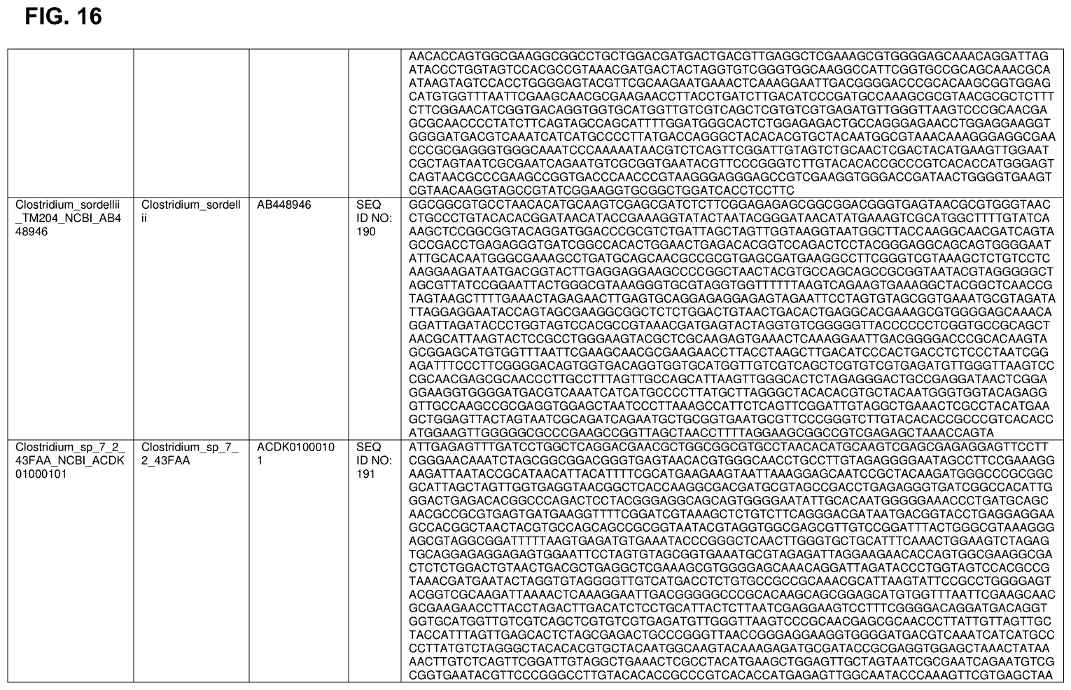

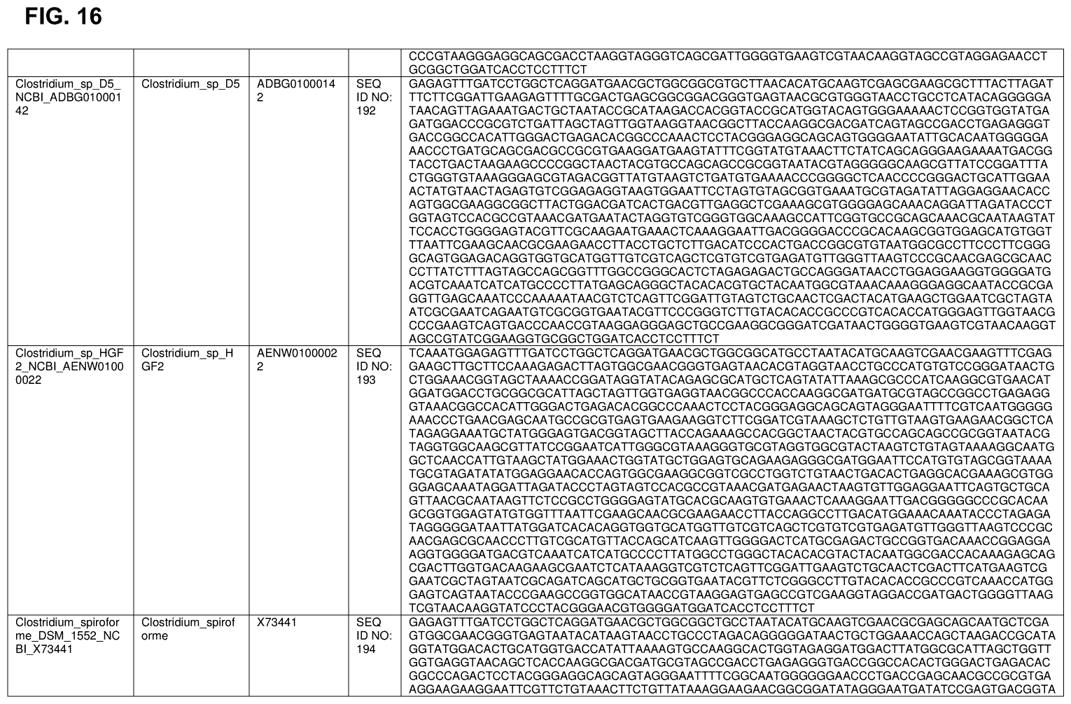

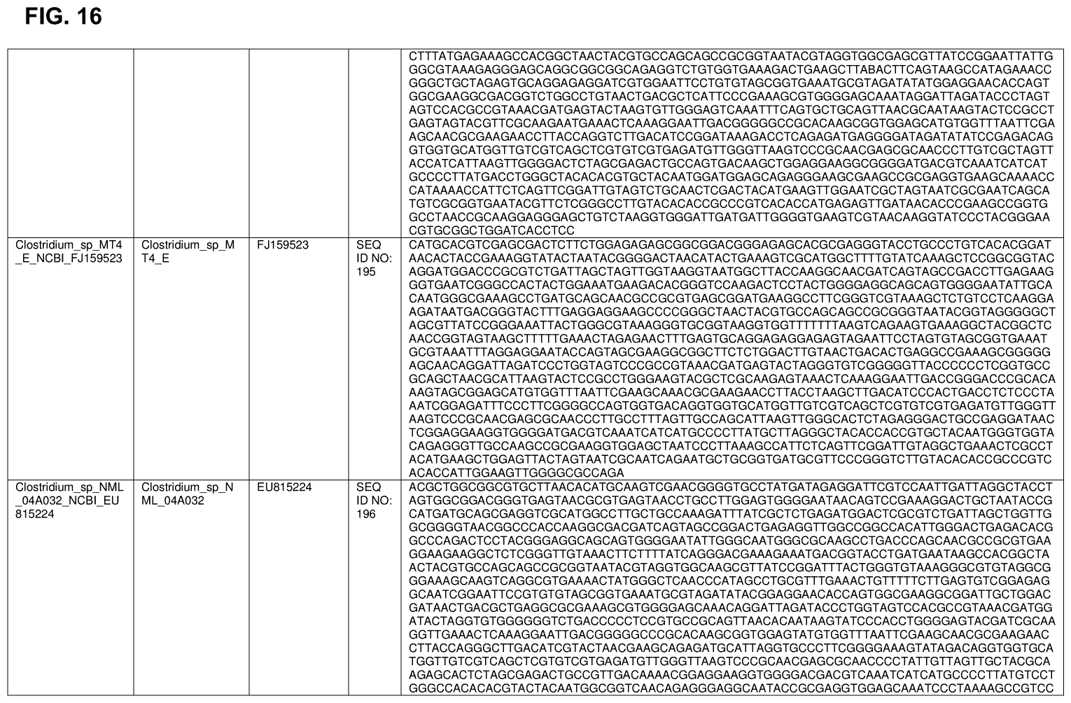

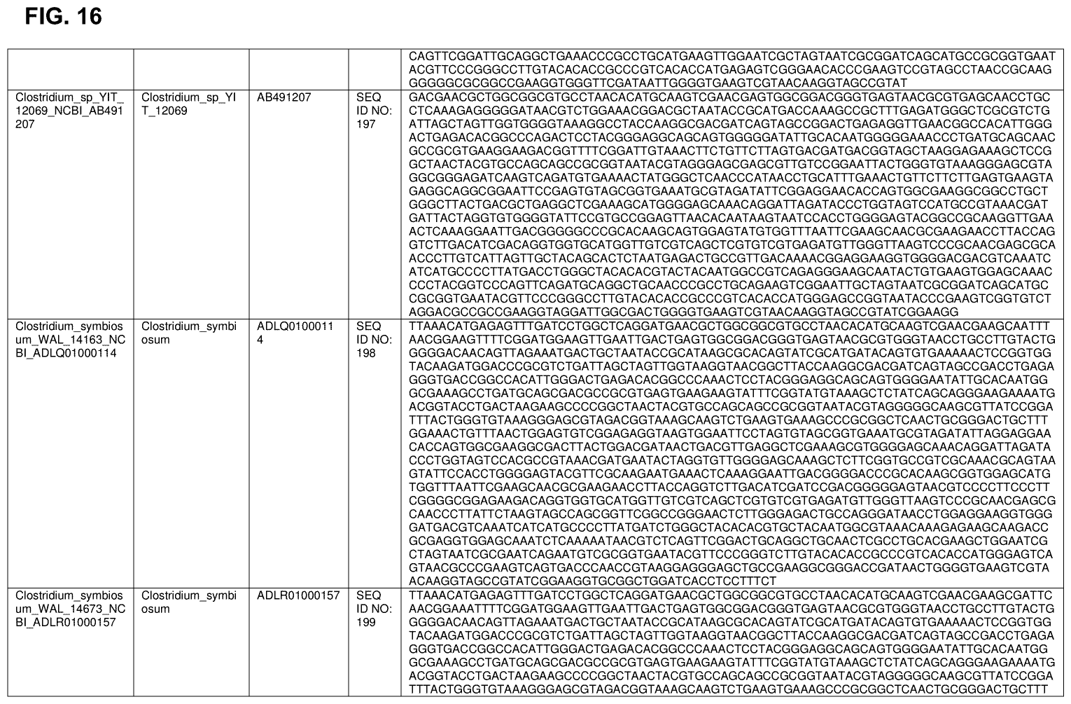

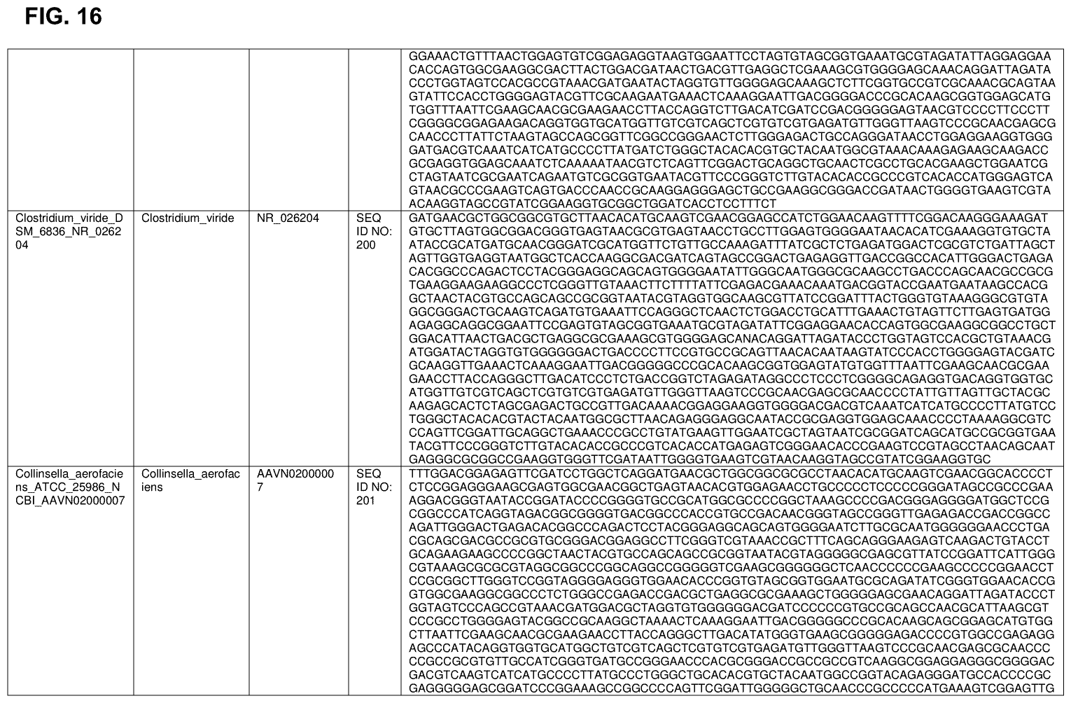

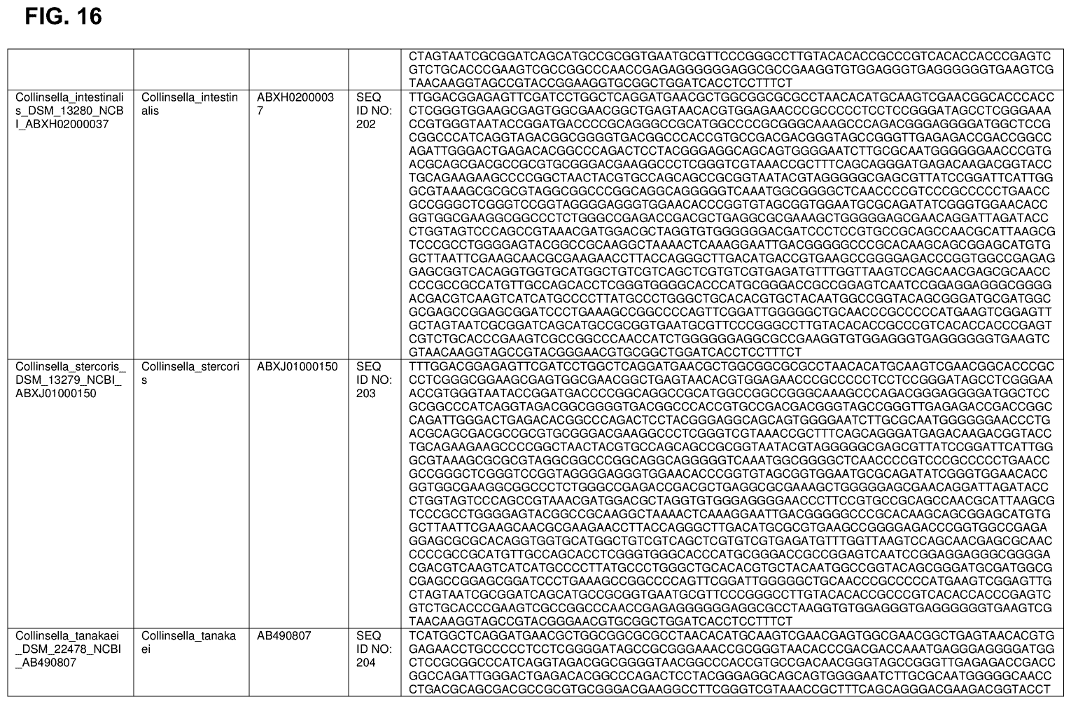

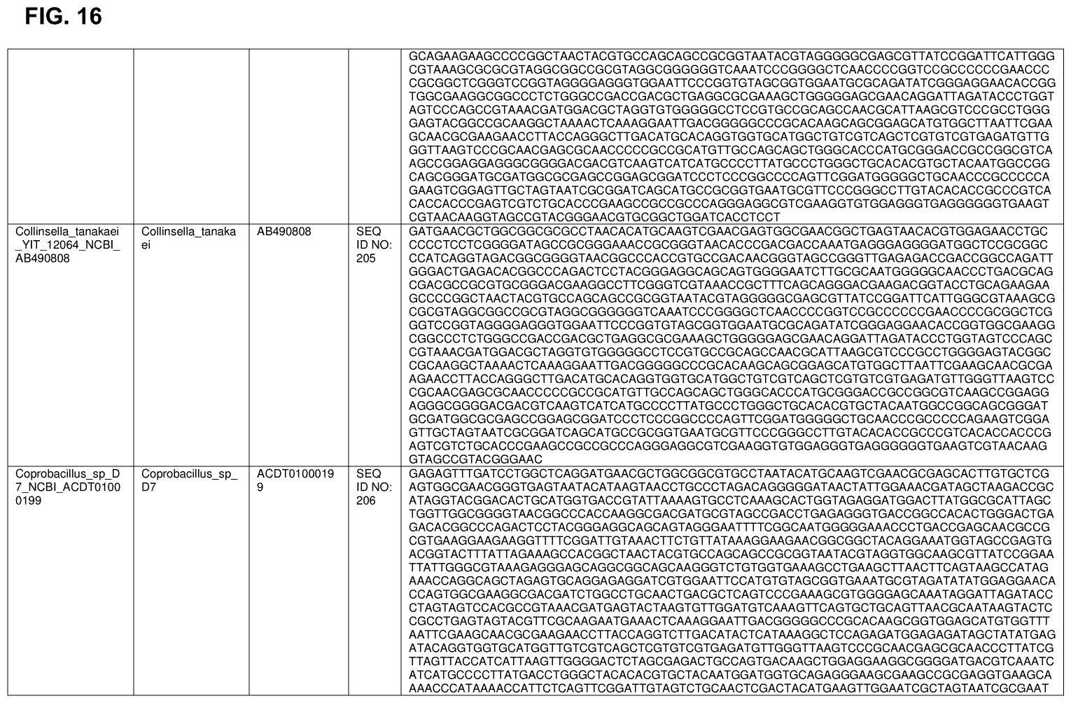

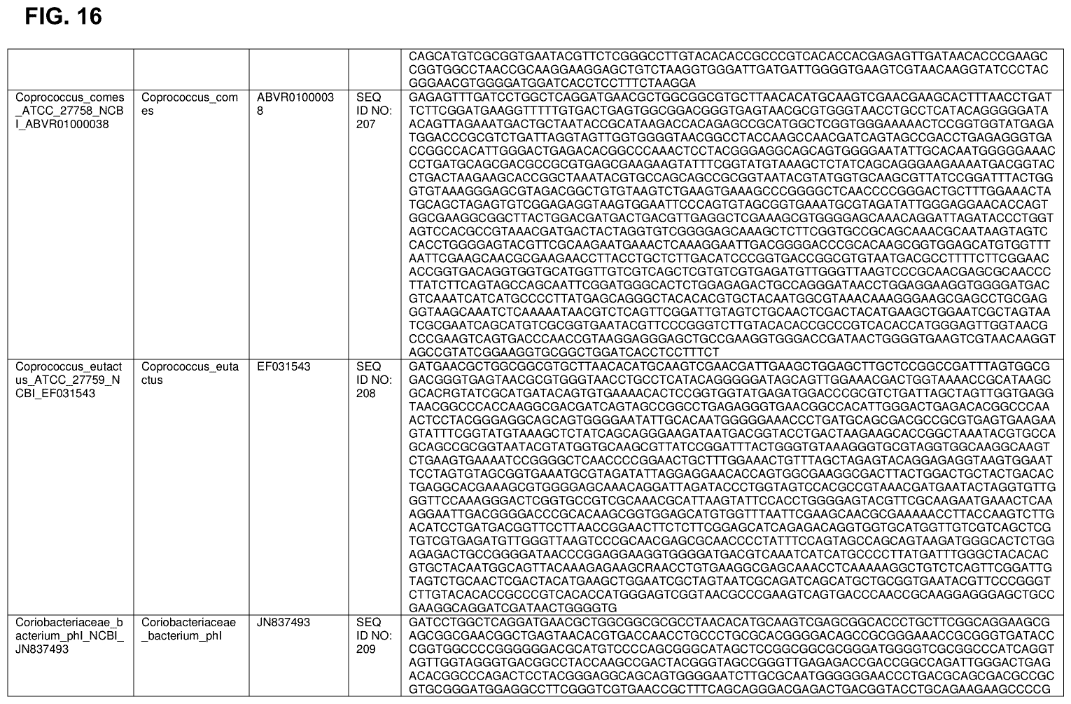

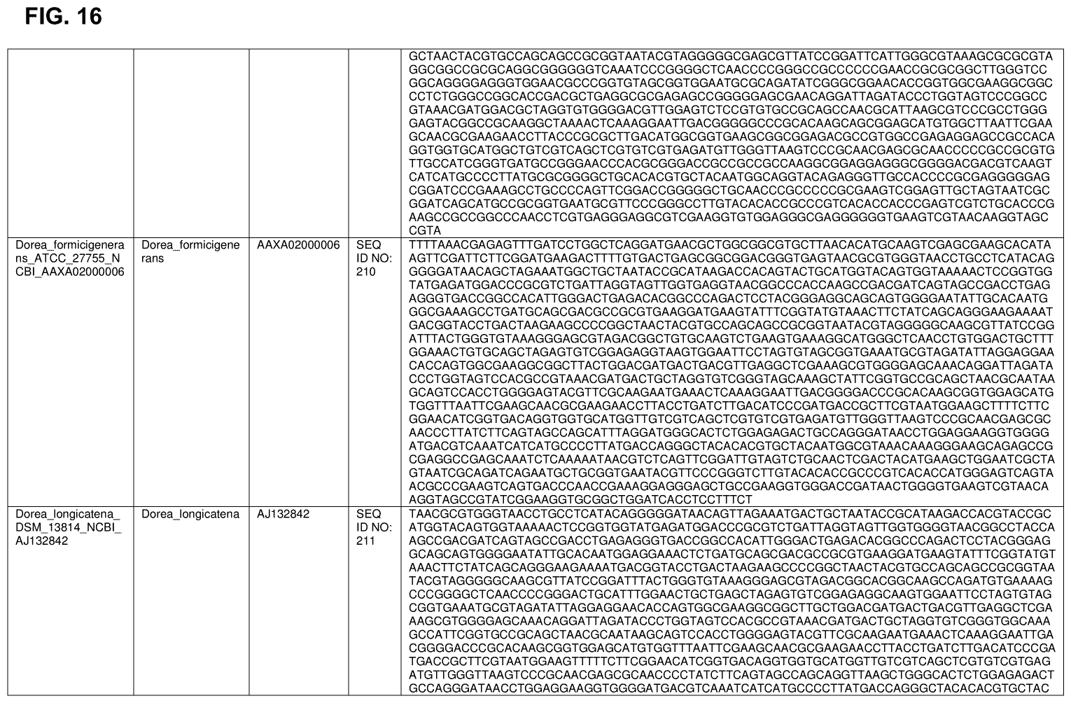

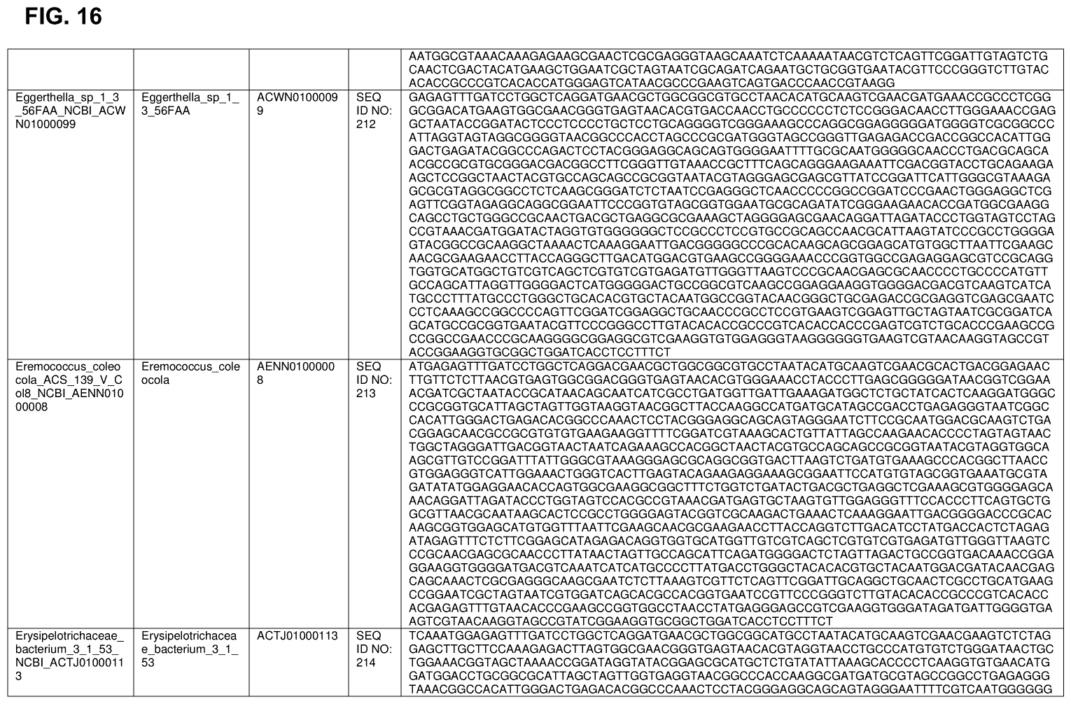

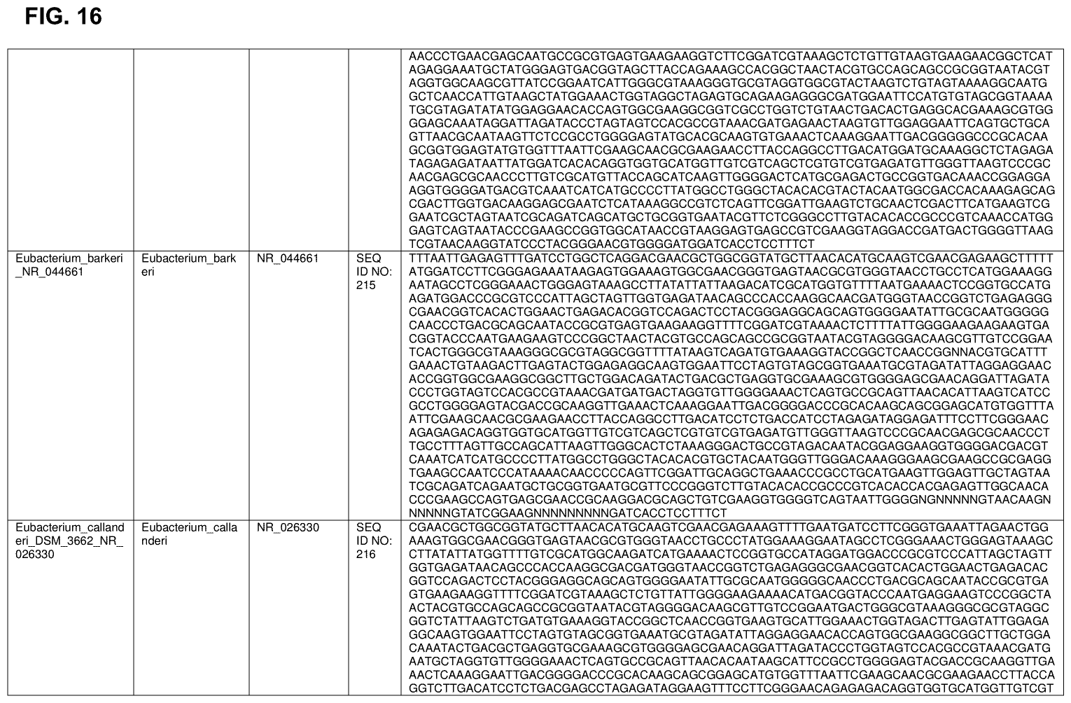

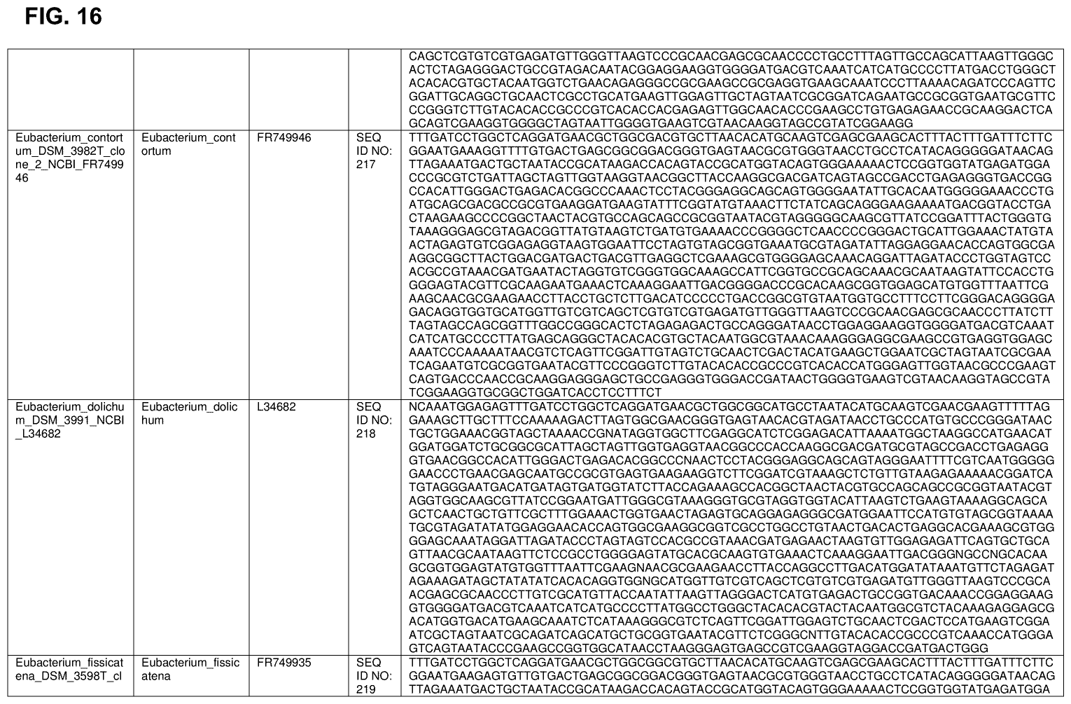

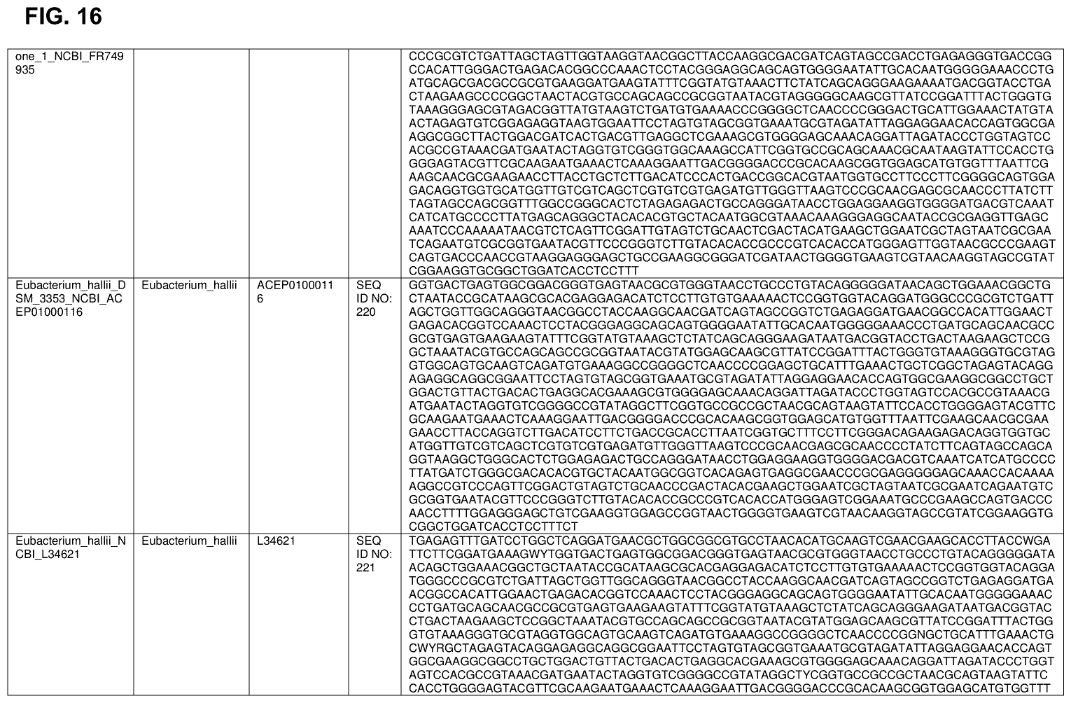

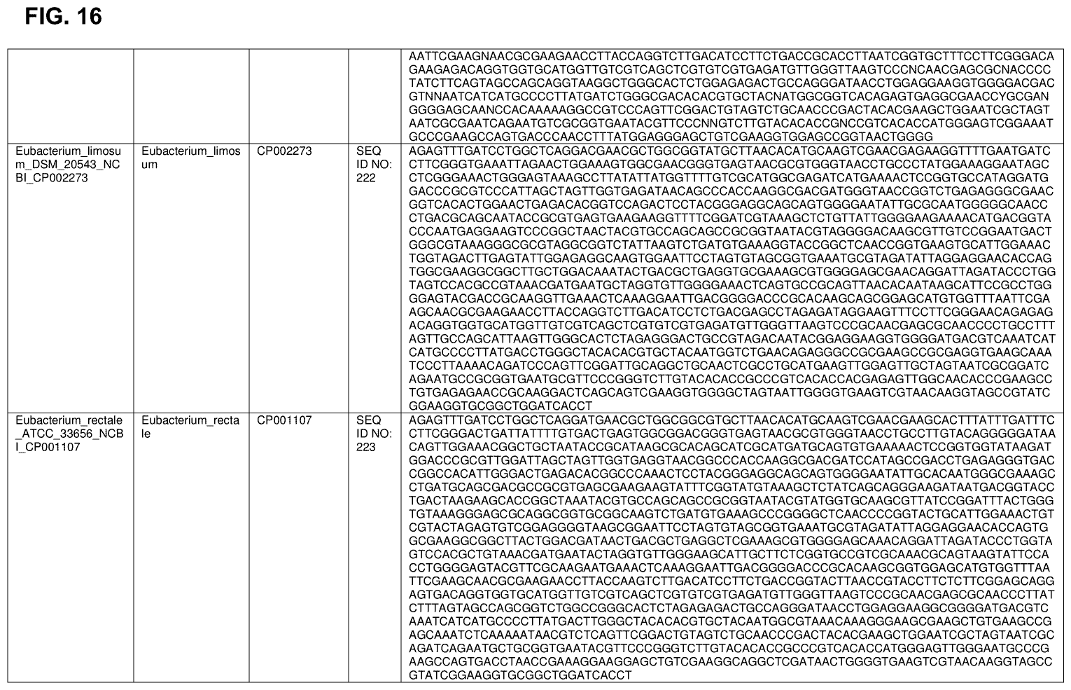

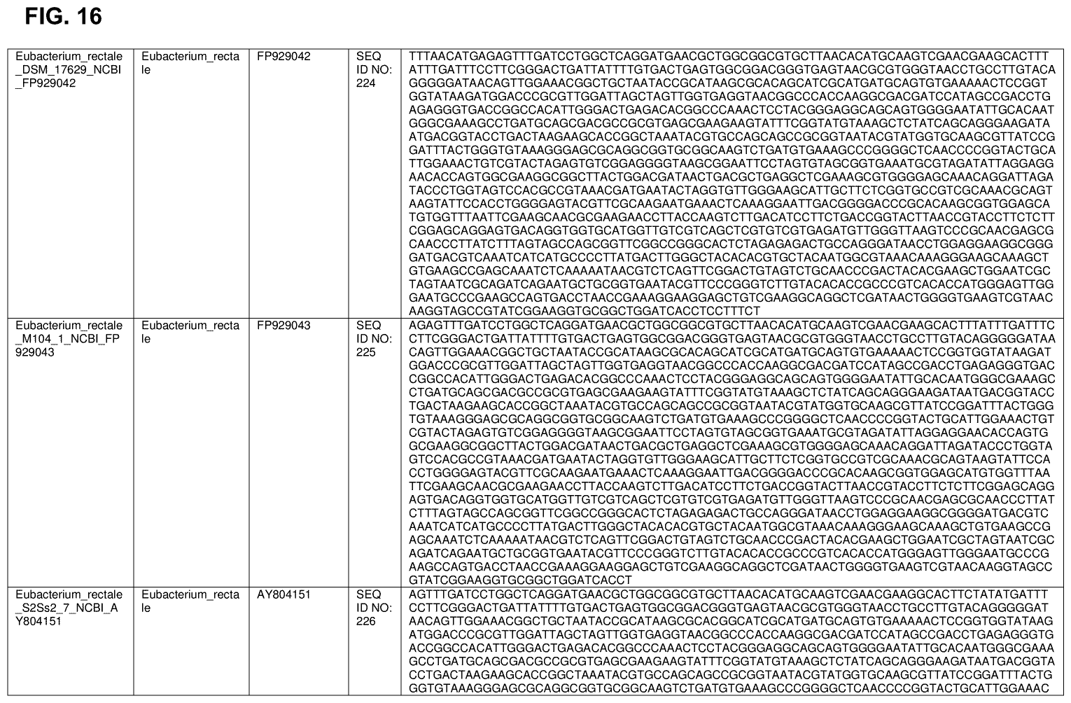

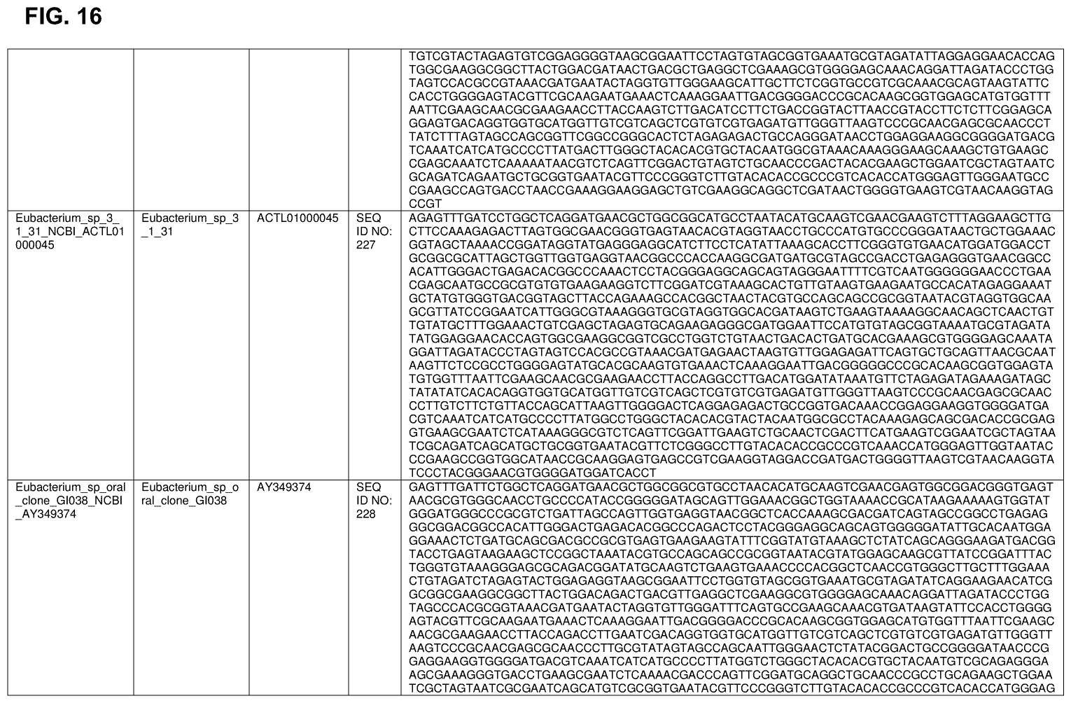

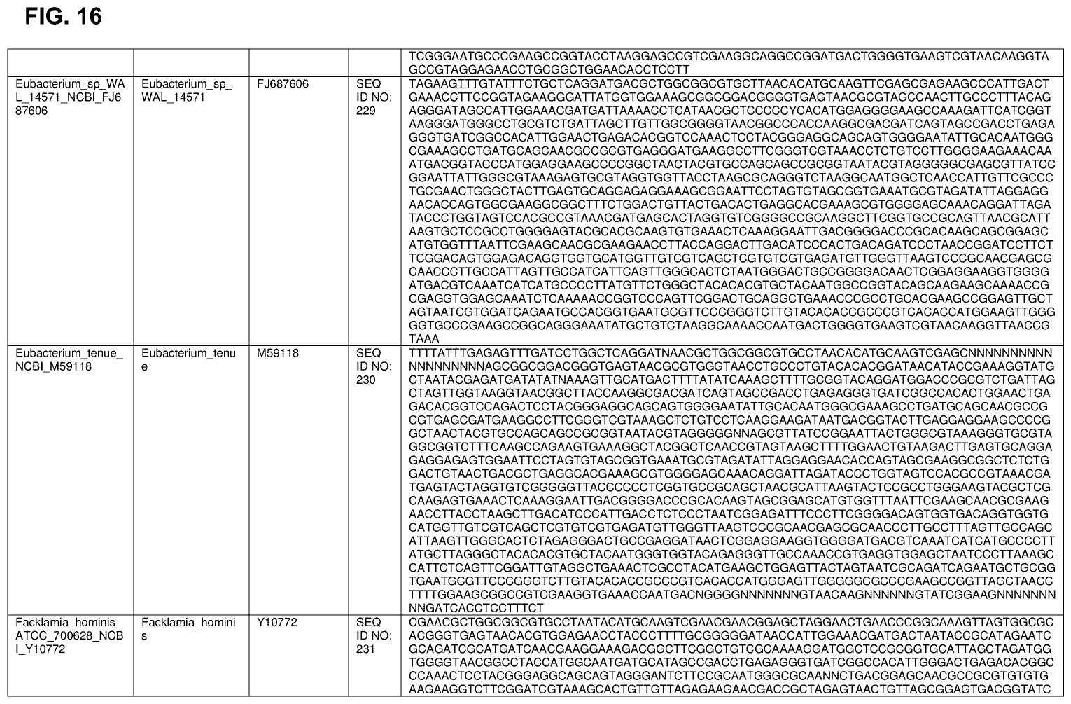

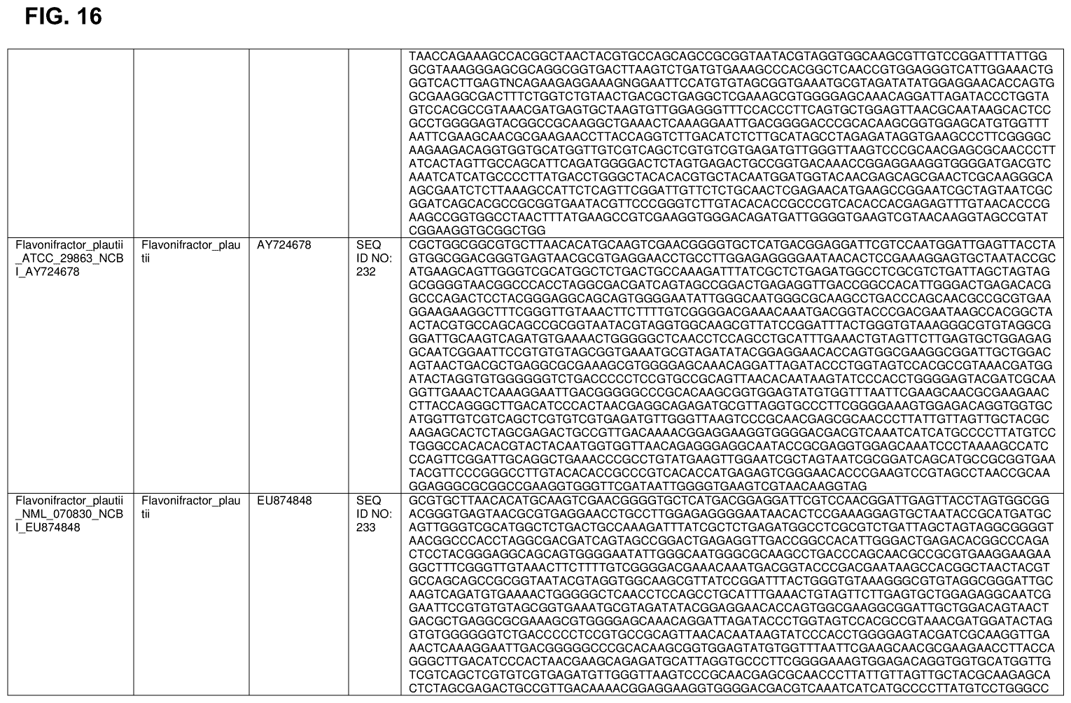

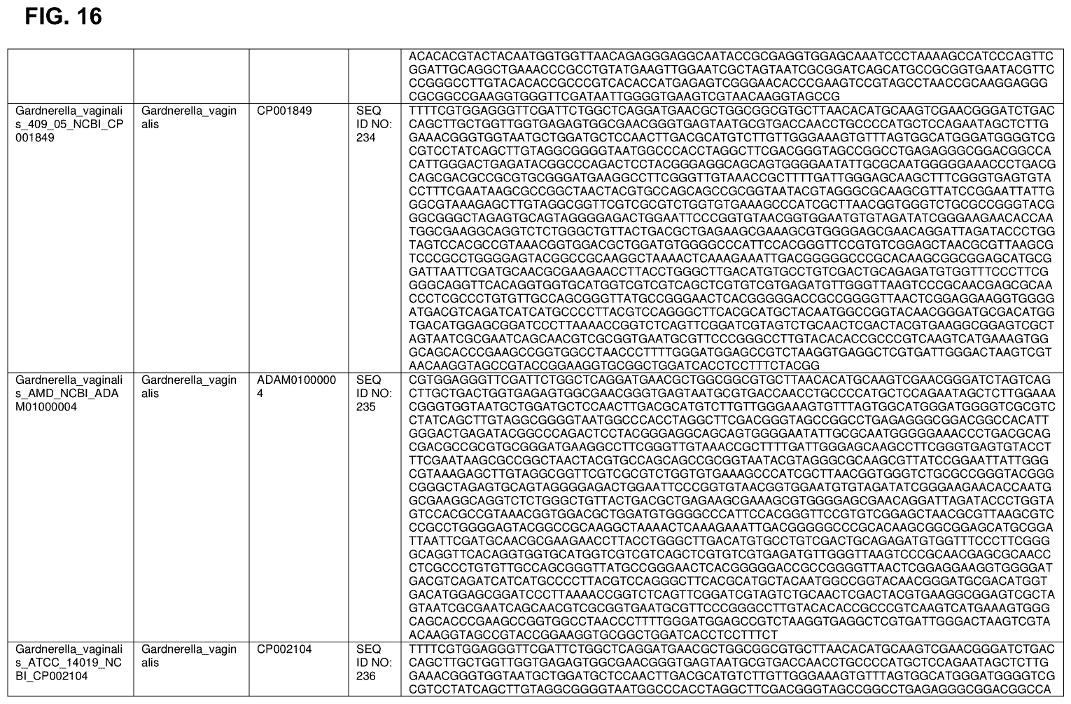

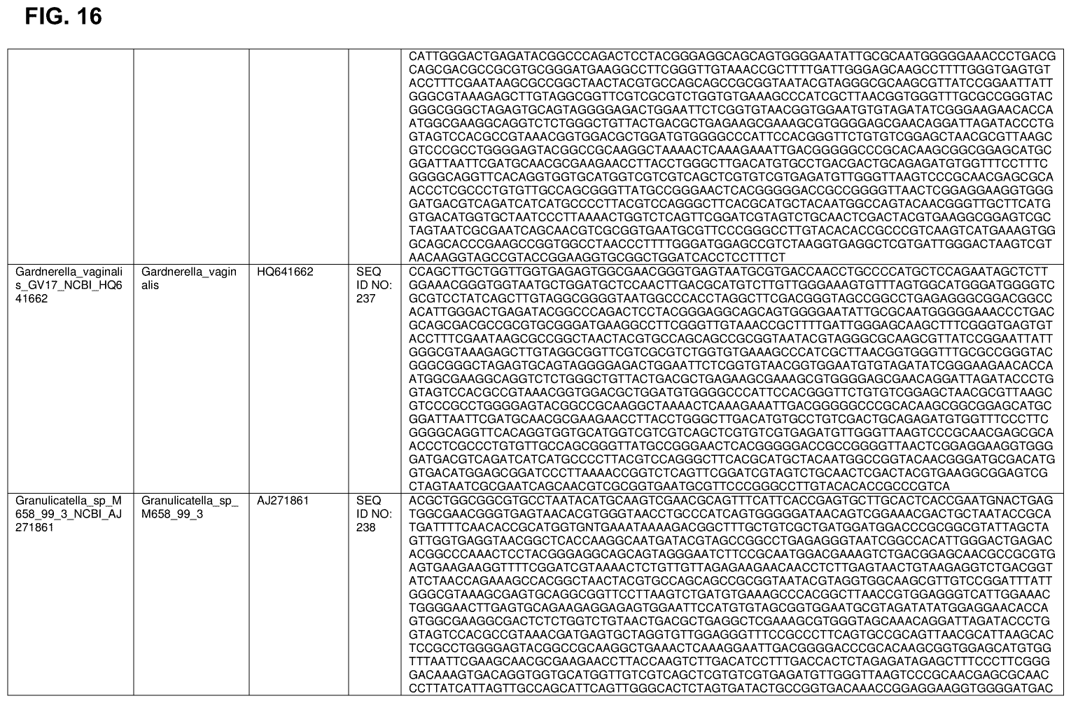

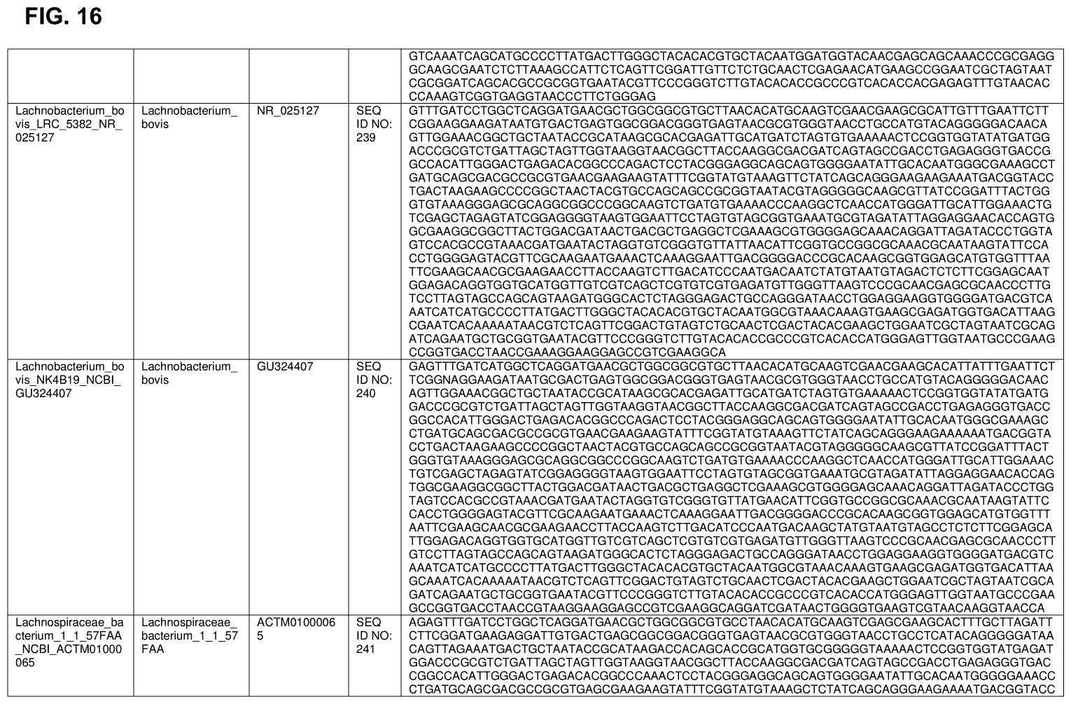

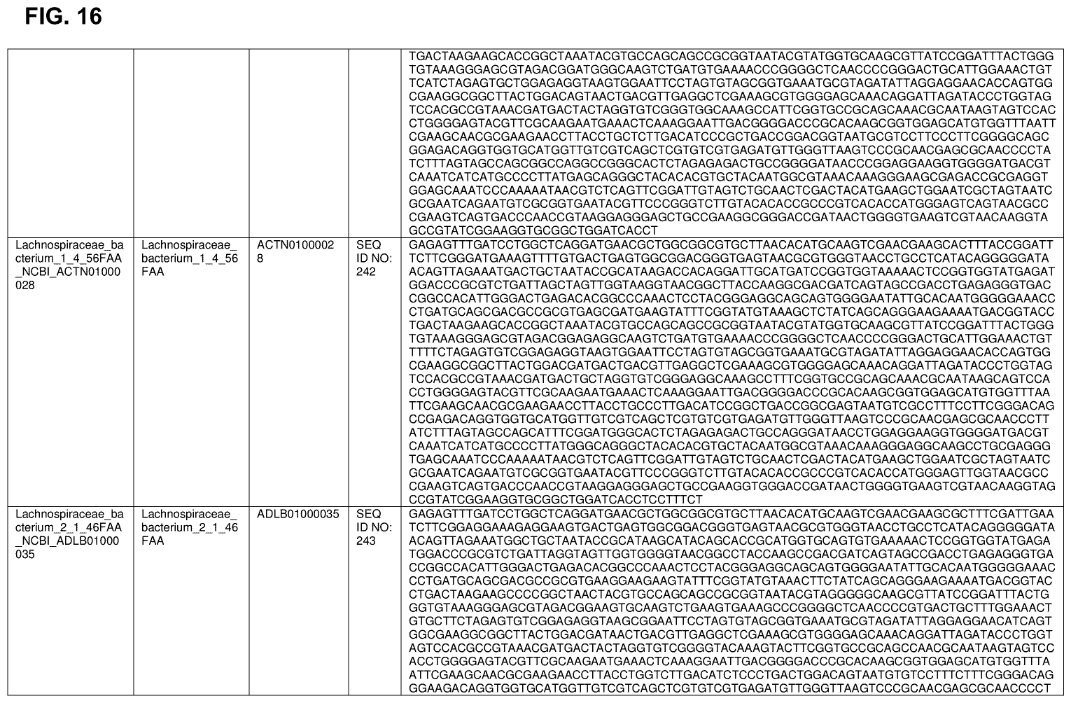

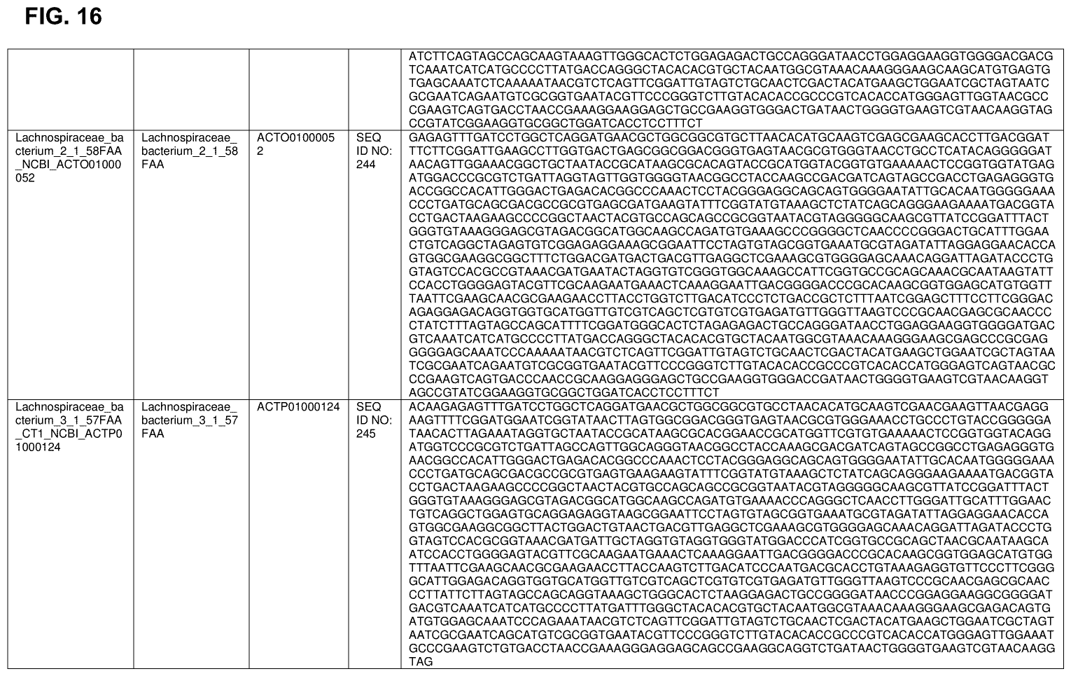

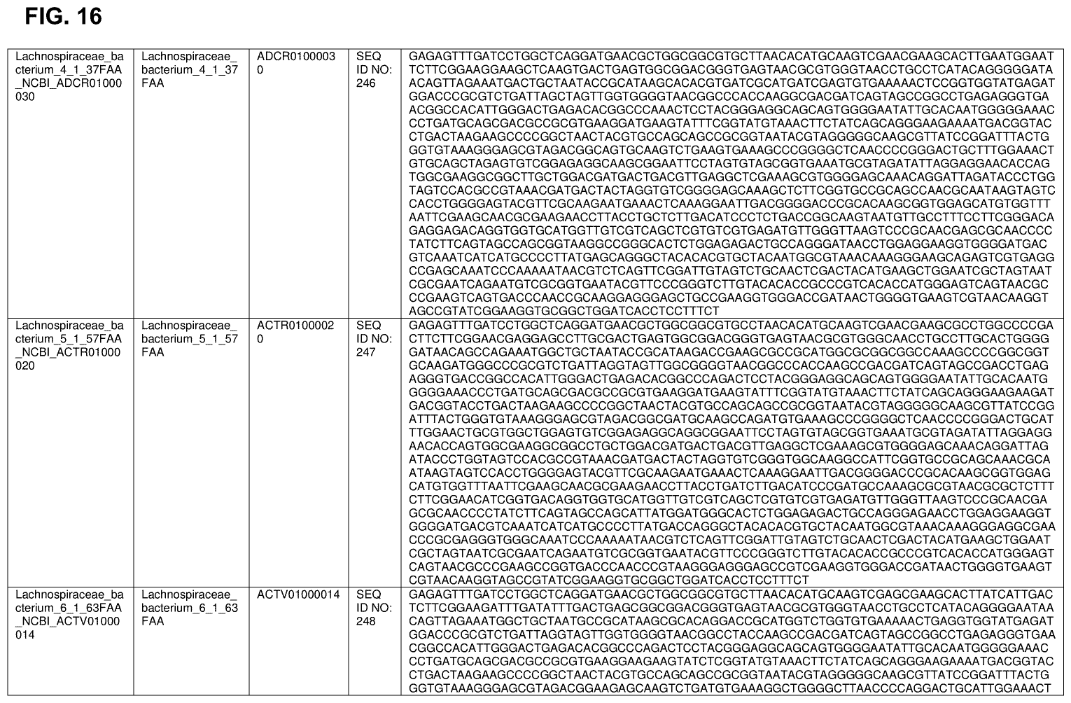

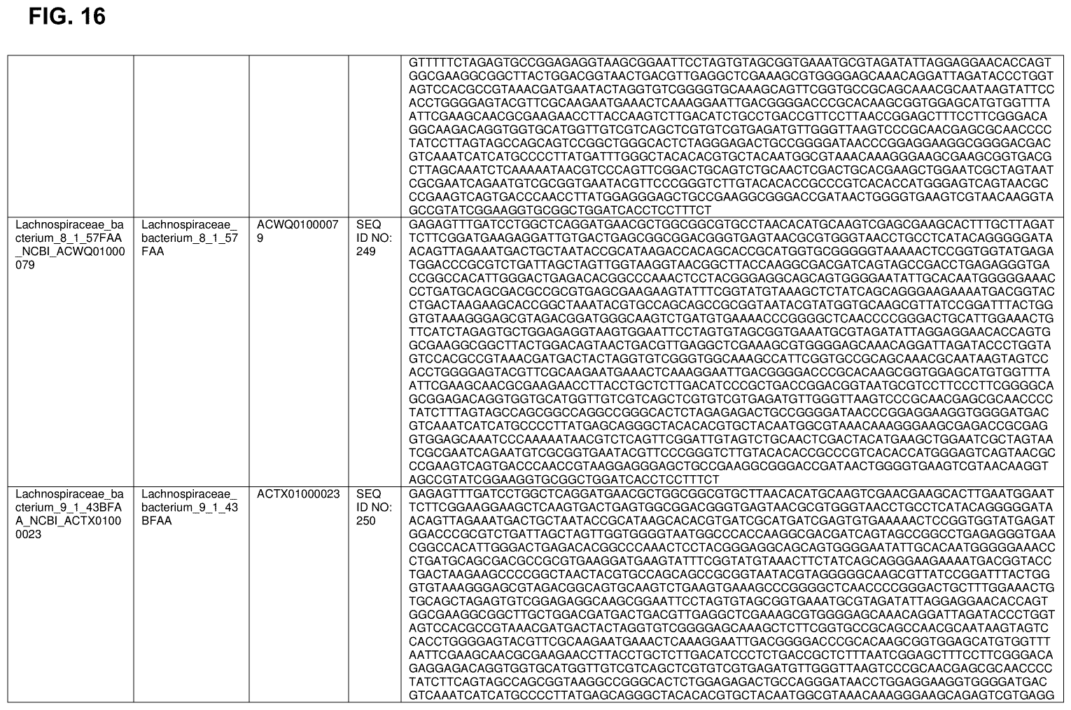

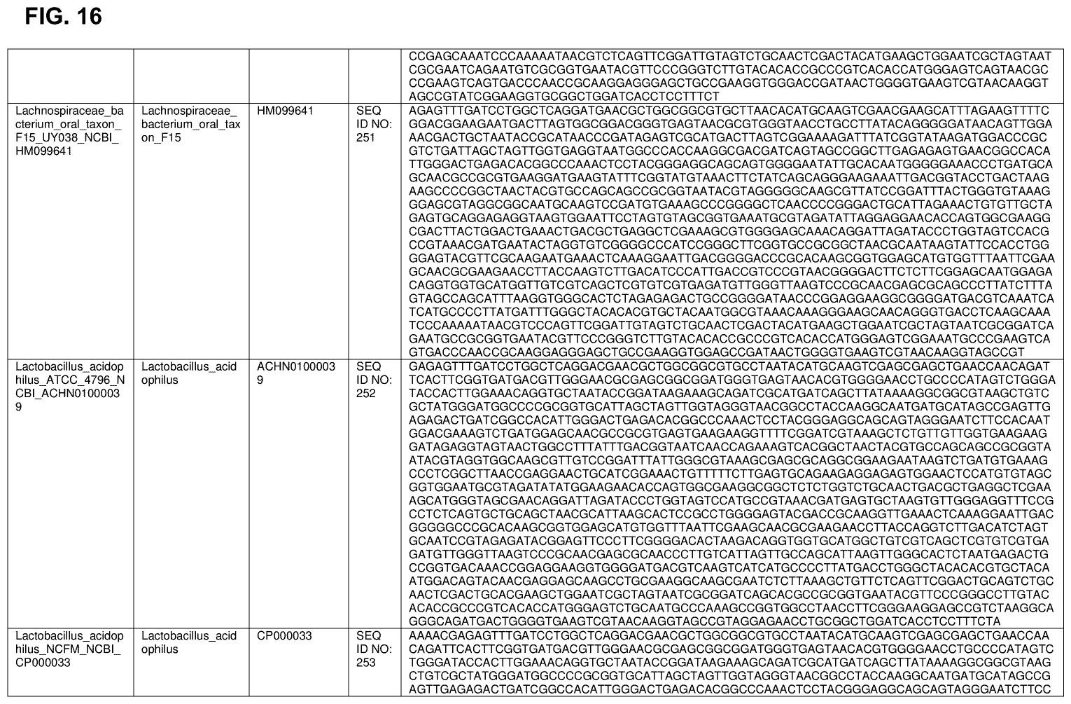

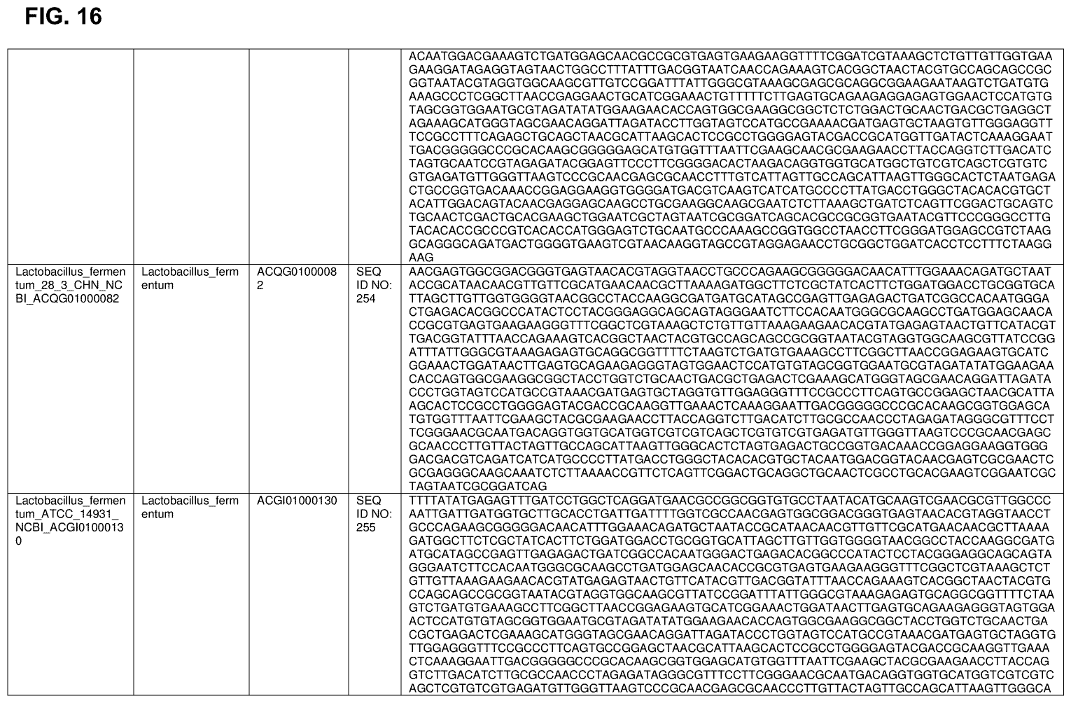

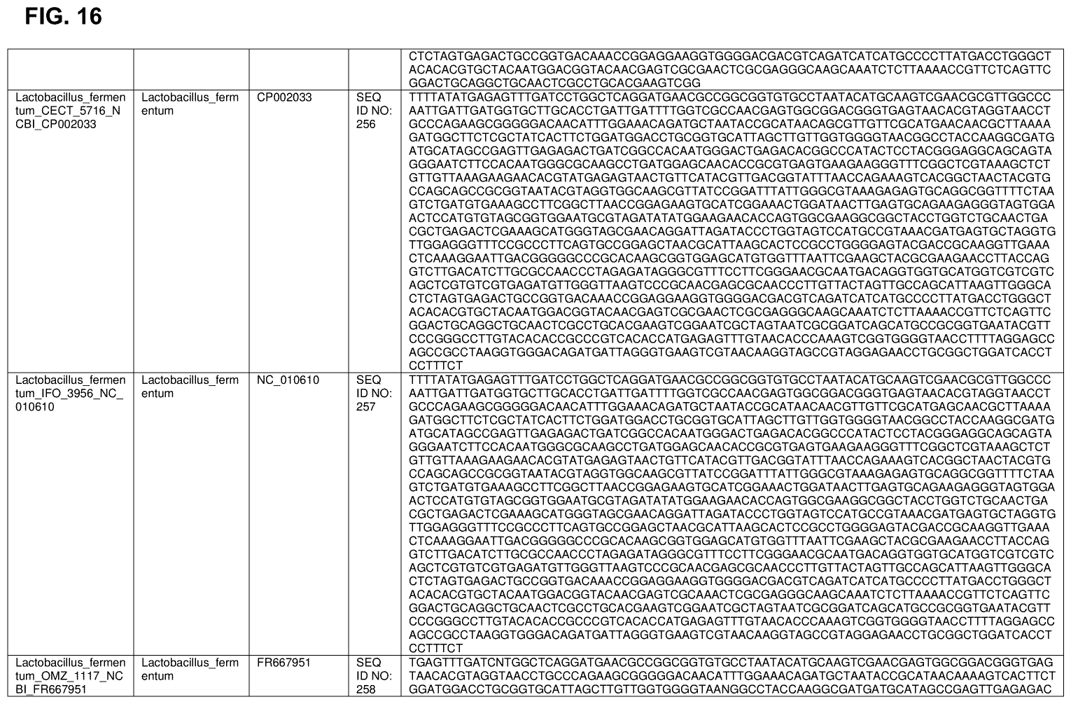

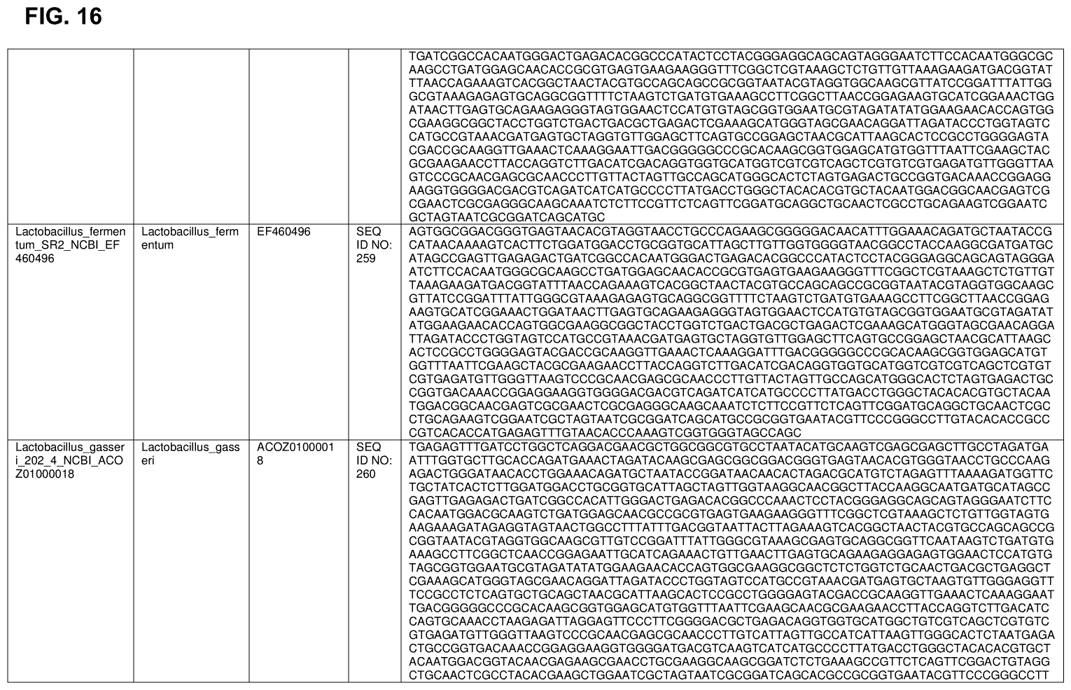

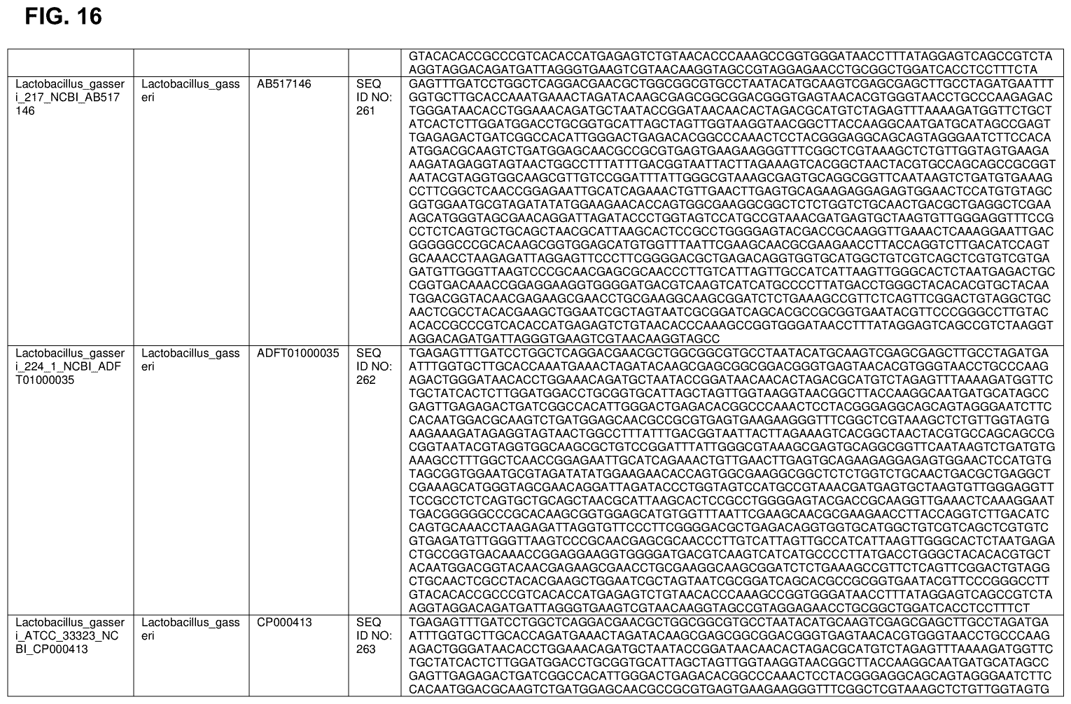

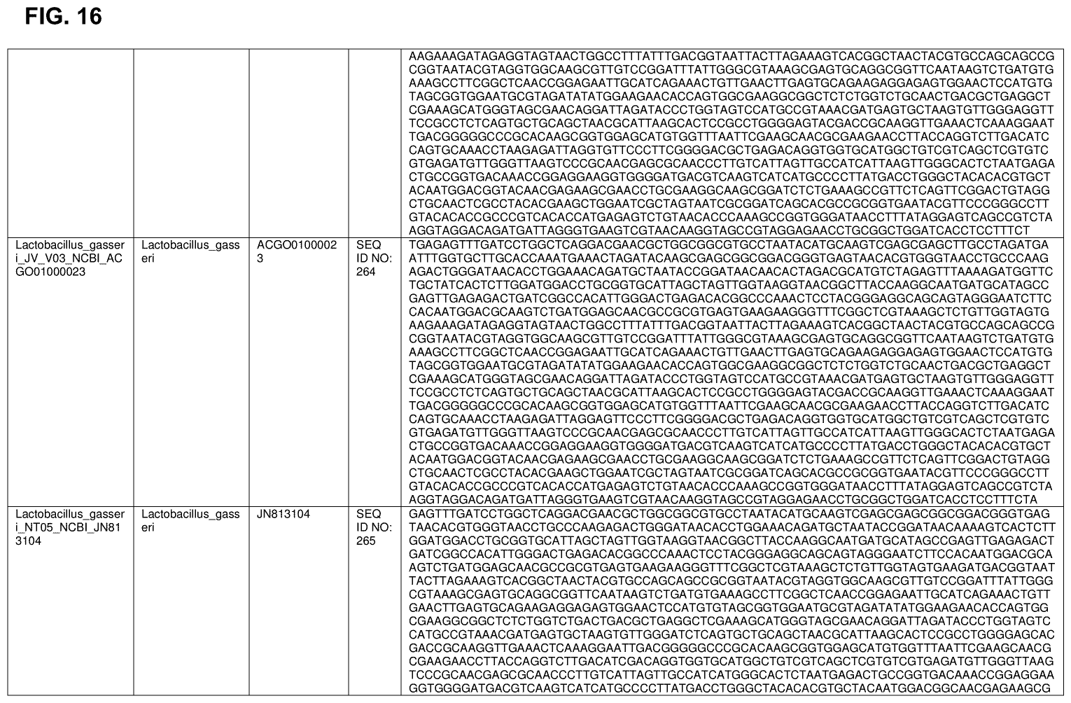

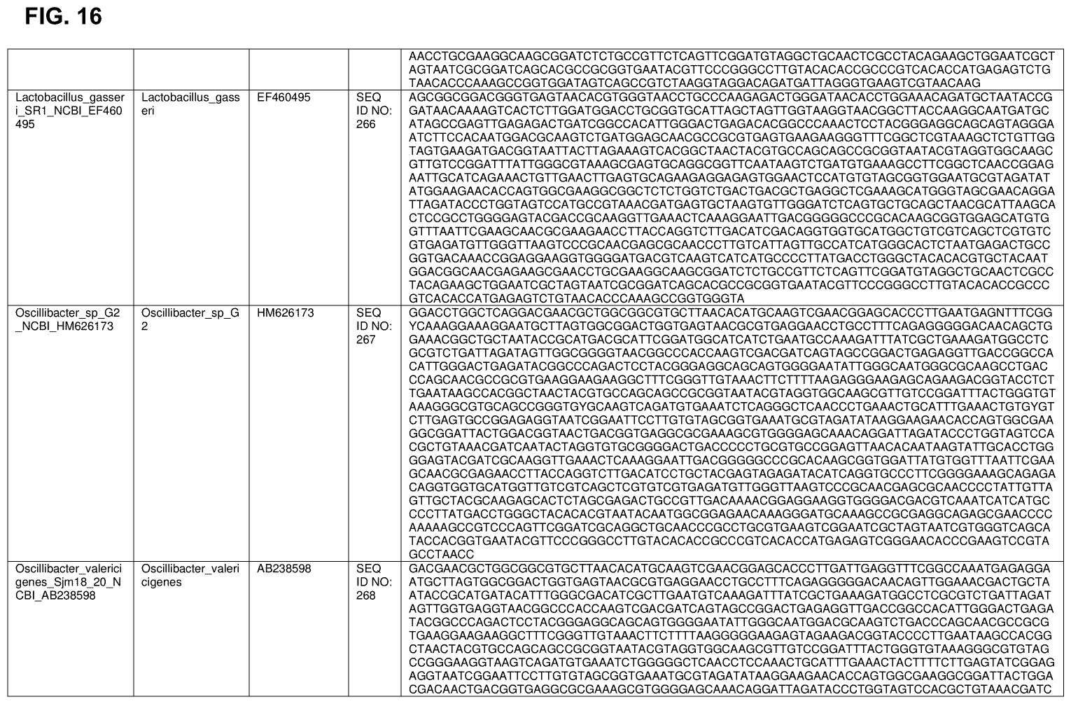

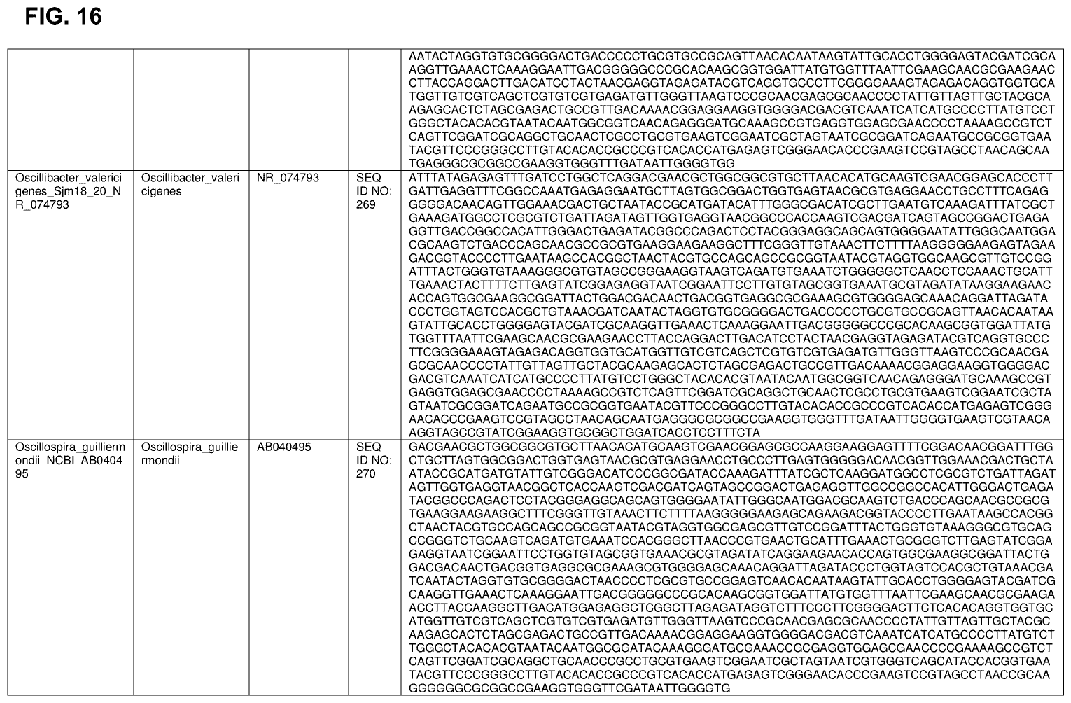

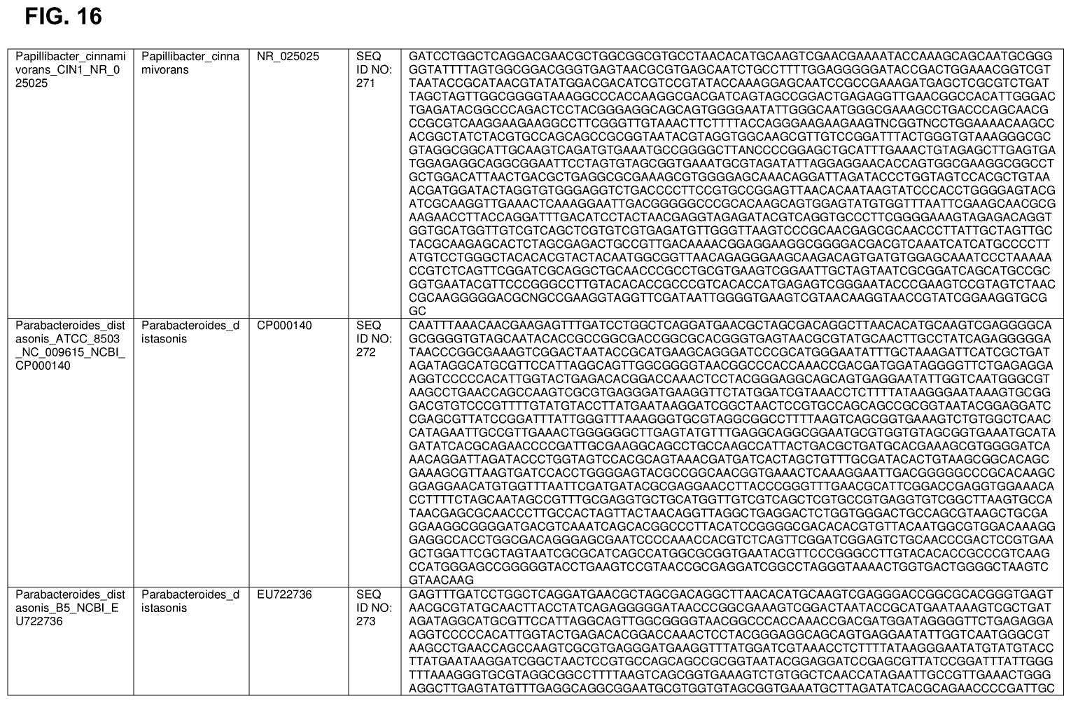

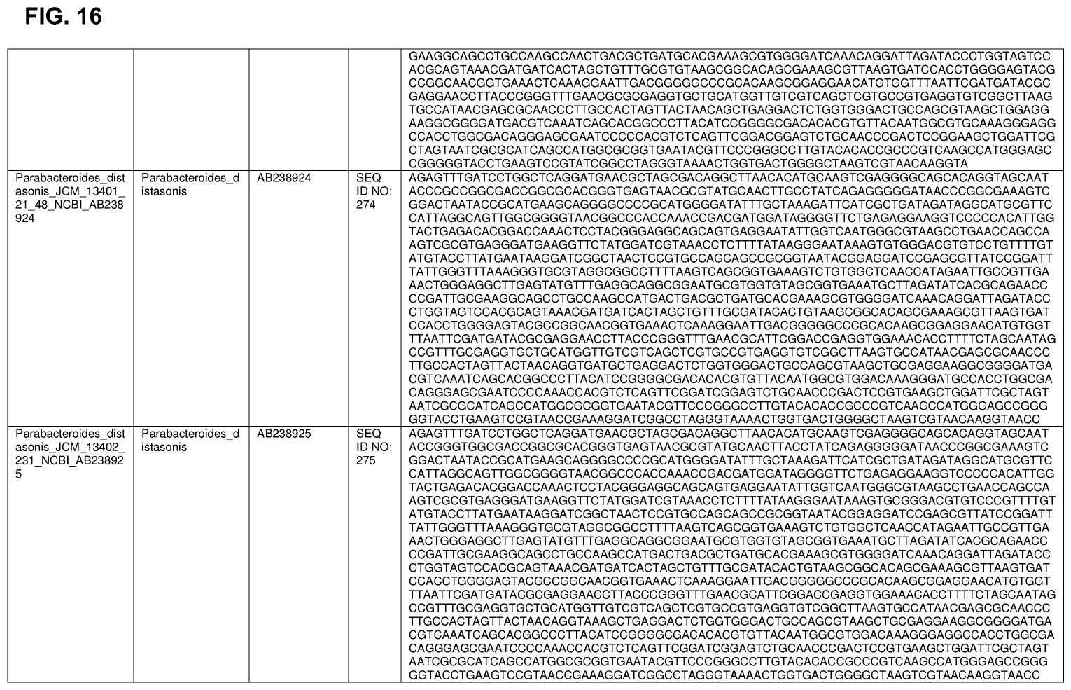

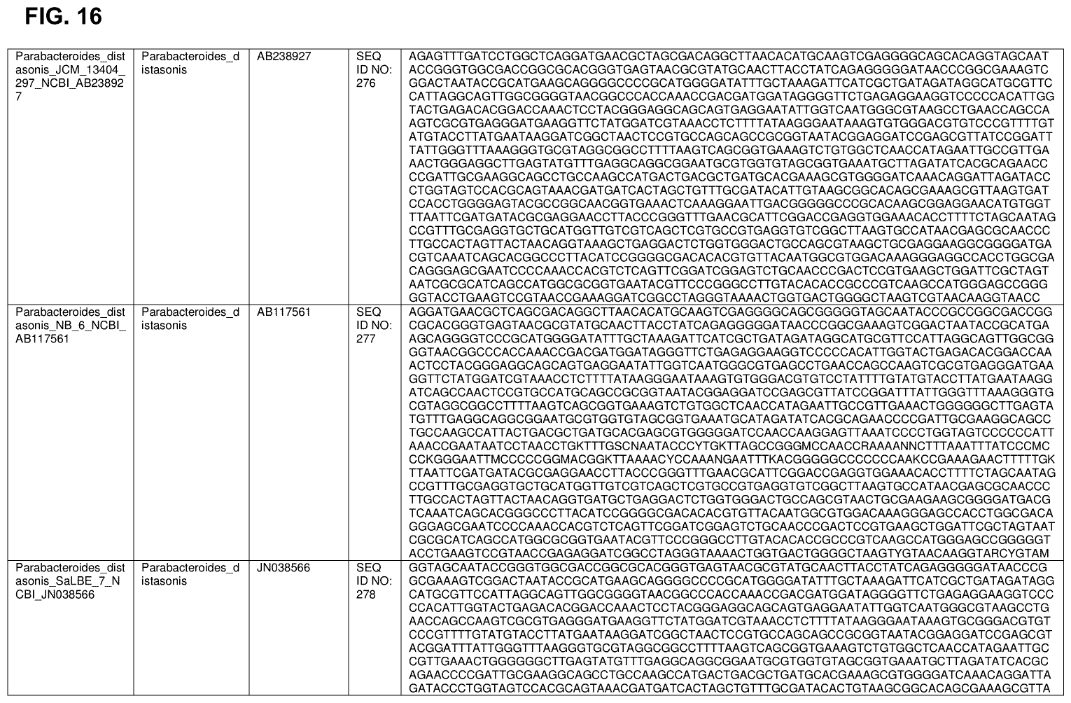

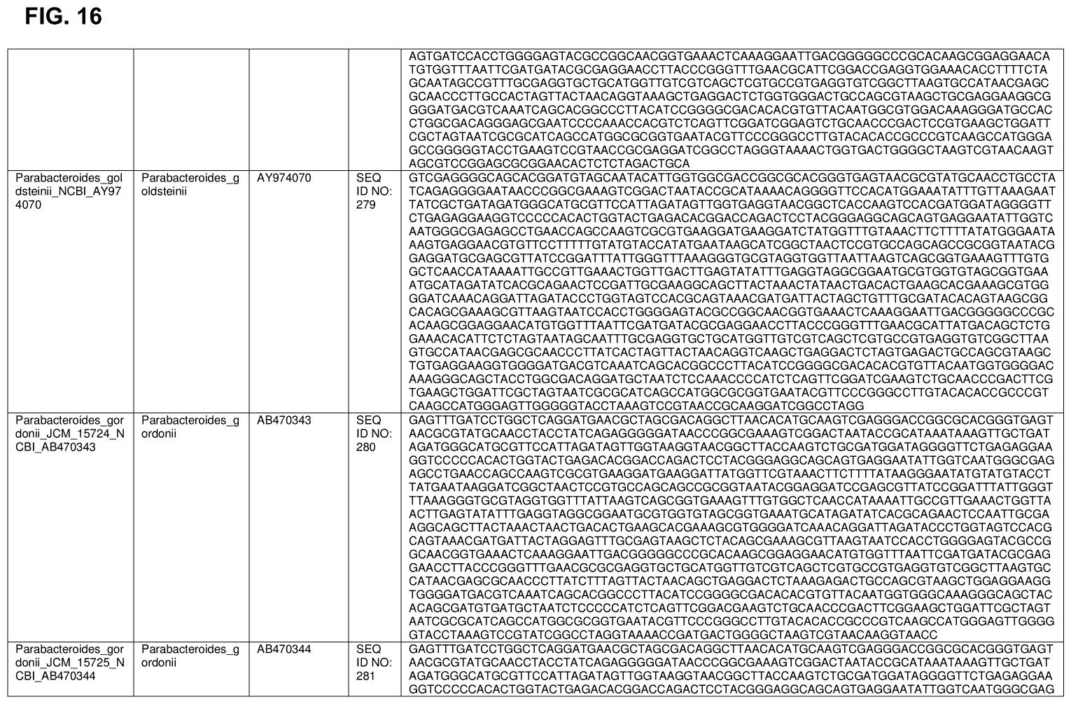

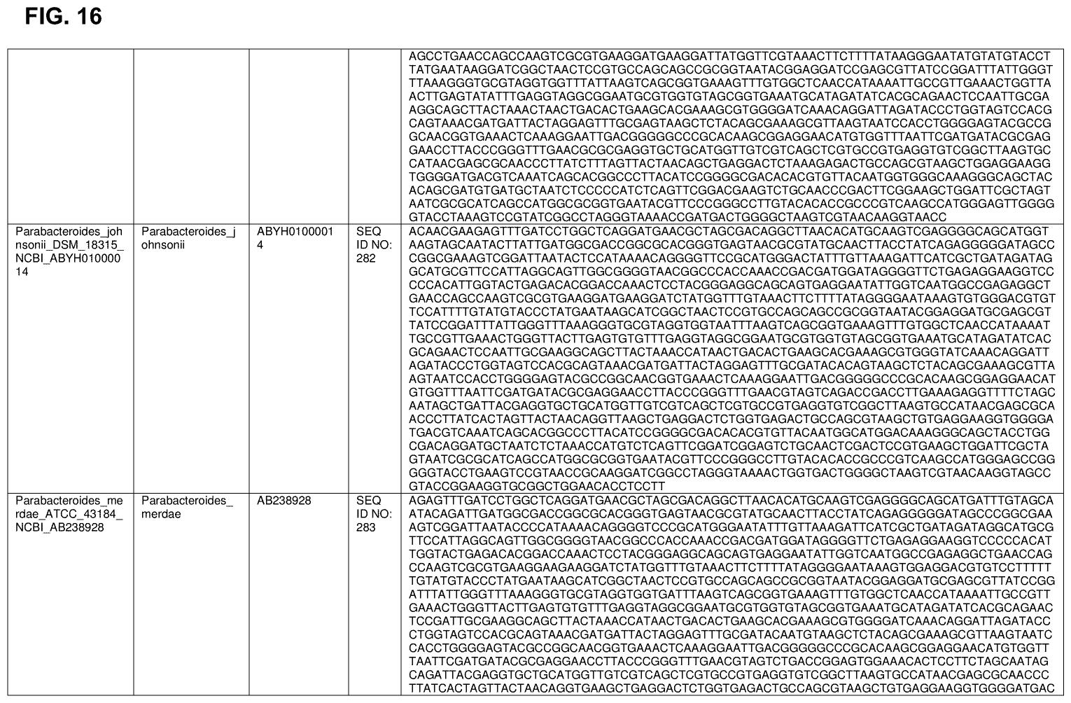

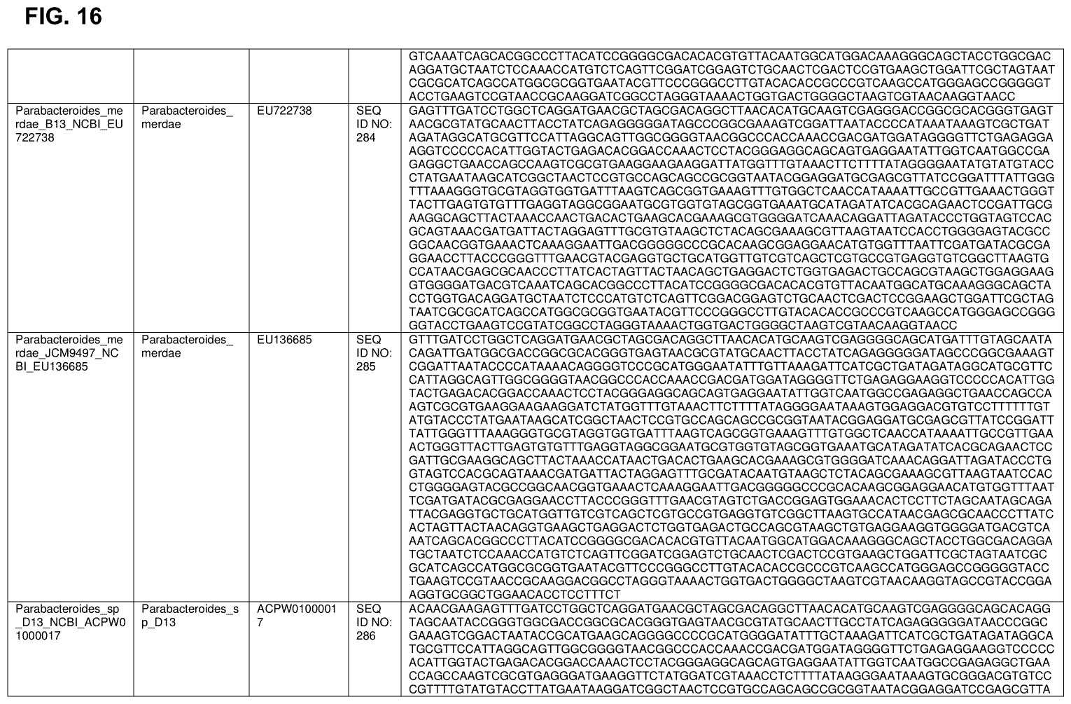

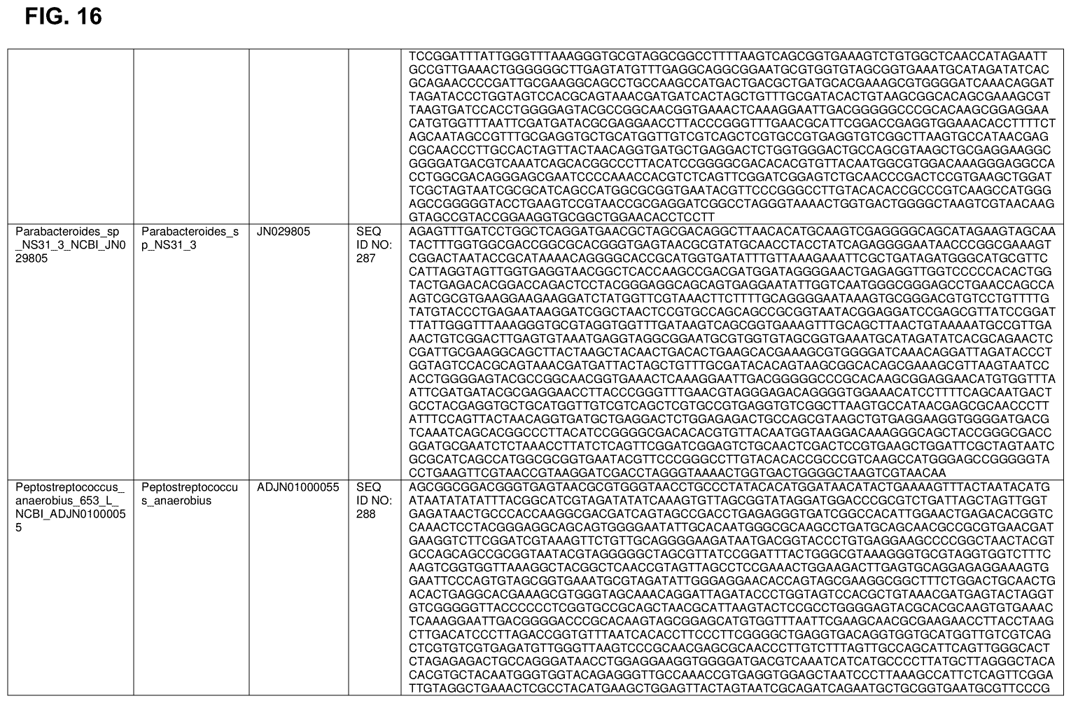

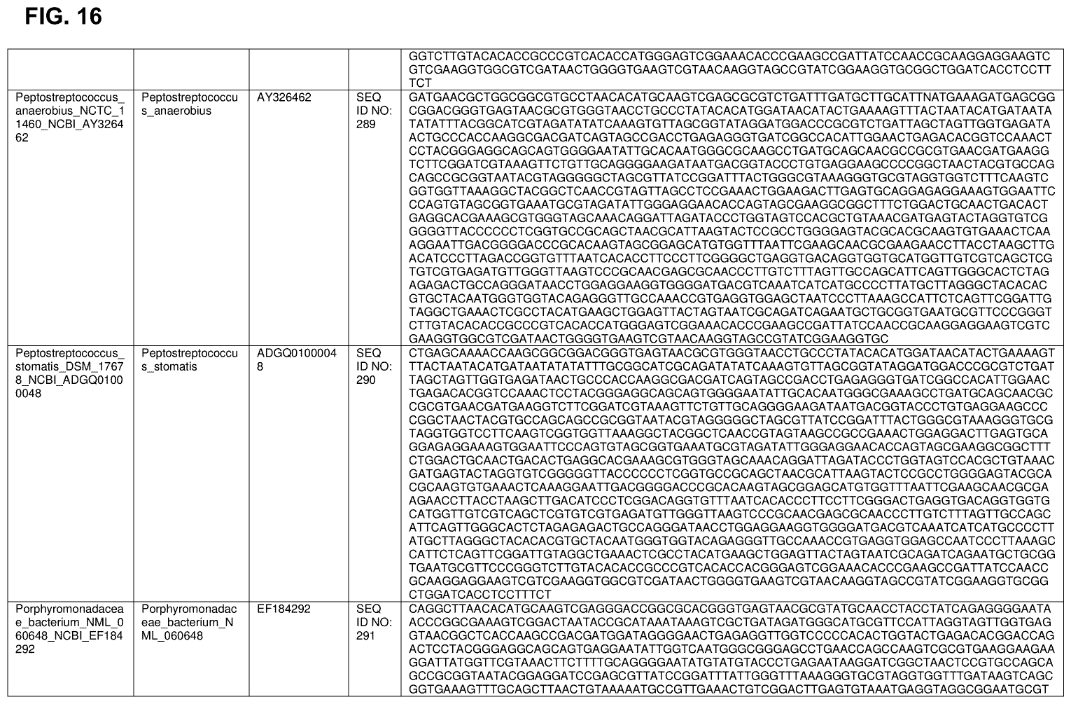

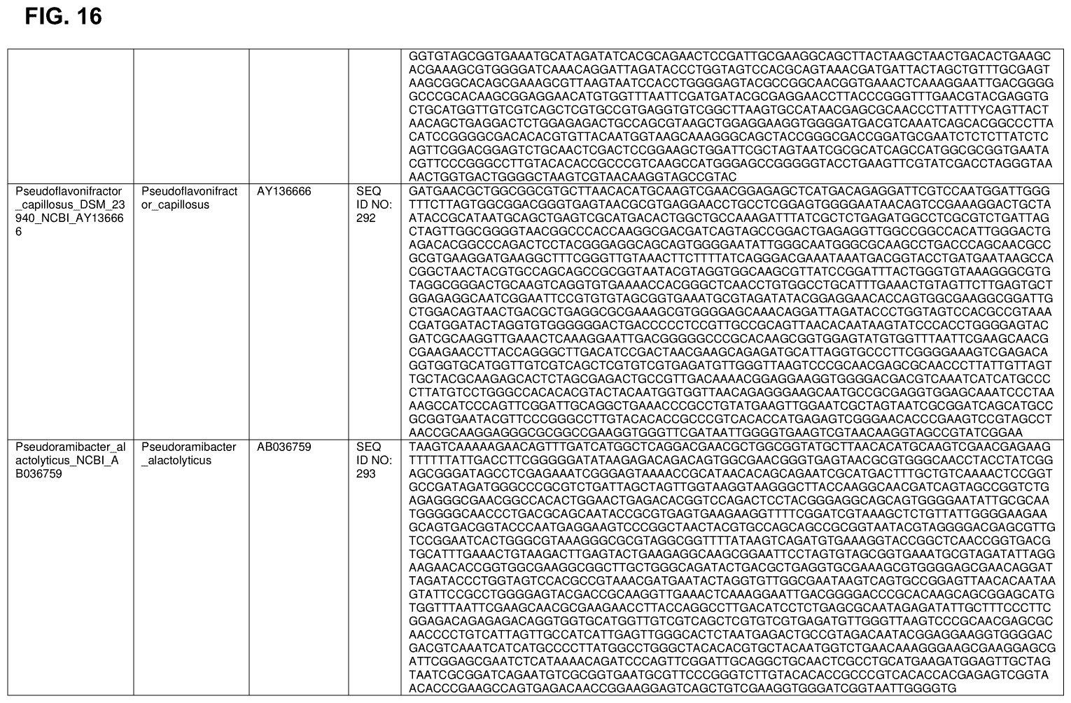

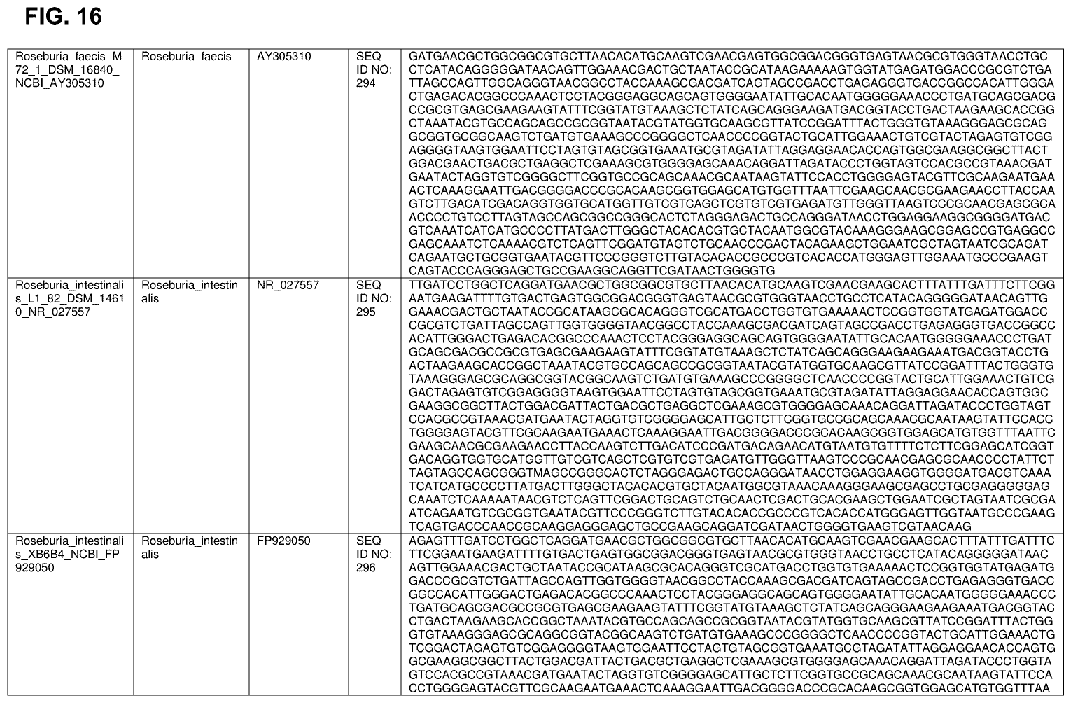

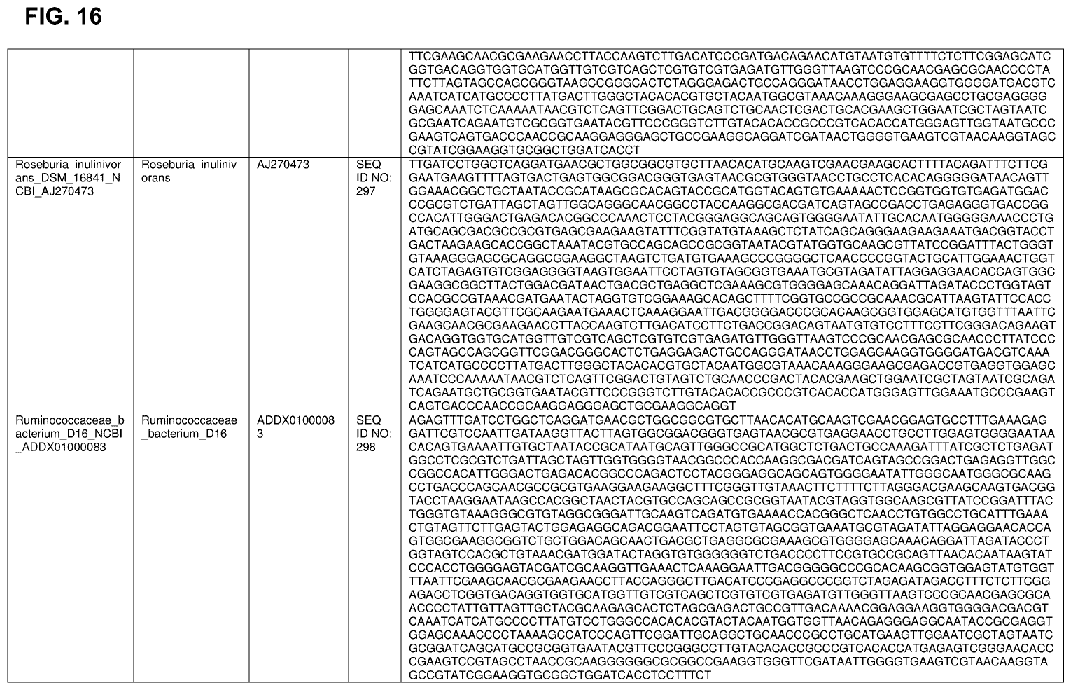

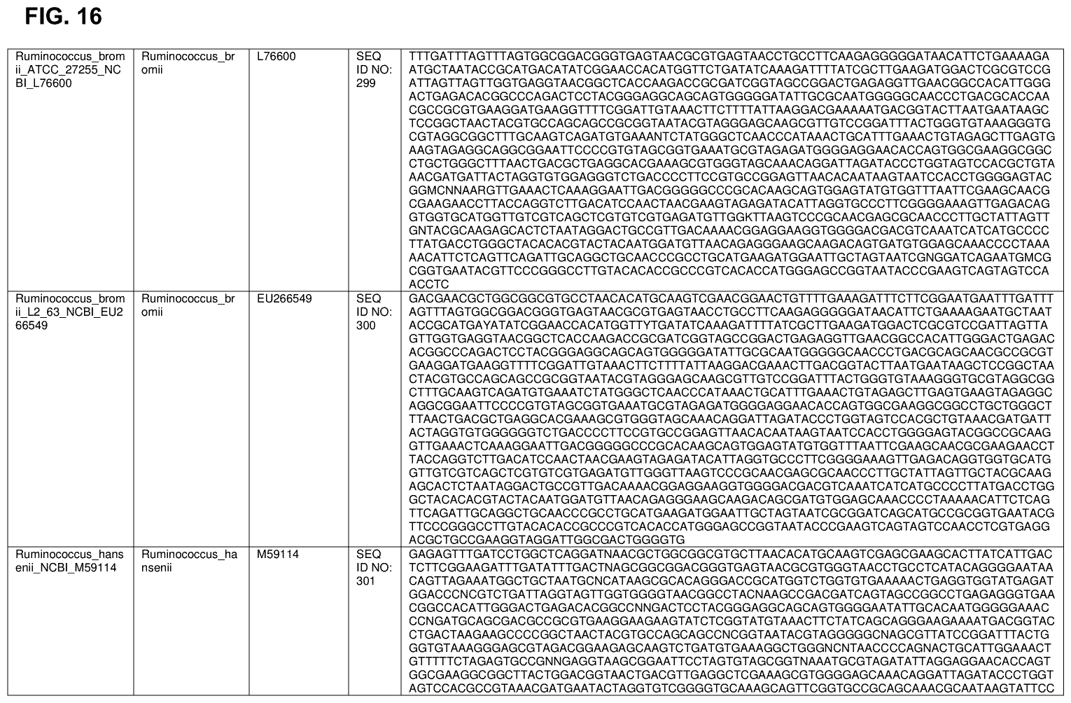

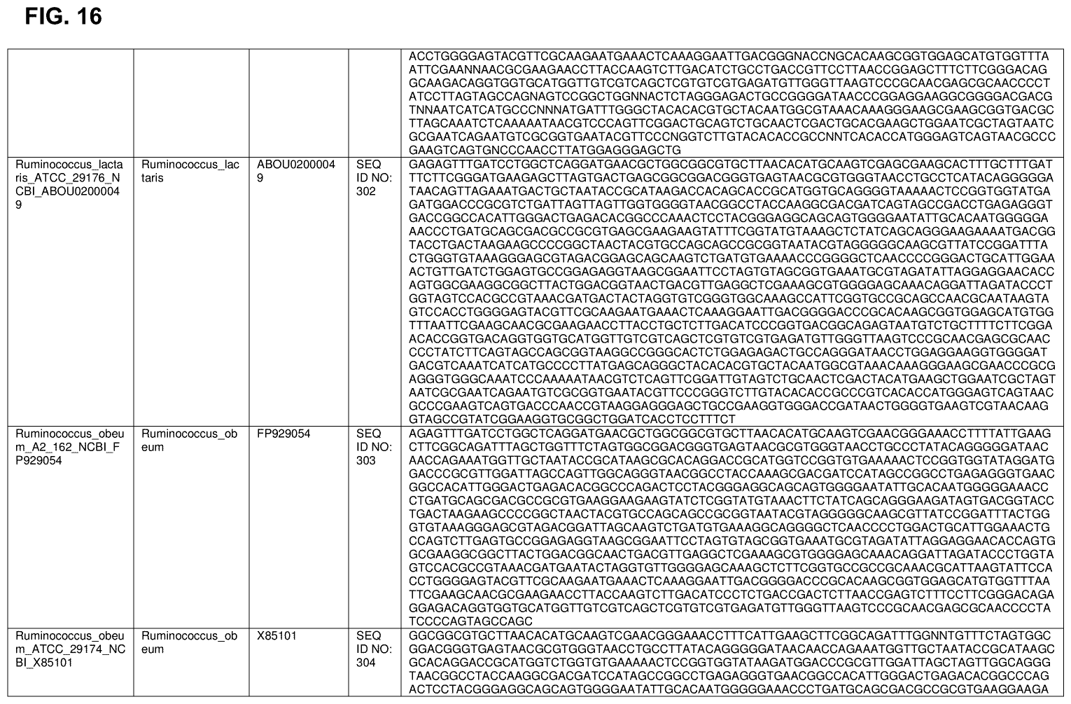

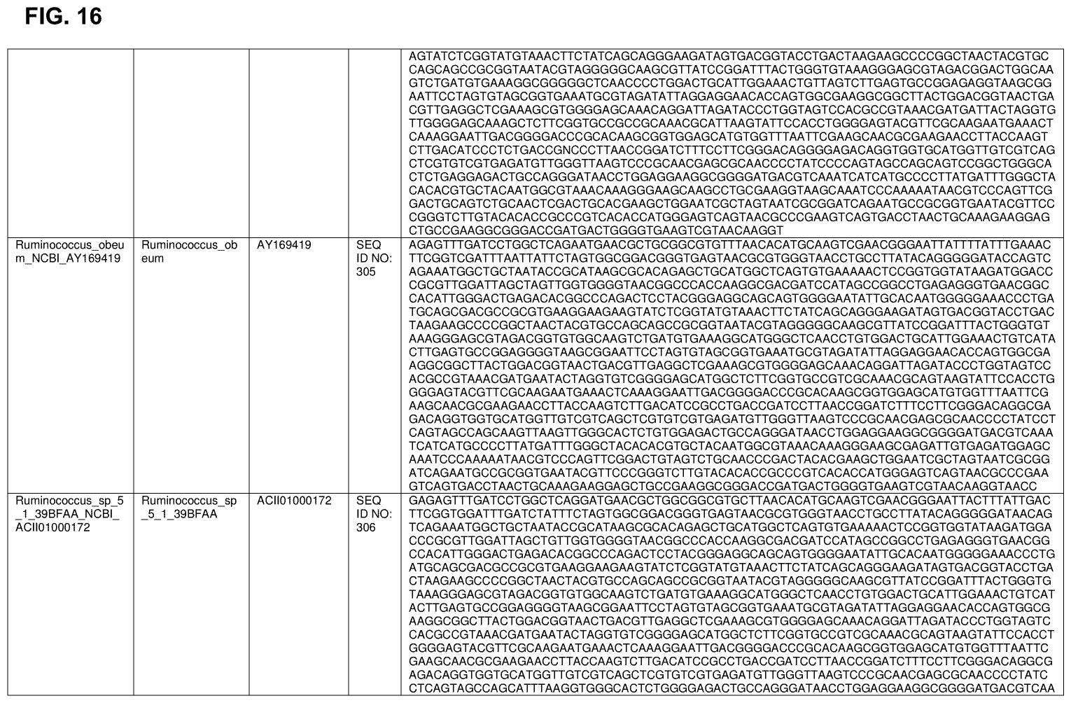

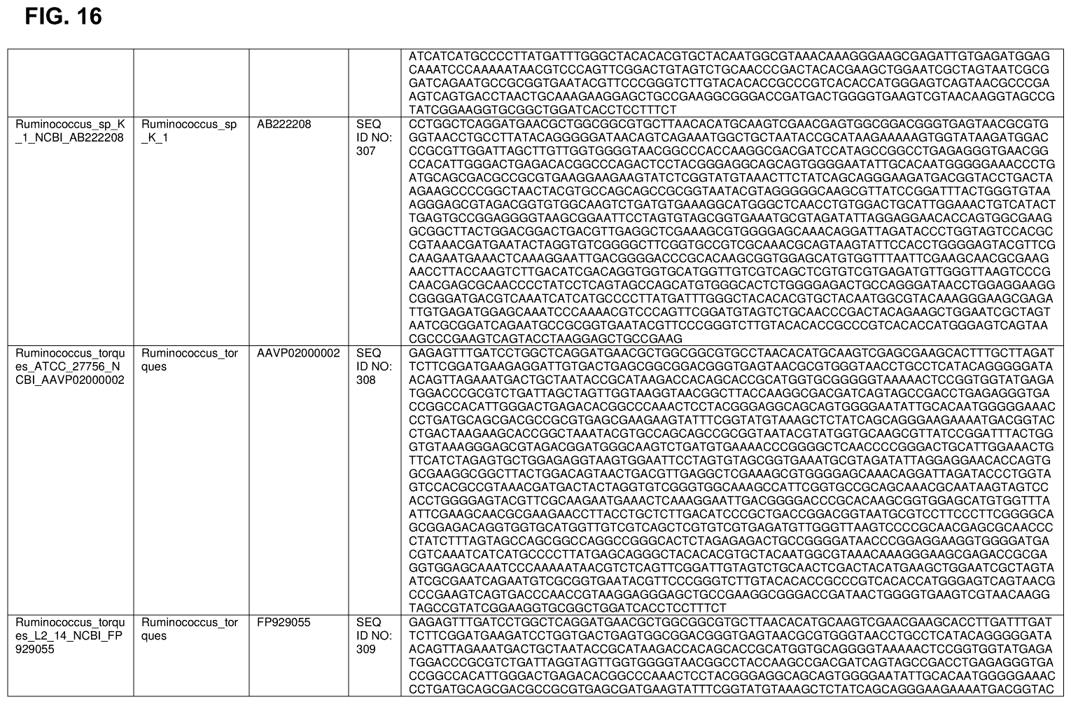

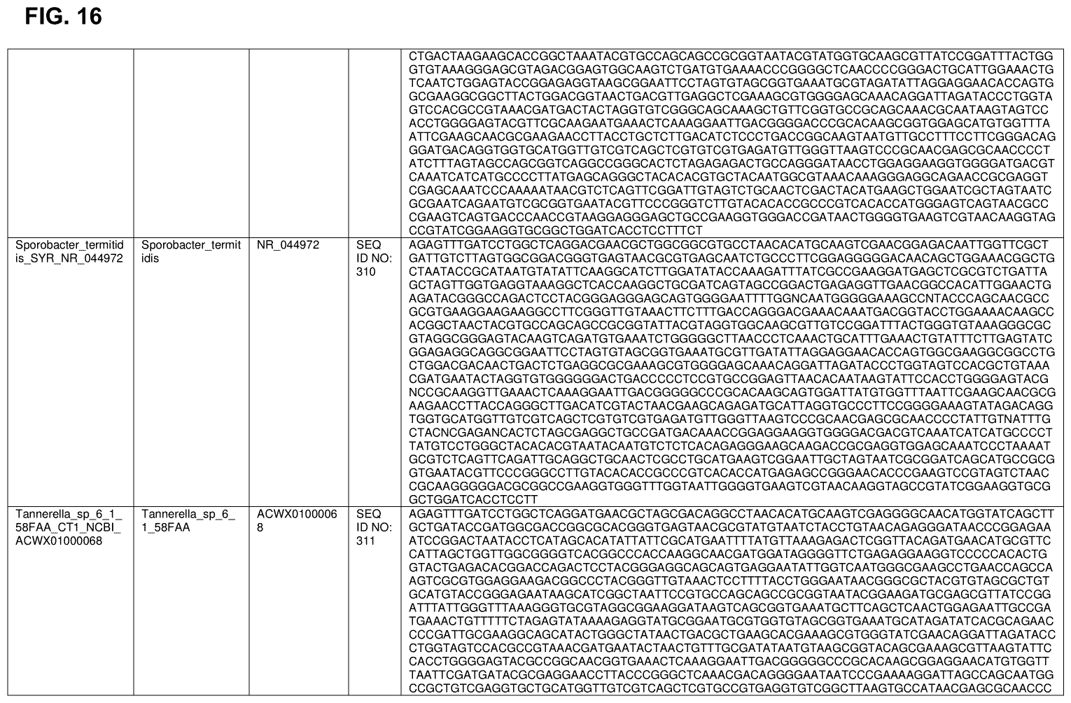

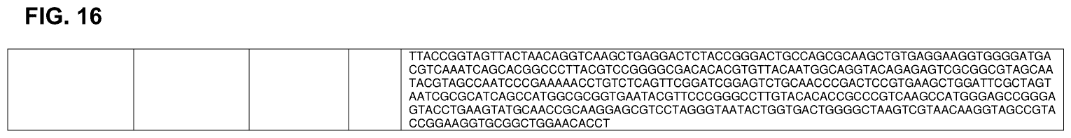

Compositions described herein generally comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 types of bacteria. A bacterial type can be a family, genus, clade, species, or strain. In one example a composition comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 different bacterial species. In another example, a composition comprises bacteria from at least 2, 3, 4, 5, 6, 7, 8, 9, 10, 11, 12, 13, 14, 15, 16, 17, 18, 19, or 20 different clades. In more specific examples, a composition comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 different species from part B or C of Table 1, Table 2, or Table 3; or species from at least 5, 10, 15, or all 18 of each of clades the clades listed in Table 1: 1, 6, 86, 87, 90, 100, 101, 164, 195, 196, 197, 203, 204, and 297. In some embodiments, a bacterial species is identified by homology to a reference sequence (e.g., a 16S rDNA sequence). In general, a bacterial strain having at least 97% identity (e.g., at least 98%, at least 99%, or 100% identity) to a 16S rDNA sequence (the entire sequence or to a variable region(s) such as V4 or V1-3) of a species' reference sequence is the same species as the reference species. An exemplary list of such reference sequences is provided in FIG. 16. In some embodiments, the number of different OTUs or species in the compositions is fewer than 60, 50, 30, 20, or 15. Typically, a composition is formulated in a pharmaceutically acceptable excipient (see below).

For purposes of the inventions described herein, clades are groupings of evolutionarily related bacterial species. Because of their relatedness, bacteria within a clade have a higher than average likelihood of sharing functional features such as bile acid metabolism. Clades are defined based on the topology of a phylogenetic tree that is constructed from full-length 16S sequences using maximum likelihood methods familiar to individuals with ordinary skill in the art of phylogenetics. Clades are constructed to ensure that all OTUs in a given clade are: (i) within a specified number of bootstrap supported nodes from one another, and (ii) within 5% genetic similarity. OTUs that are within the same clade can be distinguished as genetically and phylogenetically distinct from OTUs in a different clade based on 16S-V4 sequence data, while OTUs falling within the same clade are closely related. Compositions substituting one species or OTU with another from the same clade are likely to have conserved ecological function and therefore are useful in the present invention. In some embodiments, bacteria useful in the invention can be selected based on presence in a clade containing one or more bacteria demonstrated to exhibit a specific function and further testing as is known in the art and exemplified herein. In some embodiments, the composition comprises one, two, or three species from five, ten, fifteen, or all 18 clades in Table 1. An exemplary list of species in the clades is provided in Table 2. Table 3 is a list of different bacterial species and an indication of the bile acid metabolizing activities of each. When it is indicated herein that species from Table 3 are included in a composition or formulation of the invention, optionally the species includes one or more of the indicated bile metabolizing activities noted in Table 3. Compositions may also be identified by selecting related organisms based on clades and then testing them for the desired activity according to the methods used to identify bacteria in Table 3. Note that in Table 3, blank cells indicate activities that were not tested for in the corresponding strain.

TABLE 1

| Genus | Species | Clade |
|---|---|---|
| A. No bile acid composition | | |
| Bacteroides | Bacteroides stercoris | 203 |
| Parabacteroides | Parabacteroides merdae | 164 |
| Tyzzerella | Clostridium nexile (T. nexile) | 87 |
| B. Bile salt hydrolase (BSH) composition | | |
| Bacteroides | Bacteroides sp. D20 | 204 |
| Bacteroides | Bacteroides uniformis | 204 |
| Blautia | Blautia wexlerae | 197 |
| Bifidobacterium | Bifidobacterium pseudocatenulatum | 297 |
| Eubacterium | Eubacterium rectale | 6 |
| Ruminococcus | Ruminococcus lactaris | 86 |
| Parabacteroides | Parabacteroides distasonis | 164 |
| C. Maximal activity composition | | |
| Bacteroides | Bacteroides sp. 3_1_23 | 196 |
| Collinsella | Collinsella aerofaciens | 100 |
| Eubacterium | Eubacterium limosum | 1 |
| Blautia | Blautia wexlerae | 197 |
| Bacteroides | Bacteroides sp. D20 | 204 |
| Bacteroides | Bacteroides uniformis | 204 |
| Pseudoflavonifractor | Pseudoflavonifractor capillosus | 101 |
| Terrisporobacter | Clostridium glycolicum | 195 |
| Peptoclostridium | Clostridium ghonii | 195 |
| Clostridia | Lachnospiraceae bacterium | 90 |
| | 5_1_57FAA (isolate a) | |
| Clostridia | Lachnospiraceae bacterium | 90 |
| | 5_1_57FAA (isolate b) | |
| Bifidobacterium | Bifidobacterium pseudocatenulatum | 297 |
| Eubacterium | Eubacterium rectale | 6 |
| Ruminococcus | Ruminococcus lactaris | 86 |
| Parabacteroides | Parabacteroides distasonis | 164 |

TABLE 2
Exemplary species in bacterial clades

| Clade | Exemplary Species |
|---|---|
| clade_1 | Anaerofustis_stercorihominis, |
| clade_1 | Eubacterium_barkeri |
| clade_1 | Eubacterium_callanderi |
| clade_1 | Eubacterium_limosum |
| clade_1 | Pseudoramibacter_alactolyticus |
| clade_100 | Collinsella_aerofaciens |
| clade_100 | Collinsella_intestinalis |
| clade_100 | Collinsella_stercoris |
| clade_100 | Collinsella_tanakaei |
| clade_100 | Coriobacteriaceae_bacterium_phl |
| clade_101 | Clostridium_orbiscindens |
| clade_101 | Clostridium_sp_NML_04A032 |
| clade_101 | Clostridium_viride |
| clade_101 | Flavonifractor_plautii |
| clade_101 | Oscillibacter_sp_G2 |
| clade_101 | Oscillibacter_valericigenes |
| clade_101 | Oscillospira_guilliermondii |
| clade_101 | Papillibacter_cinnamivorans |
| clade_101 | Pseudoflavonifractor_capillosus |
| clade_101 | Ruminococcaceae_bacterium_D16 |
| clade_101 | Sporobacter_termitidis |
| clade_164 | Bacteroides_sp_20_3 |
| clade_164 | Bacteroides_sp_3_1_19 |
| clade_164 | Bacteroides_sp_3_2_5 |
| clade_164 | Parabacteroides_distasonis |
| clade_164 | Parabacteroides_goldsteinii |
| clade_164 | Parabacteroides_gordonii |
| clade_164 | Parabacteroides_johnsonii |
| clade_164 | Parabacteroides_merdae |
| clade_164 | Parabacteroides_sp_D13 |
| clade_164 | Parabacteroides_sp_NS31_3 |
| clade_164 | Porphyromonadaceae_bacterium_NML_060648 |
| clade_195 | Clostridium_bartlettii |
| clade_195 | Clostridium_bifermentans |
| clade_195 | Clostridium_difficile |
| clade_195 | Clostridium_ghonii |
| clade_195 | Clostridium_glycolicum |
| clade_195 | Clostridium_hiranonis |
| clade_195 | Clostridium_irregulare |
| clade_195 | Clostridium_mayombei |
| clade_195 | Clostridium_sordellii |
| clade_195 | Clostridium_sp_MT4_E |
| clade_195 | Eubacterium_tenue |
| clade_195 | Peptostreptococcus_anaerobius |
| clade_195 | Peptostreptococcus_stomatis |
| clade_196 | Bacteroides_acidifaciens |
| clade_196 | Bacteroides_caccae |
| clade_196 | Bacteroides_finegoldii |
| clade_196 | Bacteroides_ovatus |
| clade_196 | Bacteroides_sp_1_1_30 |
| clade_196 | Bacteroides_sp_2_1_22 |
| clade_196 | Bacteroides_sp_2_2_4 |
| clade_196 | Bacteroides_sp_3_1_23 |
| clade_196 | Bacteroides_sp_D1 |
| clade_196 | Bacteroides_sp_D2 |
| clade_196 | Bacteroides_sp_D22 |
| clade_196 | Bacteroides_xylanisolvens |
| clade_197 | Blautia_coccoides |
| clade_197 | Blautia_glucerasea |
| clade_197 | Blautia_glucerasei |
| clade_197 | Blautia_hansenii |
| clade_197 | Blautia_hydrogenotrophica |
| clade_197 | Blautia_luti |
| clade_197 | Blautia_producta |
| clade_197 | Blautia_schinkii |
| clade_197 | Blautia_sp_M25 |
| clade_197 | Blautia_stercoris |
| clade_197 | Blautia_wexlerae |
| clade_197 | Clostridium_coccoides |
| clade_197 | Lachnospiraceae_bacterium_6_1_63FAA |
| clade_197 | Ruminococcus_hansenii |
| clade_197 | Ruminococcus_obeum |
| clade_197 | Ruminococcus_sp_5_1_39BFAA |
| clade_197 | Ruminococcus_sp_K_1 |
| clade_204 | Bacteroides_fluxus |
| clade_204 | Bacteroides_helcogenes |
| clade_204 | Bacteroides_sp_4_1_36 |
| clade_204 | Bacteroides_sp_AR20 |
| clade_204 | Bacteroides_sp_D20 |
| clade_204 | Bacteroides_sp_F_4 |
| clade_204 | Bacteroides_uniformis |
| clade_297 | Bifidobacterium_adolescentis |
| clade_297 | Bifidobacterium_angulatum |
| clade_297 | Bifidobacterium_animalis |
| clade_297 | Bifidobacterium_bifidum |
| clade_297 | Bifidobacterium_breve |
| clade_297 | Bifidobacterium_catenulatum |
| clade_297 | Bifidobacterium_dentium |
| clade_297 | Bifidobacterium_gallicum |
| clade_297 | Bifidobacterium_infantis |
| clade_297 | Bifidobacterium_kashiwanohense |
| clade_297 | Bifidobacterium_longum |
| clade_297 | Bifidobacterium_pseudocatenulatum |
| clade_297 | Bifidobacterium_pseudolongum |
| clade_297 | Bifidobacterium_scardovii |
| clade_297 | Bifidobacterium_sp_HM2 |
| clade_297 | Bifidobacterium_sp_HMLN12 |
| clade_297 | Bifidobacterium_sp_M45 |
| clade_297 | Bifidobacterium_sp_MSX5B |
| clade_297 | Bifidobacterium_sp_TM_7 |
| clade_297 | Bifidobacterium_thermophilum |
| clade_297 | Gardnerella_vaginalis |
| clade_6 | Abiotrophia_defectiva |
| clade_6 | Abiotrophia_sp_oral_clone_P4PA |
| clade_6 | Aerococcus_sanguinicola |
| clade_6 | Aerococcus_urinae |
| clade_6 | Aerococcus_urinaeequi |
| clade_6 | Aerococcus_viridans |
| clade_6 | Butyrivibrio_fibrisolvens |
| clade_6 | Catonella_genomosp_P1_oral_clone |
| clade_6 | Catonella_morbi |
| clade_6 | Catonella_sp_oral_clone_FL037 |
| clade_6 | Eremococcus_coleocola |
| clade_6 | Eubacterium_rectale |
| clade_6 | Eubacterium_sp_oral_clone_GI038 |
| clade_6 | Facklamia_hominis |
| clade_6 | Granulicatella_sp_M658_99_3 |
| clade_6 | Lachnobacterium_bovis |
| clade_6 | Roseburia_inulinivorans |
| clade_86 | Clostridium_glycyrrhizinilyticum |
| clade_86 | Clostridium_oroticum |
| clade_86 | Clostridium_sp_D5 |
| clade_86 | Eubacterium_contortum |
| clade_86 | Eubacterium_fissicatena |
| clade_86 | Lachnospiraceae_bacterium_1_1_57FAA |
| clade_86 | Lachnospiraceae_bacterium_1_4_56FAA |
| clade_86 | Lachnospiraceae_bacterium_8_1_57FAA |
| clade_86 | Ruminococcus_lactaris |
| clade_86 | Ruminococcus_torques |
| clade_90 | Clostridium_hylemonae |
| clade_90 | Clostridium_scindens |
| clade_90 | Dorea_formicigenerans |
| clade_90 | Dorea_longicatena |
| clade_90 | Lachnospiraceae_bacterium_2_1_46FAA |
| clade_90 | Lachnospiraceae_bacterium_4_1_37FAA |
| clade_90 | Lachnospiraceae_bacterium_5_1_57FAA |
| clade_90 | Lachnospiraceae_bacterium_9_1_43BFAA |

TABLE 3

| | | BSH activity | | | | | | | |
|---|---|---|---|---|---|---|---|---|---|
| Genus | Species | gCA | tCA | gCDCA | tCDCA | t-aMCA | t-bMCA | gHCA | tHCA |
| Alistipes | finegoldii | − | +/− | + | + | + | + | + | + |
| Bacteroides | uniformis | − | + | + | + | + | + | | |
| Bacteroides | vulgatus | − | + | + | + | + | + | | |
| Bacteroides | sp_3_1_19 | − | + | + | + | + | + | | + |
| Bacteroides | intestinalis | − | + | + | + | + | + | + | + |
| Bacteroides | sp_20_3 | − | + | + | + | + | + | + | − |
| Bacteroides | intestinalis | − | +/− | + | + | + | + | + | + |
| Bacteroides | caccae | − | − | − | − | + | + | + | + |
| Barnesiella | intestinihominis | − | + | + | + | + | + | + | + |
| Citrobacter | youngae | − | − | − | − | − | − | − | − |
| Citrobacter | youngae | − | − | − | − | − | − | − | − |
| Clostridiales | sp_SM4_1 | − | +/− | − | +/− | +/− | − | − | +/− |
| Clostridium | lavalense | − | +/− | − | + | + | + | +/− | − |
| Clostridium | innocuum | − | +/− | − | + | + | − | +/− | − |
| Clostridium | sp_NML_04A032 | − | − | − | − | − | − | − | − |
| Clostridium | asparagiforme | − | +/− | − | + | + | + | +/− | − |
| Clostridium | spiroforme | − | +/− | − | +/− | +/− | − | − | − |
| Clostridium | sp_NML_04A032 | − | − | − | − | − | − | − | − |
| Coprococcus | comes | − | − | +/− | − | − | − | − | +/− |
| Eggerthella | sp_1_3_56FAA | − | − | − | − | − | − | − | − |
| Erysipelotrichaceae | bacterium_3_1_53 | − | − | − | − | − | − | − | − |
| Eubacterium | dolichum | − | − | − | − | − | − | − | − |
| Eubacterium | sp_3_1_31 | − | +/− | − | +/− | − | − | − | − |
| Lachnospiraceae | bacterium_6_1_63FAA | − | − | − | − | − | − | − | − |
| Lachnospiraceae | bacterium_3_1_57FAA | − | − | − | − | − | − | − | − |
| Lactobacillus | fermentum | − | − | − | − | − | − | − | − |
| Ruminococcus | bromii | − | − | − | − | − | − | − | − |
| Tannerella | sp_6_1_58FAA | − | + | +/− | + | + | + | + | − |
| Clostridium | sp_HGF2_NCBI_AENW01000022 | − | − | − | − | − | − | − | − |
| Clostridium | hylemonae | − | − | − | − | | | − | − |
| Clostridium | hylemonae | − | − | − | − | − | − | − | − |
| Bacteroides | faecis | + | + | + | + | + | + | + | + |
| Bacteroides | sp_3_1_23 | + | + | + | + | + | + | | |
| Bacteroides | sp_D20 | + | + | + | + | + | + | | |
| Bacteroides | eggerthii | + | + | + | + | + | + | | |
| Bifidobacterium | Pseudocatenulatum | + | + | + | + | + | + | | |
| Blautia | schinkii | + | + | + | + | | | + | + |
| Blautia | wexlerae | + | + | + | + | + | + | | |
| Blautia | sp_M25 | + | + | + | + | + | + | + | + |
| Clostridium | sordellii | + | + | + | + | + | + | | |
| Clostridium | sp_YIT_12069 | + | + | + | + | | | | |
| Clostridium | paraputrificum | + | + | + | + | + | + | + | + |
| Clostridium | glycolicum | + | + | + | + | + | + | | |
| Clostridium | paraputrificum | + | + | + | + | +/− | − | + | + |
| Collinsella | aerofaciens | + | + | + | + | + | + | | |
| Coprobacillus | sp_D7 | + | − | + | +/− | − | +/− | − | − |
| Coprococcus | comes | + | + | + | + | + | + | + | + |
| Coprococcus | eutactus | + | + | + | + | + | + | + | + |
| Dorea | longicatena | + | + | + | + | + | + | + | + |
| Dorea | Formicigenerans | + | − | + | − | − | − | | |
| Eubacterium | sp_WAL_14571 | + | + | + | + | + | + | + | + |
| Eubacterium | limosum | + | + | + | + | + | + | | |
| Eubacterium | hallii | + | + | − | + | + | + | | |
| Eubacterium | rectale | + | + | + | + | + | + | | |
| Lachnospiraceae | bacterium_2_1_58FAA | + | + | + | + | + | + | + | − |
| Lachnospiraceae | bacterium_9_1_43BFAA | + | + | + | + | | | + | + |
| Lachnospiraceae | bacterium_3_1_57FAA | + | + | + | + | | | + | + |
| Lactobacillus | acidophilus | + | + | + | + | + | + | + | + |
| Lactobacillus | acidophilus | + | + | + | + | + | + | + | + |
| Lactobacillus | gasseri | + | + | + | + | + | + | + | + |
| Parabacteroides | distasonis | + | + | + | + | + | +/− | | |
| Pseudoflavonifractor | capillosus | + | + | + | + | + | + | | |
| Ruminococcus | lactaris | + | + | + | + | + | + | | |
| Ruminococcus | obeum | + | + | + | + | + | + | + | − |
| Eubacterium | rectale | + | + | + | + | + | + | + | + |
| Lachnospiraceae | bacrerium_3_1_57FAA_CT1 | + | + | + | + | + | + | + | + |
| Clostridium | ghonii | + | + | + | + | + | + | + | + |
| Ruminococcus | lactaris | + | + | + | + | − | − | − | − |
| Blautia | producta | + | + | + | + | | | + | + |
| Clostridium | lactifermentans | + | + | + | + | | | + | + |
| Clostridium | viride | + | + | | | + | + | | |
| Dorea | longicatena | + | + | + | + | + | + | | |
| Clostridium | sp_HGF2 | +/− | +/− | + | +/− | +/− | − | +/− | +/− |
| Clostridium | symbiosum | +/− | − | + | +/− | − | − | − | + |
| Dorea | Formicigenerans | +/− | − | +/− | − | − | − | − | − |
| Gemmiger | formicillis | +/− | + | + | + | + | + | + | − |
| Blautia | schinkii | | | | | | | | |
| Blautia | producta | | | | | | | | |
| Blautia | sp_M25 | | | | | | | | |
| Blautia | producta | | | | | | | | |
| Blautia | glucerasei | | | | | | | | |
| Blautia | producta | | | | | | | | |
| Blautia | coccoides | | | | | | | | |
| Citrobacter | youngae | | | | | | | | |
| Clostridium | scindens | | | | | | | | |
| Clostridium | scindens | | | | | | | | |
| Clostridium | scindens | | | | | | | | |
| Clostridium | disporicum | | | | | | | | |
| Clostridium | butyricum | | | | | | | | |
| Clostridium | orbiscindens | | | | | | | | |
| Clostridium | bolteae | | | | | | | | |
| Clostridium | sp_7_2_43FAA | | | | | | | | |
| Coprococcus | comes | | | | | | | | |
| Eubacterium | rectale | | | | | | | | |
| Eubacterium | rectale | | | | | | | | |
| Eubacterium | contortum | | | | | | | | |
| Lachnospiraceae | bacterium_5_1_57FAA | | | | | | | | |
| Lachnospiraceae | bacterium_5_1_57FAA | | | | | | | | |
| Lachnospiraceae | bacterium_5_1_57FAA | | | | | | | | |
| Lachnospiraceae | bacterium_5_1_57FAA | | | | | | | | |
| Roseburia | faecis | | | | | | | | |
| Roseburia | intestinalis | | | | | | | | |
| Ruminococcus | sp_5_1_39BFAA | | | | | | | | |
| Ruminococcus | bromii | | | | | | | | |
| Ruminococcus | hansenii | | | | | | | | |
| Clostridium | hylemonae | | | | | | | | |
| Lachnospiraceae | bacterium_5_1_57FAA | | | | | | | | |
| Lachnospiraceae | bacterium_5_1_57FAA | | | | | | | | |
| Lachnospiraceae | bacterium_oral_taxon_F15 | | | | | | | | |
| Clostridium | scindens | | | | | | | | |
| Lachnospiraceae | bacterium_5_1_57FAA | | | | | | | | |
| Lachnospiraceae | bacterium_5_1_57FAA | | | | | | | | |
| Lachnospiraceae | bacterium_5_1_57FAA | | | | | | | | |
| Dorea | longicatena | | | | | | | | |
| Clostridium | hylemonae | | | | | | | | |
| Lachnospiraceae | bacterium_5_1_57FAA | | | | | | | | |

| | | 7a-HSDH | | 3a-HSDH | | 12a-HSDH | | 7a-Dehydrox- | |
|---|---|---|---|---|---|---|---|---|---|
| | | activity | | activity | | activity | | ylation | |
| Genus | Species | CA | CDCA | CDCA | LCA | CA | DCA | CA | CDCA |
| Alistipes | finegoldii | − | − | − | − | − | − | − | − |
| Bacteroides | uniformis | − | − | − | − | − | − | − | − |
| Bacteroides | vulgatus | − | − | − | − | − | − | − | − |
| Bacteroides | sp_3_1_19 | | | | | | | | |
| Bacteroides | intestinalis | | | | | | | | |
| Bacteroides | sp_20_3 | | | | | | | | |
| Bacteroides | intestinalis | | | | | | | | |
| Bacteroides | caccae | | | | | | | | |
| Barnesiella | intestinihominis | | | | | | | | |
| Citrobacter | youngae | − | − | − | − | − | − | − | − |
| Citrobacter | youngae | − | − | − | − | − | − | − | − |
| Clostridiales | sp_SM4_1 | | | | | | | | |
| Clostridium | lavalense | + | − | − | − | − | + | − | − |
| Clostridium | innocuum | − | − | − | − | − | − | − | − |
| Clostridium | sp_NML_04A032 | − | − | − | − | − | − | − | − |
| Clostridium | asparagiforme | + | − | +/− | − | − | + | − | − |
| Clostridium | spiroforme | | | | | | | | |
| Clostridium | sp_NML_04A032 | | | | | | | | |
| Coprococcus | comes | + | − | − | − | − | + | − | − |
| Eggerthella | sp_1_3_56FAA | | | | | | | | |
| Erysipelotrichaceae | bacterium_3_1_53 | + | + | − | − | − | − | − | − |
| Eubacterium | dolichum | − | − | − | − | − | − | − | − |
| Eubacterium | sp_3_1_31 | − | − | + | + | − | + | − | − |
| Lachnospiraceae | bacterium_6_1_63FAA | | | | | | | | |
| Lachnospiraceae | bacterium_3_1_57FAA | | | | | | | | |
| Lactobacillus | fermentum | | | | | | | | |
| Ruminococcus | bromii | − | − | +/− | − | − | − | − | − |
| Tannerella | sp_6_1_58FAA | | | | | | | | |
| Clostridium | sp_HGF2_NCBI_AENW01000022 | | | | | | | | |
| Clostridium | hylemonae | + | − | + | − | + | + | + | +/− |
| Clostridium | hylemonae | +/− | − | + | | − | + | + | − |
| Bacteroides | faecis | | | | | | | | |
| Bacteroides | sp_3_1_23 | + | + | − | − | − | − | − | − |
| Bacteroides | sp_D20 | − | − | − | − | − | − | − | − |
| Bacteroides | eggerthii | − | − | − | − | − | − | − | − |
| Bifidobacterium | Pseudocatenulatum | − | − | − | − | − | − | − | − |
| Blautia | schinkii | | | | | | | | |
| Blautia | wexlerae | − | − | − | − | − | − | − | − |
| Blautia | sp_M25 | | | | | | | | |
| Clostridium | sordellii | − | − | +/− | − | − | | − | − |
| Clostridium | sp_YIT_12069 | | | | | | | | |
| Clostridium | paraputrificum | | | | | | | | |
| Clostridium | glycolicum | | | | | | | | |
| Clostridium | paraputrificum | | | | | | | | |
| Collinsella | aerofaciens | + | − | + | + | − | + | − | − |
| Coprobacillus | sp_D7 | | | | | | | | |
| Coprococcus | comes | + | − | − | − | − | + | − | − |
| Coprococcus | eutactus | + | − | +/− | − | − | + | − | − |
| Dorea | longicatena | − | − | − | − | − | − | +/− | − |
| Dorea | Formicigenerans | | | | | | | | |
| Eubacterium | sp_WAL_14571 | | | | | | | | |
| Eubacterium | limosum | + | + | − | − | − | + | − | − |
| Eubacterium | hallii | + | − | − | − | − | + | − | − |
| Eubacterium | rectale | − | − | − | − | − | − | − | − |
| Lachnospiraceae | bacterium_2_1_58FAA | − | − | + | + | − | − | − | − |
| Lachnospiraceae | bacterium_9_1_43BFAA | | | | | | | | |
| Lachnospiraceae | bacterium_3_1_57FAA | | | | | | | | |
| Lactobacillus | acidophilus | | | | | | | | |
| Lactobacillus | acidophilus | | | | | | | | |
| Lactobacillus | gasseri | | | | | | | | |
| Parabacteroides | distasonis | − | − | − | − | − | − | − | − |
| Pseudoflavonifractor | capillosus | − | − | + | +/− | − | − | − | − |
| Ruminococcus | lactaris | − | − | − | − | − | − | − | − |
| Ruminococcus | obeum | | | | | | | | |
| Eubacterium | rectale | − | − | +/− | − | − | − | − | − |
| Lachnospiraceae | bacrerium_3_1_57FAA_CT1 | | | | | | | | |
| Clostridium | ghonii | +/− | + | + | | − | − | +/− | − |
| Ruminococcus | lactaris | + | − | − | | − | − | +/− | − |
| Blautia | producta | − | − | − | | − | +/− | +/− | − |
| Clostridium | lactifermentans | − | + | + | | − | − | +/− | − |
| Clostridium | viride | − | + | − | | − | +/− | +/− | − |
| Dorea | longicatena | − | − | − | − | − | − | +/− | − |
| Clostridium | sp_HGF2 | − | − | − | − | − | − | − | − |
| Clostridium | symbiosum | + | + | +/− | − | − | − | − | − |
| Dorea | Formicigenerans | | | | | | | | |
| Gemmiger | formicillis | − | − | − | − | − | − | − | − |
| Blautia | schinkii | − | + | − | − | − | − | − | − |
| Blautia | producta | − | − | − | − | − | − | − | − |
| Blautia | sp_M25 | − | − | +/− | − | − | − | − | − |
| Blautia | producta | | | | | | | | |
| Blautia | glucerasei | | | | | | | | |
| Blautia | producta | | | | | | | | |
| Blautia | coccoides | | | | | | | | |
| Citrobacter | youngae | − | − | − | − | − | − | − | − |
| Clostridium | scindens | + | + | + | + | + | + | + | + |
| Clostridium | scindens | − | + | + | − | − | + | + | + |
| Clostridium | scindens | − | + | + | − | − | + | + | + |
| Clostridium | disporicum | − | + | − | − | − | − | − | − |
| Clostridium | butyricum | − | + | − | − | − | − | − | − |
| Clostridium | orbiscindens | − | + | − | − | − | + | − | − |
| Clostridium | bolteae | − | − | + | +/− | − | − | − | − |
| Clostridium | sp_7_2_43FAA | + | + | +/− | − | − | − | − | − |
| Coprococcus | comes | − | − | − | − | − | − | − | − |
| Eubacterium | rectale | − | − | − | − | − | − | − | − |
| Eubacterium | rectale | − | − | − | − | − | − | − | − |
| Eubacterium | contortum | − | − | +/− | − | − | − | − | − |
| Lachnospiraceae | bacterium_5_1_57FAA | +/− | +/− | + | + | +/− | | + | + |
| Lachnospiraceae | bacterium_5_1_57FAA | − | +/− | + | + | − | + | + | + |
| Lachnospiraceae | bacterium_5_1_57FAA | − | +/− | + | + | − | + | + | + |
| Lachnospiraceae | bacterium_5_1_57FAA | − | +/− | + | + | − | + | + | + |
| Roseburia | faecis | − | − | +/− | − | − | − | − | − |
| Roseburia | intestinalis | − | − | − | − | − | − | − | − |
| Ruminococcus | sp_5_1_39BFAA | − | − | − | − | − | − | − | − |
| Ruminococcus | bromii | − | − | − | − | − | − | − | − |
| Ruminococcus | hansenii | − | − | − | − | − | − | − | − |
| Clostridium | hylemonae | +/− | − | + | − | − | + | + | + |
| Lachnospiraceae | bacterium_5_1_57FAA | − | + | + | + | − | − | + | + |
| Lachnospiraceae | bacterium_5_1_57FAA | − | + | + | + | − | − | + | + |
| Lachnospiraceae | bacterium_oral_taxon_F15 | − | + | + | | − | +/− | +/− | − |
| Clostridium | scindens | + | + | +/− | − | +/− | + | + | + |
| Lachnospiraceae | bacterium_5_1_57FAA | + | + | + | − | + | − | + | + |
| Lachnospiraceae | bacterium_5_1_57FAA | − | − | + | + | − | − | + | + |
| Lachnospiraceae | bacterium_5_1_57FAA | − | − | + | + | − | − | + | + |
| Dorea | longicatena | − | − | − | − | − | − | − | − |
| Clostridium | hylemonae | +/− | − | − | − | − | − | − | − |
| Lachnospiraceae | bacterium_5_1_57FAA | + | + | − | − | − | − | − | − |

In some embodiments, all organisms in a composition are obligate anaerobes. In some embodiments, the bacteria in a composition are species that can be cultured in vitro to form spores and such spores can be germinated in vitro. In some embodiments, the bacteria in a composition are spores. In some embodiments, the bacteria in a composition are in vegetative form. It is to be understood that a composition of bacterial spores or a composition of vegetative bacteria means that while the majority of bacteria are in the specified form (i.e., spore or vegetative), a small number may be in a different form, e.g., in the case of spores, some cells in a composition may be vegetative, while in the case of vegetative bacteria, some cells may be in the form of spores. For example, the composition may be 100%, at least 99%, at least 97%, at least 95%, at least 90%, at least 85%, at least 80%, or at least 75% spores, or the composition may be 100%, at least 99%, at least 97%, at least 95%, at least 90%, at least 85%, at least 80%, or at least 75% vegetative bacteria. In some embodiments, the individual species are present as a mixture of vegetative bacteria and spores. In some embodiments, the number of a species used in a composition is determined using colony-forming unit (cfu) assay, although other methods known in the art can be used. The assessment of percent of bacteria in a vegetative or spore specific form may be referenced as of the date of preparing the composition in a dosage form or as of the date or administration of the dosage form. Methods of preparing spores are described in the art, e.g., U.S. Pat. No. 9,011,834.

The total number of bacteria effective in a treatment is far below the total number of organisms in the gastrointestinal tract of a healthy human, i.e., it is not necessary to administer a complete healthy microbiome to achieve a therapeutic effect, not only in terms of the diversity of the species provided in a composition, but also in the total number of organisms provided.

It is to be understood that if a composition is indicated as “consisting of” particular types of bacteria in this application, this refers only to the bacteria types that are present in a composition. A bacterial formulation that “consists of” a particular list of bacteria may contain additional non-bacterial materials such as one or more excipients (including, for example, one or more capsules), an aqueous or non-aqueous medium (for example, glycerol, polyethylene glycol, cocoa butter, water, and/or buffer), as well as one or more prebiotics or small molecule drugs.

### Determination of Identity

Clades, operational taxonomic units (OTUs), species, and strains are, in some embodiments, identified by 16S rDNA sequence(s). The relatedness of clades, OTUs, species, and strains can be determined by the percent identity between clades, OTUs, species, or strains. In some cases, the percent identity is determined using a 16S rDNA sequence. The 16S rDNA sequence can be full-length, one or more variable regions and be from a single sequence or a composite derived from multiple 16S rDNA sequences from a strain, species, or OTU. Percent identity between a reference and query sequence can be determined using methods known in the art. Non-limiting examples of methods for such determinations are provided below. As used herein, the relatedness between two nucleotide sequences is described by the parameter “identity.” In general, a two bacteria are the same OTU or species if they have 16S rDNA identity of at least 95%, e.g., 97%, 98%, 99%, or 100%. In some embodiments, the 16S rDNA identity is determined for the full length 16S rDNA molecule. In some embodiments, the 16S rDNA identity is determined for a fragment of a 16S rDNA molecule, such as a variable region (e.g., V4). In some embodiments, the identity is determined for a fragment of, e.g., at least 10, 15, 20, 25, 30, 35, 40, 45, 50, 60, 75, 100, 125, 150, 175, 200, 225, 250, 300, or more nucleotides in length (or in ranges between any of the numbers listed herein; or in specific values in such ranges).

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical nucleotide in the two aligned sequences on a given position in the alignment and 3) dividing the number of exact matches with the length of the reference sequence.

In another embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical nucleotide in the two aligned sequences on a given position in the alignment and 3) dividing the number of exact matches with the length of the longest of the two sequences.

In another embodiment, the degree of sequence identity between the query sequence and the reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the “alignment length,” where the alignment length is the length of the entire alignment including gaps and overhanging parts of the sequences.

Sequence identity comparisons are, generally, with the aid of a sequence comparison program. These commercially or publicly available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalizing the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.

In general, the default values of the alignment program are used for sequence comparisons. Suitable computer programs useful for determining identity include, for example, BLAST (blast.ncbi.nlm.nih.gov).

In an embodiment of the present invention, the alignment program optimizes the alignment over the full-length of selected sequences, e.g., full-length, V1-3, V4, or V6 16S rDNA sequence. A 16S rDNA sequence can be a single sequence or a composite of multiple 16S rDNA sequences from a selected strain, species, or OTU. For example, the global alignment program is based on the Needleman-Wunsch algorithm (Needleman and Wunsch, J. Mol. Biol. 48:443-453, 1970). Non-limiting examples of such programs are EMBOSS Needle and EMBOSS Stretcher programs, available at ebi.ac.uk/Tools/psa/.

In one embodiment, the sequences are aligned by a global alignment program and the sequence identity is calculated by identifying the number of exact matches identified by the program divided by the “alignment length,” where the alignment length is the length of the entire alignment including gaps and overhanging parts of the sequences. In a further embodiment, the global alignment program uses the Needleman-Wunsch algorithm and the sequence identity is calculated by identifying the number of exact matches identified by the program divided by the “alignment length,” where the alignment length is the length of the entire alignment including gaps and overhanging parts of the sequences.

In yet a further embodiment, the global alignment program is selected from the group consisting of EMBOSS Needle and EMBOSS stretcher and the sequence identity is calculated by identifying the number of exact matches identified by the program divided by the “alignment length,” where the alignment length is the length of the entire alignment including gaps and overhanging parts of the sequences.

Once the software has produced an alignment, it is possible to calculate percent (%) similarity and percent sequence identity.

### Formulations

In some embodiments, treatment includes administering a composition to a subject, for example, a patient at risk for, recently treated for, or that has been diagnosed with a cholestatic disease or condition. In some embodiments, the composition is an oral dosage form. In some embodiments, the composition comprises, as the active component a consortium of bacteria as described herein in combination with one or more pharmaceutically acceptable carriers (excipients). In making the compositions of the invention, the bacteria are typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active component. Thus, a formulation can be in the form of a tablet, pill, powder, lozenge, sachet, cachet, elixir, suspension, emulsion, solution, syrup, aerosol (as a solid or in a liquid medium), ointment containing, for example, up to 10% by weight of the active component, soft capsule, hard capsule, gel-cap, tablet, suppository, solution, or packaged powder. Suitable excipients include, for example, PBS, glycerol, cocoa butter, or polyethylene glycol.

In preparing a formulation, a solid form of the composition can be milled to provide the appropriate particle size prior to combining with the other ingredients. Moreover, the compositions can be formulated so as to provide quick, sustained or delayed release of the active component after administration to the patient, for example, for release in the colon, by employing methods and forms known in the art.

A composition can be formulated in a unit dosage form, each dosage form containing from about 10² to about 10⁹ viable OTUs, for example, about 10⁴ to about 10⁸ OTUs. In some embodiments, substantially all of the bacteria are in spore form. In some embodiments, the bacteria are in spore and vegetative form. The term “unit dosage forms” refers to physically discrete units suitable as unitary dosages for human subjects and other mammals, each unit containing a predetermined quantity of active component calculated to produce the desired therapeutic effect, in association with a suitable pharmaceutical excipient. In some cases, more than one unit dosage form constitutes a dose. For example, a single dose can be one unit dosage form, two dosage unit forms, three dosage unit forms, four unit dosage forms, five unit dosage forms or more. In some cases, the number of unit dosage forms constituting a single dose is 1, 2, 3, 4, 5, 6, 7, 8, 9, 10, 11, 12, 13, 14, 15, 20, 25, or 30 unit dosage forms. A single dose can be, e.g., 10³ to about 10⁹ spores, for example, about 10⁴ to about 10⁸ spores. In an example, a dose is 1, 2, 3, or 4 capsules containing a total of between 10² and 10⁸ spores in the dose. In the case of a single dose having multiple dosage forms, the dosage forms are generally delivered within a prescribed period, e.g., within 1 hour, 2 hours, 5 hours, 10 hours, 15 hours, or 24 hours.

A composition described herein can be effective over a wide dosage range and is generally administered in a pharmaceutically effective amount.

A tablet or pill comprising a composition described herein can be coated or otherwise compounded to provide a dosage form, for example, to ease delivery (for example, by improving swallowability) or to improve delivery to a targeted area of the gastrointestinal tract such as the colon.

In some embodiments, the tablet or pill comprises an inner component surrounding the composition and an outer component, the latter serving as an envelope over the former. The two components can be separated by an enteric coating layer that may resist disintegration in the stomach and permits the inner component to pass intact into the duodenum or to be delayed in release.

In some embodiments, a formulation comprising a composition of the invention is administered via a nasogastric route, by endoscopy or other suitable method of delivering the formulation at or near a desired site, for example, the upper intestinal tract (e.g., stomach and/or duodenum) or the lower intestinal tract (e.g., small intestine and/or large intestine). Effective doses can be extrapolated from dose-response curves derived from in vitro or animal model test systems or from clinical studies.

Furthermore, the formulations can optionally be administered in combination with antacids that are known in the art.

### Methods of Treatment

Compositions as described herein are useful for administration to a subject, e.g., a mammal such as a human in need of treatment, e.g., to prevent or treat a cholestatic disease or condition. Examples of such diseases include general cholestasis (GC), primary sclerosing cholangitis (PSC), primary biliary cholangitis (PBS), progressive familial intrahepatic cholestasis (PFIC), non-alcoholic fatty liver disease (NAFLD), non-alcoholic steatohepatitis (NASH), cholestasis of pregnancy, cholangitis, hepatitis, alcoholic liver disease, hepatocellular carcinoma, liver cirrhosis, cystic fibrosis, and graft-versus-host disease (GVHD). In addition, subjects having a blockage of an extrahepatic bile duct due to, e.g., gall stone, inflammatory stricture, cancer, or pancreatitis, can be treated by the methods of the invention. For example, a Max BA activity composition that can provide BSH, 7α-dehydroxylation, and hydroxysteroid dehydrogenation activities (elevated activity compared to a reference having low activity), can be used to treat cholestatic disease patients that show abnormal bile acid composition, e.g., an abnormal accumulation of conjugated primary bile acids.

In some embodiments, patients responding to OCA and/or UDCA treatment, but experiencing undesirable side-effects (e.g., severe (intolerable) pruritus, elevations in liver-related adverse reactions and/or biochemical tests associated with OCA treatment, or an undesirable reduction in HDL-C) can be treated with a BSH only composition, or other composition described herein having at least a BSH activity, thereby permitting a reduction in the amount or frequency of administration of the OCA and/or UDCA; or increasing the patient's tolerance of a normal or elevated dose of OCA and/or UDCA. Without committing to any particular theory, it may be that by altering levels of bile acids in such patients, primary and/or secondary bile acids such as CDCA synergize with OCA or UDCA, improving patient treatment of a liver disease such as PBC.

The methods and compositions of the invention can be used to improve one or more symptoms of these diseases and conditions by improving bile acid metabolism. While the methods may not necessarily address the causal pathologies of cholestatic diseases, depleting the source of bile duct injury, both by reducing total bile acid pools and conversion of conjugated primary bile salts into primary bile acids and/or to secondary bile acids, can have a significant impact on the progression of disease and patient well-being, including in the absence of significant alternative approaches. Advantages of this approach can include treatment of otherwise intractable disease, treatment with fewer undesirable side effects compared to presently available treatments, or a decrease in undesirable side effects associated with presently available treatments, e.g., by decreasing the effective dose of an available treatment. Unless indicated otherwise, the term “total bile acids” as used herein means the sum of the dominant bile acids detected in an animal. In humans, this typically refers to at least cholic acid, glycocholic acid, deoxycholic acid, taurocholic acid, chenodeoxycholic acid, glycochenodeoxycholic acid, taurochenodeoxycholic acid, ursodeoxycholic acid, and lithocholic acid. In some embodiments, this also includes α-muricholic acid, beta-muricholic acid, gamma-muricholic acid (hyocholic acid), oxo-bile acids, and iso-bile acids. In some embodiments, the mammalian subject is a human subject who has one or more symptoms of a cholestatic disease or condition. In some embodiments, efficacy of a composition can be assessed by measuring bile acid composition, for example relative levels of selected bile acids compared to each other or concentration of one of more bile acids compared to a reference. Such measures can be made using, for example, fecal or serum bile acid levels. In some embodiments, metabolic indications of treatment can be assayed. In some embodiments, the presence or absence of bacterial species administered in a composition can be assayed. Such measures can also be used to monitor patient therapy, for example, to determine whether a patient requires additional treatment with a composition. Methods of identifying an appropriate reference are known to those in the art and include, for example, levels of one or more bile acids in a healthy patient population, levels of one of more bile acids in an untreated patient population diagnosed with the disease, or an improvement in levels of one or more bile acids in a patient after treatment compared with levels prior to treatment.

In some embodiments, effective treatment decreases alkaline phosphatase (ALP) activity or concentration, e.g., in liver, blood, or serum, compared to ALP activity or concentration in the patient prior to treatment; or decreases concentration or activity compared to a reference. Consistent with FDA labeling of Ocaliva® (obeticholic acid), such a decrease is sufficient to justify treatment even in the case where no statistically significant decrease in symptoms is observed. Methods of assaying ALP are known in the art.

In some embodiments, efficacy of a composition can be assessed by a decrease in bilirubin compared to bilirubin levels prior to treatment. For example, in some embodiments, an effective treatment results in lowering bilirubin levels below 25 milligrams per deciliter in the patient's urine. Bilirubin can also be assayed in blood, for example, total bilirubin is less than 1.0 mg/dL. Methods of assaying bilirubin levels are known in the art.

In some cases, effective treatment with a composition of the invention results in at least one of the following; reduction of alkaline phosphatase levels to <1.67× upper limit of normal (ULN), reduction of total bilirubin to ≤ULN, or alkaline phosphatase reduction by ≥15% from baseline.

The amount and frequency of a therapeutic composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight, and general condition of the patient, and the like. Reference is made to the dosage information noted above in the section relating to formulations.

In some embodiments, the subject receives an antibiotic treatment prior to administration of the composition. In some embodiments, the subject receives an antibiotic treatment and does not receive the composition until at least one day, two days, three days, 5 days, one week, two weeks, three weeks, or four weeks has elapsed since the antibiotic treatment and prior to administration of the composition. In some embodiments, the subject receives multiple doses of the composition to ensure coverage of the dosing period. In some embodiments, the subject has symptoms of a cholestatic disease prior to administration of the composition. In other embodiments, the subject does not exhibit symptoms of the cholestatic disease prior to administration of the composition, e.g., the composition is administered prophylactically to reduce the risk that a cholestatic disease will result in clinical symptoms.

In some embodiments, a composition is administered only once prior to improvement of the disease, disorder, or condition. In some embodiments, the therapeutic composition is administered at intervals greater than two days, such as once every three, four, five or six days, or every week or less frequently than every week, e.g., every two weeks, every three weeks, every 4 weeks, every six weeks, every eight weeks, every twelve weeks, once per month, once per two months, once per three months, once per four months, or once per six months. In some cases, the composition is administered intermittently according to a set schedule, e.g., once a day, once weekly, or once monthly, or when the subject relapses from the primary illness. In another embodiment, the composition is administered on a long-term basis to individuals who are at risk for cholestatic disease.

In some embodiments, the composition is generally administered enterally. For example, administration can be oral administration via a swallowed form (e.g., a pill, sachet, capsule, syrup or the like), or by an oral or nasal tube (including nasogastric, nasojejunal, oral gastric, or oral jejunal). In other embodiments, administration includes rectal administration (for example, by enema, suppository, or colonoscopy). The composition can be administered to at least one region of the gastrointestinal tract, including the mouth, esophagus, stomach, small intestine, large intestine, or rectum. A composition can be administered orally in the form of a medicament such as a powder, one or more capsules, one or more tablets, a gel or a liquid. A composition can also be administered in gel or liquid form by the oral route or through a nasogastric tube, or by the rectal route in a gel or liquid form, by enema or instillation through a colonoscope or by a suppository.

The subject may have a colonic-cleansing preparation prior to administration of a composition. Methods of colonic-cleansing are known in the art such as those used to prepare a subject for a colonoscopy. Also, the subject may optionally be treated with an antacid or buffering agent to increase stomach pH at the time of composition administration, as is known in the art and determined to be appropriate for the subject.

### Combination Therapy

As discussed above, a composition or formulation of the invention, such as those described herein, can be administered in combination with another agent useful in the treatment or prevention of cholestatic diseases. Thus, for example, the compositions or formulations of the invention can be administered in combination with one or more of the following agents: Ocaliva® (OCA, INT-747), INT-767 (FXR/TGR5 agonist), LJN452, GS-9674 (PX-102), PX-104, EDP-305, EP 024297, WAY-362450 (FXR-450) (XL335), GSK2324, GW4064, Fexaramine, endogenous bile acids (CDCA, LCA/DCA, and/or UDCA). These additional agents can be administered in separate compositions from the compositions or formulations of the invention, or can be combined with them to create additional new compositions. The additional agents can be administered at the same time as the compositions or formulations of the invention or, alternatively, can be administered within 1, 2, 4, 8, 12, 24, or more hours or days of the compositions or formulations of the invention, as determined to be appropriate by one of skill in the art. In one particular example of a combination or formulation of the invention, Ocaliva (OCA) is administered in combination with a composition of the invention that increases CDCA.

### Methods of Testing Candidate Compositions

### Murine Models

Animal models in which bile acid metabolism is adversely affected can be used to test candidate compositions for their ability to ameliorate a symptom of a cholestatic disease. Using such models, a candidate composition is administered to the model and an improvement of at least one sign or symptom of disease or a reduction in the rate of disease progression indicates the candidate can be used to treat a cholestatic.

One example of such a model is a multidrug resistance 2 knockout (mdr2−/−) mouse. mdr2 is the mouse homolog of human mdr3, a transporter that exports phosphatidylcholine (PC) from the liver into the hepatic canaliculi. The absence of PC is thought to lead to a bile composition that is abnormally high in primary bile salts that are not properly sequestered in micelles. This excess of primary bile salts is thought to cause bile duct damage that underlies cholestasis in these animals. (Smit et al., Cell 75:451-462, 1993; Fickert et al., Gastroenterology 127:261-274, 2004). mdr2−/− knockout mice develop bile duct damage having features similar to those seen in humans diagnosed with primary sclerosing cholangitis (PSC) and other disorders associated with abnormalities in bile acids, in particular, with respect to biliary strictures and liver fibrosis. These mice lack biliary phospholipids, which results in bile duct inflammation and damage due to micelles with an excess of bile acids and that develops into a pathophysiology that mimics primary sclerosing cholangitis.

LaRusso and colleagues report further development of the mdr2−/− model, based on the observation that mice having an mdr2−/− genetic background and raised germ-free (GF) develop a more severe form of the disease than conventionally-housed (CH) mdr2−/− mice (Tabibian et al., Hepatology 2015). In this model, mdr2−/− mice that are raised GF display typical alterations in intestinal tissue associated with the GF state, such as shallower colonic crypts, decreased ileal villous length, and diminished expression of the epithelial tight junction protein, zonula occludens. However, compared to conventionally raised mdr2−/− mice, differences are observed in serum biochemical markers for hepatobiliary disease at 60 days of age, including increases in aspartate aminotransferase, alkaline phosphatase, and bilirubin. Cholangiocyte senescence was assessed by p16INK4a in situ hybridization in liver tissue and was significantly increased in germ-free mice. These biochemical and histochemical markers were further confirmed by histopathology measurements. In addition, analysis of bile acid composition showed the absence of primary or secondary bile acids derived from microbial activity, as would be expected for germ-free mice. Serum biochemistries, including alkaline phosphatase, aspartate aminotransferase, and bilirubin, were all significantly higher in the GF mdr2−/− mouse. Younger GF mdr2-A mice (30 days of age) also exhibit altered serum biochemical markers indicative of more severe hepatobiliary disease than conventionally raised mdr2−/− cohorts.

A second model of cholestatic disease, developed by Fickert et al. in 2007 (Fickert et al., Am. J. Pathol. 171(2):525-536, 2007), induces bile duct injury and obstruction using the compound 3,5-diethoxycarbonyl-1,4-dihydrocollidine (DDC) leading to cholangitis and liver fibrosis. In conventional Swiss albino mice, DDC treatment resulted in inflammation, fibrosis, bile duct obstruction and chronic cholangitis, accompanied by symptoms such as elevated alkaline phosphatase and alanine transferase levels such as those seen in PSC patients. Symptoms are visible within a 4-week period making this a rapid model to study cholestatic disease. The role of the microbiome in this chemical model had not, however, been explored, and the effect of microbial bile acid metabolism on the susceptibility of germ-free mice to DDC-diet induced liver disease is elucidated in this patent.

Reportedly, bile acid profiles show no significant differences between GF and CH mdr2−/− mice with respect to primary bile acids (by HPLC). However, total serum bile acid levels were significantly higher in mdr2−/− mice. Similarly, total serum bile acids were elevated in the DDC-diet model of cholestasis, but levels of biliary bile acids were not altered with disease. Germ-free mice are unable to make secondary bile acids due to lack of microbial activity. Thus, germ-free models enable evaluation of various bacterial combinations designed to highlight specific bile acid enzymatic activity, such as deconjugation of primary bile acids or conversion of primary to secondary bile acids. In addition, this screen also enables the assessment of a specific primary or secondary bile acid or combinations of bile acids to identify those that can mediate changes in the disease phenotype in vivo. In some embodiments, a bacterial composition tested in this model or other models described herein alters total levels of primary and secondary bile acids, enabling measurement of the effects of altering bile acid composition on the disease phenotype.

Additional methods of evaluating the efficacy of a composition in a murine PSC/bile acid disease model include histological evaluation of mice after conventionalization with defined microbial compositions compared to germ-free littermates using the methods described by Tabibian et al. (Hepatol. 63:185-196, 2015). Germ-free mdr2−/− reportedly have a significantly higher proportion of advanced liver fibrosis compared to CH mdr2−/− mice and, in some cases, exhibit cirrhosis by 60 days while CH mdr2−/− mice are not reported as developing cirrhosis. Therefore, a reduction in liver fibrosis in a GF mdr2−/− mouse treated with a composition described herein indicates that the composition is useful for treating PSC or other disorder related to bile acid signaling such as NAFLD or NASH. Other methods of evaluating the effect of a candidate composition using GF mdr2−/− mice include detecting a decrease in ductular reaction and ductopenia, and a decrease in the proportion of senescent cholangiocytes in the presence of a composition that is useful for treating PSC or other disorder of bile acid metabolism, and a decrease in liver enzymes, such as ALP, and bilirubin in serum.

Candidate compositions can be tested in the mdr2−/− GF model, or in a germ-free version of the DDC-diet induced cholestatic disease model, as described above. Any other suitable model of an appropriate disorder of bile acid metabolism, for example, as described in the Examples (infra) may also be used. A candidate composition that is useful for treating such a disorder, i.e., a therapeutic composition, is a composition that, when administered for a suitable amount of time, decreases at least one sign or symptom of disease in an animal model of the disorder compared to an animal that did not receive the candidate composition.

Methods of identifying signs and symptoms of disease and improvement thereof are known in the art. For example, bile acid concentrations in serum can be assayed using colorimetric methods (e.g., kit from Trinity Biotech (Jamestown, NY)). Thin layer chromatography, high-performance liquid chromatography (HPLC), gas chromatography, or liquid chromatography in combination with tandem mass spectrometry (GC-MS or LC-MS/MS) can also be used to detect activity of bile acid metabolizing enzymes and changes in the composition of bile acids.

Compositions that prevent or ameliorate at least one sign or symptom associated with the selected bile acid disorder in an animal model of disease are termed “therapeutic compositions” and are useful for treating the disorder.

### EQUIVALENTS

All technical features can be individually combined in all possible combinations of such features.

The invention may be embodied in other specific forms without departing from the spirit or essential characteristics thereof. The foregoing embodiments are therefore to be considered in all respects illustrative rather than limiting on the invention described herein.

### EXAMPLES

The following non-limiting examples further illustrate embodiments of the inventions described herein.

### Example 1: Materials and Methods

### Materials

Taurocholic acid (t-CA), taurochenodeoxycholic acid (t-CDCA), glycocholic acid (gCA), glycochenodeoxycholic acid (gCDCA), cholic acid (CA), chenodeoxycholic acid (CDCA), deoxycholic acid (DCA), lithocholic acid (LCA), and 3,5-diethoxycarbonyl-1,4-dihydrocollidine (DDC) were obtained from Sigma-Aldrich (St. Louis, MO). Beta-muricholic acid (bMCA), 7-oxocholic acid, 7-oxochenodeoxycholic acid, and tauro-beta-muricholic acid (t-MCA) were obtained from Santa Cruz Biotechnology (Dallas, TX). Alpha-muricholic acid (aMCA), tauro-alpha-muricholic acid (t-MCA), 12-oxocholic acid, 12-oxodeoxycholic acid, 3-isodeoxycholic acid (3β 12α), hyodeoxycholic acid (3α 6α), hyocholic acid (HCA), and 3-oxodeoxycholic acid were obtained from Steraloids (Newport, RI). Bile acid stocks used in LC-MS based enzymatic assays were prepared by dissolving compounds in ethanol. Chenodeoxycholic acid (CDCA; Sigma-Aldrich) and obeticholic acid (OCA; MedChemExpress, NJ) used for cell-based assays were dissolved in DMSO to make a stock.

### Colonization

Germ-free and conventional mice were purchased from Taconic Biosciences (Hudson, NY) and used between 6-10 weeks of age, inclusive. Germ-free mice were dosed with 100 μL of 7.5% sodium bicarbonate via oral gavage to neutralize GI tract acids and improve survivability of administered bacteria. Approximately 10-30 minutes after receiving sodium bicarbonate, mice were dosed with 200 μL of a specific bacterial composition via oral gavage. Mice were handled in an aseptic manner, housed in germ-free isolators, and provided sterile chow and water. All studies were approved by individual Institute of Animal Care and Use Committees for each study site.

### Fecal Collections

Immediately prior to dosing, fresh fecal pellets were collected directly from mice and then at 1 day, 3 days, 7 days, or weekly post-dosing. At each time point, two fecal pellets were collected into sterile 1.7 mL microfuge tubes. One fecal pellet was frozen at −80° C. to hold for analysis by LC-MS. The second fecal pellet was homogenized into 100 μL of 15% glycerol in PBS (v/v) and then frozen at −80° C. to hold for microbiological and sequence analysis.

### Sequence Analysis

Sequences were analyzed using 16S rDNA V4 sequences using Next-Generation sequencing (NGS; high throughput sequencing) and mapped to identify the closest corresponding OTU. Sequence identity was at least 97% (to the nearest 0.1%) for all species calls. An internal proprietary manually curated reference OTU database was used for assign species identities.

### Assay for Bile Salt Hydrolase (BSH) Activity

Bacterial whole cell suspensions in PBS were incubated with mixtures of conjugated bile acids, each at a final concentration of 150 μg/ml. Reaction mixtures in 96-well plates were incubated for 4 hours at 37° C. under anaerobic conditions. After incubation, samples were removed from the anaerobic chamber. An equal volume of acetonitrile was added to samples to extract bile acids, plates were centrifuged to pellet bacteria, and the resulting supernatant filtered through a 0.2 μm filter, generating a sample for LC-MS analysis.

### Assays for Hydroxysteroid Dehydrogenase and 7α-Dehydroxylation Activities

To assay hydroxysteroid dehydrogenase (HSDH) and 7α-dehydroxylation activity, bacterial suspensions in brain-heart infusion (BHI) medium were incubated with cholic acid or chenodeoxycholic acid individually, each at a concentration of 100 μM, for 4 hours at 37° C. in an anaerobic chamber. After incubation, samples were removed from the anaerobic chamber. An equal volume of acetonitrile was added to samples to extract bile acids, plates were centrifuged to pellet bacteria, and the resulting supernatant filtered through a 0.2 μm filter, generating a sample for LC-MS analysis.

### Designed Compositions

A collection of research cell banks (RCBs) was used to create designed compositions (DE) for in vivo studies. The vegetative titer of each cell bank (CFU/mL) was used to calculate the volumes of each needed to create a designed composition with a final titer 1.00E+07 CFU/strain for each dose. Volume of an individual RCB to be added to the designed composition was calculated as follows:

[total vegetative titer needed for all doses (e.g., for 10 doses at 1.00E+07=1.00E+08 CFU total)]/[RCB vegetative titer in CFU/mL]=volume needed for 1.00E+07 CFU/dose for 10 doses

This was repeated for each RCB in the defined DE to determine volumes, after which the calculated volume of each RCB was combined in an anaerobic chamber to formulate the RCB, vortexed, spun down, and resuspended in a final volume of 15% glycerol-PBS.
Extraction of Bile Acids from Mouse Fecal and Liver Samples

Mouse fecal pellets were weighed, homogenized in 10× w/v extraction buffer (50% methanol in water), and extracted on ice for 1 hour. Liver tissue samples were similarly weighed, homogenized in 2×w/v extraction buffer (50% methanol in water), and extracted on ice for 1 hour. Post incubation, samples were further extracted with an equal volume of cold acetonitrile, centrifuged, and the supernatant filtered through a 0.22 μm filter prior to loading on an LC-MS for analysis.

Extraction of Bile Acids from Portal, Serum and Bile Samples

Peripheral serum samples were diluted 1:1 in acetonitrile, centrifuged, and the supernatant filtered through a 0.22 μm filter prior to loading on an LC-MS for analysis. Portal serum samples were diluted 1:10 in acetonitrile and similarly centrifuged and filtered prior to LC-MS analysis. Finally, bile samples were diluted 1:100 in acetonitrile, centrifuged, and filtered through a 0.22 μm filter for LC-MS analysis. The same procedures were utilized for both human and mouse samples.

### LC-MS Analysis of Bile Acids

Bile acids were separated using an Agilent 1260 HPLC equipped with a Microsolv bidentate C18 column preceded by a 0.2 μm pre-column filter. Separation was achieved using a water and acetonitrile gradient with 0.1% formic acid at a flow rate of 0.4 ml/minute. Samples were injected at a volume of 5 μL. The HPLC system was coupled to a Bruker Compass™ qTOF mass spectrometer calibrated to a mass range of 50 to 1700 m/z using the Agilent low-mass tuning mix. Each run was additionally calibrated to a reference mass solution injected at the beginning of each run. Bile acids were detected in negative mode, and identified by unique m/z and retention times compared to known pure standards and area under the peak determined using Bruker data analysis software. Metabolites were quantified using calibration curves generated from pure standards, ranging in concentration from 0.001 μM to 100 μM, in PBS. For fecal samples, individual bile acids are depicted as percent of total bile acid pool, where total bile acid pools were determined as a sum of all detected bile acids in the sample. For liver samples, the total bile acid levels were determined as the sum of all detected bile acids, normalized to sample tissue weight (nM/ng). Bile acids detected by LC-MS are listed in Table 4, as follows.

TABLE 4

| Conjugated | | | Conjugated. |
|---|---|---|---|
| 1° Bile Acids | 1° Bile Acids | 2° Bile Acids | 2° Bile Acids |
| Taurocholic acid (t-CA) | Cholic acid (CA) | Deoxycholic acid (DCA) | Tauro-deoxycholic |
| Glycocholic acid (g-CA) | Chenodeoxycholic | Lithocholic acid (LCA) | acid (t-DCA) |
| Tauro-chenodeoxycholic | Acid (CDCA) | 3-Oxo-cholic acid | Glyco-deoxycholic |
| acid (t-CDCA) | α-Muricholic acid | (3-oxo CA) | acid (g-DCA) |
| Glyco-chenodeoxycholic | (αMCA) | 7-Oxo-cholic acid | Tauro-lithocholic |
| acid (g-CDCA) | β-Muricholic Acid | (7-oxo CA) | acid (t-LCA) |
| Tauro-α-muricholic | (βMCA) | 12-Oxo-cholic acid | Glyco-lithocholic |
| acid (t-αMCA) | Hyocholic acid | (12-oxo CA) | acid (g-LCA) |
| Tauro-β-muricholic | (HCA) | 3-Oxo-chenodeoxycholic | |
| acid (t-βMCA) | | acid (3-oxo CDCA) | |
| Tauro-hyocholic | | 7-Oxo-chenodeoxycholic | |
| acid (t-HCA) | | acid) 7-oxo CDCA | |
| Glyco-hyocholic | | 3-Oxo-deoxycholic acid) | |
| acid (g-HCA) | | 3-oxo DCA | |
| | | 12-Oxo-deoxycholic acid | |
| | | (12-oxo DCA) | |
| | | 3-Oxo-lithocholic acid) | |
| | | 3-oxo LCA | |
| | | Iso-deoxycholic acid | |
| | | (Iso-DCA) | |
| | | Ursodeoxycholic acid | |
| | | (UDCA) | |

### Gene Expression Profiling

Distal ileum samples, taken from mice at 3 days post-dosing, were flash frozen upon collection. RNA was isolated by homogenization followed by extraction using the Qiagen RNeasy® Plus Mini kit per manufacturer's instructions. Real-time qPCR reactions were run with an RNA-to-CT one step qPCR expression kit (Life Technologies, Carlsbad, CA), using ˜50 ng RNA and with Taqman® primers to beta-actin, NR1H4 (Mm00436425_ml), NRB02 (Mm00442278_ml), or FGF15 (Mm00433278_ml). All gene expression was normalized to expression of the housekeeping gene, beta-actin.

hFXR Luciferase Reporter Assay

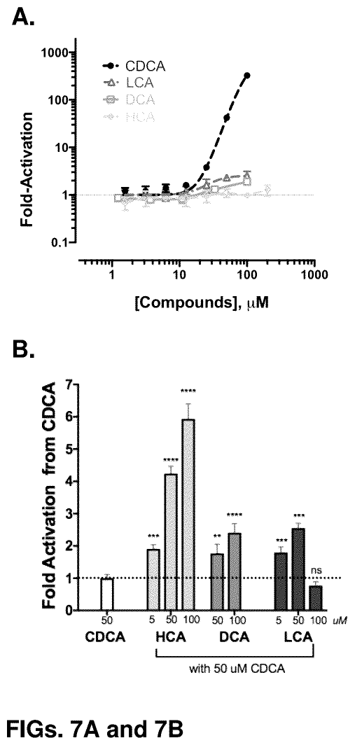

FXR reporter cell assays were performed by Indigo Biosciences as directed by Seres Therapeutics using Indigo's FXR reporter CHO cell line, which expresses an FXR receptor hybrid in which the native N-terminal DNA binding domain (OBO) has been replaced with that of a yeast Gal4 OBO. The reporter gene, firefly luciferase, is functionally linked to a Gal4 upstream activation sequence (UAS). Briefly, reporter cells were submitted to a serial dilution curve of COCA, OCA, or vehicle (0.2% DMSO) and incubated at 37° C. for 24 hours in a high-humidity, 5% CO₂ chamber. Luminescence was determined following incubation with substrate and a detection reagent. In addition, live cell count was determined within the assay using a Live Cell Multiplex (LCM) Assay (Indigo Biosciences). Data are shown as normalized to the vehicle group (FIG. 7).

### Analysis of Serum Alkaline Phosphatase Levels (ALP)

Whole blood or serum samples were used for ALP measurement. 100 μL of sample was loaded onto a VetScan mammalian liver profile disc (Abaxis) and analyzed using a VetScan VS2 series chemistry analyzed (Abaxis). ALP levels as well as additional serum biochemical analysis reports were printed out for comparison.

Methods described in this example are useful for identifying bacteria having specific bile acid metabolizing features. Other such methods are known to those in the art.

### Example 2: Designed Compositions Demonstrate Specific Bile Acid Activities In Vitro

In a germ-free mouse model, Applicants have demonstrated the targeted reduction of both total bile acid levels and specifically, the conversion of primary bile acids to their secondary counterparts using designed bacterial compositions. Applicants also demonstrated bile acid specific signaling in the intestine, influenced by the targeted restoration of secondary bile acid pathways by designed compositions. These experiments are described in further detail, below.

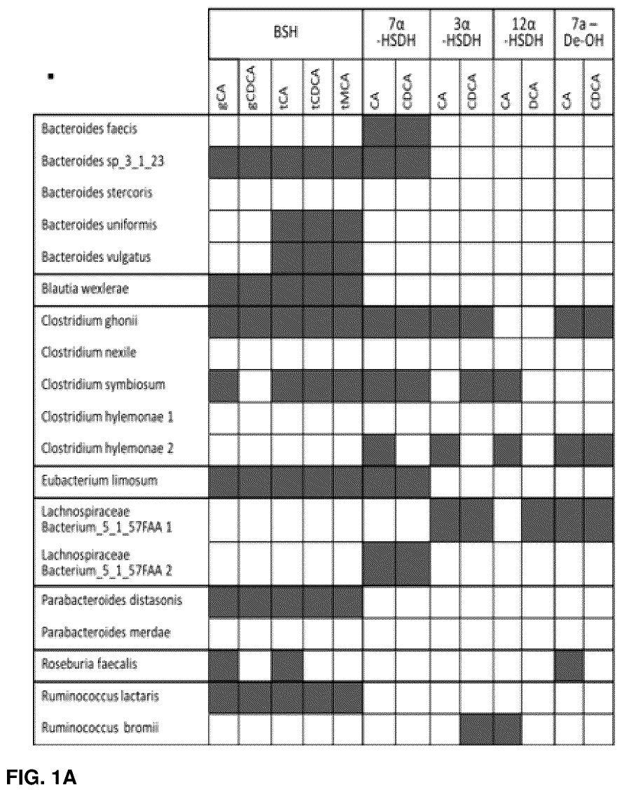

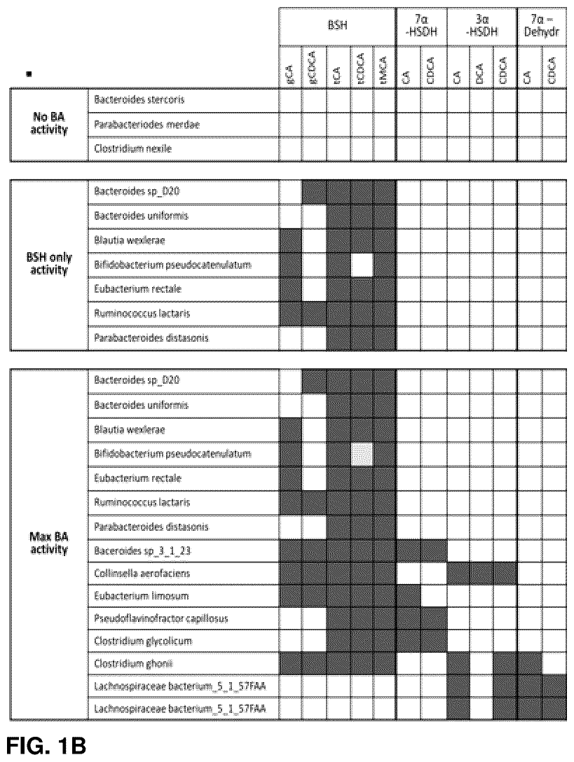

To create combinations of bacterial strains having specific metabolic activities, methods were designed for characterizing the bile acid metabolic activities of individual bacterial strains in vitro. Accordingly, an LC-MS based screening approach was used. Screening of nearly 200 strains resulted in the identification of a variety of bile acid metabolic activities including deconjugation, oxidation, and 7-alpha dehydroxylation (7α-deOH) in a number of human bacterial isolates; examples are shown in FIG. 1A (also see Table 3). Bile acid deconjugation is catalyzed by bile salt hydrolase (BSH). Different BSHs can exhibit preference for activity on different conjugated primary bile salts. In some cases, a bacterium has more than one BSH, at least two of which differ in substrate-specific activity from each other. For example, Applicants queried 1129 genomes represented in the Human Microbiome Project database for BSH sequences and discovered that 43% of those genomes had sequence corresponding to a BSH sequence and the genomes had between one and six such sequences. Accordingly, in some cases, a criterion for selecting a species for a DE is the presence of more than one BSH, e.g., the ability to metabolize multiple types of conjugated primary bile salts. In other cases, a species is selected for specificity, e.g., the ability to cleave only one specific conjugated bile salt. Oxidation reactions are catalyzed by hydroxysteroid dehydrogenase (HSDH), while 7α-dehydroxylation is a multistep process facilitated by the bai operon. Strikingly, for those cases in which multiple strains within a genus were tested, there was no obvious pattern of substrate specificity or enzyme activity for any of the three reactions tested. In addition, variations in specificity of levels of activity were also seen between multiple isolates of the same species from different donors.

As previously mentioned, the likelihood of species within a clade have similar functions remains high, providing a pool of species useful for generating compositions with specific function. The variation in activity between species and OTUs and strains (Table 3) however, makes the confirmation of activity in strains of interest, either by in vitro assays or genome analysis, a necessity for the construction of compositions with targeted bile acid metabolic capabilities.

Bacterial strains for which bile acid metabolizing activities had been determined were then tested in combinations designed to have specific bile acid metabolizing features. Three compositions with specific bile acid metabolic activities were designed and prepared (FIG. 1B). A control ‘No BA activity’ composition consisted of strains that, as single strains, showed no bile acid metabolizing activity when tested as described above. Furthermore, when used in the assay described above, the mixed composition did not exhibit any detectable bile acid metabolizing activity, suggesting that these strains, in combination with each other, had no complementary features with respect to bile acid metabolizing activity. The second composition, a ‘BSH only activity composition,’ consisted of strains whose only detected bile acid metabolizing activity was BSH activity, which resulted in a composition that was restricted to the deconjugation of primary bile salts and was unable to further modify the deconjugated, primary bile acids into their secondary derivatives. Conjugated bile salts tested included glycine and taurine conjugated cholic acid and chenodeoxycholic acid, as well as taurine conjugated α-muricholic and β-muricholic acids. The final composition, a ‘max bile acid’ (Max BA activity) composition, was designed to encompass BSH mediated deconjugation activity as well as the two secondary bile acid enzymatic activities described above oxidation, and 7α-dehydroxylation. The activity of the combination was confirmed in the in vitro assay (FIG. 1B).

These data demonstrate that in vitro methods can be used to construct compositions that are effective for altering bile acid metabolism.

Colonized Germ-Free Mice Rapidly Engraft with Species from Compositions

A germ-free mouse model of bacterial GI colonization was used to characterize the bile acid metabolic activities of a specific bacterial composition in vivo. Germ-free mice do not carry any microorganisms. Therefore, as long as the mice are maintained in a germ-free environment, introducing a specific bacterial composition into a germ-free mouse allows direct analysis of the bile acid metabolic activities of the composition.

Five germ-free mice were colonized with a “no bile acid activity” composition made up of three bacterial strains used in the in vitro experiments described supra. Fecal samples were collected prior to dosing with a composition and at 6 hours, 1 day, 3 days, and 7 days post-dosing, and analyzed by NGS to detect colonizing microbes. The colonization data below are reported at the level of bacterial clades. 16S v4 NGS sequencing allowed for accurate identification of bacterial clades with inferred species identifications. This was possible because the three species in the ‘No bile acid’ composition belong to distinct clades; in this case, analysis at the clade level provides accurate data pertaining to the strains present in the mice post-colonization.

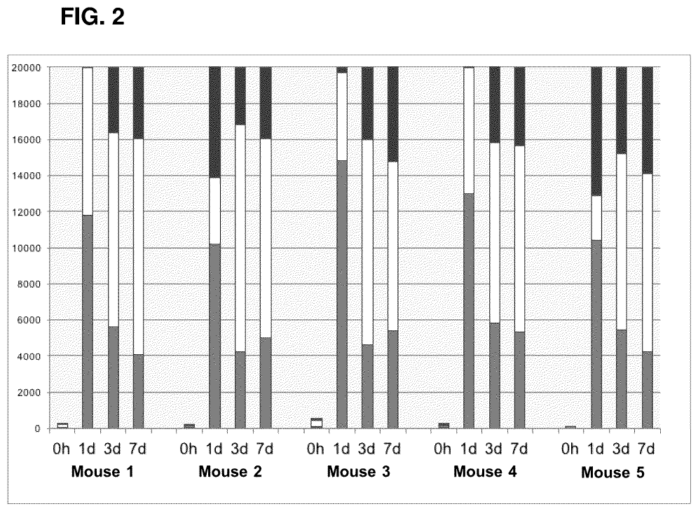

All five mice were confirmed to be germ-free at the start of the experiment based on the lack of bacterial counts observed using NGS (see FIG. 2). No bacteria were cultured from the feces of mice pre-treatment, further confirming their germ-free state. By 24 hours, the microbiome of the treated mice had only sequences from clades to which the composition bacteria belong. Later time-points (3 days and 7 days) were also found to contain only the three clades to which bacteria in the “no bile acid” composition belong. This indicates that only bacteria in the composition stably colonized the GI tract of the germ-free mice. These data demonstrate that the germ-free model can be used to test for colonization experiments to assay compositions.

### Colonized Germ-Free Mice Show Specific Fecal Bile Acid Profiles

To further examine the suitability of the murine colonization model for testing compositions related to bile acid metabolism and the ability of a bacterial composition to alter bile acid metabolism in a targeted manner, fecal samples from germ-free mice colonized with designed compositions were analyzed for bile acid composition and compared to germ-free and conventionalized control mice.

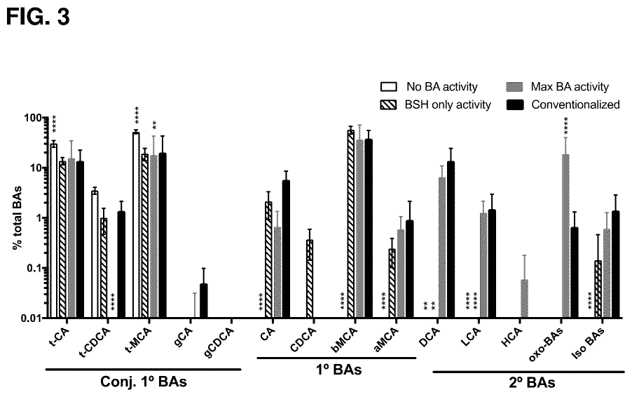

Only conjugated primary bile salts were detected in the feces of germ-free mice. Conjugated primary bile salts are synthesized by the liver, and their prevalence in the feces demonstrates a lack of the bile acid metabolism catalyzed by GI bacteria to produce (unconjugated) primary and secondary bile acids. Similarly, mice colonized with the no bile acid activity composition demonstrated a complete lack of detectable primary bile acids and secondary bile acids in fecal samples (FIG. 3), with a bile acid profile identical to untreated germ-free mice. In contrast, conventionalized mice, which are germ-free mice colonized with a fecal preparation from specific pathogen free mice (SPF mice), “Conventionalized” mice in FIG. 3, a process referred to herein as fecal microbiome transplantation (FMT), showed a diverse fecal bile acid profile (FIG. 3) similar to that seen in untreated wild-type mice, including a range of primary and secondary bile acids.

The first step in microbiome-mediated bile acid metabolism is deconjugation, the removal by a BSH of taurine or glycine residues from conjugated primary bile salts, to release free bile acids (Ridlon et al., JLR 47:247-259, 2006). Colonization of germ-free mice with a bacterial composition restricted to only BSH activity resulted in mouse fecal samples containing deconjugated primary bile acids (primary bile acids) in addition to the previously detected conjugated primary bile salts, but no downstream secondary bile acids (FIG. 3). In one case a single mouse had low levels of a mouse muricholic acid derivative, iso-bile acid (3a, 6a muricholic acid). This outlier bile acid detected in a single mouse was at levels 10 fold lower than typically seen in conventional wild-type mice and may be a byproduct of incomplete muricholic acid synthesis in the liver.

The data of this example demonstrate that conjugated primary bile salts can be deconjugated in vivo by introduction of a bacterial composition having BSH activity. Furthermore, this demonstrates that the in vivo activity can correspond to the in vitro activity of the composition (FIG. 1B).

After deconjugation of conjugated primary bile salts, the resulting primary bile acids are further modified by the GI microbiome into a range of secondary bile acids that can influence signaling and the regulation of bile acid metabolism in the liver. Colonization of germ-free mice with the Max BA activity composition for 7 days produced many deconjugated primary and secondary bile acids to levels similar to those seen in conventionalized mice colonized with the mouse SPF/FMT mouse fecal samples (FIG. 3). Secondary bile acids restored in Max BA colonized mice included 7α-dehydroxylated bile acids (DCA and LCA) and oxo-bile acids (7-oxo CA, 3-oxo CDCA, 12-oxo DCA, 3-oxo LCA) and iso-bile acids (UDCA), demonstrating that a significant portion of microbial bile acid metabolism can be restored with a designed bacterial composition. The sole observed exception was the iso-bile acid 3β, 12α-DCA, which was not detected in Max BA activity colonized mice. Without committing to any particular theory, formation of 3β,12α-DCA requires bile acid isomerization activity specific to DCA, catalyzed by the 7β-HSDH enzyme, which was not selected for in the bacterial compositions for this experiment. This activity was likely not present in the Max BA activity composition.

tCDCA was detected in the conventionalized mice and was not detected in the max-BA mice (FIG. 3). This indicates that the Max BA composition completely converted all the tCDCA to DCA and oxo-bile acids, while the conventionalized mice did not, i.e., was more efficient at conversion of the conjugated bile acid than the microbiota composition of the conventionalized mice. This further demonstrates that a DE can selectively shape the bile acid pool in vivo and this feature can be useful in patients with disease associated with defects in bile acid metabolism or signaling.

Further, as shown in FIG. 3, the Max BA composition showed the presence of HCA activity while none was detected in the conventionalized mice. In this case, it is likely that HCA is not metabolized by the Max BA composition as completely as by the conventionalized composition. These data further demonstrate the usefulness of a designed composition having selected bile acid activities for altering the composition of a patient's bile acid pools, to selectively normalize or otherwise adjust the pools to ameliorate a bile acid associated disease.

### Restoring Bacterial Bile Acid Metabolism in the GI Tract Lowered the Total Liver Bile Acid Pool

In some aspects of the invention, cholestatic disease patients can benefit from a reduction in the bile acid pool. Primary and secondary bile acids signal through FXR to regulate bile acid synthesis in the liver, regulating the expression of CYP7A1 and other bile acid synthesis genes to decrease bile acid production when present (Hylemon et al., JLF 50:1509-1520, 2009). Germ-free mice lacking a primary and secondary bile acid pool reportedly had increased bile acid production and higher total bile acid levels compared to conventional mice (Sayin et al., Cell Metab. 17:225-2235, 2013). To determine the effect of designed bacterial compositions on bile acid synthesis, total liver bile acid pools of germ-free mice, mice colonized with a designed composition, and conventionalized mice were assayed using methods described supra.

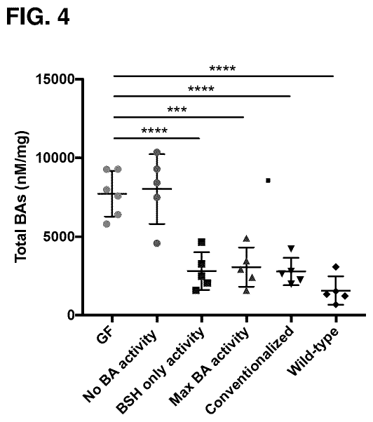

In these studies, germ-free mice had elevated liver bile acid pools compared to FMT/conventionalized mice and to wild-type mice (FIG. 4). Colonization with the no BA activity composition had no effect on total bile acid pools, which remained elevated and comparable to untreated germ-free mice. Colonization with either the BSH only or the Max BA compositions resulted in a significant decrease in total liver bile acid pool, to levels comparable to wild-type mice (FIG. 4), i.e., there is signaling through bile acid receptors by both primary and secondary bile acids, thereby impacting the bile acid pool in the liver.

FGF15 is Differentially Regulated in Response to Bacterial Compositions with Specific Bile Acid Activities

Bile acids, particularly the unconjugated primary bile acid CDCA, have been reported to signal in the ileum through the farnesoid X receptor, FXR (NR1H4), resulting in the upregulation of FGF15. FGF15 acts on the liver to reduce bile acid synthesis, and therefore reduces the total bile acids in the enterohepatic system. Accordingly, FGF15 gene expression was used as a biomarker to test whether changes in bile acid profiles induced by a designed bacterial composition can alter function, in particular, FXR signaling. As reported in the literature, FXR levels themselves were not expected to change (Sayin et al., Cell Metab. 17:225-235, 2013; Song et al., Tox. Appl. Pharmacol. 283:57-64, 2015).

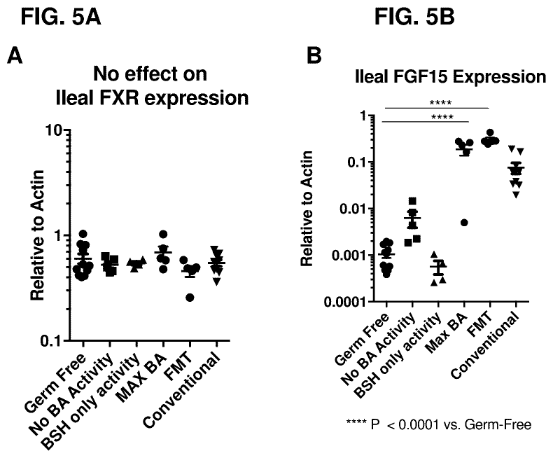

In these experiments, germ-free animals colonized with a murine-derived fecal microbiome transplant (FMT) significantly upregulated FGF15 gene expression (200×-300×) compared to germ-free animals (FIG. 5B). FGF15 levels of these conventionalized mice were comparable to those observed in wild-type conventional mice. These data confirm a role for the complex GI microbiome in regulating FXR signaling. Mice colonized with a defined bacterial composition containing maximal bile acid activity also had increased FGF15 levels compared to germ-free mice. These data demonstrate the restoration of FXR signaling in the presence of secondary bile acids (FIG. 5B). Surprisingly, animals given a composition with no bile acid activity showed small increases in FGF15 levels (˜8 fold), indicating that some FGF15 activity can be induced by the presence of a microbiome independently of secondary bile acid metabolism (FIG. 5). The levels of FGF15 in the mice colonized with the no bile acid composition did, however, remain significantly lower than the levels observed in conventional or maximum bile acid activity colonized mice. In contrast, mice colonized with the BSH only activity composition, which produced primary bile acids but no secondary bile acids and was predicted to activate FXR, showed no changes in FGF15 expression (FIG. 5B).

Co-Treatment with Primary Bile Acid CDCA Enhances Activity of OCA In Vitro

Obeticholic acid (OCA) is FDA approved for treating primary biliary cholangitis (PBC), typically in combination with ursodeoxycholic acid or, in some patients, as a single treatment regimen. It is also in clinical development as a treatment for PSC and NASH. However, OCA can have undesirable side effects. Obeticholic acid, an FXR agonist, is a synthetic derivative of CDCA that is reportedly 100× more potent in activating human FXR compared to CDCA.

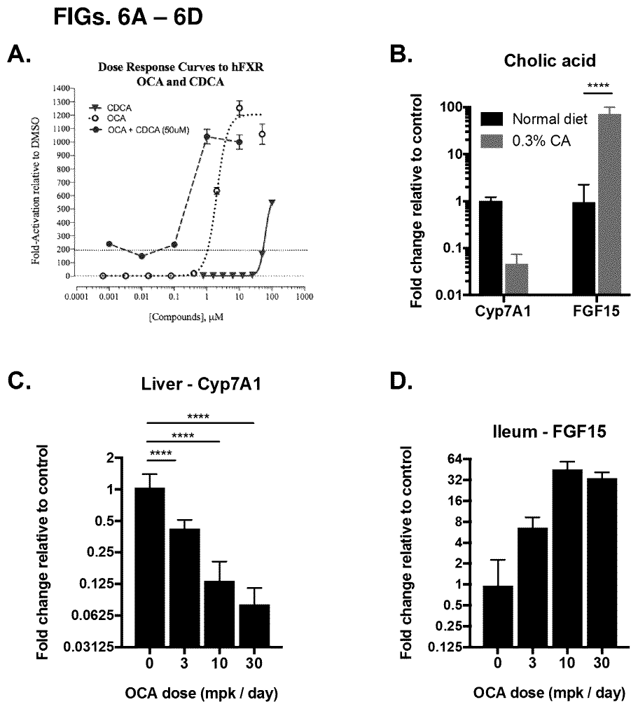

Experiments were conducted using a human FXR reporter cell line to confirm reported differential potencies of OCA and CDCA relative to vehicle (FIG. 6). In a luciferase-based assay, reporter cells were incubated with serial dilutions of either OCA or CDCA, with a readout of FXR activity. FXR binds to bile acids and then becomes active as a transcriptional regulator. OCA was almost 100× more potent compared to CDCA. We then determined the effect of co-incubation of CDCA with OCA on activation of FXR. Surprisingly, the addition of EC₅₀ (50 uM) of CDCA affected the FXR activity of OCA. In the presence of CDCA, the OCA dose-response curve was left shifted about 20-fold, indicating that addition of CDCA to OCA can increase OCA efficacy (FIG. 6A).

Given the side effects associated with current OCA dosing standards, a lower dose of OCA could significantly improve patient experience while still providing effective treatment. To determine if bile acid co-treatment improves OCA efficacy in-vivo, we first identified dose ranges for effective FXR signaling of OCA and bile acids of interest in a mouse model. In the mouse, chenodeoxycholic acid constitutes an insignificant portion of the bile acid pool, while cholic acid (CA) appears to be the more potent ligand for FXR (Song et al., Tox. Appl. Pharmacol. 283:57-64, 2015). We therefore tested a dose range of OCA as well as a fixed concentration of CA for their effects on FXR signaling in mice. Ileal and liver FXR signaling was monitored based on the expression of CYP7A1 (liver) and FGF-15 (Ileum). OCA administration resulted in a dose dependent decrease in CYP7A1 expression and an increase in FGF-15 levels (FIG. 6C-D), suggesting effective signaling through FXR in both the liver and the ileum. A cholic acid supplemented diet also decreased CYP7A1 expression and increased FGF-15 levels in the ileum, suggesting FXR activation in with CA supplementation (FIG. 6B).

We next determined if co-treatment of cholic acid with OCA can improve efficacy of OCA signaling through FXR, thus lowering the effective concentrations of OCA required for effective therapy. In PBC patients that respond to UDCA treatment, OCA is recommended as a single daily dose of 5 mg, with the option to increase dosing to 10 mg in the absence of a response after 3 months. Dosing at these concentrations has been associated with side-effects, particularly pruritus. Increasing the efficacy of OCA with a microbiome composition that produces primary bile acids such as cholic acid (CA) or chenodeoxycholic acid (CDCA), can result in equivalent efficacy at a lower dose, and may alleviate OCA-related side effects while still achieving effective treatment. Reduction in dosing amount or frequency may also increase the number of patients able to successfully tolerate treatment with OCA, thereby providing treatment to a broader population.

In view of this discovery, in some embodiments, the invention relates to a bacterial composition that can increase CDCA and/or cholic acid if administered to a patient undergoing OCA treatment. In some cases, the composition can also modulate (e.g., decrease) HSDH and 7α-dehydroxylation activity.

### CDCA Enhances the Efficacy of the Non-Active Hyocholic Acid in a Dose Dependent Manner

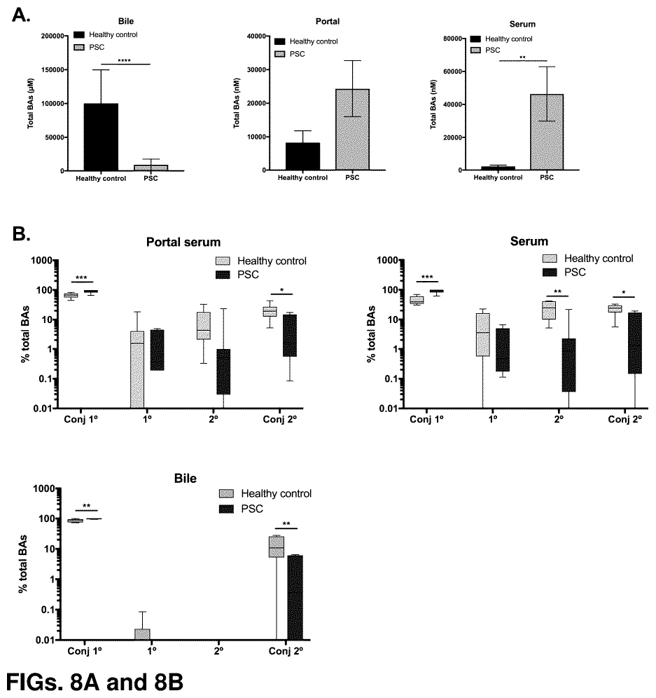

Hyocholic acid (HCA; is a 6-α-hydroxy muricholic acid; OCA is a 6-α-ethyl cholic acid) is a bile acid that is reported to be present at moderate levels in fetal bile and is present in lower levels in adult humans (Setchell et al., J. Biol. Chem. 263:16637-16644, 1988). HCA has no known functional properties. Using the in vitro reporter assay described supra, Applicants tested whether HCA had hFXR agonist activity. Results are depicted in FIG. 8.

In these experiments, HCA alone had no human FXR agonist activity at concentrations up to 200 uM compared to that of CDCA and was even less active than the relatively weak agonists, LCA and DCA (FIG. 7A). Surprisingly, in the presence of 50 uM CDCA, HCA activated human FXR in a dose-dependent and synergistic manner (FIG. 7B). This effect was not seen with CDCA in combination with DCA or LCA, which upregulated hFXR agonist activity in a more additive fashion; although the combination with LCA did show toxicity at higher concentrations (100 μM) (bottom graph). Without committing to any particular theory, it may be that CDCA potentiates bile acids modified at the 6-α position.

These data indicate that bacterial compositions that can upregulate CDCA and/or HCA can be used to enhance endogenous FXR signaling to treat cholestatic diseases.

### Example 3: Treatment of a Murine Model of Cholestatic Disease

Mice lacking the MDR2 gene develop cholestatic disease (Tabibian et al., Hepatology 63(1): 185-196, 2016). Germ-free MDR2−/− mice develop a more rapid and severe cholestasis, presumably because of cholangiocyte toxicity from bile lacking appropriate amounts of phosphatidylcholine in addition to the lack of microbiome synthesized primary and secondary bile acids. Colonizing these mice with bacteria that are either unable to deconjugate primary bile salts or to make secondary bile acids (No BSH and BSH-only compositions as above) and comparing the progression of disease with mice that are colonized with a microbiome that is able to make a full suite of secondary bile acids (Max BA as above) demonstrates the ability to control progression of cholestasis through interventions into the microbiome.

The germ-free mdr2−/− mouse can also be used to model the efficacy of a specific composition for ameliorating cholestatic disease or symptoms of cholestatic disease. Mouse models colonized with poorly protective microbiomes, such as a No BA activity composition, are expected to develop disease more rapidly or to a greater extent compared to colonizing such mice with a composition that can enhance bile acid metabolism, such as a BSH activity only composition or a Max BA activity composition. Such compositions are evaluated using a mouse model, monitoring the effects of a composition that can increase primary and/or secondary bile acid metabolism on the progression and severity of cholestatic disease. Germ-free interventional mouse models useful in such evaluation of a composition include the germ-free mdr2−/− deletion model or the DDC-induced chemical models of cholestatic disease in wild-type mice (e.g., Fickert et al., Am. J. Pathol. 171:525-536, 2007). Antibiotic treatment in conventional mdr2−/− mice can be used as an additional model; using antibiotics to deplete a dysfunctional microbiome that is then replaced with a protective composition. A microbiome that can improve secondary bile acid metabolism, such as the BSH only or Max BA composition, can slow progression of disease and reduce severity of pre-existing cholestatic disease.

### Example 4

### Decreased Secondary Bile Acid Levels in PSC Patients

In Primary Sclerosing Cholangitis (PSC), the blockage of bile ducts is thought to result in significant changes in bile acid concentrations in the hepatobiliary system and peripheral tissues of patients, leading to altered signaling and tissue damage in the liver (Chazouillers, Clin. Res. Hepatol. Gasteroentrology 36:S21-S25, 2012). Work by Trottier et al. (Trottier et al., Dig. Liver Dis. 44:303-310, 2012) has shown that there is a significant increase in total bile acids, accompanied by a drop in secondary bile acids, in the peripheral serum of patients with PSC. Changes in bile acid levels in systems linked to hepatobiliary circulation, such as bile or portal serum, however, are not well characterized. Here, we profile 25 unique bile acids in portal, bile and peripheral serum from 7 patients with PSC (end stage liver disease). These measurements were compared with those of 12 healthy donors and showed a significant change in total levels, as well of composition, of bile acids in patients with cholestatic liver disease.

Total bile acid levels were significantly elevated in peripheral serum and were also increased in portal blood compared to healthy donors (FIG. 8A). In contrast total bile acid levels in bile were significantly lower in patients with PSC, suggesting a decrease in the flow of bile from the liver gall bladder and indicative of a block in bile ducts (FIG. 8A). The corresponding increase in bile acids in serum may consequently reflect an accumulation of bile acids in the liver that are leaked out into serum. When broken down into their constituent bile acid groups, levels of conjugated and unconjugated microbial-derived secondary bile acids were decreased in peripheral and portal serum and bile (FIG. 8B). This included bile acids produced by microbial-catalyzed activities such as 7α-dehydroxylation and HSDH. Unconjugated primary bile acids, also generated by microbial BSH activity in the gut, were decreased in peripheral serum and to a smaller extent in portal serum as well. These changes were accompanied by an increase in the up-stream conjugated primary bile acids synthesized by the liver, suggesting an impairment in the primary and secondary bile acid metabolic pathways catalyzed by the gut microbiome. This decrease in microbiome synthesized bile acids suggests an altered microbiome in patients with liver disease, and potentially compromised signaling in the liver.

Mouse Model of Cholestatic Disease Shows Changes in Bile Acid Profile that are Comparable to Human PSC Patients

Two mouse models, an mdr2−/− deletion model that develops disease over the course of a year (see above), as well as the more rapid DDC-diet induced cholestasis model, are currently utilized to study the progression of cholestatic liver disease. The DDC-diet model was recently published as an alternative to the genetic mdr2−/− knock out model of cholestatic disease (Fickert et al., Am. J. Pathol. 171(2):525-536, 2007). This model demonstrates the blockage of bile ducts typically seen in PSC patients and shows the elevated ALP and ALT serum levels also associated with cholestatic disease in PSC. The effect of DDC-diet induced cholestatic disease on bile acid metabolism in mice, however, remains unknown.

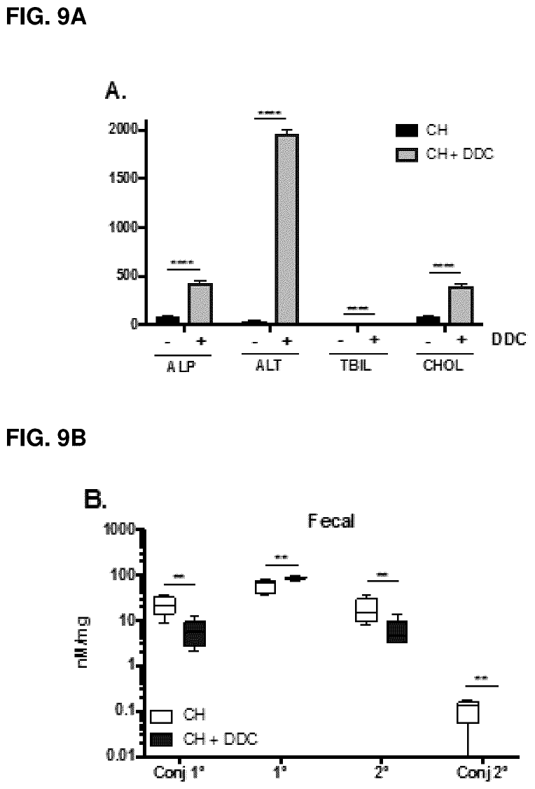

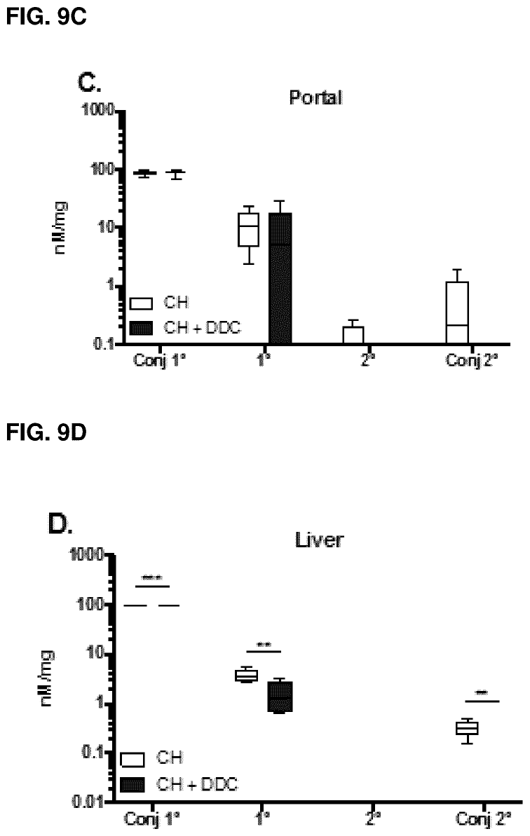

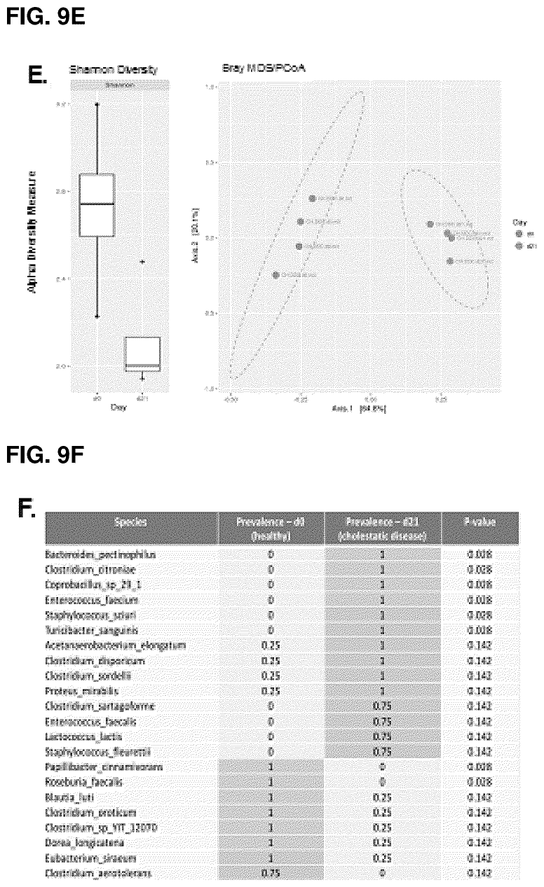

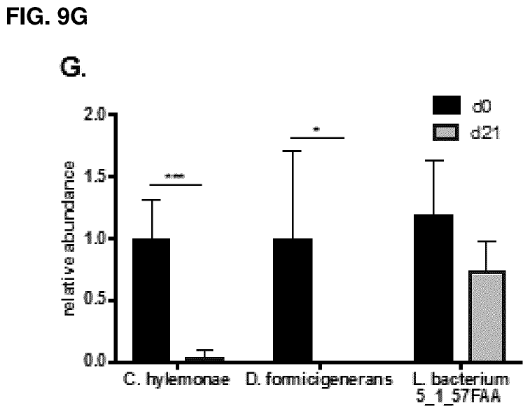

To assess changes in bile acid content, conventionally housed C57Bl/6 mice were placed on the DDC-diet and monitored for body weight loss. Disease severity was assessed based on weight loss, serum biochemistry, and overall health. At 21 days of treatment, mice on the DDC diet show elevated levels of ALP, ALT, total bilirubin, and cholesterol (FIG. 9A), confirming the development of cholestatic disease. Fecal, liver, and portal blood samples from these mice were then assessed for bile acid content compared to healthy untreated controls.

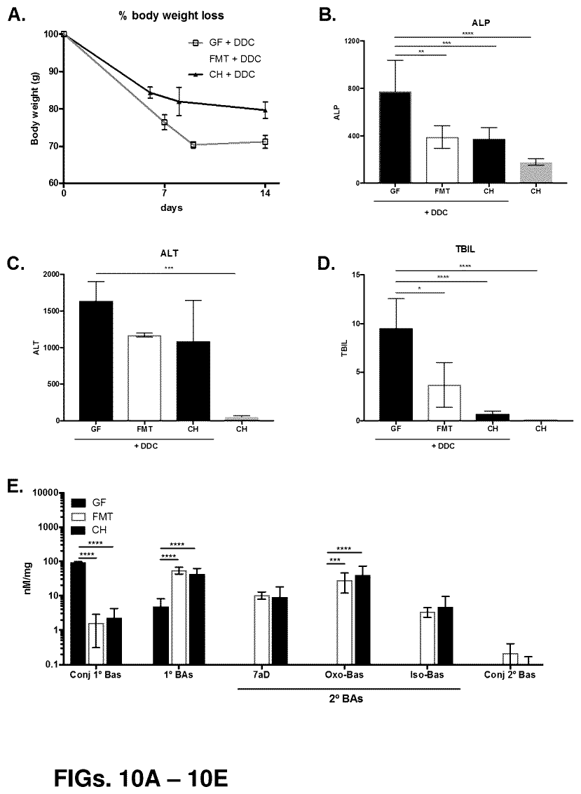

Mice treated with the DDC diet demonstrated a decrease in relative abundance of unconjugated and conjugated secondary bile acids in fecal, liver, and portal samples (FIG. 9 B-D, 2°, Conj. 2°), a trend similar to what we observed in PSC patients characterized above (FIG. 8). This decrease in microbial derived secondary bile acids again suggested an alteration to the gut microbiome with cholestatic disease. To determine the effect of liver disease on the gut microbiome, we used 16S NGS sequencing to profile the fecal microbial content of mice before (day 0) and after (day 21) the development cholestatic disease (DDC-treated. Mice with cholestatic liver disease showed a significant decrease in alpha diversity based on the Shannon diversity index, suggesting a loss in species richness with disease development (FIG. 9E). An assessment of beta-diversity also indicated a significant shift in the composition of the microbiome in mice with liver disease (FIG. 9E), with the microbiome of cholestatic mice forming a distinct cluster from those of healthy mice on a PCoA plot. FIG. 10F lists species with whose prevalence was significantly (p<0.2) altered with the development of liver disease in mice. In addition to the significant depletion of a number of species, specific strains known to possess 7α-dehydroxylation activity based on in vitro analysis, were also depleted in mice with cholestatic disease (FIG. 9G). 7α-dehydroxylation is required for the production of two major secondary bile acids—deoxycholic acid (DCA) and lithocholic acid (LCA). These same bile acids are also depleted in mice treated with the DDC-diet (FIG. 10B-10D). Changes in the bile acid composition of the DDC-mouse model thus provides a comparative model for studying cholestatic disease in humans.

### Absence of a Microbiome Increases Susceptibility to Cholestatic Disease

Previous studies have shown that in the mdr2−/− deletion model, the absence of a microbiome leads to more rapid development of cholestatic liver disease. We therefore first assessed the importance of the microbiome to disease development in the DDC-diet induced model of cholestatic disease. Germ-free Swiss albino mice were placed on a sterile DDC supplemented diet and monitored for disease development. Body weight loss, serum biochemistry and overall health were used to monitor progression of disease. In parallel, conventionally housed Swiss albino mice were also placed on the DDC diet. Germ-free mice showed rapid weight loss (FIG. 10A) and more severe alterations to liver biochemistry (FIG. 10 B-D) compared to conventional mice, suggesting that the microbiome plays a crucial role in resistance to liver disease.

Colonization with a Mouse Derived FMT Restores Bile Acid Metabolism and Resistance Cholestatic Disease in Germ-Free Mice

To further demonstrate the importance of the microbiome in resistance to cholestatic disease, we colonized germ-free Swiss albino mice with a mouse FMT derived from conventionally housed Swiss albino mice. The germ-free mice were allowed to colonize for 4 weeks followed by treatment with the DDC-supplemented diet to induce cholestatic liver disease. Mice treated with the FMT developed disease at a slower rate compared to germ-free mice and were comparable to conventional mice in their response to the DDC-diet (FIG. 10). Both body weight loss and serum ALP levels were lower in the FMT colonized mice compared to germ-free Swiss albino mice, suggesting that restoring a healthy microbiome is sufficient to extend resistance to DDC-induced cholestatic liver disease. Bile acid profiling also determined that FMT treatment was sufficient to restore bile acid composition in the gut compared to germ-free mice (FIG. 10E).

### Microbial Composition Determines Susceptibility to Cholestatic Disease

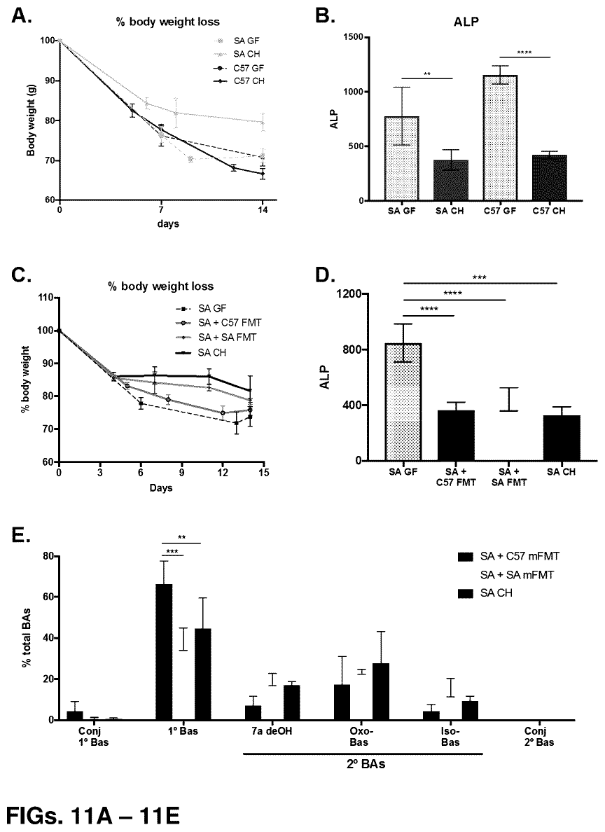

When conventional Swiss albino and C57Bl/6 background mice were placed on the DDC-supplemented diet, mice from the C57Bl/6 background were more susceptible to diet induced liver disease compared to Swiss albino mice (FIGS. 11A-11B). C57Bl/6 mice showed more rapid body weight loss compared to Swiss albino mice of the same age. Serum levels of ALP, however remained comparable. In the absence of a microbiome, however, germ-free Swiss albino mice were no longer resistant, and developed disease at a rate comparable to C57Bl/6 mice (FIGS. 11A-11B), suggesting that microbiome composition, rather than genetic background, might determine susceptibility to liver disease in mice. Germ-free Swiss albino and C57Bl/6 mice lost body weight at a similar rate and showed elevated ALP levels in the serum. The C57Bl/6 mice had slightly higher ALP levels, suggesting some further progression of disease in that genetic background.

To further compare the role of the microbiome between two genetically distinct strains, we determined the effect of a C57Bl/6 derived microbiome on the susceptibility of a Swiss albino mice to DDC-diet induced cholestatic disease. Germ-free Swiss albino mice were treated with either a C57Bl/6-derived or a Swiss albino-derived fecal microbiome transplant (FMT) and allowed to colonize for 4 weeks. Mice were then placed on a DDC-supplemented diet (0.1%) and monitored for development of cholestatic disease. Body weight loss and serum biochemistry were the primary markers of disease, while bile acid metabolism and liver histology were used as additional markers of microbiome functionality and disease development.

Swiss albino mice colonized with a C57Bl/6 derived FMT showed more rapid early weight loss compared to mice colonized with a Swiss albino derived FMT (FIG. 11C) despite comparable serum ALP levels (FIG. 11D). C57Bl/6 colonized mice looked more similar to germ-free mice in their response to the DDC diet, while Swiss albino colonized mice resembled conventional mice in their response, suggesting a link between their starting microbiomes and progression of disease. To further assess the potential role of the different microbiomes, we compared fecal bile acid profiles of mice colonized with the C57Bl/6-derived FMT to mice colonized with the Swiss albino derived FMT prior to DDC treatment. Mice treated with the C57Bl/6 sourced FMT had significantly higher levels of primary bile acids and lower levels of 7-alpha dehydroxylated and isomerized secondary bile acids compared to mice treated with a Swiss albino derived FMT at baseline (FIG. 11E). This demonstrates a significant difference in metabolic activity between the two microbiomes (C57Bl/6 vs. Swiss albino) and may account for the differences in susceptibility seen between the two mouse strains. The depletion of secondary bile acids in the more susceptible C57Bl/6 FMT colonized mice mimics the loss of secondary bile acids seen in PSC patients (FIG. 8) and in the mouse model of cholestasis (FIG. 9), again pointing to a role for microbial bile acid metabolism in progression of cholestatic liver disease.

Restoring Microbial Bile Acid Metabolism with a Designed Composition Rescues Susceptibility of Germ-Free Mice to Cholestatic Disease

The above results indicate a significant role for the microbiome in protection against DDC-diet induced liver disease. In particular, changes in bile acid metabolism with liver disease progression, and the increased susceptibility to liver disease in mice with decreased secondary bile acid levels, suggests a role for microbial bile acid metabolism and bile acids themselves in the response to cholestatic liver disease.

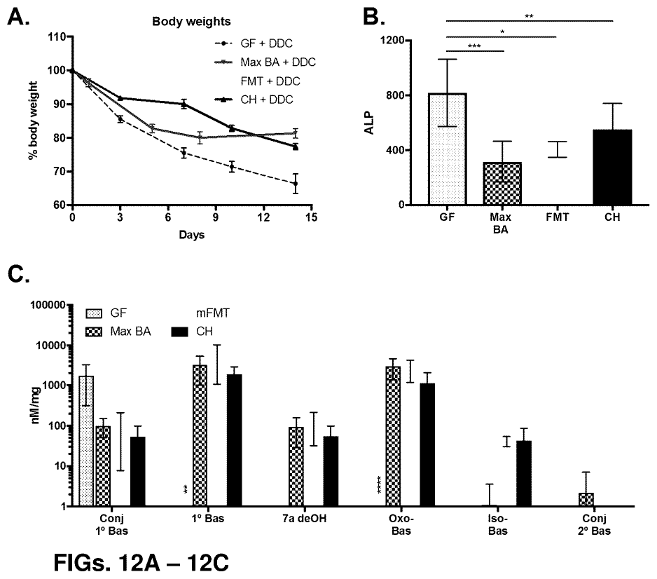

As shown in FIG. 3, bacterial compositions can be designed to specifically restore microbial bile acid metabolism, reconstituting the bile acid of the mouse gut. We tested the efficacy of one such composition, Max BA (FIGS. 2 and 3), in protecting germ-free mice from liver disease in the DDC-diet induced model cholestasis. The Max BA composition was designed to restore all major bile acid metabolic activities including BSH, 7α-dehydroxylation and HSDH (FIG. 1). Germ-free mice were treated with the Max BA composition and allowed to colonize for 4 weeks. Colonized mice were then exposed to a DDC supplemented diet and monitored for development of liver disease compared to germ-free and conventional controls exposed to a DDC diet. Mice treated with the Max BA composition showed a slower rate of body weight loss and maintained lower serum ALP levels compared to uncolonized germ-free mice on the DDC diet (FIGS. 12A-12B), suggesting a slower progression of disease in the presence of the Max BA composition. Colonization with the designed bacterial composition was as effective as FMT treatment, and comparable to conventional mice, in decreasing the rate of liver disease progression with DDC treatment.

In view of this this discovery, in some embodiments, the invention relates to a bacterial composition that can restore levels of primary and secondary bile acids when administered to patients diagnosed with cholestatic disease, e.g., treatment with such a composition can result in primary and secondary bile acids present at levels within a range observed in healthy humans.

Colonization with mFMT Rescues Early Lethality in Germ-Free Mdr2−/− C57Bl/6 Mice.

Tabiban et al. (Tabiban et al., Hepatol. 63:185-196, 2015) have previously shown that germ-free mdr2−/− mice in the FVB genetic background develop liver disease at a more rapid rate and with greater severity compared to conventional mdr2−/− mice. We chose to use C57Bl/6 mice with mdr2−/− deletion, a genetic background that has proved more sensitive to cholestatic disease in the DDC-diet induced cholestatic disease model (FIG. 11). Germ-free derivations of the mdr2−/− deletion mutant in the C57Bl/6 background resulted in early lethality, with surviving pups showing severe disease and lethality before they reached 4 weeks of age.

Given the demonstrated effectiveness of mouse-derived FMT in rescuing the susceptibility of germ-free mice in the DDC-diet induced cholestatic disease model (FIG. 10), we determined the effect of FMT treatment on survival of germ-free mdr2−/− mice in the C57Bl/6 genetic background. Foster moms were colonized with mouse FMT when pups were 2 weeks old, along with their bedding and cages. FMT colonization resulted in a 100% survival rate in treated mdr2−/− pups for greater than 12 weeks, while untreated pups that remained germ-free showed lethality within 4 weeks of age. The significant effect of colonization on survival of mdr2−/− germ-free mice further points to a role for the microbiome in ameliorating cholestatic liver disease. Testing compositions that lack bile acid activity (No BA) compared to ones reconstitute the majority of bile acid activity in the gut (Max BA) will provide insight into the specific role of bile acids in modulating the progression of cholestatic liver diseases.

### Bile Acid Supplementation is Sufficient to Increase Resistance of Germ-Free Mice to DDC-Diet Induced Liver Disease

We next explored the specific role of primary and secondary bile acid supplementation on progression of DDC-diet induced liver disease in the absence of a microbiome. Germ-free mice were fed diets supplemented with primary (CA+CDCA) or secondary (DCA+LCA) bile acids for 1 week and then exposed to DDC treatment. Bile acid supplementation was continued along with the DDC treatment to maintain signaling. Mice were monitored for rate of liver disease development based in body weight loss and serum biochemistry compared to germ-free and conventional controls on a DDC diet.

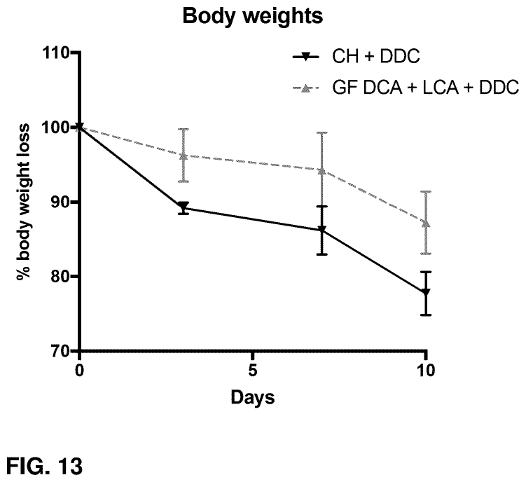

At 7 days post DDC exposure, preliminary body weight analysis showed mice fed a DCA+LCA supplemented diet have a significantly slower rate of body weight loss compared to conventional mice on the DDC diet (FIG. 13). Mice on the DCA+LCA supplemented diet show an average of 87% body weight loss compared to 77% in conventional mice on the DDC diet. This also constitutes a significant improvement over the rate of body weight loss in the more susceptible germ-free mice on DDC. This indicates a significant role for secondary bile acid signaling in modulating susceptibility to liver disease, even in the absence of a microbiome. A composition that mimics these effects by specifically restoring DCA and LCA levels in the gut could significantly decrease the progression of liver disease. Ongoing analyses will assess continuing effects of the DCA+LCA diet as well as a diet supplemented with the primary bile acids, cholic acid and chenodeoxycholic acid.

In view of this discovery, in some embodiments, the invention relates to a bacterial composition that can increase DCA and LCA levels and/or cholic acid if administered to patients diagnosed with cholestatic disease.

### Microbial Bile Acid Activity Contributes to Resistance Against DDC-Diet Induced Cholestatic Liver Disease

To determine if the ability to restore secondary bile acid metabolism is necessary for the beneficial effects of a microbiome on susceptibility to liver disease, mice are treated with a designed composition composed of bacterial strains that lack all microbial bile acid activity (No BA, FIG. 1). This composition is unable to modify bile acids in the gut, and colonized mice will retain the same bile acid composition as germ-free mice. In these experiments, mice are treated with either the No BA composition, the Max BA composition (that restores 2° bile acid metabolism), or with a mouse derived FMT and allowed to colonize for 4 weeks. Colonized mice are placed on a DDC-supplement diet and development of liver disease is monitored based on body weight loss and serum biochemistry. A more rapid progression of disease in No BA colonized mice compared to the MaxBA or FMT mice would suggest a role for the production of primary and secondary bile acids by the microbiome resistance to cholestatic liver disease.

### Example 5: Use of Gene Homology Searching Methods to Identify Strains Having Bile Acid Metabolism Activity

Two examples of gene homology searching methods that can be used to identify strains with the potential for bile acid metabolic activity based on a strain's whole genome sequence are described as follows. In one method, the protein-coding regions from a strain's whole genome sequence is pairwise compared to a database of known characterized proteins that catalyze the desired bile acid activity with BLASTp; strains containing a protein with sufficient similarity to any of the proteins in the database are identified as putatively possessing the desired bile acid activity. In a second method, the protein-coding regions from a strain's whole genome sequence are compared to a profile hidden Markov model (HMM) derived from a multiple sequence alignment of known characterized proteins that catalyze the desired bile acid activity; strains containing a protein with sufficient similarity to the profile HMM can be identified as putatively possessing the desired bile acid activity. The details of the sequence database, profile HMM, and appropriate similarity cutoffs differ across the bile acid activities being queried, which are described below.

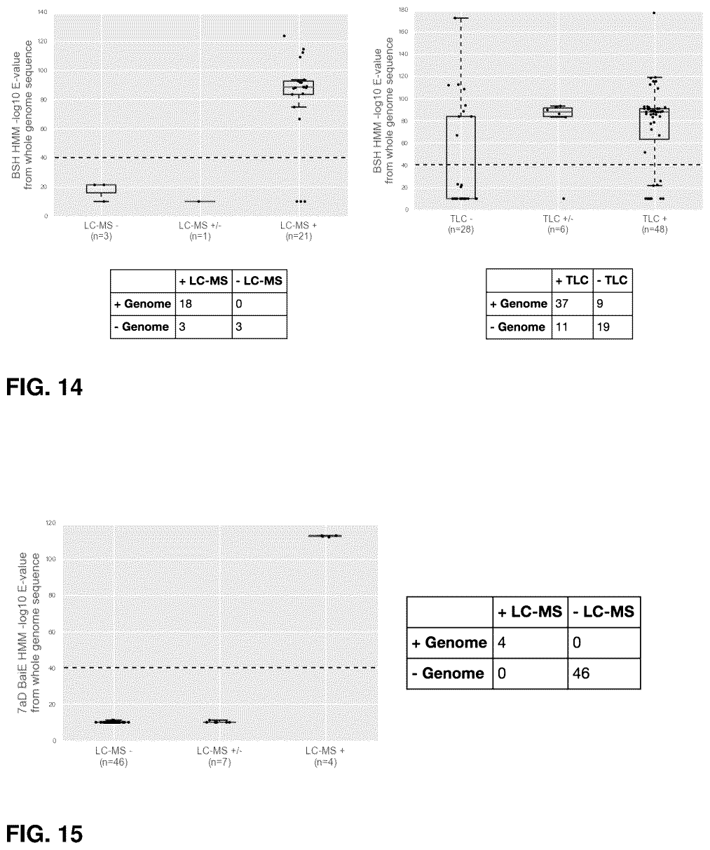

FIG. 14 shows the results of comparing bioinformatic prediction of BSH activity to in vitro assayed activity. All strains that have both been screened in vitro and have whole genome sequences available are considered. The left plot considers strains screened in vitro by LC-MS whereas the right plot considers strains that have been screened by TLC. Both plots show the e-value of the most significant alignment to the BSH HMM in that strain's genome. The significance of the genomic alignment significantly correlates with the results of the in vitro screen (Mann-Whitney U test; p=0.02 for LC-MS, p=0.0005 for TLC). A cutoff of e-value=1e-40 can be used to predict which strains will or will not have BSH activity (dotted line); the resulting confusion matrix for this classification cutoff is shown (for the LC-MS assay sensitivity=0.86, specificity=0.5, accuracy=0.88, p=0.01; for the TLC assay sensitivity=0.77, specificity=0.63, accuracy=0.74, p=0.0002). Note that due to the lower sensitivity of the TLC assay itself, the false positive rate of the genomic prediction may be overestimated. Furthermore, due to the low number of true negatives in the LC-MS assay, the specificity of the genomic predictions may be underestimated.

FIG. 15 shows the results of comparing bioinformatic prediction of 7α-dehydroxylation activity to in vitro assayed activity. All strains that have both been screened in vitro and have whole genome sequences available are considered. The box plot considers strains screened in vitro by LC-MS (none were screened by TLC). Both plots show the e-value of the most significant alignment to the BaiE HMM in that strain's genome. The significance of the genomic alignment significantly correlates with the results of the in vitro screen (Mann-Whitney U test; p<1e-8). A cutoff of e-value=1e-40 can be used to predict which strains will or will not have 7α-dehydroxylation activity (dotted line); the resulting confusion matrix for this classification cutoff is shown (sensitivity=1.0, specificity=1.0, accuracy=1.0, p<1e-5). Note that due to the low number of true positives, the true sensitivity, specificity and accuracy may be likely overestimated.

Other embodiments are within the scope of the following claims.

1. A formulation comprising (i) a plurality of viable bacteria, which comprises at least one bacterial OTU or species that is capable of exhibiting a bile acid or bile salt metabolizing activity, and (ii) a pharmaceutically acceptable excipient.

2. The formulation of claim 1, wherein the at least one bacterial OTU or species comprises a 16S rDNA sequence that is at least 95% identical to a sequence in FIG. 16.

3. The formulation of claim 1, wherein the bile acid or bile salt metabolizing activity is selected from the group consisting of bile acid or bile salt hydrolase activity, deconjugation, oxidation, dehydroxylation, and combinations thereof.

4. The formulation of claim 1, wherein the plurality of viable bacteria comprises at least two different bacterial OTUs or species.

5. The formulation of claim 1, wherein the at least one bacterial OTU or species is capable of exhibiting an oxidation activity, a dihydroxylation activity, or both.

6. The formulation of claim 1, wherein the plurality of viable bacteria comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 bacterial OTUs or species selected from at least two clades of Table 1.

7. The formulation of claim 6, wherein each of the bacterial OTUs or species of the plurality of viable bacteria comprises a 16S rDNA sequence that has at least 95% sequence identity to at least one sequence of FIG. 16, or a portion thereof.

8. The formulation of claim 1, wherein the plurality of viable bacteria comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, or 50 bacterial OTUs or species selected from at least two clades of Table 2.

9. The formulation of claim 1, wherein the plurality of viable bacteria comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, or 50 OTUs or bacterial species of part B or C of Table 1, Table 2, or Table 3.

10. The formulation of claim 1, wherein the plurality of bacteria comprises one or more bacterial OTUs or species from 5, 10, 15, or 20 clades selected from the group consisting of: 1, 6, 86, 87, 90, 100, 101, 164, 195, 196, 197, 203, 204, and 297.

11.-15. (canceled)

16. A method of treating a cholestatic disease or condition in a subject in need thereof, comprising administering to the subject a composition, which comprises at least one bacterial OTU or species that is capable of (i) deconjugating a primary bile acid or bile salt, (ii) metabolizing a primary bile acid or salt into a secondary bile acid, or (iii) both.

17.-18. (canceled)

19. A method of treating a cholestatic disease or condition in a subject in need thereof, comprising administering to the subject the formulation of claim 1.

20.-30. (canceled)

31. A method for identifying a bacterial species for use in a composition for altering bile acid metabolism in a subject, the method comprising comparing protein coding sequences of a bacterial strain to reference sequences in a database of proteins that catalyze a desired bile acid activity, wherein identification of a bacterial strain comprising a sequence with homology to a reference sequence indicates the identification of a bacterial strain for use in said composition.

32.-33. (canceled)

34. The formulation of claim 1, wherein the at least one bacterial OTU or species is selected from Alistipes finegoldii, Bacteroides uniformis, Bacteroides vulgatus, Bacteroides sp_3_1_19, Bacteroides intestinalis, Bacteroides sp_20_3, Bacteroides caccae, Bacteroides faecis, Bacteroides sp_3_1_23, Bacteroides sp_D20, Bacteroides eggerthii, Barnesiella intestinihominis, Bifidobacterium pseudocatenulatum, Blautia schinkii, Blautia wexlerae, Blautia sp_M25, Blautia producta, Clostridiales sp_SM4_1, Clostridium lavalense, Clostridium innocuum, Clostridium asparagiforme, Clostridium spiroforme, Clostridium hylemonae, Clostridium sordellii, Clostridium sp_YIT_12069, Clostridium paraputrificum, Clostridium glycolicum, Clostridium ghonii, Clostridium lactifermentans, Clostridium viride, Clostridium sp_HGF2, Clostridium symbiosum, Clostridium scindens, Clostridium disporicum, Clostridium butyricum, Clostridium orbiscindens, Clostridium bolteae, Clostridium sp_7_2_43FAA, Collinsella aerofaciens, Coprobacillus sp_D7, Coprococcus comes, Coprococcus eutactus, Dorea longicatena, Dorea formicigenerans, Erysipelotrichaceae bacterium_3_1_53, Eubacterium sp3_1_31, Eubacterium sp_WAL_14571, Eubacterium limosum, Eubacterium hallii, Eubacterium rectale, Eubacterium contortum, Gemmiger formicillis, Lachnospiraceae bacterium_2_1_58FAA, Lachnospiraceae bacterium_9_1_43BFAA, Lachnospiraceae bacterium_3_1_57FAA_CT1, Lachnospiraceae bacterium_5_1_57FAA, Lachnospiraceae bacterium_oral_taxon_F15, Lactobacillus acidophilus, Lactobacillus gasseri, Parabacteroides distasonis, Pseudoflavonifractor capillosus, Roseburia faecis, Ruminococcus obeum, Ruminococcus bromii, Ruminococcus lactaris, or Tannerella sp_6_1_58FAA.

35. A method of reducing a concentration of a primary bile acid or salt in a subject in need thereof, comprising administering to the subject the formulation of claim 1.

36. The method of claim 35, wherein the at least one bacterial OTU or species is selected from Alistipes finegoldii, Bacteroides uniformis, Bacteroides vulgatus, Bacteroides sp_3_1_19, Bacteroides intestinalis, Bacteroides sp203, Bacteroides caccae, Bacteroides faecis, Bacteroides sp_3_1_23, Bacteroides sp_D20, Bacteroides eggerthii, Barnesiella intestinihominis, Bifidobacterium pseudocatenulatum, Blautia schinkii, Blautia wexlerae, Blautia sp_M25, Blautia producta, Clostridiales sp_SM4_1, Clostridium lavalense, Clostridium innocuum, Clostridium asparagiforme, Clostridium spiroforme, Clostridium hylemonae, Clostridium sordellii, Clostridium sp_YIT_12069, Clostridium paraputrificum, Clostridium glycolicum, Clostridium ghonii, Clostridium lactifermentans, Clostridium viride, Clostridium sp_HGF2, Clostridium symbiosum, Clostridium scindens, Clostridium disporicum, Clostridium butyricum, Clostridium orbiscindens, Clostridium bolteae, Clostridium sp_7_2_43FAA, Collinsella aerofaciens, Coprobacillus sp_D7, Coprococcus comes, Coprococcus eutactus, Dorea longicatena, Dorea formicigenerans, Erysipelotrichaceae bacterium_3_1_53, Eubacterium sp_3_1_31, Eubacterium sp_WAL_14571, Eubacterium limosum, Eubacterium hallii, Eubacterium rectale, Eubacterium contortum, Gemmiger formicillis, Lachnospiraceae bacterium_2_1_58FAA, Lachnospiraceae bacterium_9_1_43BFAA, Lachnospiraceae bacterium_3_1_57FAA_CT1, Lachnospiraceae bacterium_5_1_57FAA, Lachnospiraceae bacterium_oral_taxon_F15, Lactobacillus acidophilus, Lactobacillus gasseri, Parabacteroides distasonis, Pseudoflavonifractor capillosus, Roseburia faecis, Ruminococcus obeum, Ruminococcus bromii, Ruminococcus lactaris, or Tannerella sp_6_1_58FAA.

37. A method of increasing a concentration of a secondary bile acid or salt in a subject in need thereof, comprising administering to the subject the formulation of claim 1.

38. The method of claim 37, wherein the at least one bacterial OTU or species is selected from Alistipes finegoldii, Bacteroides uniformis, Bacteroides vulgatus, Bacteroides sp_3_1_19, Bacteroides intestinalis, Bacteroides sp_20_3, Bacteroides caccae, Bacteroides faecis, Bacteroides sp_3_1_23, Bacteroides sp_D20, Bacteroides eggerthii, Barnesiella intestinihominis, Bifidobacterium pseudocatenulatum, Blautia schinkii, Blautia wexlerae, Blautia sp_M25, Blautia producta, Clostridiales sp_SM4_1, Clostridium lavalense, Clostridium innocuum, Clostridium asparagiforme, Clostridium spiroforme, Clostridium hylemonae, Clostridium sordellii, Clostridium sp_YIT_12069, Clostridium paraputrificum, Clostridium glycolicum, Clostridium ghonii, Clostridium lactifermentans, Clostridium viride, Clostridium sp_HGF2, Clostridium symbiosum, Clostridium scindens, Clostridium disporicum, Clostridium butyricum, Clostridium orbiscindens, Clostridium bolteae, Clostridium sp_7_2_43FAA, Collinsella aerofaciens, Coprobacillus sp_D7, Coprococcus comes, Coprococcus eutactus, Dorea longicatena, Dorea formicigenerans, Erysipelotrichaceae bacterium_3_1_53, Eubacterium sp3_1_31, Eubacterium sp_WAL_14571, Eubacterium limosum, Eubacterium hallii, Eubacterium rectale, Eubacterium contortum, Gemmiger formicillis, Lachnospiraceae bacterium_2_1_58FAA, Lachnospiraceae bacterium_9_1_43BFAA, Lachnospiraceae bacterium_3_1_57FAA_CT1, Lachnospiraceae bacterium_5_1_57FAA, Lachnospiraceae bacterium_oral_taxon_F15, Lactobacillus acidophilus, Lactobacillus gasseri, Parabacteroides distasonis, Pseudoflavonifractor capillosus, Roseburia faecis, Ruminococcus obeum, Ruminococcus bromii, Ruminococcus lactaris, or Tannerella sp6_1_58FAA.

39. A method of decreasing a dose and/or a frequency of a treatment for a cholestatic disease or condition required to treat the cholestatic disease or condition, comprising administering to the subject the formulation of claim 1 and the treatment for the cholestatic disease or condition.

40. The method of claim 39, wherein the at least one bacterial OTU or species is selected from Alistipes finegoldii, Bacteroides uniformis, Bacteroides vulgatus, Bacteroides sp_3_1_19, Bacteroides intestinalis, Bacteroides sp_20_3, Bacteroides caccae, Bacteroides faecis, Bacteroides sp_3_1_23, Bacteroides sp_D20, Bacteroides eggerthii, Barnesiella intestinihominis, Bifidobacterium pseudocatenulatum, Blautia schinkii, Blautia wexlerae, Blautia sp_M25, Blautia producta, Clostridiales sp_SM4_1, Clostridium lavalense, Clostridium innocuum, Clostridium asparagiforme, Clostridium spiroforme, Clostridium hylemonae, Clostridium sordellii, Clostridium sp_YIT_12069, Clostridium paraputrificum, Clostridium glycolicum, Clostridium ghonii, Clostridium lactifermentans, Clostridium viride, Clostridium sp_HGF2, Clostridium symbiosum, Clostridium scindens, Clostridium disporicum, Clostridium butyricum, Clostridium orbiscindens, Clostridium bolteae, Clostridium sp_7_2_43FAA, Collinsella aerofaciens, Coprobacillus sp_D7, Coprococcus comes, Coprococcus eutactus, Dorea longicatena, Dorea formicigenerans, Erysipelotrichaceae bacterium_3_1_53, Eubacterium sp3_1_31, Eubacterium sp_WAL_14571, Eubacterium limosum, Eubacterium hallii, Eubacterium rectale, Eubacterium contortum, Gemmiger formicillis, Lachnospiraceae bacterium_2_1_58FAA, Lachnospiraceae bacterium 9_1_43BFAA, Lachnospiraceae bacterium_3_1_57FAA_CT1, Lachnospiraceae bacterium_5_1_57FAA, Lachnospiraceae bacterium_oral_taxon_F15, Lactobacillus acidophilus, Lactobacillus gasseri, Parabacteroides distasonis, Pseudoflavonifractor capillosus, Roseburia faecis, Ruminococcus obeum, Ruminococcus bromii, Ruminococcus lactaris, or Tannerella sp6_1_58FAA.
